(19) United States
(12) Patent Application Publication
(10) Pub. No.: US 2022/0241001 A1
(43) Pub. Date: Aug. 4, 2022

(54) LUNG TUMOR ABLATION METHOD

(71) Applicant: Broncus Medical Inc., San Jose, CA (US)

(72) Inventors: Liming WANG, Hangzhou (CN); Hong XU, Hangzhou (CN); Huazhen ZHOU, Hangzhou (CN); Changjie CUI, Hangzhou (CN); Zhiyu LIU, Hangzhou (CN); Thomas M. Keast, Sunnyvale, CA (US)

(73) Assignee: Broncus Medical Inc., San Jose, CA (US)

(21) Appl. No.: 17/658,941

(22) Filed: Apr. 12, 2022

Related U.S. Application Data
(63) Continuation-in-part of application No. PCT/CN2021/076118, filed on Feb. 8, 2021.
Application No. PCT/CN2021/076118 is a continuation-in-part of application No. PCT/CN2021/072952, filed on Jan. 20, 2021.
Application No. PCT/CN2021/072952 is a continuation-in-part of application No. PCT/CN2021/072954, filed on Jan. 20, 2021.
Application No. PCT/CN2021/072954 is a continuation-in-part of application No. PCT/CN2021/072955, filed on Jan. 20, 2021.
Application No. PCT/CN2021/072955 is a continuation-in-part of application No. PCT/CN2021/072956, filed on Jan. 20, 2021.
Application No. PCT/CN2021/072956 is a continuation-in-part of application No. PCT/CN2021/072957, filed on Jan. 20, 2021.
Application No. PCT/CN2021/072957 is a continuation-in-part of application No. PCT/CN2021/072959, filed on Jan. 20, 2021.
Application No. PCT/CN2021/072959 is a continuation-in-part of application No. PCT/CN2020/140380, filed on Dec. 28, 2020.
Application No. PCT/CN2020/140380 is a continuation-in-part of application No. PCT/CN2020/118645, filed on Sep. 29, 2020.
Application No. PCT/CN2020/118645 is a continuation-in-part of application No. PCT/CN2020/118646, filed on Sep. 29, 2020.

(30) Foreign Application Priority Data
Dec. 31, 2019 (CN) 201911403420.3
Dec. 31, 2019 (CN) 201911403441.5
Dec. 9, 2020 (CN) 202011433510.X
Dec. 31, 2020 (CN) 202011637770.9
Dec. 31, 2020 (CN) 202011637865.0
Dec. 31, 2020 (CN) 202011638301.9
Dec. 31, 2020 (CN) 202011638331.X
Dec. 31, 2020 (CN) 202011640959.3
Dec. 31, 2020 (CN) 202011641170.X
Dec. 31, 2020 (CN) 202011641885.5
Dec. 31, 2020 (CN) 202011642360.3

(51) Int. Cl. A61B 18/12

(52) U.S. Cl. CPC A61B 18/12; A61B 2018/00541; A61B 2018/126; A61B 2218/002

(57) ABSTRACT

A lung tumor ablation method includes deploying a plurality of sensors in the tumor margin and monitoring ablation progress based on information from the sensors. An ablation electrode is inserted into a central region of the tumor and the site is irrigated. Power and irrigation flowrate are controlled to optimize ablation based on information from the sensors. Related apparatus and systems are described.

### BACKGROUND OF THE INVENTION

### 1. Field of the Invention

This invention relates to electrosurgery, and particularly, to radio frequency-based ablation systems for ablating lung tumors.

### 2. Description of the Related Art

Radio-frequency ablation technology is widely used in lung surgery. Radio frequency is not a band divided in wireless communications, and exert mainly a thermal effect in organisms. When a radio-frequency current frequency reaches a value (>100 kHz), charged ions in a tissue is caused to move and heat is generated from friction (60° C. to 100° C.). A frequency commonly used by a radio-frequency ablation apparatus is 200 to 500 kHz, and the output frequency is 100 to 400 kHz. An ablation electrode is a core component in a radio-frequency ablation system, because it directly affects the size and shape of coagulation necrosis. A coagulation area desirably has a spherical or spheroidic shape. A multi-needle electrode is guided by B ultrasound or CT to directly pierce into a pathological tissue mass of human, and the radio-frequency electrode needle causes the temperature in the tissue to rise to 60° C. or higher, leading to the death of cells and the formation of a necrotic area. For example, the local temperature in the tissue exceeds 100° C., causing coagulation necrosis of the tumor tissue and parenchyma surrounding organs. During treatment, a large spherical area of coagulation necrosis can be formed, and there is also a hyperthermia area of 43-60° C. outside the area of coagulation necrosis. In this area, cancer cells are killed, and normal cells can be restored.

During the treatment process, a radio-frequency electrode is inserted into a human tissue, a current is introduced to the lesion by the radio-frequency electrode, and a lot of heat is generated by the radio-frequency electrode. For example, when the temperature of the lesion reaches 40° C.-60° C. and is maintained for a period of time, the ablation operation of the lesion is completed. However, since the radio-frequency ablation system in the prior art cannot determine the working status information of the radio-frequency electrode, for example, the temperature near the radio-frequency electrode, the progress of the ablation operation can only be empirically determined and then an adjustment is made by a physician during surgery, which increases the difficulty and accuracy of the operation. Therefore, there is an urgent need in the art to provide a radio-frequency ablation system able to accurately determine whether the ablation is completed.

### SUMMARY OF THE INVENTION

Detection Mechanism, Radio-Frequency Ablation Catheter, and Radio-Frequency Ablation System

An objective of the present invention is to provide a detection mechanism, a radio-frequency ablation catheter, and a radio-frequency ablation system. During the radio-frequency ablation process, the stretching and retraction of a claw electrode can be controlled by pushing a sliding button on a handle to move back and forth. This realizes the auxiliary positioning of an electrode body, the determination of the situation around the electrode body and also by the claw electrode.

An embodiment of the present invention provides a detection mechanism, for use in a radio-frequency ablation catheter. The detection mechanism comprises a fixing base, a traction wire, a connecting base and multiple claw-shaped electrodes.

The fixing base is slidably mounted in the radio-frequency ablation catheter; and provided with multiple mounting holes, wherein each of the claw-shaped electrodes extends through the mounting hole and is fixedly connected to the fixing base.

A distal end of the traction wire is fixed on the fixing base, and a proximal end of the traction wire is fixed on a sliding button of the radio-frequency ablation catheter.

The connecting base is fixedly mounted in the radio-frequency ablation catheter; and provided with multiple guide holes, wherein the openings of adjacent guide holes extend in the same direction, each of the claw-shaped electrodes extends through the guide holes, and the guide hole allows the claw-shaped electrode to extend out of the connecting base in a dispersed manner.

Each of the claw-shaped electrodes includes: a first section, a second section, and a third section, wherein the second section is at an obtuse angle with respect to the first section, and the third section is at an obtuse angle with respect to the second section. When the claw-shaped electrode extends out of the connecting base in a dispersed manner, the first section, the second section, and the third section are opened. Distal ends of the claw-shaped electrodes are located at the same latitude.

In a feasible implementation, the detection mechanism also includes a fixing ring, having a distal end fixedly arranged on the claw electrode, and allowing a wire and the claw-shaped electrode to be electrically connected.

In a feasible implementation, the distance between adjacent guide holes of the detection mechanism is the same.

In a feasible implementation, the distance between opposing guide holes of the detection mechanism is different.

An embodiment of the present invention provides a radio-frequency ablation catheter, which includes a handle portion, a needle tube portion, a central electrode, and any detection mechanism as described above.

The needle tube portion includes a first tube sleeve and a second tube sleeve, wherein the connecting base is specifically mounted between the first tube sleeve and the second tube sleeve, the fixing base is specifically slidably mounted in the first tube sleeve, and the fixing ring and the multiple claw-shaped electrodes are located inside the first tube sleeve.

The handle portion comprises a cylinder sleeve and a sliding button, wherein the sliding button is slidably mounted on the cylinder sleeve; and a proximal end of the traction wire is fixed on the sliding button.

The central electrode comprises an electrode body, an electrode wire, and an electrode connector, wherein the electrode body is provided at a distal end of the second tube sleeve, a distal end of the electrode wire is electrically connected to the electrode body, the electrode wire extends through the cylinder sleeve, the first tube sleeve and the second tube sleeve, a proximal end of the electrode wire is electrically connected to the electrode connector, and the electrode connector is located outside the cylinder sleeve.

In a feasible implementation, the radio-frequency ablation catheter also includes: a liquid injection joint and a liquid injection tube, wherein the liquid injection tube extends through the cylinder sleeve, the fixing base and the connecting base, the electrode body is provided with a liquid inlet, and a distal end of the liquid injection tube is in communication with the liquid inlet; and the liquid injection joint is provided at a proximal end of the liquid injection tube, and located outside the cylinder sleeve.

In a feasible implementation, sprinkler channels are provided in the electrode body of the radio-frequency ablation catheter, wherein the sprinkler channels are in communication with the liquid inlet, and the sprinkler channels are used to dispersedly feed the liquid at the liquid inlet.

In a feasible implementation, the electrode body of the radio-frequency ablation catheter is externally provided with an infiltration hood, wherein the infiltration hood includes a plurality of dispersing holes distributed in a rectangular array, and the hole diameters of each row of dispersing holes decrease in a direction from the proximal end to the distal end.

In a feasible implementation, the dispersing hole and the sprinkler channel of the radio-frequency ablation catheter are arranged misaligned.

In a feasible implementation, the electrode body of the radio-frequency ablation catheter includes a cylindrical portion and a tapered portion, wherein the tapered portion is located at a distal end of the cylindrical portion; and the infiltration hood is mounted around the cylindrical portion.

In a feasible implementation, the electrode body of the radio-frequency ablation catheter is provided with a temperature sensor and a signal conduit therein, and a wiring hole is provided on the electrode body, wherein the temperature sensor is located in the tapered portion, the signal conduit extends through the wiring hole, an insulating layer is provided outside the signal conduit, a distal end of the signal conduit is electrically connected to the temperature sensor, and a proximal end of the signal conduit is electrically connected to the electrode connector.

In a feasible implementation, a slot is provided on the electrode body of the radio-frequency ablation catheter, and the distal end of the second tube sleeve is inserted into the slot.

In a feasible implementation, the infiltration hood of the radio-frequency ablation catheter is made of a high-temperature resistant insulating material.

In a feasible implementation, the electrode body of the radio-frequency ablation catheter is made of fibers.

In a feasible implementation, the second tube sleeve of the radio-frequency ablation catheter is provided with an internal thread, the connecting base is provided with an external thread, and the second tube sleeve and the connecting base are threadedly connected.

In a feasible implementation, the first tube sleeve of the radio-frequency ablation catheter is provided with a counterbore, a threaded hole is provided at the proximal end of the connecting base, and the first tube sleeve and the connecting base are connected by a bolt.

In a feasible implementation, the connecting base includes: a liquid injection hole, wherein the liquid injection hole is positioned along the center line of the connecting base, the liquid injection tube extends through the liquid injection hole, and the guide holes are symmetrically distributed with the liquid injection hole as a center. The guide hole includes a straight section and an arc section, wherein the arc section is located at a distal end of the straight section.

In a feasible implementation, an anti-skid pattern is provided on an inner side wall of the straight section of the guide hole of the radio-frequency ablation catheter.

In a feasible implementation, an anti-wear cushion is provided on an inner side wall of each arc section of the radio-frequency ablation catheter, wherein the anti-wear cushion is made of a rubber material.

In a feasible implementation, the connecting base of the radio-frequency ablation catheter is made of glass fibers.

In a feasible implementation, a mounting post is provided at the proximal end of the connecting base of the radio-frequency ablation catheter, and a spring is mounted around the mounting post, wherein when the fixing base moves toward the connecting base, a distal end of the spring presses against the connecting base, and a proximal end of the spring presses against the fixing base.

In a feasible implementation, the multiple claw-shaped electrodes of the radio-frequency ablation catheter are hollow tubes.

In a feasible implementation, an insulating layer is provided around an outer surface of the first sections of the claw-shaped electrodes of the radio-frequency ablation catheter.

In a feasible implementation, an insulating layer is provided around an outer surface of the second sections of the claw-shaped electrodes of the radio-frequency ablation catheter.

In a feasible implementation, the first section and the second section of the radio-frequency ablation catheter are both straight sections, and the third section is an arc section.

In a feasible implementation, a first groove is respectively provided on both sides of the fixing base of the radio-frequency ablation catheter, a fixture block is provided at the distal end of the traction wire, and the fixture block is inserted in the first groove, to allow the traction wire and the fixing base to be fixedly connected.

In a feasible implementation, the fixing base of the radio-frequency ablation catheter is made of an electric ceramic material.

In a feasible implementation, the fixing ring and the fixing base of the radio-frequency ablation catheter are joined by interference fit to limit the extending length of the claw-shaped electrode.

In a feasible implementation, the fixing base of the radio-frequency ablation catheter is made of a silicone rubber material.

In a feasible implementation, the fixing ring of the radio-frequency ablation catheter is a hollow cylinder, and the fixing ring is provided with a slit.

In a feasible implementation, both ends of the fixing ring of the radio-frequency ablation catheter are provided with a buffering cushion.

In a feasible implementation, the buffering cushion of the radio-frequency ablation catheter is made of an elastic rubber material.

In a feasible implementation, the fixing base of the radio-frequency ablation catheter includes a female fixing base and a male fixing ring joined to each other.

In a feasible implementation, the female fixing ring and the male fixing ring of the radio-frequency ablation catheter are hinged by a hinge shaft.

An embodiment of the present invention also provides a radio-frequency ablation system, which includes the radio-frequency ablation catheter described in any of the foregoing feasible implementations.

Based on the above implementations, it can be known that the present invention provides a detection mechanism, a radio-frequency ablation catheter, and a radio-frequency ablation system. The radio-frequency ablation catheter includes a handle portion, a needle tube portion, a central electrode, and a detection mechanism. The needle tube portion includes a first tube sleeve and a second tube sleeve, the handle portion includes a cylinder sleeve and a sliding button, the central electrode includes an electrode body, an electrode wire, and an electrode connector, and the detection mechanism includes a fixing ring, a fixing base, a traction wire, a connecting base, and multiple claw-shaped electrodes. For the detection mechanism, the radio-frequency ablation catheter, and the radio-frequency ablation system of the present invention, with a user as a reference, the end close to the user is the proximal end and the end away from the user is the distal end. The connecting base is mounted between the first tube sleeve 11 and the second tube sleeve, and the fixing base is located at a proximal end of the connecting base. The fixing base, the fixing ring and the multiple claw-shaped electrodes are all provided in the first tube sleeve. The fixing base can slidably move in the first tube sleeve, the fixing ring is mounted at a proximal end of the fixing base and fixed to the claw-shaped electrode, and the claw-shaped electrode extends in the fixing base. The claw-shaped electrodes can slide in the connecting base, and when the claw-shaped electrodes extend out of the connecting base, the claw-shaped electrodes stretch out, and are on the same latitude. The sliding button can slide on the surface of the cylinder sleeve, a proximal end of the traction wire is fixed on the sliding button; and a distal end of the traction wire is fixed to the fixing base. The electrode body is located at a distal end of the second tube sleeve, a distal end of the electrode wire is fixed in the electrode body and electrically connected, a proximal end of the electrode wire is electrically connected to the electrode connector, and the electrode connector is located outside the cylinder sleeve. The multiple claw-shaped electrodes can be pushed in and pushed out of the needle tube portion by the aid of the connecting base, the fixing base, and the traction wire. When the claw-shaped electrode needs to be pushed out of the needle tube portion for detection, the sliding button is pushed distally, so that the sliding button drives the traction wire to move distally, which in turn drives the fixing base to move distally. At this time, the claw-shaped electrodes fixed to the fixing base move distally, as the fixing base moves distally. As a result, the claw-shaped electrodes previously received in the connecting base are pushed out of the needle tube portion. When there is no need to use the claw-shaped electrodes for detection, the sliding button is pushed proximally, so the sliding button drives the traction wire to move proximally, which in turn drives the fixing base to move proximally, so as to pull the claw-shaped electrodes back. At this time, the claw-shaped electrodes previously located outside the needle tube portion are retracted inside the connecting base again. By means of the sliding button, the traction wire and the fixing base 5 are driven to move to finally realize the push-out and retraction of the claw-shaped electrodes 7. This facilitates the control of the claw-shaped electrodes by a user during surgery.

During the radio-frequency ablation process, the stretching and retraction of the claw electrodes can be controlled by pushing the sliding button on the handle to move backward and forward. This realizes the auxiliary positioning of an electrode body, the determination of the situation around the electrode body by the claw electrode, and thus the determination of the progress of ablation.

Rotatable Catheter

The embodiments of the present invention provide a radiofrequency ablation catheter, including: a handle which has a proximal end and a distal end, an outer catheter assembly which has a proximal end and a distal end, and an inner catheter assembly which has a proximal end and a distal end; wherein the inner catheter assembly comprises a peripheral electrode assembly, the peripheral electrode assembly has a proximal end and a distal end, the distal end of the peripheral electrode assembly can protrude from the distal end of the outer catheter assembly, and the peripheral electrode assembly comprises a plurality of peripheral electrodes circumferentially spaced from each other; wherein the proximal end of the outer catheter assembly is connected with the distal end of the handle; the proximal end of the inner catheter assembly is connected with the distal end of the handle; the inner catheter assembly is rotatable relative to the outer catheter assembly by driving the handle. The inner catheter assembly including the peripheral electrode assembly is configured to be driven by the handle to rotate relative to the outer catheter assembly, so that when the peripheral electrode assembly protrudes from the distal end of the outer catheter assembly, blood vessels can pass through the gap between the adjacent peripheral electrodes of the peripheral electrode assembly, thereby avoiding puncturing the blood vessels, reducing the risk of operation failure or bringing hidden treatment hazards or complications to the patient, and solving the problem that the peripheral electrode assembly in the prior art is easy to pierce the blood vessels when it enters the lesion site, causing the operation to fail or bringing the hidden treatment hazards or complications to the patient.

In some embodiments, the inner catheter assembly further comprises a support element for supporting the peripheral electrode assembly, wherein the plurality of peripheral electrodes of the peripheral electrode assembly are spaced from each other in a circumferential direction of the support element, and the support element is rotatably connected with the outer catheter assembly. The support element rotatably connected with the outer catheter assembly supports the peripheral electrode assembly, which helps to rotate the peripheral electrode assembly stably.

In some embodiments, the support element has a proximal end and a distal end, and a proximal side of the support is opposite to a distal side of the outer catheter assembly; the inner catheter assembly further comprises a rotatable element for connecting the outer catheter assembly and the support element; the rotatable element has a proximal end and a distal end, wherein the distal end of the rotatable element is fixedly connected with the support element, and the proximal end of the rotatable element is rotatably connected with the outer catheter assembly. The rotatable element rotatably connected with the outer catheter assembly ensures that the peripheral electrode assembly can rotate relative to the outer catheter assembly. At the same time, the distal end of the outer catheter assembly can also limit the proximal end of the support element to a certain extent to prevent the support element from moving toward the proximal end in the axial direction when rotating together with the peripheral electrode assembly. In other embodiments, other structures can also be used. For example, the support element and the rotatable element may also be formed as a single piece.

In some embodiments, an outer wall of the support element has a plurality of grooves circumferentially spaced from each other, and a length direction of the groove corresponds to a length direction of the support element; the rotatable element covers a section of the plurality of grooves along the length direction thereof, and the peripheral electrodes are slidably received in respective grooves, and can be received in the rotatable element or slide out from the rotatable element. The groove helps the peripheral electrodes of the peripheral electrode assembly to move stably in the axial direction relative to the support element. Furthermore, the rotatable element covers a section of the plurality of grooves along the length direction thereof, in other words, the other section of the plurality of grooves along the length direction thereof is visible. Therefore, the rotation direction of the peripheral electrode assembly can be judged according to the visible section of the grooves, so that the blood vessel can pass through the gap between adjacent grooves, so that the peripheral electrodes of the peripheral electrode assembly in the grooves avoid blood vessels too. In other embodiments, the rotation direction of the peripheral electrode assembly can also be determined in other ways.

In some embodiments, the rotatable element is tubular, an inner wall of the rotatable element is provided with a first protrusion, and the distal end of the outer catheter assembly is provided with a second protrusion, and the first protrusion is rotatably engaged with the second protrusion. Through the engagement of the first protrusion and the second protrusion, not only the rotatable element can rotate relative to the outer catheter assembly, but the rotatable element can also be prevented from moving towards the distal end along the axial direction when rotating together with the peripheral electrode assembly.

In some embodiments, the outer catheter assembly comprises an outer sheath having a proximal end and a distal end, and a connecting tube fixedly connected with the distal end of the outer sheath, and wherein the proximal end of the outer sheath is connected with the distal end of the handle, the connecting tube has a proximal end and a distal end, and the second protrusion is provided on a distal side of the connecting tube. The handle is connected with the outer sheath, and the connecting tube is connected with the rotatable element, which facilitates the assembly of the radiofrequency ablation catheter. In other embodiments, the outer catheter assembly can also use other structures. For example, the outer catheter assembly can be formed in one piece.

In some embodiments, the proximal end of the connecting tube is received in the distal end of the outer sheath, and a distal side of the outer sheath abuts against a proximal side of the rotatable member. The outer sheath abuts against the rotatable element, so that the rotatable element can be effectively prevented from moving toward the proximal end in the axial direction when rotating together with the peripheral electrode assembly.

In some embodiments, the handle comprises a housing having a proximal end and a distal end, and an end cap rotatably connected with the distal end of the housing, and wherein the proximal end of the outer catheter assembly is connected with the end cap, and the inner catheter assembly is rotatable relative to the outer catheter assembly by a rotation of the housing relative to the end cap. The inner catheter assembly is driven to rotate relative to the outer catheter assembly by rotating the housing, and the operation is stable and convenient.

In some embodiments, the handle further comprises a slidable assembly slidably connected with the housing, and the slidable assembly is connected with the peripheral electrode assembly for driving the peripheral electrode assembly to move in an axial direction, and the peripheral electrode assembly is rotatable relative to the outer catheter assembly by the rotation of the housing relative to the end cap through the slidable assembly. The slidable assembly can not only transmit the torque of the housing to the peripheral electrode assembly to allow the rotation of the peripheral electrode assembly relative to the outer catheter assembly, but can also drive the peripheral electrode assembly to shift in the axial direction to adjust the axial position of the peripheral electrode assembly.

In some embodiments, the slidable assembly comprises a plurality of slidable elements slidably connected with the housing, and each of the slidable elements is at least partially arranged inside the housing, and a proximal end of each peripheral electrode extends into the housing and is fixedly connected with a respective slidable element. By separately controlling each slidable element, each peripheral electrode of the peripheral electrode assembly can be separately controlled, so that each peripheral electrode can be moved to the desired lesion site as required to improve the positioning effect of the peripheral electrode and improve the reliability of ablation monitoring results.

In some embodiments, the slidable assembly comprises a push-pull rod at least partially arranged in the housing and slidably connected with the housing, and the inner catheter assembly further comprises a spring tube received in the outer catheter assembly, the spring tube has a proximal end and a distal end, the proximal end of the spring tube is fixedly connected with the push-pull rod, and the distal end of the spring tube is fixedly connected with the peripheral electrode. By moving the push-pull rod, the peripheral electrode assembly can be moved in the axial direction, and the operation is simple and convenient. Furthermore, the spring tube can improve the operation feel.

In some embodiments, the slidable assembly further comprises a slide button slidably connected with the housing, with a portion of the slide button protruding from an outer surface of the housing, and the other portion of the slide button received in the housing and fixedly connected with the push-pull rod. By operating the slide button, the peripheral electrode assembly can be moved, which is convenient for operation.

In some embodiments, the handle comprises a housing having a proximal end and a distal end, an end cap connected with the distal end of the housing, a rotatable ball received in the end cap and a push-pull rod anti-rotationally connected with the rotatable ball, the proximal end of the outer catheter assembly is connected with the end cap, the push-pull rod is connected with the inner catheter assembly, and the inner catheter assembly is rotatable relative to the outer catheter assembly by a rotation of the rotatable ball relative to the end cap through the push-pull rod. By rotating the rotatable ball, the peripheral electrode assembly can be rotated, and the operation is convenient and accurate.

In some embodiments, the handle further comprises a slide button slidably connected with the housing, and a fixing block rotatably connected with the slide button, and wherein the push-pull rod is fixedly connected with the fixing block and is axially slidable relative to the rotatable ball, and the push-pull rod is axially slidable by an axial movement of the slide button relative to the housing through the fixing block. By operating the slide button, the peripheral electrode assembly can be moved, which is convenient for operation.

In some embodiments, the rotatable ball is provided with a noncircular hole, and the push-pull rod passes through the rotatable ball through the noncircular hole and is engaged with walls of the noncircular holes. The simple noncircular hole not only ensures that the push-pull rod can rotate with the rotation of the rotatable ball, but also ensures that the push-pull rod can move in the axial direction relative to the rotatable ball.

The embodiments of the present invention also provide a radiofrequency ablation catheter, which includes a handle, the handle comprises a housing and a slidable assembly slidably connected with the housing, the slidable assembly comprises a plurality of slidable elements that are respectively and independently slidable in an axial direction relative to the housing, and a catheter assembly connected with the handle, the catheter assembly comprises a peripheral electrode assembly, the peripheral electrode assembly comprises a plurality of peripheral electrodes that are circumferentially spaced from each other; wherein at least some of the plurality of peripheral electrodes are configured to be driven by the respective slidable elements to slide in the axial direction. By separately operating the slidable element that can independently slide in the axial direction relative to the housing, the peripheral electrode of the peripheral electrode assembly can be independently operated, so that the peripheral electrodes can be moved to the desired lesion in the axial direction as required, thereby improving the reliability of the ablation monitoring results, solving the problem that the peripheral electrode assembly in the prior art cannot reach the desired position when entering the lesion site with a poor reliability of the ablation monitoring results.

In some embodiments, the slidable element comprises a slidable rod slidably arranged in the housing and a slide button fixedly connected with the slidable rod and protruding from an outer surface of the housing, and the peripheral electrode is fixedly connected with the slidable rod, and the slide button is slidably connected with the housing. The slide button is operated to slide in the axial direction relative to the housing, thereby driving the peripheral electrode fixedly connected with the slidable rod to slide in the axial direction, and the operation is convenient.

In some embodiments, the housing has a plurality of elongated holes circumferentially spaced from each other, and the elongated hole radially passes through the housing, and a length direction of the elongated hole corresponds to a length direction of the housing, and the slide button of each slidable element protrudes from the outer surface of the housing through a respective elongated hole. The elongated holes can limit the movement of the slide buttons, so that the peripheral electrodes can slide along the axial direction stably along the predetermined paths.

In some embodiments, one radially inside end of the slide button of each slidable element is vertically connected with the slidable rod, and the slide button has a recessed avoidance groove at a side wall thereof adjacent to the radially inside end thereof.

In some embodiments, the slidable rod is provided with a first receiving hole axially extending therethrough, and the peripheral electrode passes through the first receiving hole. The peripheral electrodes respectively pass through the slidable rods through the first receiving holes, which can effectively prevent the peripheral electrodes from crossing with each other.

In some embodiments, the slidable element has a substantially T-shaped configuration, the slide button is arranged adjacent to a proximal end of the slidable rod, and the slidable element further comprises a wedge disposed adjacent to the proximal end of the slidable rod and positioned on a side of the slidable rod opposite to the slide button, and the slidable rod, the slide button, and the wedge together form the substantially T-shaped configuration. The wedge not only helps the slidable element to slide stably in the axial direction, but also helps to avoid interference between adjacent slidable elements.

In some embodiments, the handle further comprises at least one support seat housed in the housing, the support seat abuts against an inner wall of the housing, and the slidable rods slidably passes through the at least one support seats. The support seat can not only support the housing to a certain extent and prevent the housing from being deformed, but also help the slidable rods to slide in the axial direction stably along the predetermined paths.

In some embodiments, an outer periphery of the support seat has at least one groove passing therethrough in the axial direction, and the inner wall of the housing encloses the groove to form an accommodating space. The accommodating space can be used to receive wires such as saline pipelines to prevent the wires from being crossing with each other.

In some embodiments, the handle comprises three support seats which are housed in the housing and spaced from each other in the axial direction, and the slidable rods slidably pass through the middle support seat and the distal support seat. Two support seats support the slidable rods to help the slidable rods to slide steadily in the axial direction. Furthermore, the proximal support seat can also be used for the conducting wires and sensor wires of the respective peripheral electrodes to pass through to prevent the wires from crossing with each other. In addition, the plurality of support seats can support the housing and prevent the housing from deforming.

In some embodiments, the catheter assembly comprises an outer catheter assembly connected with the handle and an inner catheter assembly connected with the handle, the inner catheter assembly is rotatable relative to the outer catheter assembly by driving the handle, the inner catheter assembly comprises a peripheral electrode assembly, and the peripheral electrode assembly comprises a plurality of peripheral electrodes circumferentially spaced from each other. The inner catheter assembly including the peripheral electrode assembly can be driven by the handle to rotate relative to the outer catheter assembly, so that the blood vessels can pass through the gaps between the adjacent peripheral electrodes of the peripheral electrode assembly, thereby avoiding puncturing the blood vessels, reducing operation failure or bringing the hidden treatment hazards or complications to the patient.

In some embodiments, the inner catheter assembly further comprises a support rod received in the outer catheter assembly, the support rod has a plurality of second receiving holes extending therethrough in the axial direction, and the peripheral electrodes respectively pass through the respective second receiving holes. The second receiving holes can be used for accommodating the peripheral electrodes of the peripheral electrode assembly to prevent the peripheral electrodes from crossing with each other. In some embodiments, the second receiving holes can also be used to receive other wires of the radiofrequency ablation catheter, such as saline pipeline, etc., so as to more effectively prevent the wires from crossing with each other.

In some embodiments, the handle further comprises an end cap rotatably connected with the housing, the outer catheter assembly is connected with the end cap, and the peripheral electrode assembly is rotatable relative to the outer catheter assembly by a rotation of the housing relative to the end cap through the slidable assembly. The inner catheter assembly is driven to rotate relative to the outer catheter assembly by rotating the housing, and the operation is stable and convenient.

The embodiments of the present invention also provide a radiofrequency ablation system, including the aforementioned radiofrequency ablation catheter. Since the radiofrequency ablation system includes the aforementioned radiofrequency ablation catheter, the aforementioned effects can also be achieved, which would not be repeated here.

Perfusion Control

Embodiments of the present invention provide a method, apparatus and system for controlling the perfusion of a syringe pump and a non-transitory computer readable storage medium, which may realize dynamic adjustment on a perfusion volume of the syringe pump and improve the timeliness and the accuracy of perfusing a liquid in a process of performing an ablation task.

One aspect of the embodiments of the present invention provides a method for controlling the perfusion of a syringe pump, including: when an ablation task is triggered, controlling the syringe pump to perform a perfusion operation on an ablation object, and obtaining an impedance value and/or a temperature value of the ablation object in real time; analyzing the obtained impedance value and/or the obtained temperature value to obtain real-time change information of the impedance value and/or the temperature value; and dynamically adjusting a perfusion volume of the syringe pump according to the real-time change information obtained from the analysis.

One aspect of the embodiments of the present invention further provides an apparatus for controlling the perfusion of the syringe pump, including: a control module, which is configured to when an ablation task is triggered, control the syringe pump to perform a perfusion operation on an ablation object, and obtain an impedance value and/or a temperature value of the ablation object in real time; an analysis module, which is configured to analyze the obtained impedance value and/or the obtained temperature value to obtain real-time change information of the impedance value and/or the temperature value; and an adjustment module, which is configured to dynamically adjust a perfusion volume of the syringe pump according to the real-time change information obtained from the analysis.

One aspect of the embodiments of the present invention further provides an electronic apparatus, including a non-transitory memory and a processor, wherein the non-transitory memory stores an executable program code; the processor is electrically coupled with the non-transitory memory, a temperature acquisition apparatus and an impedance acquisition apparatus; the processor calls the executable program code stored in the non-transitory memory to perform the method for controlling the perfusion of the syringe pump according to the foregoing embodiment.

One aspect of the embodiments of the present invention further provides a system for controlling the perfusion of a syringe pump, including a syringe pump, a temperature acquisition apparatus and an impedance acquisition apparatus, wherein the syringe pump includes a controller, a syringe, a push rod and a drive apparatus, wherein the controller is electrically coupled with the drive apparatus, the temperature acquisition apparatus and the impedance acquisition apparatus, and configured to perform steps of the method for controlling the perfusion of the syringe pump according to the foregoing embodiment; the drive apparatus is configured to drive the push rod to move in a direction pointed by the control instruction of the controller at a speed indicated by the control instruction according to the control instruction so as to control and adjust a perfusion volume of a syringe; the temperature acquisition apparatus is configured to acquire a temperature value of the ablation object and transmit it to the controller; and the impedance acquisition apparatus is configured to acquire an impedance value of the ablation object and transmit it to the controller.

One aspect of the embodiments of the present invention further provides a radio frequency ablation system, including a radio frequency ablation control apparatus, a radio frequency ablation catheter, a neutral electrode, a syringe pump, a temperature acquisition apparatus and an impedance acquisition apparatus, wherein the radio frequency ablation control apparatus is configured to perform steps in the method for controlling the perfusion of the syringe pump according to the foregoing embodiment; the radio frequency ablation catheter is configured to perform an ablation operation on the ablation object according to a control instruction of the radio frequency ablation control apparatus.

The temperature acquisition apparatus is configured to acquire a temperature value of the ablation object and transmit it to the radio frequency ablation control apparatus; and the impedance acquisition apparatus is configured to acquire an impedance value of the ablation object and transmit it to the radio frequency ablation control apparatus.

One aspect of the embodiments of the present invention further provides a non-transitory computer readable storage medium on which a computer program is stored, wherein the computer program, when run by a processor, implements the method for controlling the perfusion of a syringe pump according to the embodiments mentioned above.

When the ablation task is triggered, the syringe pump is controlled to perform the perfusion operation on the ablation object, and the impedance value and/or the temperature value of the ablation object are obtained in real time; and then the obtained impedance value and/or the obtained temperature value are/is analyzed, and the perfusion volume of the syringe pump is dynamically adjusted according to real-time change information of the impedance value and/or the temperature value obtained from the analysis. Accordingly, automatic dynamic adjustment on the perfusion volume of the syringe pump is realized based on the analysis of changes in the impedance value and/or the temperature value of the ablation object in the process of performing the ablation task. Since the perfusion volume of the syringe pump is automatically adjusted dynamically as the impedance value and/or the temperature value of the ablation object changes, operation delays and errors caused by manual determination can be reduced, and the timeliness and the accuracy of perfusing the liquid in the process of performing the ablation task can be improved. Accordingly, the injury of the ablation operation to the ablation object is reduced, and the safety of the radio frequency ablation treatment is improved.

Data Adjustment Method in Radio-Frequency Operation and Radio-Frequency Host

An embodiment of the present invention provides a data adjustment method in a radio-frequency operation and a radio-frequency host. During the radio-frequency operation, the radio-frequency output power or a preset range for physical characteristic data of a subject of the radio-frequency operation is adjusted to improve the safety and effectiveness of radio-frequency operation.

In an aspect, an embodiment of the present invention provides a data adjustment method in a radio-frequency operation, which includes: acquiring set power data corresponding to a radio-frequency operation, setting an output power of a radio-frequency signal according to the set power data, and outputting the radio-frequency signal to a subject of the radio-frequency operation; detecting physical characteristic data of the subject in real time, and determining whether the physical characteristic data exceeds a preset range; adjusting the radio-frequency output power if the physical characteristic data exceeds the preset range; and adjusting the preset range according to the physical characteristic data detected in real time in a preset period of time before a current moment if the physical characteristic data does not exceed the preset range.

In an aspect, an embodiment of the present invention also provides a radio-frequency host, which includes an acquisition module, configured to acquire set power data corresponding to a radio-frequency operation; a transmitting module, configured to set an output power of a radio-frequency signal according to the set power data, and output the radio-frequency signal to a subject of the radio-frequency operation; a detection module, configured to detect physical characteristic data of the subject in real time, and determine whether the physical characteristic data exceeds a preset range; and an adjustment module, configured to adjust the radio-frequency output power if the physical characteristic data exceeds the preset range, and adjust the preset range according to the physical characteristic data detected in real time in a preset period of time before a current moment if the physical characteristic data does not exceed the preset range.

In an aspect, an embodiment of the present invention also provides a radio-frequency host, which includes a storage and a processor, wherein the storage stores an executable program code; and the processor is coupled to the storage, and configured to call the executable program code stored in the storage, and implement the data adjustment method in a radio-frequency operation as described above.

As can be known from the above embodiments of the present invention, set power data corresponding to a radio-frequency operation is acquired, an output power of a radio-frequency signal is set according to the set power data, and the radio-frequency signal is outputted physical characteristic data of a subject of the radio-frequency operation is detected in real time during the radio-frequency operation, and whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; and if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

Method for Protecting Radio Frequency Operation Abnormality, Radio Frequency Mainframe, and Radio Frequency Operation System

Embodiments of the present invention provide a method for protecting radio frequency operation abnormality, a radio frequency mainframe, and a radio frequency operation system, which can implement dual protection by stopping outputting radio frequency energy and cutting off a radio frequency energy path when a radio frequency operation is abnormal, thereby improving safety of radio frequency operations.

In one aspect, an embodiment of the present invention provides a method for protecting radio frequency operation abnormality, comprising: when detecting that a radio frequency mainframe continuously outputs radio frequency energy, detecting preset kinds of detection data of a radio frequency operation in real time; determining whether detected preset kinds of detection data meets a preset abnormal state; and if the preset abnormal state is met, controlling a radio frequency generating apparatus to stop outputting radio frequency energy and controlling an emergency stop apparatus to cut off a radio frequency energy output path of the radio frequency mainframe.

In one aspect, an embodiment of the present invention provides a radio frequency mainframe comprising a detecting apparatus, a radio frequency generating apparatus, and an emergency stop apparatus; wherein the detecting apparatus is configured to: when it is detected that the radio frequency mainframe continuously outputs radio frequency energy, detect preset kinds of detection data of a radio frequency operation in real time; the detecting apparatus is further configured to: determine whether detected preset kinds of detection data meets a preset abnormal state; and the detecting apparatus is further configured to: if the preset abnormal state is met, control the radio frequency generating apparatus to stop outputting radio frequency energy and control the emergency stop apparatus to cut off a radio frequency energy output path.

In one aspect, an embodiment of the present invention provides a radio frequency mainframe, comprising: a memory and a processor; wherein the memory stores executable program codes; the processor coupled with the memory calls the executable program codes stored in the memory to execute the aforesaid method for protecting radio frequency operation abnormality.

In one aspect, an embodiment of the present invention provides a radio frequency operation system, comprising: a radio frequency mainframe and an injection pump; wherein the radio frequency mainframe is configured to execute the aforesaid method for protecting radio frequency operation abnormality; and the injection pump is configured to inject liquid with a preset function to a radio frequency operated object under control of the radio frequency mainframe.

From the above embodiments of the present invention, it can be known that: when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

Method and Apparatus for Dynamically Adjusting Radio Frequency Parameter and Radio Frequency Host

Embodiments of the present invention provide a method and an apparatus for dynamically adjusting a radio frequency parameter and a radio frequency host, which may realize that radio frequency data of a radio frequency object is dynamically adjusted by comparing the detected radio frequency data of an operation object with a preset radio frequency data standard range and a preset radio frequency data limit range, so as to improve the success rate and the safety of the radio frequency operation.

In one aspect, embodiments of the present invention provide a method for dynamically adjusting a radio frequency parameter, including: confirming an operation stage in which a radio frequency operation is and acquiring a radio frequency data standard range and a radio frequency data limit range corresponding to an operation object of the radio frequency operation and the operation stage, wherein the radio frequency data standard range is within the radio frequency data limit range; detecting radio frequency data of the operation object in real time, and comparing the radio frequency data of the operation object with the radio frequency data standard range and the radio frequency data limit range, respectively; if the radio frequency data detected in real time exceeds the radio frequency data standard range but does not exceed the radio frequency data limit range and lasts for a preset duration, controlling the radio frequency data to be within the radio frequency data standard range by controlling an injection volume of a syringe pump to the operation object; and if the radio frequency data detected in real time exceeds the radio frequency data limit range, stopping radio frequency energy from being output.

In one aspect, embodiments of the present invention further provide an apparatus for dynamically adjusting a radio frequency parameter, including: an acquisition module, which is configured to confirm an operation stage in which a radio frequency operation is and acquire a radio frequency data standard range and a radio frequency data limit range corresponding to an operation object of the radio frequency operation and the operation stage, wherein the radio frequency data standard range is within the radio frequency data limit range; a detection module, which is configured to detect radio frequency data of the operation object in real time; a comparison module which is configured to compare the detected radio frequency data with the radio frequency data standard range and the radio frequency data limit range; and a control module, which is configured to if the radio frequency data detected in real time exceeds the radio frequency data standard range but does not exceed the radio frequency data limit range and lasts for a preset duration, control the radio frequency data to be within the radio frequency data standard range by controlling an injection volume of a syringe pump to the operation object, and if the radio frequency data detected in real time exceeds the radio frequency data limit range, stop radio frequency energy from being output.

In one aspect, embodiments of the present invention further provide a radio frequency host, including: a memory and a processor, wherein the memory stores an executable program code; and the processor coupled with the memory calls the executable program code stored in the memory to perform the method for dynamically adjusting the radio frequency parameter as described above.

It may be known from the above embodiments of the present invention that the radio frequency data standard range corresponding to the operation object of the radio frequency operation and the current operation stage is acquired, and the radio frequency data detected in real time is compared with the radio frequency data standard range and the radio frequency data limit range in real time, respectively. If the radio frequency data detected in real time exceeds the radio frequency data standard range but does not exceed the radio frequency data limit range and lasts for the preset duration, the radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the radio frequency data detected in real time exceeds the radio frequency data limit range, it is confirmed that problems occur in the radio frequency host of the current radio frequency operation or the operation object, and the radio frequency energy is stopped from being output. Therefore, the radio frequency host and the operation object are prevented from being damaged, and the safety of the radio frequency operation is improved.

Method and Apparatus for Safety Control of Radio Frequency Operation, and Radio Frequency Mainframe

Embodiments of the present invention provides a method and apparatus for safety control of radio frequency operation, and a radio frequency mainframe, which can improve safety and intelligence of radio frequency operations when connection between a radio frequency mainframe and a radio frequency operated object does not meet a standard.

An aspect of embodiments of the present invention provides a method for safety control of radio frequency operation, comprising: when connecting ends of a plurality of radio frequency circuits connects an operated object to a radio frequency mainframe, acquiring detected values of the plurality of radio frequency circuits; determining whether change amounts of the detected values reach a preset value range; if a quantity of target radio frequency circuits of which the change amounts of the detected values reach the preset value range is not less than a preset quantity, selecting the preset quantity of target radio frequency circuits from the target radio frequency circuits according to a preset selection rule as radio frequency input circuits, and inputting radio frequency energy into the radio frequency input circuits; if the quantity of target radio frequency circuits of which the change amounts of the detected values reach the preset value range is less than the preset quantity, not inputting radio frequency energy into any radio frequency circuit.

An aspect of embodiments of the present invention further provides an apparatus for safety control of radio frequency operation, comprising: an acquiring module configured to: when connecting ends of a plurality of radio frequency circuits connects an operated object to a radio frequency mainframe, acquire detected values of the plurality of radio frequency circuits; a determining module configured to determine whether change amounts of the detected values reach a preset value range; and a processing module configured to: if a quantity of target radio frequency circuits of which the change amounts of the detected values reach the preset value range is not less than a preset quantity, select the preset quantity of target radio frequency circuits from the target radio frequency circuits according to a preset selection rule as radio frequency input circuits, and input radio frequency energy into the radio frequency input circuits; wherein the processing module is further configured to: if the quantity of target radio frequency circuits of which the change amounts of the detected values reach the preset value range is less than the preset quantity, not input radio frequency energy into any radio frequency circuit.

An aspect of embodiments of the present invention further provides a radio frequency mainframe, comprising: a memory and a processor, wherein the memory stores executable program codes; the processor coupled with the memory calls the executable program codes stored in the memory to execute the aforesaid method for safety control of radio frequency operation.

From the above embodiments of the present invention, it can be known that: when an operated object is connected to a radio frequency mainframe through connecting ends of radio frequency circuits, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation.

Ablation Operation Prompting Method, Electronic Device and Computer-Readable Storage Medium

An object of the embodiments of the present invention is to provide an ablation operation prompting method, an electronic device, and a computer-readable storage medium, with which the status changes of an ablation site can be displayed in real time and intuitively during the implementation of the ablation operation, thereby improving the effectiveness and relevance of information prompts.

In an aspect, an embodiment of the present invention provides an ablation operation prompting method, applicable to a computer terminal. The method includes: acquiring an image of an ablation site and display the image on a screen, when an ablation task is triggered; acquiring position data of a currently-being-ablated target ablation point, and marking the target ablation point in the image according to the position data; acquiring the elapsed ablation time and the temperature of the target ablation point in real time, and determining the ablation status of the target ablation point according to the elapsed ablation time and the temperature; and generating a schematic real-time dynamic change diagram of the target ablation point according to the ablation status, and displaying the schematic diagram on the screen, to indicate the real-time ablation status change of the target ablation point.

In an aspect, an embodiment of the present invention further provides an ablation operation prompting device, which includes: an image display module, configured to acquire an image of an ablation site and display the image on a screen, when an ablation task is triggered; a marking module, configured to acquire position data of a currently-being-ablated target ablation point, and mark the target ablation point in the image according to the position data; an ablation status determination module, configured to acquire the elapsed ablation time and the temperature of the target ablation point in real time, and determine the ablation status of the target ablation point according to the elapsed ablation time and the temperature; and an ablation status prompting module, configured to generate a schematic real-time dynamic change diagram of the target ablation point according to the ablation status, and display the schematic diagram on the screen, to indicate the real-time ablation status change of the target ablation point.

In an aspect, an embodiment of the present invention further provides an electronic device, which includes a storage and a processor, wherein the storage stores an executable program code; and the processor is coupled to the storage, and configured to call the executable program code stored in the storage, and implement the ablation operation prompting method provided in the above embodiments.

In an aspect, an embodiment of the present invention further provides a non-transitory computer-readable storage medium, on which a computer program is stored, wherein when the computer program is executed by the processor, the ablation operation prompting method provided in the above embodiments is implemented.

According to various embodiments provided in the present invention, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic real-time dynamic change diagram of the ablation status of the target ablation point is generated and displayed, so that the status changes of an ablation site can be displayed in real time and intuitively during the implementation of the ablation operation, thereby improving the effectiveness and relevance of information prompts.

Radio-Frequency Operation Prompting Method, Electronic Device, and Computer-Readable Storage Medium

An embodiment of the present invention provides a radio-frequency operation prompting method, an electronic device, and a computer-readable storage medium, with which visual prompting of the change of range of a to-be-operated area is realized, thereby improving the effectiveness of information prompts, and thus improving the success rate and effect of the radio-frequency operation.

In an aspect, an embodiment of the present invention provides a radio-frequency operation prompting method, applicable to a computer terminal. The method includes: acquiring physical characteristic data of an operating position in a subject of a radio-frequency operation in real time by multiple probes; obtaining a physical characteristic field of the subject of the radio-frequency operation according to the physical characteristic data acquired in real time; and obtaining the change of range of a to-be-operated area in a target operating area according to an initial range of the target operating area in the subject of the radio-frequency operation and the change of value of the physical characteristic data in the physical characteristic field, and displaying the change of range by a three-dimensional model.

In an aspect, an embodiment of the present invention further provides a radio-frequency operation prompting device, which includes: an acquisition module, configured to acquire physical characteristic data of an operating position in a subject of a radio-frequency operation in real time by multiple probes; a processing module, configured to obtain a physical characteristic field of the subject of the radio-frequency operation according to the physical characteristic data acquired in real time, and obtain the change of range of a to-be-operated area in a target operating area according to an initial range of the target operating area in the subject of the radio-frequency operation and the change of value of the physical characteristic data in the physical characteristic field; and a display module, configured to display the change of range by a three-dimensional model.

In an aspect, an embodiment of the present invention further provides an electronic device, which includes a storage and a processor, wherein the storage stores an executable program code; and the processor is coupled to the storage, and configured to call the executable program code stored in the storage, and implement the radio-frequency operation prompting method provided in the above embodiments.

In an aspect, an embodiment of the present invention further provides a non-transitory computer-readable storage medium, on which a computer program is stored, wherein when the computer program is executed by a processor, the radio-frequency operation prompting method provided in the above embodiments is implemented.

According to various embodiments provided in the present invention, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

According to various embodiments provided in the present invention, an electrosurgical method for ablating a target lesion in the lung comprises: identifying a lesion to ablate; identifying an interior and a periphery of the lesion; advancing an ablation electrode into the interior of the lesion;

commencing ablation from the ablation electrode; and sensing information at the periphery of the lesion during ablation.

In an aspect, an embodiment of the present invention further comprises sensing information in the interior of the lesion.

In an aspect, an embodiment of the present invention further comprises guiding sensors into or beyond the periphery of the lesion.

In an aspect of an embodiment of the present invention, the sensors are operable to sense temperature.

In an aspect of an embodiment of the present invention, the sensors are operable to sense impedance.

In an aspect of an embodiment of the present invention, the ablation electrode applies RF energy.

In an aspect of an embodiment of the present invention, the ablation electrode is arranged with a neutral electrode in a monopolar configuration.

In an aspect, an embodiment of the present invention further comprises stopping ablation based on the sensed information.

In an aspect, an embodiment of the present invention further comprises adjusting ablation based on sensor information.

In an aspect, an embodiment of the present invention further comprises irrigating the ablation electrode.

In an aspect, an embodiment of the present invention further comprises adjusting the flowrate of irrigating based on the sensed information.

In an aspect, an embodiment of the present invention further comprises evaluating progress of the ablation of the lesion based on imaging.

In an aspect of an embodiment of the present invention, the imaging is based on the sensed information or an external imaging technique.

In an aspect of an embodiment of the present invention, the ablation electrode is inserted into the center of mass of the lesion.

In an aspect of an embodiment of the present invention, the lesion is a tumor.

In an aspect of an embodiment of the present invention, the irrigating comprises ejecting a liquid from a plurality of discrete ports arranged along the ablation electrode from the proximal end to the distal end.

In an aspect of an embodiment of the present invention, the discrete ports are configured to eject the liquid at an equal flowrate along the ablation electrode.

In an aspect of an embodiment of the present invention, the ablation electrode is arranged with a return electrode in a bipolar configuration.

According to various embodiments provided in the present invention, a method for treating lung cancer comprises: advancing an ablation catheter into a lung and to a tumor to be ablated; positioning an active electrode of the ablation catheter inside or adjacent the tumor; deploying a plurality of sensors in a peripheral region surrounding the tumor and spaced a distance from the active electrode; irrigating the active electrode and tumor; activating the active electrode while irrigating; and adjusting the power delivered to the active electrode based on information detected from the sensors.

In an aspect of an embodiment of the present invention, the deploying comprises adjustably deploying the plurality of sensors according to the size of the tumor.

In an aspect of an embodiment of the present invention, the adjusting comprises extension of the sensors from the catheter shaft and/or rotation of the sensors relative to the catheter shaft.

In an aspect of an embodiment of the present invention, the deploying comprises penetrating the lesion with a plurality of elongate supports, and wherein at least one sensor is arranged on each elongate support.

In an aspect, an embodiment of the present invention further comprises adjusting the irrigating based on information from the sensors.

The description, objects and advantages of embodiments of the present invention will become apparent from the detailed description to follow, together with the accompanying drawings.

### BRIEF DESCRIPTION OF THE DRAWINGS

FIG. 1 is a schematic structural view showing the overall structure of a radio-frequency ablation catheter according to a first embodiment of the present invention;

FIG. 2 is a partially enlarged view of a needle tube portion in the radio-frequency ablation catheter according to the first embodiment of the present invention;

FIG. 3 shows a fixing base connected to a fixing ring in the radio-frequency ablation catheter according to the first embodiment of the present invention;

FIG. 4 is a schematic structural view of the fixing base in the radio-frequency ablation catheter according to the first embodiment of the present invention;

FIG. 5 is a schematic view showing the positions of the connecting base and a spring in the radio-frequency ablation catheter according to the first embodiment of the present invention;

FIG. 6 is a first sectional view of the connecting base in the radio-frequency ablation catheter according to the first embodiment of the present invention;

FIG. 7 is a second sectional view of the connecting base in the radio-frequency ablation catheter according to the first embodiment of the present invention;

FIG. 8 is a schematic structural view showing the overall structure of an infiltration hood in the radio-frequency ablation catheter according to the first embodiment of the present invention;

FIG. 9 is a sectional view of the infiltration hood in the radio-frequency ablation catheter according to the first embodiment of the present invention;

FIG. 10 is a schematic view showing a second tube sleeve connected to the infiltration hood in the radio-frequency ablation catheter according to the first embodiment of the present invention;

FIG. 11 is a left side view of the fixing base in the radio-frequency ablation catheter according to the first embodiment of the present invention; and

FIG. 12 is a schematic structural view of a claw-shaped electrode in the radio-frequency ablation catheter according to the first embodiment of the present invention.

FIG. 13 is a schematic view of a radiofrequency ablation catheter according to a first embodiment of the present invention;

FIG. 14 is a partially exploded schematic view of the catheter assembly of the radiofrequency ablation catheter shown in FIG. 13;

FIG. 15 is a partial cross-sectional view of the catheter assembly of the radiofrequency ablation catheter shown in FIG. 13;

FIG. 16 is a partially exploded schematic view of the handle of the radiofrequency ablation catheter shown in FIG. 13;

FIG. 17 is a schematic view of the radiofrequency ablation catheter according to a second embodiment of the present invention;

FIG. 18 is a partial exploded schematic view of the handle of the radiofrequency ablation catheter shown in FIG. 17;

FIG. 19 is a schematic view of the radiofrequency ablation catheter according to a third embodiment of the present invention;

FIG. 20 is a partial exploded schematic view of the handle of the radiofrequency ablation catheter shown in FIG. 19;

FIG. 21 is a partially exploded schematic view of the catheter assembly of the radiofrequency ablation catheter shown in FIG. 19;

FIG. 22 is a schematic view of a radiofrequency ablation system according to a fourth embodiment of the present invention.

FIG. 23 is a diagram showing an application environment of a method for controlling the perfusion of a syringe pump according to an embodiment of the present invention;

FIG. 24 is a schematic diagram showing a structure of a system for controlling the perfusion of a syringe pump according to an embodiment of the present invention;

FIG. 25 is a schematic diagram showing a structure of a syringe pump in a system for controlling the perfusion of a syringe pump as shown in FIG. 24;

FIG. 26 is a flow chart of an implementation of a method for controlling the perfusion of a syringe pump according to an embodiment of the present invention;

FIG. 27 is a flow chart of an implementation of a method for controlling the perfusion of a syringe pump according to another embodiment of the present invention;

FIG. 28 is a flow chart of an implementation of a method for controlling the perfusion of a syringe pump according to further embodiment of the present invention;

FIG. 29 is a flow chart of an implementation of steps S603 to S605 in an embodiment as shown in FIG. 28;

FIG. 30 is a flow chart of an implementation of a step S604 in an embodiment as shown in FIG. 28;

FIG. 31 is a flow chart of an implementation of a step S605 in an embodiment as shown in FIG. 28;

FIG. 32 is a schematic diagram showing a structure of an apparatus for controlling the perfusion of a syringe pump according to an embodiment of the present invention;

FIG. 33 is a schematic diagram showing a hardware structure of an electronic apparatus according to an embodiment of the present invention; and

FIG. 34 is a schematic diagram showing a structure of a radio frequency ablation system according to an embodiment of the present invention.

FIG. 35 is a schematic diagram showing an application scenario of a data adjustment method in a radio-frequency operation provided in an embodiment of the present invention;

FIG. 36 is a schematic flow chart of a data adjustment method in a radio-frequency operation provided in an embodiment of the present invention;

FIG. 37 is a schematic flow chart of a data adjustment method in a radio-frequency operation provided in another embodiment of the present invention;

FIG. 38 is a schematic structural diagram of a radio-frequency host provided in an embodiment of the present invention; and

FIG. 39 is a schematic diagram showing a hardware structure in a radio-frequency host provided in an embodiment of the present invention.

FIG. 40 is a schematic diagram of an application scene of a method for protecting radio frequency operation abnormality provided by an embodiment of the present invention.

FIG. 41 is a schematic flow chart of a method for protecting radio frequency operation abnormality provided by an embodiment of the present invention.

FIG. 42 is a schematic flow chart of a method for protecting radio frequency operation abnormality provided by another embodiment of the present invention.

FIG. 43 is a schematic flow chart of a method for protecting radio frequency operation abnormality provided by another embodiment of the present invention.

FIG. 44 is a structural schematic diagram of a radio frequency mainframe provided by an embodiment of the present invention.

FIG. 45 is a structural schematic diagram of a radio frequency mainframe provided by another embodiment of the present invention.

FIG. 46 is a structural schematic diagram of a radio frequency mainframe provided by another embodiment of the present invention.

FIG. 47 is a structural schematic diagram of a radio frequency operation system provided by an embodiment of the present invention.

FIG. 48 is a schematic diagram showing an application scenario of a method for dynamically adjusting a radio frequency parameter according to an embodiment of the present invention;

FIG. 49 is a schematic flow chart of a method for dynamically adjusting a radio frequency parameter according to an embodiment of the present invention;

FIG. 50 is a schematic flow chart of a method for dynamically adjusting a radio frequency parameter according to another embodiment of the present invention;

FIG. 51 is a schematic diagram showing a structure of an apparatus for dynamically adjusting a radio frequency parameter according to an embodiment of the present invention; and

FIG. 52 is a schematic diagram showing a structure of a radio frequency host according to an embodiment of the present invention.

FIG. 53 is a schematic diagram of an application scene of a method for safety control of radio frequency operation provided by an embodiment of the present invention.

FIG. 54 is a schematic flow chart of a method for safety control of radio frequency operation provided by an embodiment of the present invention.

FIG. 55 is a schematic flow chart of a method for safety control of radio frequency operation provided by another embodiment of the present invention.

FIG. 56 is a structural schematic diagram of a radio frequency circuit in a method for safety control of radio frequency operation provided by an embodiment of the present invention.

FIG. 57 is a schematic flow chart of a method for safety control of radio frequency operation provided by another embodiment of the present invention.

FIG. 58 is a structural schematic diagram of an impedance detection circuit in a method for safety control of radio frequency operation provided by an embodiment of the present invention.

FIG. 59 is a structural schematic diagram of an apparatus for safety control of radio frequency operation provided by an embodiment of the present invention.

FIG. 60 is a structural schematic diagram of a radio frequency mainframe provided by an embodiment of the present invention.

FIG. 61 is a schematic diagram of an existing ablation operation prompting interface;

FIG. 62 shows an application environment of an ablation operation prompting method provided in an embodiment of the present invention;

FIG. 63 shows a flow chart of an ablation operation prompting method provided in an embodiment of the present invention;

FIG. 64 and FIG. 65 are schematic diagrams showing an image of an ablation site and the real-time dynamic change of the ablation status of a target ablation point in an ablation operation prompting method provided in an embodiment of the present invention;

FIG. 66 shows a flow chart of an ablation operation prompting method provided in another embodiment of the present invention;

FIG. 67 is a schematic diagram showing an operation track in an ablation operation prompting method provided in an embodiment of the present invention;

FIG. 68 is a schematic diagram showing a real-time temperature change curve and a real-time impedance change curve in an ablation operation prompting method provided in an embodiment of the present invention;

FIG. 69 is a schematic diagram showing the real-time impedance in an ablation operation prompting method provided in an embodiment of the present invention;

FIG. 70 is a schematic diagram showing the real-time impedance change in an ablation operation prompting method provided in an embodiment of the present invention;

FIG. 71 is a schematic diagram showing a picture of a screen on which all the schematic views are displayed in an ablation operation prompting method provided in an embodiment of the present invention;

FIG. 72 is a schematic structural diagram of an ablation operation prompting device provided in an embodiment of the present invention; and

FIG. 73 is a schematic diagram showing a hardware structure of an electronic device provided in an embodiment of the present invention.

FIG. 74 is a schematic diagram of an existing radio-frequency operation prompting interface;

FIG. 75 shows an application environment of a radio-frequency operation prompting method provided in an embodiment of the present invention;

FIG. 76 shows a flow chart of a radio-frequency operation prompting method provided in an embodiment of the present invention;

FIG. 77 is a schematic view of a tip of a radio-frequency operation catheter in a radio-frequency operation prompting method provided in an embodiment of the present invention;

FIG. 78 shows a flow chart of a radio-frequency operation prompting method provided in another embodiment of the present invention;

FIG. 79 is a schematic structural diagram of a radio-frequency operation prompting device provided in an embodiment of the present invention;

FIG. 80 is a schematic diagram showing a hardware structure of an electronic device provided in an embodiment of the present invention; and

FIG. 81 is an illustration of a bronchoscopic method for ablating a lung tumor in accordance with an embodiment of the invention.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

The following are incorporated herein by reference in their entirety for all purposes:

PCT/CN2020/118645, filed Sep. 29, 2020, entitled “RADIO-FREQUENCY ABLATION CATHETER AND RADIO-FREQUENCY ABLATION SYSTEM”; PCT/CN2020/118646, filed Sep. 29, 2020, entitled “DETECTION MECHANISM, RADIO FREQUENCY ABLATION CATHETER AND RADIO FREQUENCY ABLATION SYSTEM”; PCT/CN2020/140380, filed Dec. 28, 2020, entitled “INJECTION PUMP PERFUSION CONTROL METHOD, DEVICE, SYSTEM AND COMPUTER READABLE STORAGE MEDIUM”; PCT/CN2021/072952, filed Jan. 20, 2021, entitled “PROTECTION METHOD FOR ABNORMAL RADIO FREQUENCY OPERATION, RADIO FREQUENCY HOST AND RADIO FREQUENCY OPERATING SYSTEM”; PCT/CN2021/072953, filed Jan. 20, 2021, entitled “INJECTION PUMP MULTI-PATH PERFUSION CONTROL METHOD, DEVICE, INJECTION PUMP AND STORAGE MEDIUM”; PCT/CN2021/072954, filed Jan. 20, 2021, entitled “METHOD, DEVICE AND RADIO FREQUENCY HOST FOR DYNAMICALLY ADJUSTING RADIO FREQUENCY PARAMETERS”; PCT/CN2021/072955, filed Jan. 20, 2021, entitled “METHOD FOR PROMPTING ABLATION OPERATION, ELECTRONIC DEVICE AND COMPUTER READABLE STORAGE MEDIUM”; PCT/CN2021/072956, filed Jan. 20, 2021, entitled “DATA ADJUSTMENT METHOD IN RADIO FREQUENCY OPERATION AND RADIO FREQUENCY HOST”; PCT/CN2021/072957, filed Jan. 20, 2021, entitled “RADIO FREQUENCY OPERATION SAFETY CONTROL METHOD, DEVICE AND RADIO FREQUENCY HOST”; PCT/CN2021/072959, filed Jan. 20, 2021, entitled “RADIO FREQUENCY OPERATION PROMPT METHOD, ELECTRONIC DEVICE AND COMPUTER READABLE STORAGE MEDIUM”; PCT/CN2021/076118, filed Feb. 8, 2021, entitled “RADIO-FREQUENCY ABLATION CATHETER AND RADIO-FREQUENCY ABLATION SYSTEM”; and PCT/CN2021/123705, filed Oct. 14, 2021, entitled “RADIOFREQUENCY ABLATION CATHETER AND SYSTEM THEREOF.”

Described herein are ablation methods and related systems.

Detection Mechanism, Radio-Frequency Ablation Catheter, and Radio-Frequency Ablation System

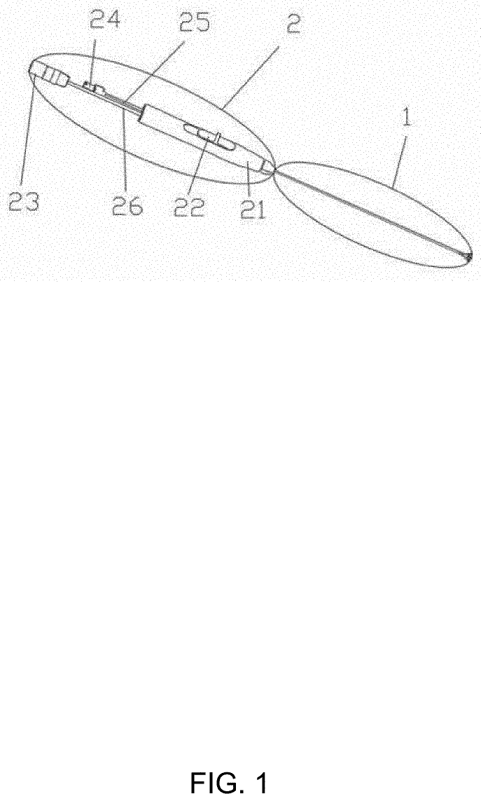

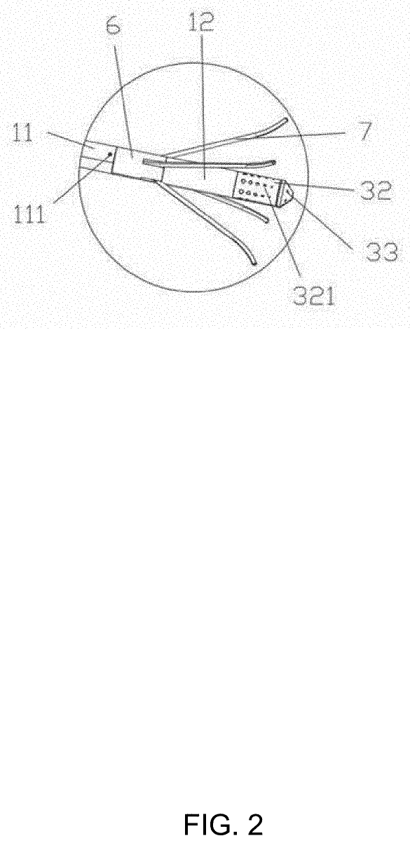

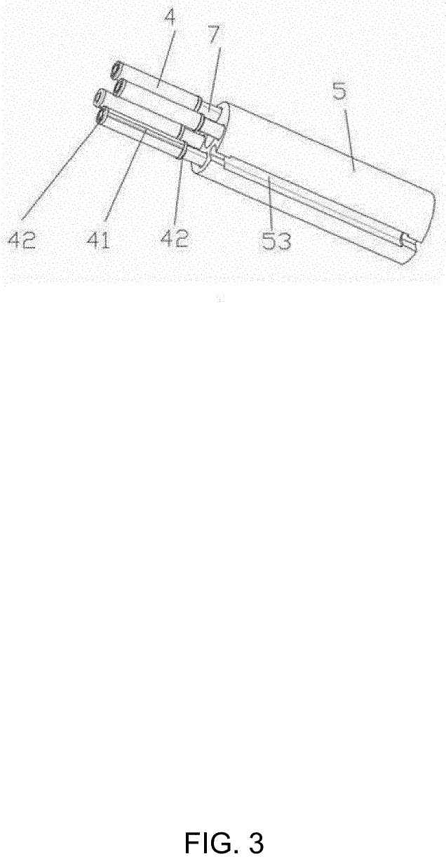

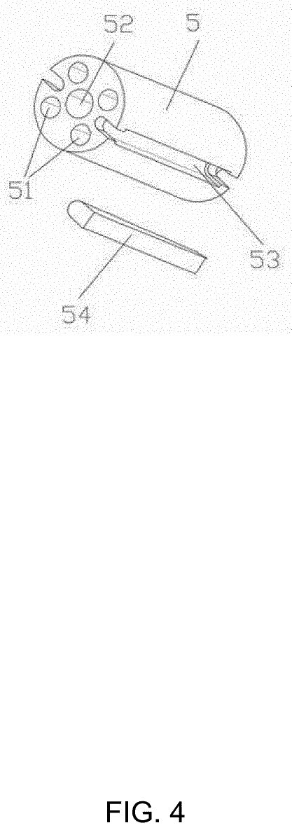

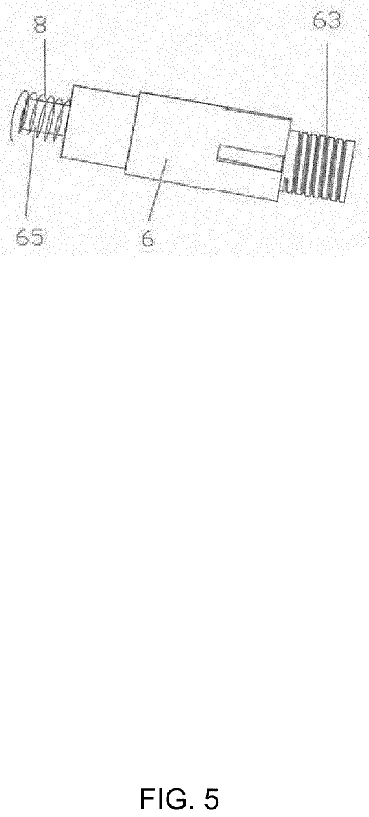

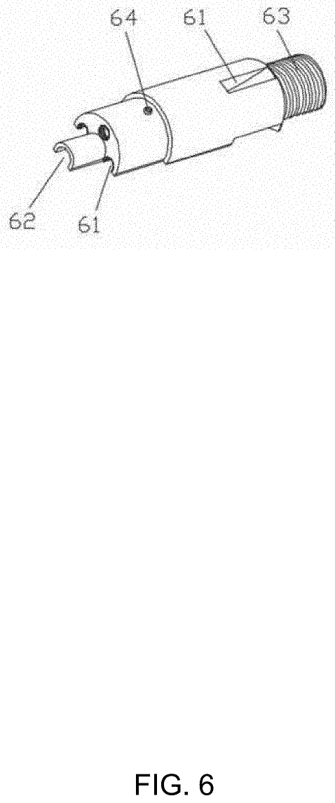

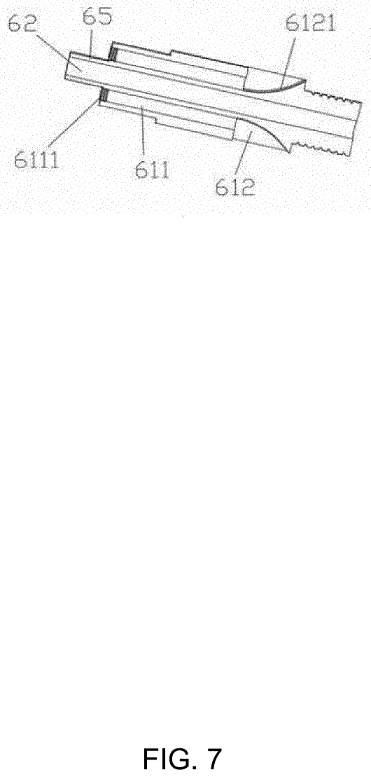

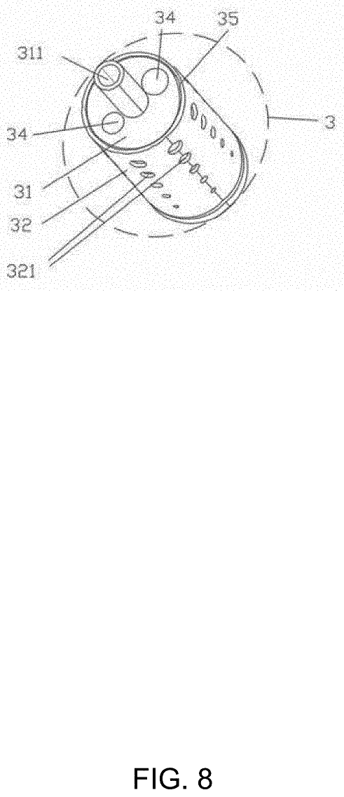

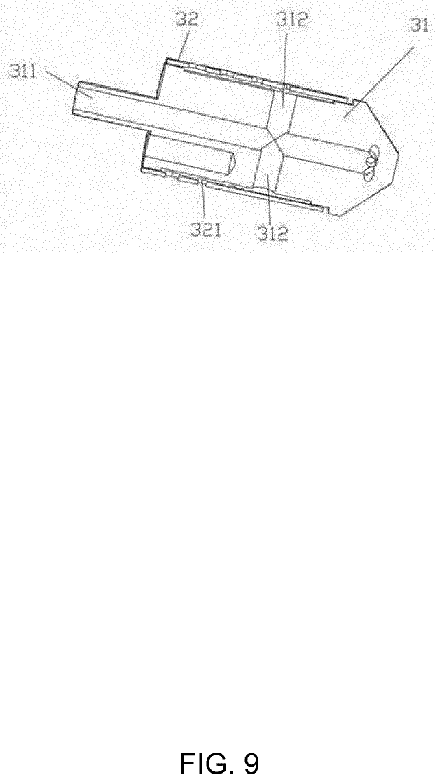

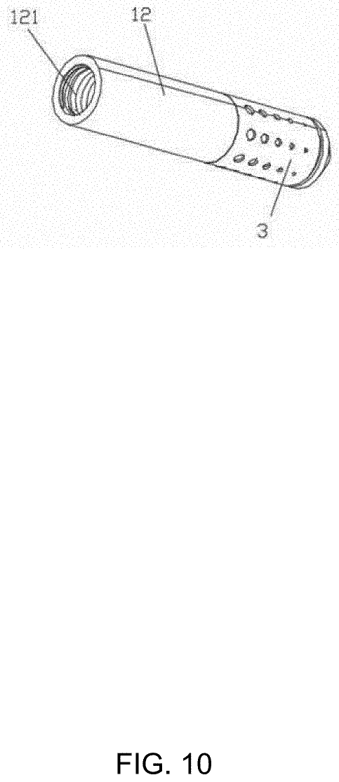

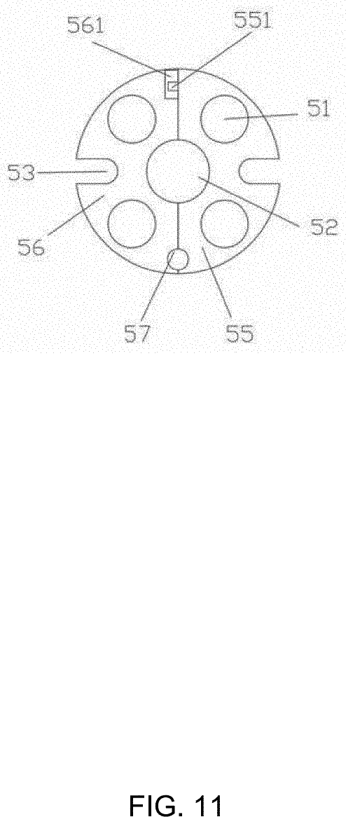

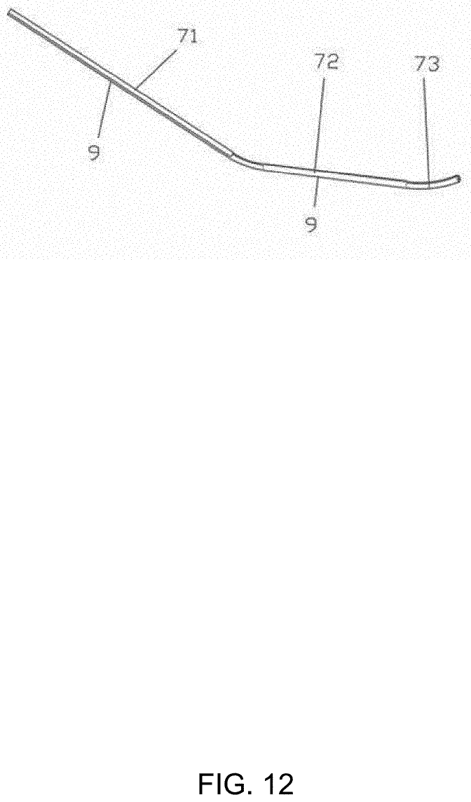

FIG. 1 is a schematic structural view showing the overall structure of a radio-frequency ablation catheter according to a first embodiment of the present invention; FIG. 2 is a partially enlarged view of a needle tube portion in the radio-frequency ablation catheter according to the first embodiment of the present invention; FIG. 3 shows a fixing base connected to a fixing ring in the radio-frequency ablation catheter according to the first embodiment of the present invention; FIG. 4 is a schematic structural view of the fixing base in the radio-frequency ablation catheter according to the first embodiment of the present invention; FIG. 5 is a schematic view showing the positions of the connecting base and a spring in the radio-frequency ablation catheter according to the first embodiment of the present invention; FIG. 6 is a first sectional view of the connecting base in the radio-frequency ablation catheter according to the first embodiment of the present invention; FIG. 7 is a second sectional view of the connecting base in the radio-frequency ablation catheter according to the first embodiment of the present invention; FIG. 8 is a schematic structural view showing the overall structure of an infiltration hood in the radio-frequency ablation catheter according to the first embodiment of the present invention; FIG. 9 is a sectional view of the infiltration hood in the radio-frequency ablation catheter according to the first embodiment of the present invention; FIG. 10 is a schematic view showing a second tube sleeve connected to the infiltration hood in the radio-frequency ablation catheter according to the first embodiment of the present invention; FIG. 11 is a left side view of the fixing base in the radio-frequency ablation catheter according to the first embodiment of the present invention; and FIG. 12 is a schematic structural view of a claw-shaped electrode in the radio-frequency ablation catheter according to the first embodiment of the present invention.

As shown in FIG. 1, a radio-frequency ablation catheter according to an embodiment includes a handle portion 2, a needle tube portion 1, a central electrode 3 and a detection mechanism. The needle tube portion 1 includes a first tube sleeve 11 and a second tube sleeve 12. The handle portion 2 includes a cylinder sleeve 21 and a sliding button 22. The central electrode 3 includes an electrode body 31, an electrode wire 26, and an electrode connector 23. The detection mechanism includes a fixing ring 4, a fixing base 5, a traction wire (not shown), a connecting base 6, and multiple claw-shaped electrodes 7. With a user as a reference, the end close to the user is the proximal end and the end away from the user is the distal end. The radio-frequency ablation catheter includes sequentially, from a proximal end to a distal end, the cylinder sleeve 21, the first tube sleeve 11, the fixing ring 4, the fixing base 5, the connecting ring 6, the second tube sleeve 12, and the central electrode 3. The cylinder sleeve 21 is located at a proximal end of the needle tube portion 1, the first tube sleeve 11 is sleeved at a distal end of the cylinder sleeve 21, the electrode connector 23 is located at a proximal end of the cylinder sleeve 21, a proximal end of the electrode wire 26 is electrically connected to the electrode connector 23, and a distal end of the electrode wire 26 is inserted into the electrode body 31, and electrically connected to the electrode body 31. The electrode body 31 is located at a distal end of the needle tube portion 1, and fixed at a distal end of the second tube sleeve 12. The electrode body 31 is made of fibers. The electrode wire 26 extends through the cylinder sleeve 21, the first tube sleeve 11, the fixing base 5, the connecting base 6, the second tube sleeve 12 and the electrode body 31, respectively.

As shown in FIG. 12, the claw-shaped electrode 7 includes a first section 71, a second section 72, and a third section 73. The first section 71 is a proximal section, the second section 72 is a middle section, and the third section 73 is a distal section. The first section 71 is at an obtuse angle with respect to the second section 72, and the second section 72 is at an obtuse angle with respect to the third section 73. The claw-shaped electrode can obtain an impedance value of a contact position, or the claw-shaped electrode itself is made of a heat-sensitive material, to obtain the highest temperature around the claw-shaped electrode. In other words, by means of the claw-shaped electrode, the detection mechanism can detect the temperature or impedance at the claw-shaped electrode, and thus determine the progress of ablation.

As shown in FIG. 4, the fixing base 5 is located in the first tube sleeve 11, and the fixing base 5 can slidably move in the first tube sleeve 11. Four mounting holes 51 are provided on the fixing base 5, and the four mounting holes 51 are used to fix the claw-shaped electrodes 7. Optionally, the claw-shaped electrode 7 is fixed in the mounting hole 51 by dispensing. That is, the claw-shaped electrode 7 is fixedly provided in the mounting hole 51 by an adhesive. Alternatively the claw-shaped electrode 7 is fixed in the mounting hole 51 by welding. That is, the claw-shaped electrode 7 is welded to the mounting hole 51. A through hole 52 is provided along the center line of the fixing base 5, and the four mounting holes 51 are symmetrically distributed with the through hole 52 as the center. The through hole 52 is provided for accommodating a signal conduit and a liquid injection tube 25. The traction wire (not shown) is fixed at a distal end of the fixing base 5.

As shown in FIG. 3, the fixing ring 4 is located in the first tube sleeve 11 at a proximal end of the fixing base 5, a distal end of the fixing ring 4 is fixedly on the claw electrode 7, and a proximal end of the fixing ring 4 is used to fix an external wire, and allows the wire and the claw-shaped electrode to be electrically connected. The external wire extends through the cylinder sleeve 21 and the first tube sleeve 11, a proximal end of the external wire is fixed to the electrode connector 23, and a distal end of the external wire is fixed to the fixing ring 4. The first section 71 of the claw-shaped electrode 7 extends out of the fixing base 5, and enter the inside of the fixing ring 4. The distal end of the external wire enters the inside of the fixing ring 4 from a proximal end of the fixing ring 4. In the fixing ring 4, the first section 71 of the claw-shaped electrode 7 is in contact with the distal end of the external wire, to form an electrical connection. The electrode connector 23 is externally connected to a radio-frequency ablation system. A current is flowed to the claw-shaped electrode 7 through the external wire, and then the current is released into the human tissue by the claw-shaped electrode 7. The sliding button 22 is mounted on the surface of the cylinder sleeve 21, and the sliding button 22 can slidably move on the surface of the cylinder sleeve 21. A distal end of the traction wire (not shown) is fixed to the fixing base 5, and a proximal end of the traction wire (not shown) is fixed to the sliding button 22. By pushing the sliding button 22 to move forward or backward, the traction wire is driven to move forward or backward. Since one end of the traction wire is fixed to the fixing base 5, the traction wire (not shown) drives the fixing base 5 to slide in the first tube sleeve 11. The claw-shaped electrode 7 is fixed in the fixing base 5. As the fixing base 5 slides forward or backward, the claw-shaped electrode 7 fixed in the fixing base 5 will also move forward or backward with the fixing base 5. Optionally, the claw-shaped electrode 7 is fixedly connected to the fixing base 5 by an adhesive, and they move as a whole.

As shown in FIG. 7, the connecting base 6 is located between the first tube sleeve 11 and the second tube sleeve 12, and the first tube sleeve 11 and the second tube sleeve 12 are respectively fixed at a proximal and distal end of the connecting base 6. Four guide holes 61 are provided on the connecting base 6, and the openings of each two adjacent guide holes 61 extend in same direction. The four guide holes 61 are respectively located in four directions on the connecting base 6, and the four guide holes 61 are distributed in a circumferential array with the central axis of the connecting base 6 as a center. Therefore, among the four guide holes 61, the distance between each two adjacent guide holes 61 is the same; and the distance between each pair of opposing guide holes 61 is different. The claw-shaped electrodes 7 slide through the guide holes 61 in the connecting base 6, and these guide holes 61 make the claw-shaped electrodes 7 extend out of the connecting base 6 in a dispersed manner. The four claw-shaped electrodes 7 are separate from each other and do not interfere with each other. Since the claw-shaped electrode 7 is divided into the first section 71, the second section 72 and the third section 73, when the claw-shaped electrode 7 extends out of the connecting base 6, the first section 71 is still located inside the guide hole 61, and the second section 72 and the third section 73 extend out of the guide hole 61 of the connecting base 6 under the pushing force of the fixing base 5, Because of the obtuse angles between the first section 71 and the second section 72 and between the second section 72 and the third section 73, the claw-shaped electrodes 7 stretch out when they extend out of the connecting base 6, and they have the same length when they extend out of the needle tube portion 1. Since the positions of the four guide holes 61 in the connecting base 6 correspond to each other and the positions of the distal openings of the guide holes 61 are the same relative to the connecting base 6, when the claw-shaped electrodes 7 extend out of the guide holes 61, the distal ends of the claw-shaped electrodes 7 are at the same latitude.

The radio-frequency ablation catheter is used in a radio-frequency ablation system. The radio-frequency ablation system includes a radio-frequency generator (ablation instrument), wherein the radio-frequency generator (ablation instrument) is used to connect to the radio-frequency ablation catheter, and provides an electrical signal to the electrode connector of the radio-frequency ablation catheter, to allow the central electrode and the claw-shaped electrode to work.

In view of the foregoing description, it can be easily found that the radio-frequency ablation catheter of the present invention includes a handle portion 2, a needle tube portion 1, a central electrode 3, and a detection mechanism. The needle tube portion 1 includes a first tube sleeve 11 and a second tube sleeve 12. The handle portion 2 includes a cylinder sleeve 21 and a sliding button 22. The central electrode 3 includes an electrode body 31, an electrode wire 26, and an electrode connector 23. The detection mechanism includes a fixing ring 4, a fixing base 5, a traction wire, a connecting base 6, and multiple claw-shaped electrodes 7. For the detection mechanism, radio-frequency ablation catheter, and radio-frequency ablation system of the present invention, with a user as a reference, the end close to the user is the proximal end and the end away from the user is the distal end. The connecting base 6 is mounted between the first tube sleeve 11 and the second tube sleeve 12, and the fixing base 5 is located at a proximal end of the connecting base 6. The fixing base 5, the fixing ring 4 and the multiple claw-shaped electrodes 7 are all provided in the first tube sleeve 11. The fixing base 5 can slidably move in the first tube sleeve 11, the fixing ring 4 is mounted at a proximal end of the fixing base 5 and fixed to the claw-shaped electrode 7, and the claw-shaped electrodes 7 are inserted in the fixing base 5. The claw-shaped electrodes 7 can slide in the connecting base 6, and when the claw-shaped electrodes 7 extend out of the connecting base 6, the claw-shaped electrodes 7 stretch out, and are on the same latitude. The sliding button 22 can slide on the surface of the cylinder sleeve 21. A proximal end of the traction wire is fixed to the sliding button 22, and a distal end of the traction wire is fixed to the fixing base 5. The electrode body 31 is located at a distal end of the second tube sleeve 12, a distal end of the electrode wire 26 is fixed in the electrode body 31 and electrically connected, a proximal end of the electrode wire 26 is electrically connected to the electrode connector 23, and the electrode connector 23 is located outside the cylinder sleeve 21. The multiple claw-shaped electrodes 7 can be pushed in and pushed out of the needle tube portion 1 by the aid of the connecting 6, the fixing base 5, and the traction wire. When the claw-shaped electrode 7 needs to be pushed out of the needle tube portion 1 for detection, the sliding button 22 is pushed distally, so the sliding button 22 drives the traction wire to move distally, which in turn drives the fixing base 5 to move distally. At this time, the claw-shaped electrodes 7 fixed to the fixing base 5 move distally, as the fixing base 5 moves distally. As a result, the claw-shaped electrodes 7 previously received in the connecting base 6 are pushed out of the needle tube portion 1. When there is no need to use the claw-shaped electrodes 7 for detection, the sliding button 22 is pushed proximally, so the sliding button 22 drives the traction wire to move proximally, which in turn drives the fixing base 5 to move proximally, so as to pull the claw-shaped electrodes 7 back. At this time, the claw-shaped electrodes 7 previously located outside the needle tube portion 1 are retracted inside the connecting base 6 again. By means of the sliding button 22, the traction wire and the fixing base 5 are driven to move, to finally realize the push-out and retraction of the claw-shaped electrodes 7. This facilitates the control of the claw-shaped electrodes 7 by a user during surgery. When this detection device is used during surgery, the needle tube portion is inserted into the human body, for example, into the human trachea, then the claw-shaped electrodes are stretched out by using the sliding button, and the claw-shaped electrodes prop against the inner wall of human trachea, to stabilize the central electrode. At the same time, the claw-shaped electrodes can obtain the temperature or impedance data of the human trachea, so as to get the progress of ablation at the central electrode. Therefore, the detection device not only realizes the auxiliary positioning of the electrode body, but also get the progress of ablation by determining the temperature or impedance of the claw-shaped electrodes by the claw-shaped electrodes.

Optionally, in this embodiment, the radio-frequency ablation catheter also includes: a liquid injection joint 24 and a liquid injection tube 25, wherein the liquid injection joint 24 is located at a proximal end of the cylinder sleeve 21, and the liquid injection joint 24 is connected to an external injector. A proximal end of the liquid injection tube 25 is connected to the liquid injection joint 24, and a distal end of the liquid injection tube 25 is inserted into the electrode body 31. The liquid injection tube 25 is inserted in the cylinder sleeve 21, the first tube sleeve 11 and the second tube sleeve 12, and extends through the cylinder sleeve 21, the fixing base 5 and the connecting base 6 respectively. A liquid inlet 311 is provided on the electrode body 31, wherein the liquid inlet 311 is located in the middle of the electrode body 31, and the liquid inlet 311 is connected to a distal end of the liquid injection tube 25. Saline is discharged from the injector, flows through the liquid injection joint 24, the liquid injection tube 25 and the liquid inlet 311, and finally enters the interior of the electrode body 31.

As shown in FIG. 9, optionally, in this embodiment, the electrode body 31 includes: a cylindrical portion and a tapered portion, wherein the tapered portion is located at a distal end of the cylindrical portion. An infiltration hood 32 is provided outside the electrode body 31, wherein the infiltration hood 32 is made of high-temperature resistant insulating material, and the infiltration hood 32 is mounted around the cylindrical portion of the electrode body 31. Sprinkler channels 312 are provided inside the electrode body 31, and two sets of sprinkler channels 312 are distributed in a cross shape. The two sets of sprinkler channels are in communication with the liquid inlet 311. The saline flowing through the liquid injection tube 25 is concentrated in the liquid inlet 311 and then dispensed into the sprinkler channels 312. On the surface of the infiltration hood 32, multiple dispersing holes 321 are provided in a rectangular pattern, and the hole diameters of each row of dispersing holes 321 decrease in a direction from the proximal end to the distal end. The multiple dispersing holes 321 are in communication with the sprinkler channels 312, and arranged misaligned with the sprinkler channels 312. There is a gap between the infiltration hood 32 and the surface of the electrode body 31, and the saline in the sprinkler channel 312 flows through the gap to the infiltration hood 32. Due to the multiple dispersing holes 321 present on the surface of the infiltration hood 32, the saline flows out through the dispersing holes 321, to diffuse inside the body tissue.

Optionally, in this embodiment, the electrode body 31 is also provided with a temperature sensor 33 and a signal conduit therein. A wiring hole 34 is provided on the electrode body 31, and the wiring hole 34 is located on the side with the liquid inlet 311, wherein the signal conduit is inserted into the wiring hole 34. The temperature sensor 33 is located on the surface of the tapered portion of the electrode body 31, the temperature sensor 33 is electrically connected to a distal end of the signal conduit, and a proximal end of the signal conduit is fixed on the electrode connector 23 and electrically connected to the electrode connector 23. A rubber insulating layer 9 is provided outside the signal conduit. The temperature sensor 33 can be a thermistor, which is sensitive to temperature, and shows different resistance at different temperatures, wherein the resistance decreases as the temperature increases. After the saline is infiltrated into the human tissue from the dispersing holes 321, the ablation process starts. With the continuous increase of ablation saline, the ablation area continues to increase, and the temperature of the ablation area changes constantly. When the temperature becomes higher and higher, the resistance of the thermistor in a local area decreases as the temperature increases, and the thermistor and the signal conduit are electrically conducted. When the resistance of the thermistor changes, the resistance change is transmitted to the radio-frequency ablation system by the signal conduit (not shown). A distal end of the signal conduit is connected to the thermistor, and a proximal end of the signal conduit is fixed on the electrode connector 23. According to the resistance change on the radio-frequency ablation system, the local temperature change corresponding to the resistance is calculated. In this way, the temperature can be controlled by the flow rate of the brine. Moreover, the temperature change inside human tissue can be intuitively observed on the radio-frequency ablation system.

As shown in FIG. 8, optionally, in this embodiment, a slot 35 is provided at a proximal end of the electrode body 31, and an outer wall of the second tube sleeve 12 is inserted into the slot 35, to fix the electrode body 31 and the second tube sleeve 12 together. An internal thread 121 is provided at a proximal end of the second tube sleeve 12, an external thread 63 is provided at the distal end of the connecting base 6, and the connecting base 6 and the second tube sleeve 12 are threadedly connected.

Optionally, in this embodiment, a threaded hole 64 is provided at the proximal end of the connecting base 6, a counterbore 111 is provided at a distal end of the first tube sleeve 11, and a bolt is inserted into the counterbore 111 and the threaded hole 64 to fix the connecting base 6 and the first tube sleeve 11 together. As the bolt is continuously tightened, the connecting base 6 and the first tube sleeve 11 are fastened. When the bolt is fully screwed into the threaded hole 64, the surface of the bolt is parallel to the surface of the connecting base 6, and the bolt is tucked into the counterbore 111.

Optionally, in this embodiment, a liquid injection hole 62 is provided along the center line of the connecting base 6. The liquid injection tube 25 and the signal conduit are inserted in the liquid injection hole 62. The four guide holes 61 on the connecting base 6 are symmetrically distributed with the liquid injection hole 62 as a center. The liquid injection hole 62 is used to define the position of the liquid injection tube 25, making the sliding path of the liquid injection tube 25 not deviate. The guide hole 61 includes a straight section 611 and an arc section 612, wherein the straight section 611 is located at a proximal end of the arc section 612. Since the first section 71, the second section 72 and the third section 73 of the claw-shaped electrode 7 are connected and have an obtuse angle therebetween, the connection between the straight section 611 and the arc section 612 of the guide hole 61 can facilitate the claw-shaped electrode 7 to slide. The arc-shaped section 612 is convenient for the independent and smooth stretching and retraction of the claw-shaped electrode 7, to increase the supporting force and stretching range of the claw-shaped electrode 7. An anti-skid pattern 6111 is provided on an inner side wall of the straight section 611 of the guide hole 61. When the claw-shaped electrode 7 extends out of the needle tube portion 1, the anti-skid pattern 6111 in the straight section 611 acts to fix the position of the claw-shaped electrode 7 to keep it stable. An anti-wear cushion 6121 made of a silicone material is provided on the inner side wall of each arc section 612, and the anti-wear cushion 6121 is located between the arc-shaped section 612 and the claw-shaped electrode 7. When the claw-shaped electrode 7 is pushed out of or pulled into the connecting base 6, no friction that damages the surface of the claw-shaped electrode 7 occurs between the claw-shaped electrode 7 and the surface of the arc section 612. Therefore, the presence of the anti-wear cushion 6121 can relieve the wear on the surface of the claw-shaped electrode 7.

As shown in FIG. 5, optionally, in this embodiment, a mounting post 65 is provided at the proximal end of the connecting base 6, and a spring 8 is mounted around the mounting post 65. When the claw-shaped electrode 7 extends out of the needle tube portion 1, the fixing base 5 moves toward the connecting base 6. When the fixing base 5 moves toward the connecting base 6, a distal end of the spring 8 presses against the proximal end of the connecting base 6, and a proximal end of the spring 8 presses against the fixing base 5. The presence of the spring 8 increases the buffer capacity when the claw-shaped electrode 7 extends out of the connecting base 6, and keep the claw-shaped electrode 7 stable. As shown in FIG. 12, optionally, in this embodiment, the claw-shaped electrode 7 is hollow, and the surfaces of the first section 71 and the second section 72 of the claw-shaped electrode 7 are both provided with an insulating layer 9 made of a rubber material. When a current flows to the claw-shaped electrode 7 via a conductive tube, signal interference is caused. The presence of a layer of rubber on the surface of the claw-shaped electrode 7 can reduce signal interference, by signal shielding. The first section 71 and the second section 72 of the claw-shaped electrode 7 are both a straight section 611, and the third section 73 is an arc section.

As shown in FIG. 4, optionally, in this embodiment, two first grooves 53 are provided on the surface of the fixing base 5, a fixture block 54 is provided at the distal end of the traction wire, and the first groove 53 is used to fix the traction wire. When the traction wire needs to be clamped into the first groove 53, the fixture block 54 on the traction wire can be directly pushed into the first groove 53, to allow the fixture block 54 on the traction line to be right clamped into the first groove 53. Through the cooperation of the block 54 and the first groove 53, the traction wire is fixed on the fixing base 5.

As shown in FIG. 11, optionally, in this embodiment, the fixing base 5 includes a male fixing ring 554 and a female fixing ring 564. The fixing base 5 is divided into a front fixing ring 4 and a rear fixing ring 4. The male fixing ring 554 and the female fixing ring 564 are joined to form a complete fixing base 5. The male fixing ring 554 and the female fixing ring 564 are hinged at one end by a hinge shaft 57. A second groove 561 is provided on the other end of the female fixing ring 564, a bump 551 is provided at the other end of the male fixing ring 554, and the male fixing ring 554 and the female fixing ring 564 are fastened by inserting the bump 551 into the second grove 561.

The second embodiment is an alternative to the first embodiment, with the difference that the connecting base 6 is made of a fiberglass material, having good insulation performance, strong heat resistance, high anti-corrosion effect, and high mechanical strength.

The third embodiment is an alternative to the first embodiment, with the difference that the fixing base 5 is made of an electric ceramic material, which is a porcelain insulating material having good insulation performance and mechanical strength, excellent mechanical performance, good electrical performance, and high environmental resistance.

The fourth embodiment is an alternative to the first embodiment, with the difference that the fixing ring 4 is made of a silicone rubber material, having high-temperature stability, and able to maintain a certain degree of flexibility and elasticity in a high-temperature environment.

Radiofrequency Ablation Catheter and Radiofrequency Ablation System

A radiofrequency ablation catheter previously developed by the inventor includes a handle and a catheter assembly connected with the handle. A distal end of the catheter assembly is provided with a central electrode and a peripheral electrode assembly surrounding the central electrode. The central electrode can be percutaneously punctured and transmit radiofrequency to the cell tissue around the puncture site, so that the tumor cells contacted by the central electrode undergo coagulation, degeneration, and necrosis. The peripheral electrode assembly is slidable in the axial direction and includes a plurality of peripheral electrodes spaced from each other in the circumferential direction. Through the peripheral electrode assembly, the conditions around the central electrode, such as temperature and/or impedance, can be obtained, and then the ablation progress can be monitored and determined.

On this basis, the inventor of the present invention further discovered in research that the peripheral electrode assembly of this radiofrequency ablation catheter is likely to puncture/pierce the blood vessel when it enters the lesion site, resulting failure of the operation or bringing treatment risks or complications to the patient. To solve this problem, the inventor tried to rotate the handle of the radiofrequency ablation catheter to rotate the catheter assembly and thus the peripheral electrode assembly so that blood vessels can pass through the gaps between the peripheral electrodes of the electrode assemblies, thereby avoiding puncturing the blood vessels. However, because the catheter assembly of the radiofrequency ablation catheter is long and thin, it is difficult to rotate the peripheral electrode assembly located at the distal end of the catheter assembly by directly rotating the handle.

In addition, the inventor of the present invention also found that because the peripheral electrode assembly of the radiofrequency ablation catheter slides integrally in the axial direction, some of the peripheral electrodes cannot be inserted into the desired area for detecting the lesion, and the positioning effect is poor, resulting a poor reliability for monitoring the ablation results.

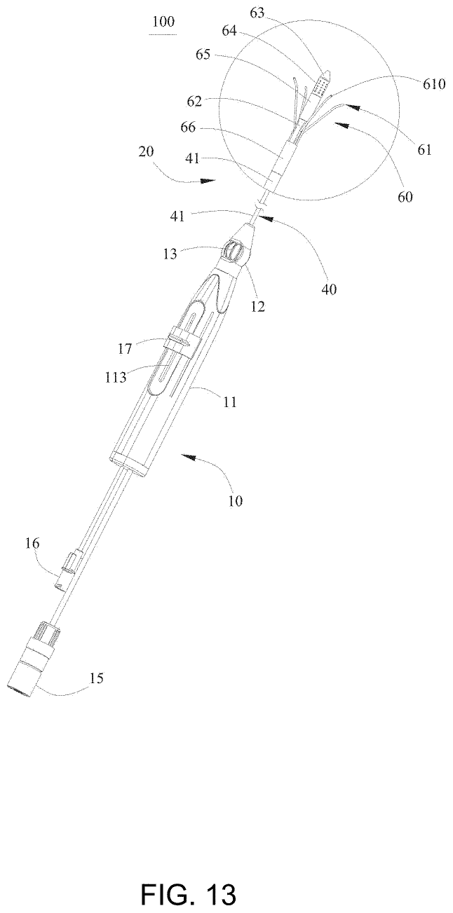

Referring to FIG. 13, a radiofrequency ablation catheter 100 according to a first embodiment of the present invention includes a handle 10 and a catheter assembly 20 connected with the handle 10. The catheter assembly 20 includes an outer catheter assembly 40 connected with the handle 10 and an inner catheter assembly 60 connected with the handle 10. The inner catheter assembly 60 is configured to be driven by the handle 10 to rotate relative to the outer catheter assembly 40. Specifically, a proximal end of the outer catheter assembly 40 is connected with a distal end of the handle 10. The inner catheter assembly 60 includes a peripheral electrode assembly 61. A distal end of the peripheral electrode assembly 61 can be driven to protrude from a distal end of the outer catheter assembly 40. Preferably, the distal end of the peripheral electrode assembly 61 is configured to be driven by operating the handle 10 to protrude from the distal end of the outer catheter assembly 40. More preferably, the distal end of the peripheral electrode assembly 61 is configured to be driven to expand outwardly and protrude from the distal end of the outer catheter assembly 40 in a claw shape. The peripheral electrode assembly 61 includes a plurality of peripheral electrodes 610 spaced from each other in the circumferential direction.

It can be concluded that a user can operate the handle 10 to rotate the peripheral electrode assembly 61 relative to the outer catheter assembly 40. Therefore, during introducing the catheter assembly 20 of the radiofrequency ablation catheter 100 into the lung under the guidance of B-scan ultrasonography or CT, the blood vessel is allowed to pass through the gap between the adjacent peripheral electrodes 610 of the peripheral electrode assembly 61, thereby preventing the peripheral electrode assembly 61 from puncturing the blood vessel. Furthermore, rotating the outer catheter assembly 40 is not required, which is convenient for operation.

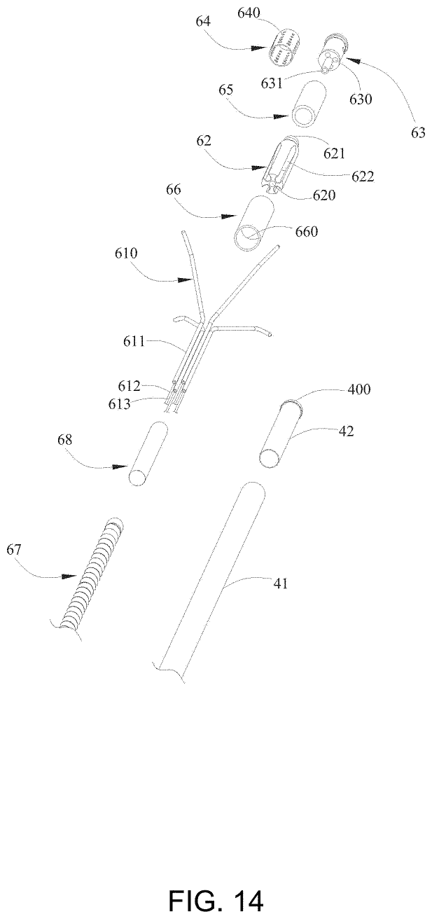

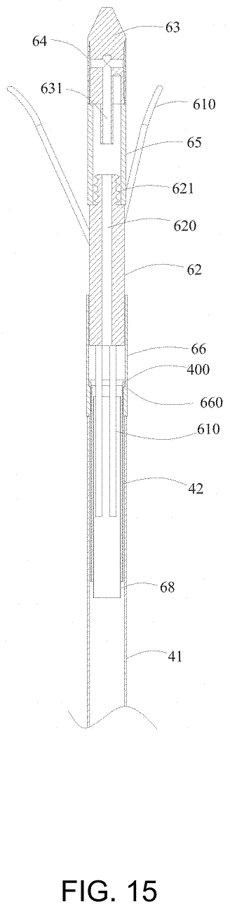

Referring to FIGS. 13 to 15, in this embodiment, each peripheral dispersedly electrode 610 of the peripheral electrode assembly 61 includes a probe 611, and a sensor wire 612 and a conducting wire 613 which are connected with the probe 611. After the peripheral electrodes 610 are introduced into the human tissue, such as the lung, the offset angles of the distal ends of the probes 611 of the peripheral electrodes 610 relative to the central axis of the radiofrequency ablation catheter 100 can be the same or different.

Optionally, the inner catheter assembly 60 further includes a support element 62 for supporting the peripheral electrode assembly 61. The support element 62 is generally cylindrical, and the peripheral electrodes 610 of the peripheral electrode assembly 61 are spaced from each other along the outer periphery of the support element 62. In this embodiment, the support element 62 is arranged at the distal end of the outer catheter assembly 40 and is rotatable, together with the peripheral electrode assembly 61, relative to the outer catheter assembly 40. Furthermore, the peripheral electrode assembly 61 is axially slidable relative to the support element 62.

In this embodiment, the inner catheter assembly 60 further includes a central electrode 63 fixedly connected with a distal end of the support element 62. Optionally, the support element 62 is provided with a through hole 620 extending therethrough in the axial direction at the center thereof for accommodating a sensor wire and a conducting wire for the central electrode 63 and a saline pipeline (not shown). A proximal side of the central electrode 63 is recessed to form a plurality of connecting grooves 630. The sensor wire and the conducting wire for the central electrode 63 are respectively received in respective connecting grooves 630 and then enter the through hole 620 of the support element 62. A hollow liquid inlet post 631 is provided on and protrudes from the center of the proximal side of the central electrode 63 for accommodating the saline pipeline extending from the through hole 620 of the support element 62. Preferably, the inner catheter assembly 60 further includes an infiltration cover 64 surrounding the outer peripheral surface of the central electrode 63, and a plurality of infiltration holes 640 are provided on and radially pass through the infiltration cover 64. Preferably, the infiltration holes 640 are evenly spaced from each other in the circumferential direction and the axial direction. The saline in the saline pipeline enters the central electrode 63 through the liquid inlet post 631, and flows out from the infiltration holes 640 through channels in the central electrode 63 (not shown), thereby being dispersed in the human tissue, such as the lung tissue.

In this embodiment, the inner catheter assembly 60 further includes a first connecting sleeve 65 for connecting the support element 62 and the central electrode 63. The first connecting sleeve 65 has a shape of a hollow cylinder, and preferably, is made of plastic. Preferably, a distal end of the first connecting sleeve 65 is melted and fitted with the infiltration cover 64 and/or the central electrode 63. More preferably, a metal wire (not shown) is used for connecting the first connecting sleeve 65 and the central electrode 63 to strengthen the connection therebetween. One end of the metal wire can be fixed within one of the connecting grooves 630 on the proximal side of the central electrode 63, the other end can be melted and connected with the first connecting sleeve 65. Preferably, a hollow threaded post 621 aligned with the through hole 620 can be provided on and protrude from a distal end of the support element 62. A proximal end of the first connecting sleeve 65 is melted and fitted to the outer circumference of the threaded post 621 to strengthen the connection between the first connecting sleeve 65 and the support element 62 and prevent the first connecting sleeve 65 and the elements connected with the first connecting sleeve 65, such as the central electrode 63, from falling off from the support element 62.

In this embodiment, a proximal side of the support element 62 is opposite to a distal side of the outer catheter assembly 40. Optionally, the proximal side of the support element 62 and the distal side of the outer catheter assembly 40 can abut against each other, so that the outer catheter assembly 40 can stably support the support element 62 in the axial direction, thereby avoiding a proximal movement of the support element 62 when rotating together with the peripheral electrode assembly 61. Alternatively, a certain distance may also be reserved between the proximal side of the support element 62 and the distal side of the outer catheter assembly 40, so that the outer catheter assembly 40 can support the support element 62 (through a rotatable element 66 which will be described below), and the support element 62 is also allowed to freely rotate together with the peripheral electrode assembly 61.

In this embodiment, the inner catheter assembly 60 further includes a rotatable element 66 for connecting the support element 62 and the outer catheter assembly 40. The rotatable element 66 is generally tubular. A distal end of the rotatable element 66 is fixedly connected with the support element 62, for example, by interference fit or welding. A proximal end of the rotatable element 66 is rotatably connected with the outer catheter assembly 40.

Preferably, the outer wall of the support element 62 has a plurality of first grooves 622 spaced in the circumferential direction, and the length direction of the first grooves 622 corresponds to the length direction of the support element 62. In this embodiment, the length direction corresponds to the axial direction. Each peripheral electrode 610 is slidably received in a respective first groove 622, and the distal end of each peripheral electrode 610 can slide into or out from the rotatable element 66. The first groove 622 helps the peripheral electrode 610 to slide steadily in the axial direction, thereby avoiding deflection of the peripheral electrode 610. Preferably, the rotatable element 66 covers a section of the plurality of first grooves 622 along the length direction, so that the distal section of the plurality of first grooves 622 is still visible, which is convenient for the user to determine the rotation direction of the support element 62 and thus the rotation direction of the peripheral electrode assembly 61, so that the peripheral electrode assembly 61 is allowed to conveniently avoid the blood vessels.

Preferably, the inner wall of the rotatable element 66 is provided with a first protrusion 660 protruding radially and inwardly, and the distal end of the outer catheter assembly 40 is provided with a second protrusion 400 protruding radially and outwardly. The second protrusion 400 is rotatably supported at a distal end of the first protrusion 660, so that the rotatable element 66 is allowed to rotate with the support element 62 and the peripheral electrode assembly 61 relative to the outer catheter assembly 40, and a distal movement of the rotatable element 66 when rotating together with the support element 62 and the peripheral electrode assembly 61 relative to the outer catheter assembly 40 is also avoided. Furthermore, the rotatable element 66 and the elements connected, such as the support element 62, are also prevented from falling off from the outer catheter assembly 40.

Preferably, the outer catheter assembly 40 includes an outer sheath 41 and a connecting tube 42 that are fixedly connected with each other. A proximal end of the outer sheath 41 is connected with the distal end of the handle 10. Preferably, the proximal end of the outer sheath 41 is fixedly connected with the distal end of the handle 10. The connecting tube 42 is received within a distal section of the outer sheath 41 and is fixedly connected with the outer sheath 41 by, for example, interference fit or welding. The second protrusion 400 is provided at a distal side of the connecting tube 42 for connecting with the rotatable element 66.

More preferably, a distal side of the outer sheath 41 abuts against a proximal side of the rotatable element 66, thereby avoiding a proximal movement of the rotatable element 66 when rotating together with the support element 62 and the peripheral electrode assembly 61.

Optionally, in this embodiment, the inner catheter assembly 60 further includes a spring tube 67 received within the outer catheter assembly 40, and a distal end of the spring tube 67 is fixedly connected with the probes 611 of the peripheral electrode assembly 61 directly or indirectly. In this embodiment, the inner catheter assembly 60 further includes a second connecting sleeve 68 for connecting the spring tube 67 and the probes 611 of the peripheral electrode assembly 61. A proximal end of the second connecting sleeve 68 surrounds around a distal outer periphery of the spring tube 67 and is fixedly connected with the distal section of the spring tube 67. A distal end of the second connecting sleeve 68 surrounds around proximal ends of the probes 611 of the peripheral electrode assembly 61 and is fixedly connected with the proximal ends of the probes 611 of the peripheral electrode assembly 61.

In this embodiment, the conducting wires 613 and the sensor wires 612 of the peripheral electrode assembly 61 extend inside of the spring tube 67 towards the proximal end (handle 10). The saline pipeline and the sensor wire and conducting wire for the central electrode 63 extend proximally from the through hole 620 of the support element 62 and through an annular gap between the second connecting sleeve 68 and the connecting tube 42, and further extend through an annular gap between the spring tube 67 and the outer sheath 41 towards the proximal end (handle 10).

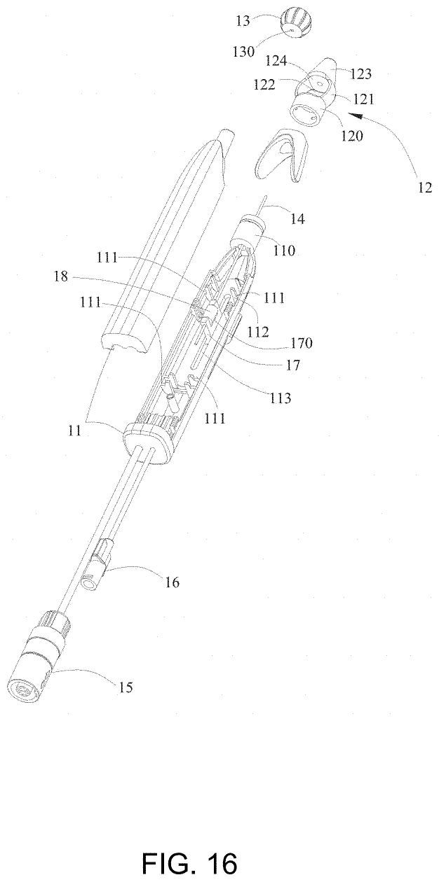

Referring to FIGS. 13 and 16, in this embodiment, the handle 10 includes a housing 11, an end cap 12 connected with a distal end of the housing 11, a rotatable ball 13 rotatably received in the end cap 12, and a push-pull rod 14 connected with the rotatable ball 13 in an anti-rotation manner.

Specifically, in this embodiment, the housing 11 generally has a shape of a hollow cylinder, and includes two half-housings that are connected with each other in a snap-fit connection. A distal end of one of the half-housings has a cylindrical inner connector 110. The end cap 12 includes a hollow cylindrical outer connector 120 that covers an outer circumference of the inner connector 110 and is connected with the inner connector 110, a plurality of arc-shaped connecting pieces 121 that are fixedly connected with a distal end of the outer connector 120 and enclose a space 122, and a tapered portion 123 fixedly connected with distal ends of the plurality of arc-shaped connecting pieces 121. An opening 124 is defined between adjacent arc-shaped connecting pieces 121. The outer sheath 41 is inserted into the tapered portion 123 and is fixedly connected with the tapered portion 123 in this embodiment. The rotatable ball 13 is received in the space 122, and the user can rotate the rotatable ball 13 at the opening 124. A proximal end of the push-pull rod 14 is received in the housing 11, and a distal end thereof passed through the inner connector 110, the rotatable ball 13 into the outer sheath 41, and is connected with the spring tube 67 directly or indirectly. In this embodiment, the push-pull rod 14 is directly connected with the spring tube 67.

Preferably, the push-pull rod 14 is hollow, and the conducting wires 613 and the sensor wires 612 of the peripheral electrode assembly 61 extend proximally from the aforementioned spring tube 67 into the push-pull rod 14 and further extend proximally until connected with an electrode connector 15 that is fixed with a proximal end of the housing 11. More preferably, the housing 11 has an accommodating space 111 for accommodating the saline pipeline and the sensor wires and the conducting wires for the central electrode 63 that extend proximally from the annular gap between the spring tube 67 and the outer sheath 41 into the housing 11 as described above. Preferably, the accommodating space 111 is enclosed by an inner wall of the housing 11 and a curved post 112 (such as an L-shaped post) protruding from the inner wall. More preferably, the housing 11 has two accommodating spaces 111 which are opposite to each other in the radial direction. The sensor wire and conducting wire for the central electrode 63 are accommodated in one accommodating space 111 and extend proximally until connected with the electrode connector 15 that is fixed with the proximal end of the housing 11. The saline pipeline is accommodated in the other accommodating space 111 and extends proximally until connected with a saline pipeline connector 16 that is fixed with the proximal end of the housing 11.

When a rotation of the peripheral electrode assembly 61 is required, the user can rotate the rotatable ball 13. As the rotatable ball 13 is connected with the push-pull rod 14 in an anti-rotation manner, the push-pull rod 14 will rotate together with the rotatable ball 13. The rotation of the push-pull rod 14 drives the inner catheter assembly 60 (including the spring tube 67, the second connecting sleeve 68, the peripheral electrode assembly 61, the support element 62, the rotatable element 66, the first connecting sleeve 65, and the central electrode 63 and the infiltration cover 64 connected with the connecting sleeve 65) to rotate.

In this embodiment, the peripheral electrode assembly 61 can be driven to move in the axial direction relative to the support element 62 and the rotatable element 66 by driving the push-pull rod 14 to move in the axial direction, so that the distal ends of the probes 611 of the peripheral electrode assembly 61 can move out from the rotatable element 66 or into the rotatable element 66.

Specifically, the handle 10 further includes a slidable assembly which includes a slide button 17 slidably connected with the housing 11, a fixing block 18 rotatably connected with the slide button 17, and the push-pull rod 14. More specifically, a side wall of the housing 11 has an elongated hole 113 that passes through the housing 11 in the radial direction. The slide button 17 is received in the elongated hole 113, with a portion thereof protruding from an outer surface of the housing 11 for the user to move the slide button 17, and the other portion thereof received in the housing 11. The other portion of the slide button 17 has a receiving groove 170 that is opened at a radial inside thereof. The fixing block 18 is received in the receiving groove 170 and preferably abuts against two opposite walls of the receiving groove 170 in the axial direction. More preferably, the fixing block 18 is cylindrical, so that the fixing block 18 can rotate stably relative to the slide button 17. The proximal end of the push-pull rod 14 passes through the slide button 17 and the fixing block 18, in which the push-pull rod 14 is fixedly connected with the fixing block 18 by, for example, interference fit, and the push-pull rod 14 is movably connected with the slide button 17 by, for example, clearance fit, so that the push-pull rod 14 is allowed to rotate relative to the slide button 17, and move by the movement of the slide button 17 through the fixing block 18, thereby driving the spring tube 67, the second connecting sleeve 68, and the peripheral electrode assembly 61 to move in the axial direction through the push-pull rod 14.

Preferably, the rotatable ball 13 has a noncircular hole 130 at the center thereof, such as a square hole, an elliptical hole, etc., and the push-pull rod 14 passes through the rotatable ball 13 through the noncircular hole 130 and engages with the wall of the noncircular hole 130, so that the push-pull rod 14 is allowed to move in the axial direction relative to the rotatable ball 13 and to rotate together with the rotatable ball 13.

When using the ablation catheter according to the first embodiment, the distal end of the peripheral electrode assembly 61 is initially received in the rotatable element 66. The distal end of the catheter assembly 20 (i.e., the central electrode 63) can be used to percutaneously puncture into the target tumor. Then, the rotatable ball 13 can be rotated, wherein the rotation direction can be determined through the visible section (distal section) of the four first grooves 622 of the support element 62, so that the four first grooves 622 are allowed to avoid blood vessels. After the central electrode 63 is rotated to a safe and desired position, the central electrode 63 can be inserted into the lesion. Thereafter, the slide button 17 can be pushed to push the peripheral electrode assembly 61 out of the rotatable element 66 to obtain the surrounding conditions of the central electrode 63, such as temperature and impedance, thereby determining the progress of ablation. Since the first grooves 622 avoid the blood vessels, the peripheral electrodes 610 of the peripheral electrode assembly 61 pushed out from the rotatable element 66 will also avoid the blood vessels without puncturing the blood vessel.

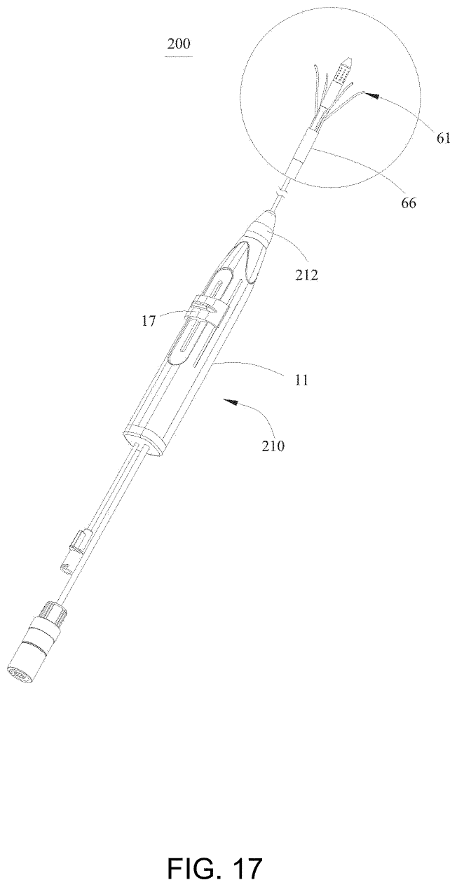

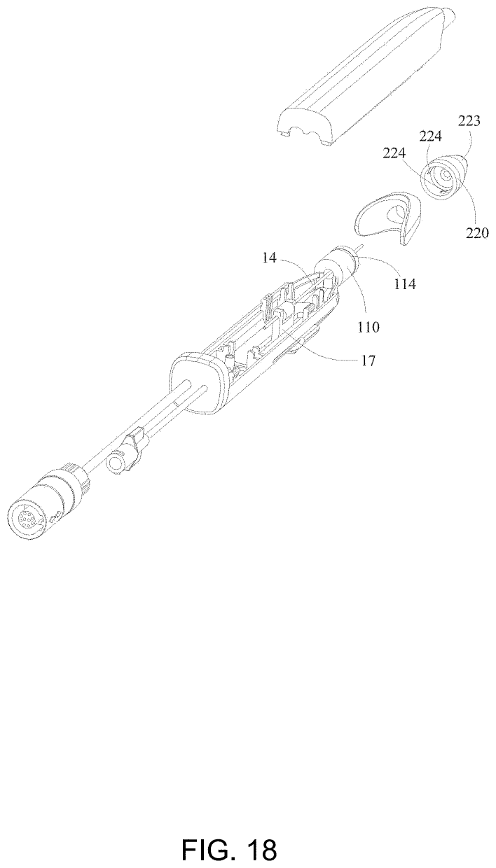

Referring to FIGS. 17 and 18, the radiofrequency ablation catheter 200 according to a second embodiment of the present invention is similar to the radiofrequency ablation catheter 100 according to the first embodiment, and the radiofrequency ablation catheter 200 according to a second embodiment of the present invention differs from the radiofrequency ablation catheter 100 according to the first embodiment in the configuration for rotating the peripheral electrode assembly 61.

Specifically, the handle 210 of the radiofrequency ablation catheter 200 according to the second embodiment of the present invention no longer includes the end cap 12 and the rotatable ball 13 as shown in the first embodiment. In this second embodiment, the end cap 212 of the handle 210 only includes a hollow cylindrical outer connector 220 that covers the outer circumference of the inner connector 110 and is rotatably connected with the inner connector 110, and a tapered portion 223 fixedly connected with a distal end of the outer connector 220. The outer sheath 41 is inserted into the tapered portion 223, and is fixedly connected with the tapered portion 223 in this embodiment.

The inner connector 110 and the outer connector 220 can be rotatably connected using the following structures: the outer circumference of the inner connector 110 has an annular recessed groove 114, and an inner wall of the outer connector 220 has one or more arc-shaped sliding rings 224 or an annular sliding ring 224 protruding therefrom, and the sliding ring 224 is rotatably received in the annular groove 114. Alternatively, in other embodiments, the inner connector 110 and the outer connector 220 can be rotatably connected using other structures.

Furthermore, the push-pull rod 14 in this embodiment is no longer in clearance fit with the slide button 17. Instead, the push-pull rod 14 in this embodiment is fixedly connected with the slide button 17 by, for example, interference fit.

In use, the user can hold the end cap 212 with one hand and rotate the housing 11 of the handle 210 with the other hand. Since the push-pull rod 14 is fixedly connected with the slide button 17 in this embodiment, the push-pull rod 14 will also rotate together with the housing 11, and then the peripheral electrode assembly 61 will be rotated through the spring tube 67 and the second connecting sleeve 68. When a movement of the peripheral electrode assembly 61 is required, the slide button 17 can be pushed or pulled to push or pull the distal ends of the probes 611 of the peripheral electrode assembly 61 out of the rotatable element 66 or into the rotatable element 66.

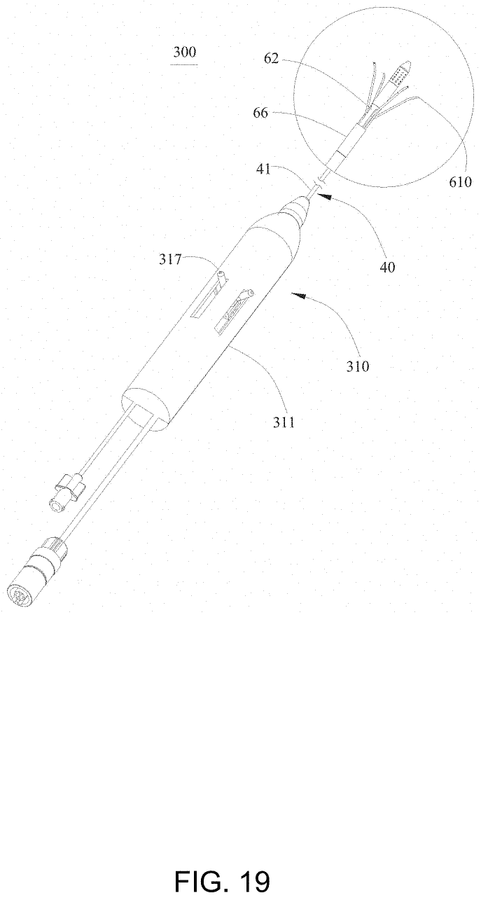

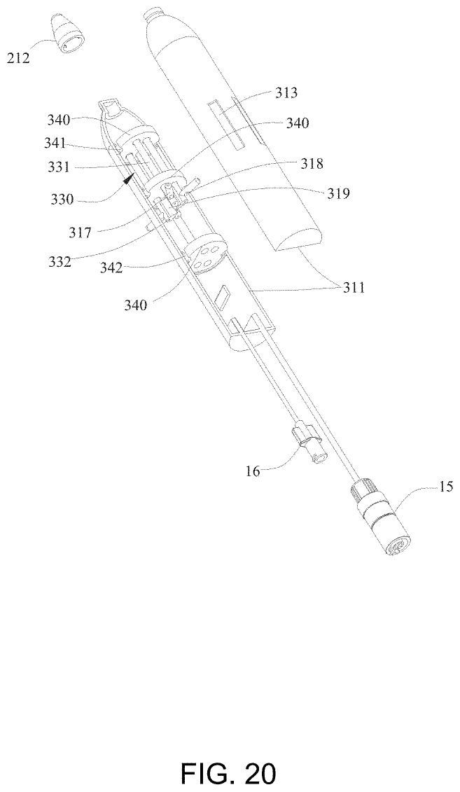

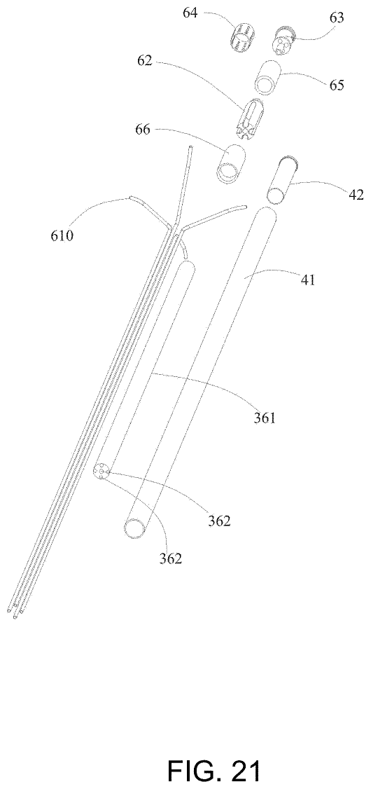

Referring to FIGS. 19-21, the radiofrequency ablation catheter 300 according to a third embodiment of the present invention is similar to the radiofrequency ablation catheter 200 according to the second embodiment, and the radiofrequency ablation catheter 300 according to the third embodiment of the present invention mainly differs from the radiofrequency ablation catheter 200 according to the second embodiment in the slidable assembly.

Specifically, in this embodiment, the slidable assembly of the radiofrequency ablation catheter 300 includes a plurality of slidable elements 330 that are independently slidable relative to the housing 311 in the axial direction. Each peripheral electrode 610 of the peripheral electrode assembly 61 is fixedly connected with a respective slidable element 330 and is slidable relative to the housing 311 in the axial direction.

It can be concluded that when using the radiofrequency ablation catheter 300 according to this embodiment, each peripheral electrode 610 can be respectively controlled to move in the axial direction so that each peripheral electrode 610 can be respectively controlled by controlling each slidable element 330 separately to move to a desired lesion site as required, thereby improving the positioning effect of each peripheral electrode 610 and thus improving the reliability of ablation monitoring results.

Optionally, the slidable element 330 includes a slidable rod 331 slidably arranged in the housing 311 and a slide button 317 fixedly connected with the slidable rod 331 and protruding from the outer surface of the housing 311. The peripheral electrode 610 is fixedly connected with a respective slidable rod 331. The slide button 317 is slidably connected with the housing 311.

Preferably, the handle 310 further includes at least one support seat 340 received in the housing 311. In this embodiment, the support seat 340 generally has a shape of a disc, and its outer peripheral wall abuts against and is fixedly connected with the inner peripheral wall of the housing 311. The slidable rod 331 axially passes through the at least one support seat 340 and is slidably connected with the at least one support seat 340. In this embodiment, the handle 310 includes three support seats 340 axially spaced from each other. The slidable rod 331 is slidably connected with the middle support seat 340 and the distal support seat 340. The support seats 340 can not only limit the movement of the slidable elements 330, so that the slidable elements 330 can move stably in the axial direction without deflection, but the support seats 340 can also support the housing 311 to prevent deformation of the housing 311.

More preferably, the outer periphery of the support seat 340 has at least one second groove 341 through the support seat 340 in the axial direction, and the inner wall of the housing 311 encloses the second groove 341 to form an accommodating space 342. In this embodiment, the outer periphery of each support seat 340 has two second grooves 341 opposite in the radial direction. The saline pipeline extends proximally from the through hole 620 of the support element 62 through the connecting tube 42 and the outer sheath 41 of the outer catheter assembly 40 into the housing 311, and through the accommodating spaces 342 at one radial side enclosed by the respective support seats 340 and the housing 311 until connected with the saline pipe connector 16 that is fixed with the proximal end of the housing 311. The sensor wire and conducting wire for the central electrode 63 extend proximally from the through hole 620 of the support element 62 through the connecting tube 42 and the outer sheath 41 of the outer catheter assembly 40 into the housing 311, and through the accommodating spaces 342 at the other radial side enclosed by the respective support seats 340 and the housing 311 until connected with the electrode connector 15 fixed with the proximal end of the housing 311. The accommodating spaces 342 can prevent the wires of the radiofrequency ablation catheter 300 from crossing with each other.

Preferably, the slidable rod 331 is provided with a first receiving hole 332 extending therethrough in the axial direction. The probes 611 of the peripheral electrodes 610 extend from the rotatable element 66 through the connecting tube 42 and the outer sheath 41 of the outer catheter assembly 40 to the respective first receiving holes 332 of the respective slidable rods 331 in the housing 311 and fixedly connected with the respective slidable rods 331. The sensor wires 612 and the conducting wires 613 of the respective peripheral electrodes 610 further extend proximally from the first receiving holes 332 through the proximal support seat 340 until connected with the electrode connector 15 fixed with the proximal end of the housing 311. The first receiving holes 332 and the proximal support seat 340 can effectively prevent the wires of the radiofrequency ablation catheter 300 from crossing with each other.

Preferably, the housing 311 has a plurality of elongated holes 313 spaced from each other along the circumferential direction. Each elongated hole 313 radially passes through the housing 311, and the length direction of each elongated hole 313 corresponds to the length direction of the housing 311. In this embodiment, the length direction corresponds to the axial direction. The slide button 317 of each slidable element 330 protrudes from the outer surface of the housing 311 through a respective elongated hole 313. The slide button 317 is limited by the elongated hole 313, so that the slide button 317 can stably slide in the elongated hole 313 in the axial direction, thereby effectively preventing the slide button 317 from deflection in the circumferential direction.

In this embodiment, one end of the slide button 317 of each slidable element 330 is vertically connected with the respective slidable rod 331, and preferably, the slide button 317 is arranged adjacent to the proximal end of the respective slidable rod 331. The side wall of the slide button 317 adjacent to its radially inside end has a recessed avoidance groove 318, and preferably the avoidance groove 318 is located on a side wall of the slide button adjacent to the proximal end of the slidable rod 331 to avoid interference between the slidable element 330 and the surrounding elements and/or wiring etc.

Preferably, the slidable element 330 further includes a wedge 319 arranged adjacent to the proximal end of the slidable rod 331 and located on the side of the slidable rod 331 opposite to the slide button 317. The slidable rod 331, the slide button 317, and the wedge 319 together form a substantially T-shaped structure. Specifically, the wedge 319 gradually tapers from the side wall of the slidable rod 331 toward the center of the slidable assembly (i.e., the center of the four slidable elements 330 or the center axis of the handle 310). The wedges 319 not only help the slidable elements 330 to slide stably in the axial direction, but also help to avoid interference between adjacent slidable elements 331.

More preferably, the inner catheter assembly 360 of the radiofrequency ablation catheter 300 according to this embodiment further includes a support rod 361 housed in the outer catheter assembly 40, and the support rod 361 has a plurality of second receiving holes 362 extending therethrough in the axial direction. Preferably, the support rod 361 has a second central receiving hole 362, and a plurality of second peripheral receiving holes 362 surrounding the second central receiving hole 362. Each of the peripheral electrodes 610 passes through a respective second peripheral receiving hole 362. The saline pipeline and the sensor wire and conducting wire for the central electrode 63 pass through the second central receiving hole 362. In other words, in this embodiment, the inner catheter assembly 360 no longer includes the spring tube 67 and the second connecting sleeve 68. The second receiving holes 362 can effectively prevent the wires of the radiofrequency ablation catheter 300 from crossing with each other.

In use, the user can hold the end cap 212 with one hand and rotate the housing 311 of the handle 310 with the other hand. Since the peripheral electrode assembly 61 is fixedly connected with the slidable assembly of the handle 310, the peripheral electrode assembly 61 will also rotate together with the housing 311. When a movement of one or more peripheral electrodes 610 of the peripheral electrode assembly 61 is required, the respective one or more slidable elements 330 can be pushed or pulled to push or pull the distal ends of the probe 611 of the respective peripheral electrodes 610 out of the rotatable element 66 or into the rotatable element 66.

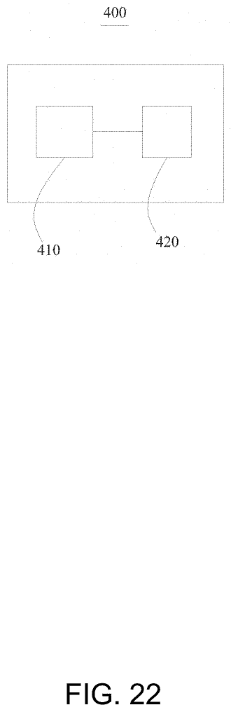

Referring to FIG. 22, a fourth embodiment of the present invention provides a radiofrequency ablation system 400 which includes a radiofrequency generator 410, and a radiofrequency ablation catheter 420 connected with the radiofrequency generator 410 according to any one of the foregoing embodiments. The radiofrequency generator 410 provides electrical signals for the central electrode 63 and the peripheral electrode assembly 61 of the radiofrequency ablation catheter 420 to make the central electrode 63 and the peripheral electrode assembly 61 work.

Perfusion Control

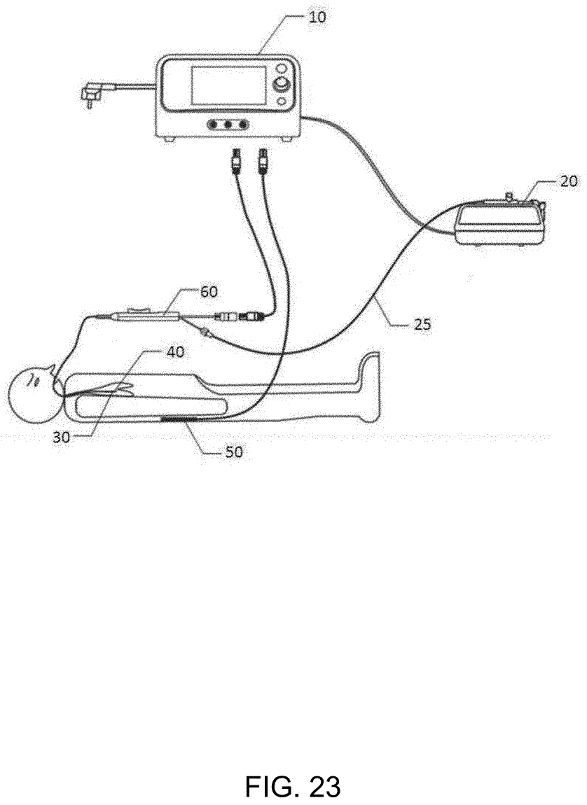

Reference is made to FIG. 23, which is a schematic diagram showing an application scenario of a method for controlling the perfusion of a syringe pump according to an embodiment of the present invention. The method for controlling the perfusion of the syringe pump may be implemented by a radio frequency ablation control apparatus 10 or a syringe pump 20 in FIG. 23. Optionally, the method for controlling the perfusion of the syringe pump may be implemented by other computer devices other than the radio frequency ablation control apparatus 10 or the syringe pump 20, such as a server, a desktop computer, a notebook computer, a laptop computer, a tablet computer, a personal computer and a smart phone.

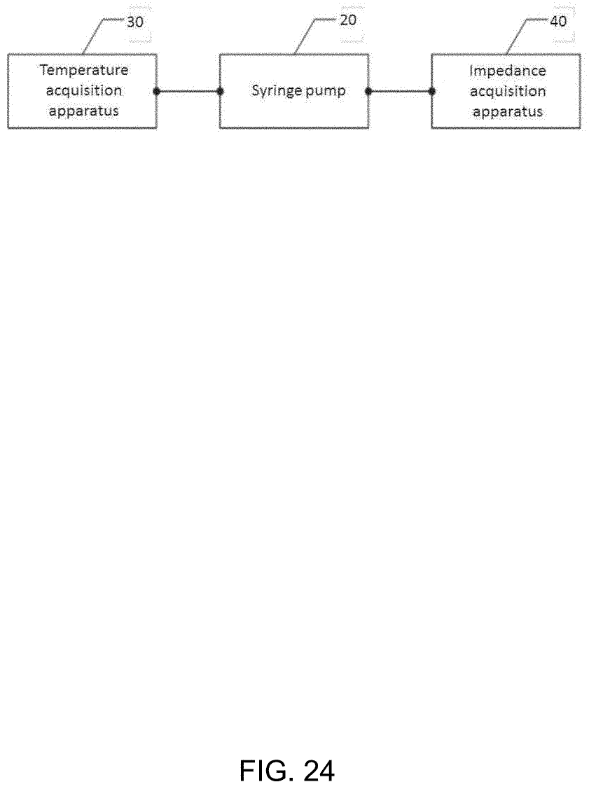

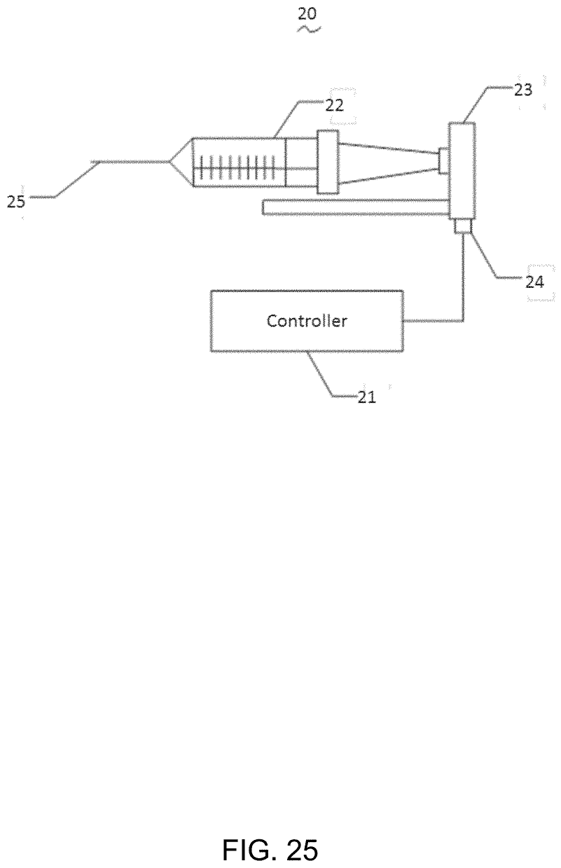

Particularly, when the method for controlling the perfusion of the syringe pump is implemented by the syringe pump 20, reference is made to FIG. 24 and FIG. 25, wherein FIG. 24 is a schematic diagram showing a structure of a system for controlling the perfusion of a syringe pump according to an embodiment of the present invention, and FIG. 25 is a schematic diagram showing a syringe pump in the system for controlling the perfusion of the syringe pump. As shown in FIG. 24, the system for controlling the perfusion of the syringe pump includes a syringe pump 20, a temperature acquisition apparatus 30 and an impedance acquisition apparatus 40. The temperature acquisition apparatus 30 and the impedance acquisition apparatus 40 are electrically coupled to the syringe pump 20.

As shown in FIG. 25, the syringe pump 20 includes a controller 21, a syringe 22, a push rod 23 and a drive apparatus 24. For ease of description, FIG. 25 only shows parts related to the embodiments of the present invention.

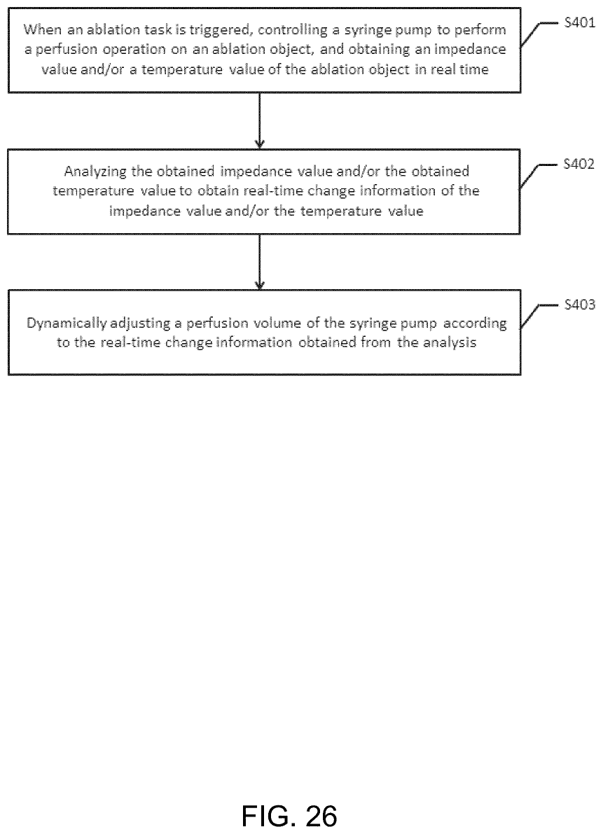

The temperature acquisition apparatus 30 and the impedance acquisition apparatus 40 are electrically coupled to the controller 21. The temperature acquisition apparatus 30 is configured to acquire a temperature value of an ablation object and transmit it to the controller 21. The impedance acquisition apparatus 40 is configured to acquire an impedance value of the ablation object and transmit it to the controller 21. The controller 21 is configured to perform steps in the method for controlling the perfusion of the syringe pump according to the following embodiments as shown in FIG. 26 to FIG. 31 so as to realize dynamic adjustment on the perfusion volume of the syringe pump.

The drive apparatus 24 is configured to drive the push rod 23 to move in a direction pointed by a control instruction transmitted by the controller 21 at a speed indicated by the control instruction according to the control instruction so as to control and adjust the perfusion volume of the syringe 22.

Further, the syringe pump 20 further includes an extension tube 25. As shown in FIG. 23, one end of the extension tube 25 is connected to a needle of the syringe 22 and the other end thereof is inserted into the ablation object and is close to an ablation site.

Optionally, the temperature acquisition apparatus 30 and the impedance acquisition apparatus 40 may be disposed at one end of the extension tube 25 close to the ablation site. Alternatively, the temperature acquisition apparatus 30 and the impedance acquisition apparatus 40 may further be disposed on other apparatuses, inserted into the ablation object through the other apparatuses and abutted against the ablation site.

In combination with FIG. 23 to FIG. 25, in practical applications, firstly, the radio frequency ablation catheter 60 configured to generate and output radio frequency energy and the extension tube 25 are inserted into the ablation object (such as a lung cancer patient) and reach the ablation site. Then, a neutral electrode 50 is brought into contact with the skin surface of the ablation object. A radio frequency current flows through the radio frequency ablation catheter 60, tissues of the patient and the neutral electrode 50, thereby forming a loop. When the ablation task is triggered, the radio frequency ablation control apparatus 10 controls the radio frequency ablation catheter 60 to output the radio frequency energy to the ablation site by means of unipolar discharge, so as to perform an ablation operation on the ablation site.

Meanwhile, the controller 21 of the syringe pump 20 controls the syringe pump 20 to perform a perfusion operation on the ablation object for perfusing saline into the ablation site, and obtains the impedance value and/or the temperature value of the ablation site through the temperature acquisition apparatus 30 and the impedance acquisition apparatus 40 in real time. The controller 21 obtains real-time change information of the impedance value and/or the temperature value by analyzing the obtained impedance value and/or the obtained temperature value, and then according to the real-time change information obtained from the analysis, instructs the drive apparatus 24 to drive the push rod 23 to move in the pointed direction at the indicated speed, so as to dynamically adjust the perfusion volume of the syringe pump 20.

Reference is made to FIG. 26, which is a flowchart of an implementation of a method for controlling the perfusion of a syringe pump according to an embodiment of the present invention. The method may be implemented by the syringe pump 20 in FIG. 23, or may be implemented by the radio frequency ablation control apparatus 10 in FIG. 23, or may be implemented by other computer devices electrically coupled to the syringe pump. As shown in FIG. 26, the method particularly includes the following steps.

In step S401, when the ablation task is triggered, the syringe pump is controlled to perform the perfusion operation on the ablation object, and the impedance value and/or the temperature value of the ablation object are/is obtained in real time.

Particularly, the ablation task may be triggered when for example preset trigger time is reached, a trigger instruction transmitted by the radio frequency ablation apparatus is received, or a notification event that a user performs an operation for triggering the ablation task is detected. Among them, the operation for triggering the ablation task is for example to press a physical or virtual button for triggering the ablation task.

Optionally, after each start of the syringe pump, a perfusion parameter is set to a preset initial value. The perfusion parameter may include but is not limited to: a perfusion flow rate, a total perfusion volume, perfusion time, and the like.

When the ablation task is triggered, the syringe pump performs the perfusion operation on the ablation object according to the above-mentioned initial value. Meanwhile, at least one of the impedance value and the temperature value of the ablation object is obtained in real time by the temperature acquisition apparatus and the impedance acquisition apparatus, as reference data for dynamically adjusting the perfusion volume of the syringe pump. The impedance value and the temperature value of the ablation object may be for example the impedance value and the temperature value of the ablation site. The impedance value may be a resistance value, for example.

Optionally, the impedance value and/or the temperature value of the ablation object may further be obtained in real time in the following fashions.

Firstly, an impedance sample value and/or a temperature sample value of the ablation object are/is obtained in real time, and the obtained impedance sample value and/or the obtained temperature sample value are/is filtered. Then, it is determined whether the filtered impedance sample value and/or the filtered temperature sample value exceed/exceeds a preset warning value range. On the one hand, if the filtered impedance sample value and/or the filtered temperature sample value exceed/exceeds the preset warning value range, alarm information is output to remind a user that the operation is abnormal and whether the user needs to stop the ablation operation. On the other hand, if the filtered impedance sample value and/or the filtered temperature sample value do/does not exceed the preset warning value range, a lowest or average value of the filtered impedance sample value and/or the filtered temperature sample value within a preset period (for example, within 10 seconds) is taken as the resistance value and/or the temperature value obtained in real time.

In step S402, the obtained impedance value and/or the obtained temperature value are/is analyzed to obtain real-time change information of the impedance value and/or the temperature value.

Particularly, at least one of the impedance value and the temperature value is analyzed to obtain the real-time change information of at least one of the impedance value and the temperature value. Optionally, the real-time change information may include but is not limited to a real-time change trend and a real-time change amplitude. Among them, the real-time change trend is a change direction, for example, the value increases or decreases. The real-time change amplitude is a change degree, which may be an absolute value or a ratio, for example, an increased value, or an increased percentage.

It should be noted that the real-time change trend and the real-time change amplitude mentioned above refer to an overall change trend and an overall change amplitude.

In step S403, the perfusion volume of the syringe pump is dynamically adjusted according to the real-time change information obtained from the analysis.

Particularly, the perfusion volume of the syringe pump is dynamically adjusted according to an analysis result from the step S402. If obtained from the analysis is the real-time change information of the temperature value, the perfusion volume of the syringe pump is dynamically adjusted according to the real-time change information of the temperature value. If obtained from the analysis is the real-time change information of the impedance value, the perfusion volume of the syringe pump is dynamically adjusted according to the real-time change information of the impedance value. If obtained from the analysis is the real-time change information of the temperature value and the real-time change information of the impedance value, the perfusion volume of the syringe pump is dynamically adjusted according to the real-time change information of the temperature value and the real-time change information of the impedance value at the same time.

It should be understood that the impedance of a human body is the total impedance containing resistance and capacitance, and the total impedance includes impedances of skin, blood, muscle, cell tissues and their joints of the human body, and is mainly determined by the resistance of the human body. When the human body is brought into contact with a charged body, the human body is regarded as a circuit element to be connected into the loop. In a cancer cell ablation process, in addition to the temperature affected by the radio frequency energy, the impedance of the human body will change therewith. Therefore, using the impedance and/or the temperature as a reference value for adjusting the perfusion volume may more accurately determine the impact of the radio frequency energy on the human body, thereby making the volume of the liquid perfused into the ablation site more accurate and more in line with ablation requirements.

Optionally, the step S403 may be particularly implemented by the following steps.

In S11, according to the real-time change information obtained from the analysis, it is determined whether the temperature value presents a rising trend or a falling trend.

In S12, if the temperature value presents the rising trend, the syringe pump is controlled to increase a perfusion flow rate according to a preset increase and a trend change in the impedance value or the temperature value.

In S13, if the temperature value presents the falling trend, the syringe pump is controlled to decrease the perfusion flow rate according to a preset decrease and the trend change in the impedance value or the temperature value.

Optionally, the step S403 may further be particularly implemented in the following fashions.

In S21, according to the real-time change information obtained from the analysis, it is determined whether the impedance value presents a rising trend or a falling trend.

In S22, if the impedance value presents the rising trend, the syringe pump is controlled to increase the perfusion flow rate according to a preset increase and a trend change in the impedance value or the temperature value.

In S23, if the impedance value presents the falling trend, the syringe pump is controlled to decrease the perfusion flow rate according to a preset decrease and the trend change in the impedance value or the temperature value.

Optionally, the step S403 may further be particularly implemented by the following steps:

In S31, it is analyzed whether the real-time change trend and the real-time change amplitude of the impedance value and the real-time change trend and the real-time change amplitude of the temperature value both meet respective corresponding adjustment conditions.

In S32, if the real-time change trend and the real-time change amplitude of the impedance value and the real-time change trend and the real-time change amplitude of the temperature value meet their respective corresponding adjustment conditions, then the perfusion volume of the syringe pump is dynamically adjusted according to the real-time change trend and the real-time change amplitude of the impedance value.

In S33, otherwise, if the real-time change trend and the real-time change amplitude of only one parameter of two parameters (i.e., the impedance value and the temperature value) meet the corresponding adjustment conditions, then the perfusion volume of the syringe pump is dynamically adjusted according to the real-time change trend and the real-time change amplitude of the parameter that meets the corresponding adjustment conditions.

The adjustment conditions mentioned above are conditions respectively met by the real-time change trend and the real-time change amplitude of the impedance value and the temperature value when the perfusion volume needs to be adjusted, for example, whether the real-time change trend and the real-time change amplitude of the impedance value and the temperature value present the rising trend or the falling trend, whether an increase reaches a preset amplitude and the like.

For the specific fashion for adjusting the perfusion volume in the steps S31 to S33, reference may be made to the relevant description of dynamically adjusting the perfusion volume of the syringe pump according to the real-time change information of the temperature value or the impedance value in the foregoing and following embodiments, which will be omitted here.

Optionally, the step S403 may further be particularly implemented by the following steps.

A current ablation stage is determined according to the real-time change trend and the real-time change amplitude of the impedance value, and then the perfusion volume of the syringe pump is adjusted according to a target adjustment logic corresponding to the current ablation stage.

That is, the whole ablation process is divided into multiple ablation stages according to the real-time change trend and the real-time change amplitude of the impedance value, and then the perfusion volume of the syringe pump is adjusted according to the corresponding adjustment logic based on a change characteristic of the impedance value of each ablation stage, so as to make the adjustment more targeted, thereby further improving the accuracy of the perfusion.

Further, that the current ablation stage is determined according to the real-time change trend and the real-time change amplitude of the impedance value, and then the perfusion volume of the syringe pump is adjusted according to the target adjustment logic corresponding to the current ablation stage may particularly include the following steps.

In S41, when the syringe pump starts to perfuse a liquid into the ablation object, it is determined that the ablation object enters a first ablation stage and a perfusion volume of the syringe pump is adjusted according to a first target adjustment logic based on the real-time change trend and the real-time change amplitude of the impedance value.

In S42, in the first ablation stage, when the impedance value presents a first change trend and reaches a first change amplitude, it is determined that the ablation object enters a second ablation stage and the perfusion volume of the syringe pump is adjusted according to a second target adjustment logic.

In S43, in the second ablation stage, when the impedance value presents a second change trend and reaches a second change amplitude, it is determined that the ablation object enters a third ablation stage and the perfusion volume of the syringe pump is adjusted according to a third target adjustment logic.

In S44, in the third ablation stage, when the impedance value presents a third change trend and reaches a third change amplitude, it is determined that the ablation object enters a fourth ablation stage and the perfusion volume of the syringe pump is adjusted according to a fourth target adjustment logic.

Optionally, in another embodiment of the present invention, in the entire process of performing the perfusion operation, when it is detected that the temperature value is greater than a first abnormal value or less than a second abnormal value for more than a first preset duration, the syringe pump is controlled to stop the perfusion operation and the ablation task is suspended, wherein the first preset duration is greater than or equal to zero. When the first preset duration is equal to 0, that is, as long as it is detected that the temperature value is greater than the first abnormal value or less than the second abnormal value, the syringe pump is immediately controlled to stop the perfusion operation.

Optionally, in another embodiment of the present invention, in the entire process of performing the perfusion operation, when it is detected that the impedance value is greater than a third abnormal value or less than a fourth abnormal value for more than a second preset duration, the syringe pump is controlled to stop the perfusion operation and the ablation task is suspended, wherein the second preset duration is greater than or equal to zero. When the second preset duration is equal to 0, that is, as long as it is detected that the impedance value is greater than the third abnormal value or less than the fourth abnormal value, the syringe pump is immediately controlled to stop the perfusion operation.

In this way, when it is detected that the temperature value or the impedance value exceeds a preset range for more than the preset duration, the syringe pump is controlled to immediately stop the perfusion operation, and potential safety hazards may be discovered and eliminated in time, and thus the safety of the ablation operation is improved.

It should be noted that the step S402 and the step S403 in this embodiment are not limited by their sequence numbers, and may be executed synchronously in practical applications.

Optionally, in another embodiment of the present invention, when obtained in real time are the impedance value and the temperature value of the ablation object, all adjustment amplitudes such as the increase and the decrease involved in the present invention may be determined according to the change information of the impedance value and the temperature value. In this way, the adjustment amplitude is determined by integrating changes in the impedance value and the temperature value, which may further improve the accuracy of adjusting the perfusion volume.

Particularly, different base values and weight values are assigned to the real-time change amplitude of the impedance value and the real-time change amplitude of the temperature value. In the process of dynamically adjusting the perfusion volume of the syringe pump, the adjustment amplitude is calculated according to the base value and the weight value, wherein the weight value corresponding to the real-time change amplitude of the temperature value is smaller than the weight value corresponding to the real-time change amplitude of the impedance value. Adjusting the adjustment amplitude according to the base value and the weight value may particularly employ a weighted average fashion or a weighted fashion.

In the embodiment of the present invention, when the ablation task is triggered, the syringe pump is controlled to perform the perfusion operation on the ablation object, and the impedance value and/or the temperature value of the ablation object are obtained in real time; and then the obtained impedance value and/or the obtained temperature value are/is analyzed, and the perfusion volume of the syringe pump is dynamically adjusted according to real-time change information of the impedance value and/or the temperature value obtained from the analysis. Accordingly, automatic dynamic adjustment on the perfusion volume of the syringe pump is realized based on the analysis of changes in the impedance value and/or the temperature value of the ablation object in the process of performing the ablation task. Since the perfusion volume of the syringe pump is automatically adjusted dynamically as the impedance value and/or the temperature value of the ablation object changes, operation delays and errors caused by manual determination can be reduced, and the timeliness and the accuracy of perfusing the liquid in the process of performing the ablation task can be improved. Accordingly, the injury of the ablation operation to the ablation object is reduced, and the safety of the radio frequency ablation treatment is improved.

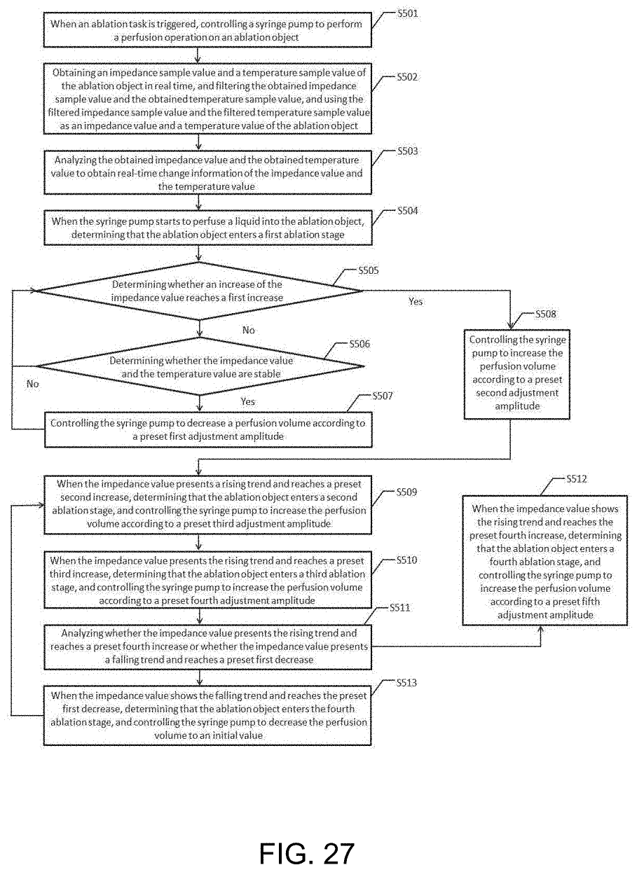

Reference is made to FIG. 27, which is a flowchart of an implementation of a method for controlling the perfusion of a syringe pump according to another embodiment of the present invention. The method may be implemented by the syringe pump 20 in FIG. 23, or may be implemented by the radio frequency ablation control apparatus 10 in FIG. 23, or may be implemented by other computer devices electrically coupled to the syringe pump. As shown in FIG. 27, the method particularly includes the following steps.

In step S501, when an ablation task is triggered, the syringe pump is controlled to perform a perfusion operation on an ablation object.

In step S502, an impedance sample value and a temperature sample value of the ablation object are obtained in real time, the obtained impedance sample value and the obtained temperature sample value are filtered, and the filtered impedance sample value and the filtered temperature sample value are used as the impedance value and the temperature value of the ablation object.

Particularly, the ablation task may be triggered when for example preset trigger time is reached, a trigger instruction transmitted by the radio frequency ablation apparatus is received, or a notification event that a user performs an operation for triggering the ablation task is detected. Among them, the operation for triggering the ablation task is for example to press a physical or virtual button for triggering the ablation task.

Optionally, after each start of the syringe pump, a perfusion parameter is set to a preset initial value. The perfusion parameter may include but is not limited to a perfusion flow rate, a total perfusion volume, perfusion time, and the like.

When the ablation task is triggered, the syringe pump performs the perfusion operation on the ablation object according to the initial value mentioned above. Meanwhile, the impedance sample value and the temperature sample value of the ablation object are obtained in real time by the temperature acquisition apparatus and the impedance acquisition apparatus, and the obtained impedance sample value and the obtained temperature sample value are filtered to remove an abnormal value in the sample values. Then, the filtered impedance sample value and the filtered temperature sample value are used as the impedance value and temperature value of the ablation object.

It should be understood that filtering is to process noise and invalid values in acquired data due to the interference of a sample circuit and an error code in a process of transmitting the sample data, so as to reduce an acquisition error to a preset range.

In this way, the referenceability of the acquired data may be further improved by removing the abnormal value in the sample values by means of the filtering.

Optionally, in another embodiment of the present invention, after the impedance sample value and the temperature sample value of the ablation object are obtained in real time and the obtained impedance sample value and the obtained temperature sample value are filtered, it is further determined that the filtered impedance sample value and the filtered temperature sample value exceed a preset warning value range. If the filtered impedance sample value and the filtered temperature sample value exceed the preset warning value range, alarm information will be output to remind the user that the operation is abnormal and whether the user needs to stop the ablation operation. If the filtered impedance sample value and the filtered temperature sample value do not exceed the preset warning value range, a lowest or average value of the filtered impedance sample value and the filtered temperature sample value within a preset period is used as the impedance value and temperature value obtained in real time, for example, the lowest value in the impedance sample value after being filtered within 10 s is taken as the impedance value obtained in real time, and the lowest value in the temperature sample value after being filtered within 10 s is taken as the temperature value obtained in real time.

In this way, the safety of the ablation operation may be further improved by alarming when it is detected that the filtered impedance sample value and the filtered temperature sample value exceed the preset warning value range. In addition, the referenceability of the acquired data may be further improved by using the lowest or average value within the preset period as the impedance value and the temperature value obtained in real time.

In step S503, the obtained impedance value and the obtained temperature value are analyzed to obtain real-time change information of the impedance value and the temperature value.

Particularly, the real-time change information may include but is not limited to a real-time change trend and a real-time change amplitude. The real-time change trend is a change direction, for example, the value increases or decreases. The real-time change amplitude is a change degree, which may be an absolute value or a ratio, for example, an increased value, or an increased percentage.

In step S504, when the syringe pump starts to perfuse a liquid into the ablation object, it is determined that the ablation object enters a first ablation stage.

In step S505, according to the real-time change information of the impedance value, it is determined whether an increase of the impedance value reaches a first increase.

If the increase of the impedance value does not reach the first increase, the method performs a step S506 of determining whether the impedance value and the temperature value are stable according to the real-time change information of the impedance value and the temperature value.

If the impedance value and the temperature value are stable, the method performs a step S507 of controlling the syringe pump to decrease the perfusion volume according to a preset first adjustment amplitude, and returns to perform the step S505.

If the impedance value and the temperature value are not stable, the method performs the step S505.

If the increase of the impedance value reaches the first increase, the method performs a step S508 of controlling the syringe pump to increase the perfusion volume according to a preset second adjustment amplitude.

Particularly, it is determined whether the real-time obtained impedance value of the ablation object presents a rising trend and reaches a preset first increase, for example, whether the real-time obtained impedance value increases by 1%. It should be understood that the increase involved in the present invention represents a rising degree, which may be an absolute value or a proportional value. The trend involved in the present invention may be an overall trend. In practical applications, obtaining real-time impedance values and temperature values in real time, analyzing and determining the real-time change information, and adjusting the perfusion volume may be performed in parallel by multiple threads.

On the one hand, if the impedance value presents an overall rising trend but does not reach the preset first increase, it is determined whether the impedance value and the temperature value are stable according to the real-time change information of the impedance value and the temperature value, that is, it is determined whether the impedance value and the temperature value are respectively within or are maintained within their respective corresponding preset impedance value ranges and preset temperature value ranges. If the impedance value and the temperature value are respectively within or are maintained within their respective corresponding preset impedance value ranges and preset temperature value ranges, then it is determined that the impedance value and the temperature value are stable; and otherwise, it is determined that the impedance value and the temperature value are not stable.

If the impedance value and the temperature value are stable, indicating that the current perfusion volume is slightly large and the increase or rising speed of the impedance value and the temperature value does not meet the specified requirements, then in order to avoid unnecessary waste of the perfused liquid and over perfusion of the liquid, the syringe pump is controlled to decrease the perfusion volume according to the preset first adjustment amplitude, and the method returns to perform the step S505 of determining whether the increase of the impedance value reaches the first increase according to the real-time change information of the impedance value.

If the impedance value and the temperature value are not stable, indicating that the current perfusion volume is moderate and no adjustment is required, then the method returns to perform the step S505 of determining whether the increase of the impedance value reaches the first increase according to the real-time change information of the impedance value.

It should be understood that in the process of performing the ablation operation, the impedance value of the ablation site changes with the increase of the radio frequency energy (heat), and presents a rising trend as a whole. As the temperature of the ablation site increases, protein is denatured and carbonized, and its inherent physical impedance characteristics will change therewith. That is, as the ablation operation is performed continuously, the impedance value and the temperature value of the ablation site will change therewith, thereby presenting an unstable state. The purposes of perfusing the saline are to lower the temperature so as to avoid injury to the patient due to excessive temperature and to reduce the impedance such that an ablation power is in a normal range. As the ablation operation is performed continuously, it is necessary to make the impedance value and the temperature value of the ablation site change in stages as required for ablation. For example, in the first and second ablation stages, it is necessary to keep the impedance value and the temperature value rise as a whole. If the actual impedance value and the actual temperature value are in a stable state, that is, are maintained within a certain range, the perfusion volume is too large to meet the requirement for the current ablation.

On the other hand, if the impedance value presents a rising trend and reaches the preset first increase, the syringe pump is controlled to increase the perfusion volume according to the preset second adjustment amplitude. Particularly, the perfusion volume may be increased by increasing the perfusion flow rate of the liquid.

In step S509, when the impedance value presents the rising trend and reaches the preset second increase, it is determined that the ablation object enters a second ablation stage, and the syringe pump is controlled to increase the perfusion volume according to a preset third adjustment amplitude.

Particularly, in the first ablation stage, the real-time change trend and the real-time change amplitude of the impedance value are continuously analyzed, and when the impedance value present the rising trend and reaches the preset second increase (for example, increases by a second percentage point), it is determined that the ablation object enters the second ablation stage, and the syringe pump is controlled to increase the perfusion volume according to the preset third adjustment amplitude.

In step S510, when the impedance value presents the rising trend and reaches a preset third increase, it is determined that the ablation object enters a third ablation stage, and the syringe pump is controlled to increase the perfusion volume according to a preset fourth adjustment amplitude.

Particularly, in the second ablation stage, the real-time change trend and the real-time change amplitude of the impedance value are continuously analyzed, and when the impedance value presents the rising trend and reaches the preset third increase (for example, increases by a third percentage point), it is determined that the ablation object enters the third ablation stage, and the syringe pump is controlled to increase the perfusion volume according to a preset fourth adjustment amplitude.

In step S511, it is analyzed whether the impedance value presents the rising trend and reaches a preset fourth increase or whether the impedance value presents a falling trend and reaches a preset first decrease.

In step S512, when the impedance value presents the rising trend and reaches the preset fourth increase, it is determined that the ablation object enters a fourth ablation stage, and the syringe pump is controlled to increase the perfusion volume according to a preset fifth adjustment amplitude.

In step S513, when the impedance value presents the falling trend and reaches the preset first decrease, it is determined that the ablation object enters the fourth ablation stage, and the syringe pump is controlled to decrease the perfusion volume to an initial value.

Particularly, in the third ablation stage, the real-time change trend and the real-time change amplitude of the impedance value are continuously analyzed. It should be noted that in the third ablation stage, in addition to analyzing whether the impedance value presents the rising trend, it is further necessary to analyze whether the impedance value presents the falling trend. That is, when the impedance value presents the rising trend and reaches a preset fourth increase (for example, increases by a fourth percentage point) or the impedance value presents the falling trend and reaches the preset first decrease (for example, decreases by a fourth percentage point), it is determined that the ablation object enters the fourth ablation stage.

However, a processing logic varies according to different reasons for confirming that the ablation object enters the fourth ablation stage. When it is determined that the ablation object enters the fourth ablation stage because the impedance value presents the rising trend and reaches the preset fourth increase, the syringe pump is controlled to increase the perfusion volume according to the preset fifth adjustment amplitude. When it is determined that the ablation object enters the fourth ablation stage because the impedance value presents the falling trend and reaches the preset first decrease, the syringe pump is controlled to decrease the perfusion volume to the initial value, and it is determined that the ablation object enters the first ablation stage, and then a step S509 of determining that the ablation object enters the second ablation stage when the impedance value presents the rising trend and reaches the preset second increase and controlling the syringe pump to increase the perfusion volume according to the preset third adjustment amplitude until the control period is ended is performed.

It should be understood that controlling the syringe pump to decrease the perfusion volume to the initial value may be done at one time, or may be done step by step. The above-mentioned control period may refer to a completion period of the entire ablation task, or a single ablation task may be divided into multiple control periods as well.

It should be understood that values of the preset first increase to the preset fourth increase and the preset first decrease may be the same or different in specific applications. Similarly, values of the first adjustment amplitude to the fifth adjustment amplitude may be the same or different in specific applications. The specific values may be set and adjusted at any time according to a custom operation of a user in practical applications.

It should be noted that the determinations involved in the embodiments of the present invention are all based on the real-time change information of the corresponding parameters.

In the embodiment of the present invention, the entire ablation process is divided into multiple ablation stages according to the real-time change trend and the real-time change amplitude of the impedance value of the ablation object, and the perfusion volume of the syringe pump is dynamically adjusted according to a corresponding adjustment logic based on change characteristics of the impedance value and the temperature value of the ablation object obtained in real time in each ablation stage, which makes the adjustment more targeted. As a result, the accuracy of automatically controlling the perfusion is improved.

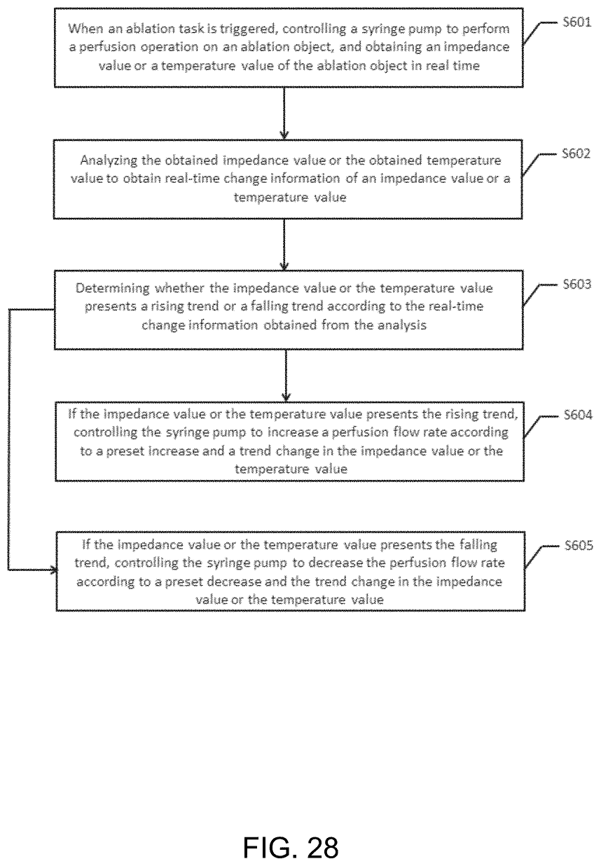

Reference is made to FIG. 28, which is a flow chart of an implementation of a method for controlling the perfusion of a syringe pump according to another embodiment of the present invention. The method may be implemented by the syringe pump 20 in FIG. 23, or may be implemented by the radio frequency ablation control apparatus 10 in FIG. 23, or may be implemented by other computer devices electrically coupled to the syringe pump. As shown in FIG. 28, the method particularly includes the following steps.

In step S601, when an ablation task is triggered, the syringe pump is controlled to perform a perfusion operation on an ablation object, and an impedance value or a temperature value of the ablation object is obtained in real time.

Particularly, the ablation task may be triggered when for example preset trigger time is reached, a trigger instruction transmitted by the radio frequency ablation apparatus is received, or a notification event that a user performs an operation for triggering the ablation task is detected. Among them, the operation for triggering the ablation task is for example to press a physical or virtual button for triggering the ablation task.

Optionally, after each start of the syringe pump, a perfusion parameter is set to a preset initial value. The perfusion parameter may include but is not limited to a perfusion flow rate, a total perfusion volume, perfusion time, and the like.

When the ablation task is triggered, the syringe pump performs the perfusion operation on the ablation object according to the above-mentioned initial value. Meanwhile, the impedance value or the temperature value of the ablation object is obtained in real time by the temperature acquisition apparatus and the impedance acquisition apparatus, as reference data for dynamically adjusting the perfusion volume of the syringe pump. The impedance value and the temperature value of the ablation object may be for example the impedance value and the temperature value of the ablation site. The impedance value may be a resistance value, for example.

Optionally, the impedance value or the temperature value of the ablation object may further be obtained in real time particularly by the following steps.

An impedance sample value or a temperature sample value of the ablation object is obtained in real time, and the obtained impedance sample value or the obtained temperature sample value is filtered.

It is determined that the filtered impedance sample value or the filtered temperature sample value exceeds a preset warning value range.

If the filtered impedance sample value or the filtered temperature sample value exceeds the preset warning value range, alarm information will be output to remind the user that the operation is abnormal and whether the user needs to stop the ablation operation.

If the filtered impedance sample value or the filtered temperature sample value does not exceed the preset warning value range, a lowest or average value of the filtered impedance sample value and the filtered temperature sample value within a preset period (for example, within 10 s) is used as the impedance value or temperature value obtained in real time.

In step S602, the obtained impedance value or the obtained temperature value is analyzed to obtain real-time change information of the impedance value or the temperature value.

Particularly, the real-time change information may include but is not limited to a real-time change trend and a real-time change amplitude. The real-time change trend is a change direction, for example, the value increases or decreases. The real-time change amplitude is a change degree, which may be an absolute value or a proportional value, for example, an increased value, or an increased percentage.

Optionally, in another embodiment of the present invention, the impedance value and the temperature value of the ablation object may be obtained in real time simultaneously, and the obtained impedance value and the obtained temperature value may be analyzed. At this time, all the adjustment amplitudes such as the increase and the decrease involved in the following may be determined according to analysis results.

Particularly, different base values and weight values are assigned to the real-time change amplitude of the impedance value and the real-time change amplitude of the temperature value. In the process of dynamically adjusting the perfusion volume of the syringe pump, the adjustment amplitude is calculated according to the base value and the weight value, wherein the weight value corresponding to the real-time change amplitude of the temperature value is smaller than the weight value corresponding to the real-time change amplitude of the impedance value. Adjusting the adjustment amplitude according to the base value and the weight value may particularly employ a weighted average fashion or a weighted fashion.

In step S603, it is determined whether the impedance value or the temperature value presents a rising trend or a falling trend according to the real-time change information obtained from the analysis.

In step S604, if the impedance value or the temperature value presents the rising trend, the syringe pump is controlled to increase the perfusion flow rate according to a preset increase and a trend change in the impedance value or the temperature value.

In step S605, if the impedance value or the temperature value presents the falling trend, the syringe pump is controlled to decrease the perfusion flow rate according to a preset decrease and the trend change in the impedance value or the temperature value.

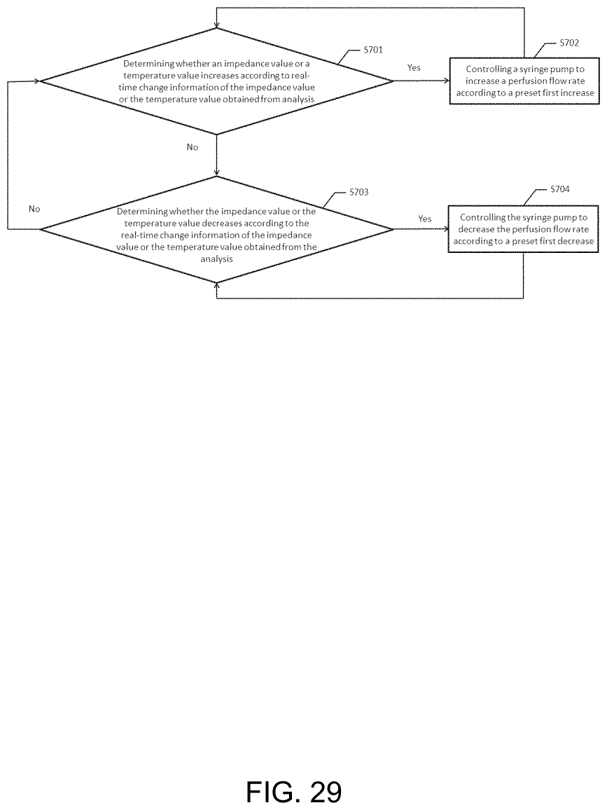

Particularly, as shown in FIG. 29, the step S603 to the step S605 may be implemented through the following steps.

In step S701, it is determined whether the impedance value or the temperature value increases according to the real-time change information of the impedance value or the temperature value obtained from the analysis.

If the impedance value or the temperature value increases, the step S603 to the step S605 perform a step S702 of controlling the syringe pump to increase a perfusion flow rate according to a preset first increase so as to increase the perfusion volume, and then return to perform the step S701.

If the impedance value or the temperature value does not increase, the step S603 to the step S605 perform a step S703 of determining whether the impedance value or the temperature value decreases according to the real-time change information.

If the impedance value or the temperature value decreases, the step S603 to the step S605 perform a step S704 of controlling the syringe pump to decrease the perfusion flow rate according to a preset first decrease so as to decrease the perfusion volume, and return to perform the step S703.

If the impedance value or the temperature value does not decrease, the step S603 to the step S605 return to perform the step S701. This cycle repeats until the control period is ended.

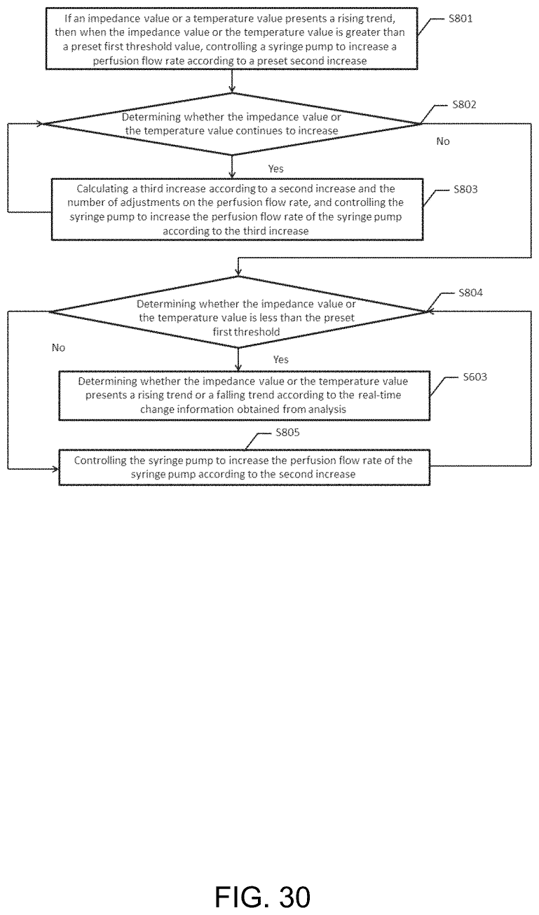

Optionally, in another embodiment of the present invention, as shown in FIG. 30, the step S604 may be further implemented by the following steps.

In step S801, if an impedance value or a temperature value presents a rising trend, then when the impedance value or the temperature value is greater than a preset first threshold, the syringe pump is controlled to increase a perfusion flow rate according to a preset second increase.

In step 802, it is determined whether the impedance value or the temperature value continues to increase.

If the impedance value or the temperature value continues to increase, the step S604 performs a step S803 of calculating a third increase according to the second increase and the number of adjustments on the perfusion flow rate and controlling the syringe pump to increase the perfusion flow rate of the syringe pump according to the third increase, returns to perform the step S802 of determining whether the impedance value or the temperature value continues to increase until the perfusion flow rate increases to a preset maximum flow rate or until the impedance value or temperature value is less than the preset first threshold.

If the impedance value or the temperature value does not continue to increase, the step S604 performs a step S804 of determining whether the impedance value or the temperature value is less than the preset first threshold.

If the impedance value or the temperature value is not less than the preset first threshold, the step S604 performs a step S805 of controlling the syringe pump to increase the perfusion flow rate of the syringe pump according to the second increase until the perfusion flow rate increases to the preset maximum flow rate or until the impedance value or temperature value is less than the preset first threshold.

If the impedance value or the temperature value is less than the preset first threshold, the step S604 returns to perform the step S603 of determining whether the impedance value or the temperature value presents the rising trend or the falling trend according to the real-time change information obtained from the analysis.

This cycle repeats until the control period is ended.

Optionally, calculating the third increase according to the second increase and the number of the adjustments on the perfusion flow rate in the step S803 may particularly be using a value obtained by multiplying the second increase by the number of the adjustments as the third increase; or the third increase may be calculated according to a geometric incremental rule based on a preset ratio, the second increase, and the number of the adjustments on the perfusion flow rate. The number of the adjustments on the perfusion flow rate may be understood as the number of operations performed to determine whether the impedance value or the temperature value continues to increase.

Further, when the perfusion flow rate increases to the preset maximum flow rate, reminding information is output to remind the user that the perfusion flow rate of the syringe pump has reached a limit value, wherein the reminding information may be output in at least one form of speech, text, image, animation and light.

It should be understood that the purpose of adjusting the perfusion volume is to keep the impedance value and the temperature value of the ablation object stable within a safe range. On the one hand, if the impedance value or the temperature value does not continue to increase, it is effective to increase the perfusion flow rate of the syringe pump according to the previous increase. At this time, the perfusion flow rate of the syringe pump is continuously increased according to the increase, which may avoid excessive adjustment. As a result, the accuracy of the perfusion may be improved.

On the other hand, if the impedance value or the temperature value continues to increase, the effect of increasing the perfusion flow rate of the syringe pump according to the previous increase is poor, and the increase may not meet the ablation demand. At this time, the syringe pump is controlled to increase the perfusion flow rate of the syringe pump according to a larger increase, such that the perfusion volume is quickly adjusted to a desired degree. Accordingly, the timeliness of the perfusion is improved.

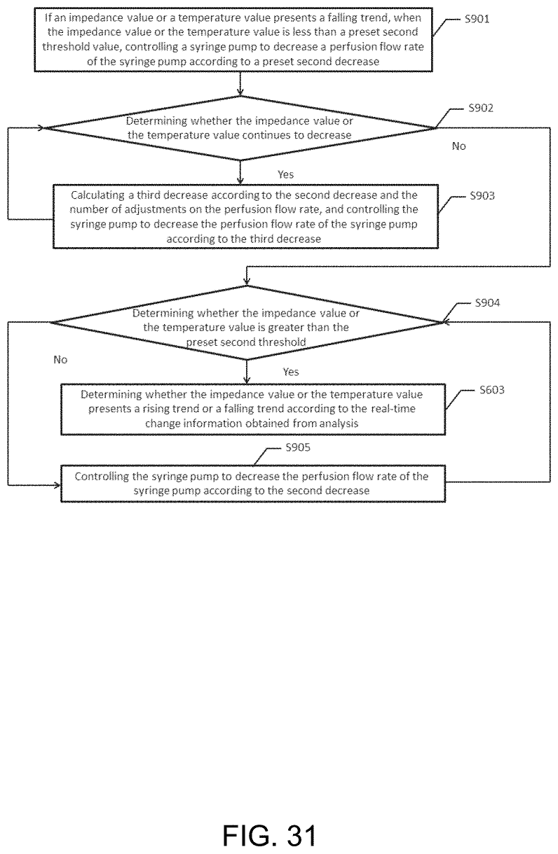

Optionally, in another embodiment of the present invention, as shown in FIG. 31, the step S605 may be implemented by the following steps.

In step S901, if an impedance value or a temperature value presents a falling trend, when the impedance value or the temperature value is less than a preset second threshold, a syringe pump is controlled to decrease a perfusion flow rate of the syringe pump according to a preset second decrease.

In step S902, it is determined whether the impedance value or the temperature value continues to decrease.

If the impedance value or the temperature value continues to decrease, the step S605 performs a step S903 of calculating a third decrease according to the second decrease and the number of adjustments on the perfusion flow rate and controlling the syringe pump to decrease the perfusion flow rate according to the third decrease, and returns to perform the step S902 of determining whether the impedance value or the temperature value continues to decrease until the perfusion flow rate decreases a preset minimum flow rate or until the impedance value or the temperature value is greater than the preset second threshold.

If the impedance value or the temperature value does not continue to decrease, the step S605 performs step S904 of determining whether the impedance value or the temperature value is greater than the preset second threshold.

If the impedance value or the temperature value is not greater than the preset second threshold, the step S605 performs a step S905 of controlling the syringe pump to decrease the perfusion flow rate according to the second decrease, and returns to perform the step S904 of determining whether the temperature value or the temperature value is greater than the preset second threshold until the perfusion flow rate decreases to the preset minimum flow rate or until the impedance value or the temperature value is greater than the preset second threshold.

If the impedance value or the temperature value is greater than the preset second threshold, the step S605 returns to perform the step S603 of determining whether the impedance value or the temperature value presents the rising trend or the falling trend according to the real-time change information obtained from the analysis.

It should be understood that if the impedance value or the temperature value does not continue to decrease, it is effective to decrease the perfusion flow rate of the syringe pump according to the previous decrease. At this time, the perfusion flow rate of the syringe pump is continuously decreased according to the decrease, which may avoid excessive. As a result, the accuracy of the perfusion may be improved. On the other hand, if the impedance value or the temperature value continues to decrease, the previous decrease is not enough. At this time, the decrease is improved, such that the perfusion volume may be rapidly adjusted to a desired degree, and thus the timeliness of the perfusion is improved.

This cycle repeats until the control period is ended.

Optionally, calculating the third increase according to the second increase and the number of the adjustments on the perfusion flow rate in the step S903 may particularly be using a value obtained by multiplying the second decrease by the number of the adjustments as the third decrease, or the third decrease may be calculated according to a geometric degressive rule based on a preset ratio, the second decrease, and the number of the adjustments on the perfusion flow rate. The number of the adjustments on the perfusion flow rate may be understood as the number of operations performed to determine whether the impedance value or the temperature value continues to decrease.

Further, when the perfusion flow rate decreases to the preset minimum flow rate, reminding information is output to remind the user that the perfusion flow rate of the syringe pump has been decreased to a limit value, wherein the reminding information may be output in at least one form of speech, text, image, animation and light.

It should be understood that the above-mentioned preset first threshold is a preset highest limit, and the above-mentioned preset second threshold is a preset lowest limit. Exceeding these two limits indicates that the injury may be brought to the ablation object. Therefore, it is necessary to adjust the perfusion volume. In addition, the preset first threshold and the preset second threshold are a collective term. In practical applications, specific values and units of the preset first threshold and the preset second threshold are determined by types of objects to which they are applied, and may be set according to user-defined operations. That is, when applied to the determination of the impedance value and when applied to the determination of the temperature value, the specific values and units of the preset first threshold and the preset second threshold are different. For example, when applied to the determination of the impedance value, the unit of the preset first threshold is ohm; but when applied to the determination of the temperature value, the unit of the preset first threshold is degrees Celsius.

In addition, all the increase and decrease involved in the present invention represent a degree, which may be an absolute value or a proportional value, such as an increased value or an increased percentage.

In the embodiment of the present invention, when the ablation task is triggered, the syringe pump is controlled to perform the perfusion operation on the ablation object, and the impedance value and/or the temperature value of the ablation object are obtained in real time; and then the obtained impedance value and/or the obtained temperature value are/is analyzed, and the perfusion volume of the syringe pump is dynamically adjusted according to real-time change information of the impedance value and/or the temperature value obtained from the analysis. Accordingly, automatic dynamic adjustment on the perfusion volume of the syringe pump is realized based on the analysis of changes in the impedance value and/or the temperature value of the ablation object in the process of performing the ablation task. Since the perfusion volume of the syringe pump is automatically adjusted dynamically as the impedance value and/or the temperature value of the ablation object changes, operation delays and errors caused by manual determination can be reduced, and the timeliness and the accuracy of perfusing the liquid in the process of performing the ablation task can be improved. Accordingly, the injury of the ablation operation to the ablation object is reduced, and the safety of the radio frequency ablation treatment is improved.

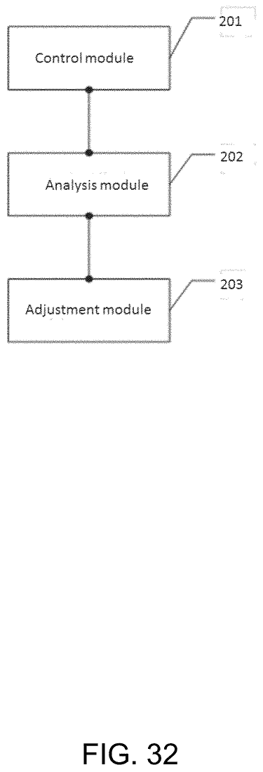

Reference is made to FIG. 32, which is a schematic diagram showing a structure of an apparatus for controlling the perfusion of a syringe according to an embodiment of the present invention. For ease of description, only parts related to the embodiments of the present invention are shown. The apparatus may be disposed in the syringe pump 20 shown in FIG. 23, or in the radio frequency ablation control apparatus 10, or may be disposed in other computer devices. The apparatus includes a control module 201, an analysis module 202 and an adjustment module 203.

The control module 201 is configured to when an ablation task is triggered, control a syringe pump to perform a perfusion operation on an ablation object, and obtain an impedance value and/or a temperature value of the ablation object in real time.

The analysis module 202 is configured to analyze the obtained impedance value and/or the obtained temperature value to obtain real-time change information of the impedance value and/or the temperature value.

The adjustment module 203 is configured to dynamically adjust a perfusion volume of the syringe pump according to the real-time change information obtained from the analysis.

Optionally, wherein the real-time change information includes a real-time change trend and a real-time change amplitude.

Optionally, the adjustment module 203 is further configured to determine a current ablation stage according to the real-time change trend and the real-time change amplitude of the impedance value, and adjust the perfusion volume of the syringe pump according to a target adjustment logic corresponding to the current ablation stage.

Optionally, the adjustment module 203 includes: a first adjustment module, which is configured to when the syringe pump starts to perfuse a liquid into the ablation object, determine that the ablation object enters a first ablation stage and adjust a perfusion volume of the syringe pump according to a first target adjustment logic based on the real-time change trend and the real-time change amplitude of the impedance value; a second adjustment module, which is configured to in the first ablation stage, when the impedance value presents a first real-time change trend and reaches a first real-time change amplitude, determine that the ablation object enters a second ablation stage and adjust the perfusion volume of the syringe pump according to a second target adjustment logic; a third adjustment module, which is configured to in the second ablation stage, when the impedance value presents a second real-time change trend and reaches a second real-time change amplitude, determine that the ablation object enters a third ablation stage and adjust the perfusion volume of the syringe pump according to a third target adjustment logic; and a fourth adjustment module, which is configured to in the third ablation stage, when the impedance value presents a third real-time change trend and reaches a third real-time change amplitude, determine that the ablation object enters a fourth ablation stage and adjust the perfusion volume of the syringe pump according to a fourth target adjustment logic.

Optionally, when the real-time obtained data includes the impedance value and the temperature value, the first adjustment module is further configured to determine whether the impedance value presents a rising trend and reaches a preset first increase; if the impedance value presents the rising trend but does not reach the preset first increase, determine whether the impedance value and the temperature value are stable; if the impedance value and the temperature value are stable, control the syringe pump to decrease the perfusion volume according to the preset first adjustment amplitude and return to the step of determining whether the impedance value presents the rising trend and reaches the preset first increase; and if the impedance value presents the rising trend and reaches the preset first increase, control the syringe pump to increase the perfusion volume according to a preset second adjustment amplitude.

Optionally, the second adjustment module is further configured to in the first ablation stage, when the impedance value presents the rising trend and reaches the preset second increase, determine that the ablation object enters the second ablation stage, and control the syringe pump to increase the perfusion volume according to a preset third adjustment amplitude.

Optionally, the third adjustment module is further configured to in the second ablation stage, when the impedance value presents the rising trend and reaches the preset third increase, determine that the ablation object enters the third ablation stage, and control the syringe pump to increase the perfusion volume according to a preset fourth adjustment amplitude.

Optionally, the fourth adjustment module is further configured to in the third ablation stage, when the impedance value presents the rising trend and reaches the preset fourth increase, determine that the ablation object enters the fourth ablation stage, and control the syringe pump to increase the perfusion volume according to a preset fifth adjustment amplitude; alternatively, when the impedance value presents a falling trend and reaches a preset first decrease, determine that the ablation object enters the fourth ablation stage and control the syringe pump to decrease the perfusion volume to an initial value.

Optionally, the adjustment module 203 is further configured to analyze whether the impedance value or the temperature value presents the rising trend or the falling trend; if the impedance value or the temperature value presents the rising trend, control the syringe pump to increase a perfusion flow rate according to a preset increase and a trend change in the impedance value or the temperature value; and if the impedance value or the temperature value presents the falling trend, control the syringe pump to decrease the perfusion flow rate according to a preset decrease and the trend change in the impedance value or the temperature value.

Optionally, the adjustment module 203 is further configured to determine whether the impedance value or the temperature value increases according to the real-time change information; if the impedance value or the temperature value increases, control the syringe pump to increase the perfusion flow rate according to the preset first increase and return to perform the step of determining that the impedance value or the impedance value decreases according to the real-time change information; if the impedance value or the temperature value does not increase, determine whether the impedance value or the temperature value decreases according to the real-time change information; if the impedance value or the temperature value decreases, control the syringe pump to decrease the perfusion flow rate according to the preset first decrease and return to perform the step of determining whether the impedance value or the temperature value decreases according to the real-time change information; and if the impedance value or the temperature value does not decrease, return to perform the step of determining whether the impedance value or the temperature value increases according to the real-time change information.

Optionally, the adjustment module 203 is further configured to when the impedance value or the temperature value is greater than a preset first threshold, control the syringe pump to increase the perfusion flow rate according to a preset second increase; determine whether the impedance value or the temperature value continues to increase; if the impedance value or the temperature value continues to increase, calculate a third increase according to the second increase and the number of adjustments on the perfusion flow rate; control the syringe pump to increase the perfusion flow rate according to the third increase and return to perform the step of determining whether the impedance value or the temperature value continues to increase until the perfusion flow rate increases to a preset maximum flow rate; if the impedance value or the temperature value does not continue to increase, determine whether the impedance value or the temperature value is less than the preset first threshold; if the impedance value or the temperature value is not less than the preset first threshold, control the syringe pump to increase the perfusion flow rate according to the second increase until the perfusion flow rate increases to the preset maximum flow rate; and if the impedance value or the temperature value is less than the preset first threshold, return to perform the step of determining whether the impedance value or the temperature value presents the rising trend or the falling trend according to the real-time change information obtained from the analysis.

Optionally, the adjustment module 203 is further configured to when the impedance value or the temperature value is less than a preset second threshold, control the syringe pump to decrease the perfusion flow rate of the syringe pump according to a preset second decrease; determine whether the impedance value or the temperature value continues to decrease; if the impedance value or the temperature value continues to decrease, calculate a third decrease according to the second decrease and the number of adjustments on the perfusion flow rate; control the syringe pump to decrease the perfusion flow rate according to the third decrease and return to perform the step of determining whether the impedance value or the temperature value continues to decrease until the perfusion flow rate decreases a preset minimum flow rate; if the impedance value or the temperature value does not continue to decrease, determine whether the impedance value or the temperature value is greater than the preset second threshold; if the impedance value or the temperature value is not greater than the preset second threshold, control the syringe pump to decrease the perfusion flow rate according to the second decrease and return to perform the step of determining whether the temperature value or the temperature value is greater than the preset second threshold until the perfusion flow rate decreases to the preset minimum flow rate; and if the impedance value or the temperature value is greater than the preset second threshold, return to perform the step of determining whether the impedance value or the temperature value presents the rising trend or the falling trend according to the real-time change information obtained from the analysis.

Optionally, the control module 201 includes:

a first data obtaining module, which is configured to obtain an impedance sample value and/or a temperature sample value of the ablation object in real time; and a first filtering module, which is configured to filter the obtained impedance sample value and/or the obtained temperature sample value and use the filtered impedance sample value and/or the filtered temperature sample value as the impedance value and/or the temperature value.

Optionally, the control module 202 further includes: a second data obtaining module, which is configured to obtain an impedance sample value and/or a temperature sample value of the ablation object in real time; a second filtering module, which is configured to filter the obtained impedance sample value and/or the obtained temperature sample value; a determination module, which is configured to determine whether the filtered impedance sample value and/or the filtered temperature sample value exceed/exceeds a preset warning value range; an alarm module, which is configured to if the filtered impedance sample value and/or the filtered temperature sample value exceed/exceeds the preset warning value range, output alarm information; and the second data obtaining module, which is configured to if the filtered impedance sample value and/or the filtered temperature sample value do/does not exceed the preset warning value range, use a lowest or average value among the filtered impedance sample value and/or the filtered temperature sample value within a preset period as the impedance value and/or the temperature value.

Optionally, the adjustment module 203 further includes: a fifth adjustment module, which is configured to when the real-time change information includes the real-time change trend and the real-time change amplitude of the impedance value and the real-time change trend and the real-time change amplitude of the temperature value, analyze whether the real-time change trend and the real-time change amplitude of the impedance value and the real-time change trend and the real-time change amplitude of the temperature value meet an adjustment condition; and if the real-time change trend and the real-time change amplitude of the impedance value and the real-time change trend and the real-time change amplitude of the temperature value meet the adjustment condition, dynamically adjust the perfusion volume of the syringe pump according to the real-time change trend and the real-time change amplitude of the impedance value.

Optionally, the apparatus further includes: a calculation module, which is configured to when the real-time change information includes the real-time change trend and the real-time change amplitude of the impedance value and the real-time change trend and the real-time change amplitude of the temperature value, assign different base values and weight values to the real-time change amplitude of the impedance value and the real-time change amplitude of the temperature value, and calculate an adjustment amplitude according to the base value and the weight value in a process of dynamically adjusting the perfusion volume of the syringe pump, wherein the weight value corresponding to the real-time change amplitude of the temperature value is smaller than the weight value corresponding to the real-time change amplitude of the impedance value.

Optionally, the apparatus further includes: a first emergency control module, which is configured to when detecting that the temperature value is greater than a first abnormal value or less than a second abnormal value for more than a first preset duration, control the syringe pump to stop the perfusion operation, wherein the first preset duration is greater than or equal to zero.

Optionally, the apparatus further includes: a second emergency control module, which is configured to when detecting that the impedance value is greater than a third abnormal value or less than a fourth abnormal value for more than a second preset duration, control the syringe pump to stop the perfusion operation, wherein the second preset duration is greater than or equal to zero.

For a specific process for the above modules to realize their respective functions, reference is made to relevant contents in the embodiments shown in FIG. 26 to FIG. 31, which will be omitted here.

In the embodiment of the present invention, when the ablation task is triggered, the syringe pump is controlled to perform the perfusion operation on the ablation object, and the impedance value and/or the temperature value of the ablation object are obtained in real time; and then the obtained impedance value and/or the obtained temperature value is analyzed, and the perfusion volume of the syringe pump is dynamically adjusted according to real-time change information of the impedance value and/or the temperature value obtained from the analysis. Accordingly, automatic dynamic adjustment on the perfusion volume of the syringe pump is realized based on the analysis of changes in the impedance value and/or the temperature value of the ablation object in the process of performing the ablation task. Since the perfusion volume of the syringe pump is automatically adjusted dynamically as the impedance value and/or the temperature value of the ablation object changes, operation delays and errors caused by manual determination can be reduced, and the timeliness and the accuracy of perfusing the liquid in the process of performing the ablation task can be improved. Accordingly, the injury of the ablation operation to the ablation object is reduced, and the safety of the radio frequency ablation treatment is improved.

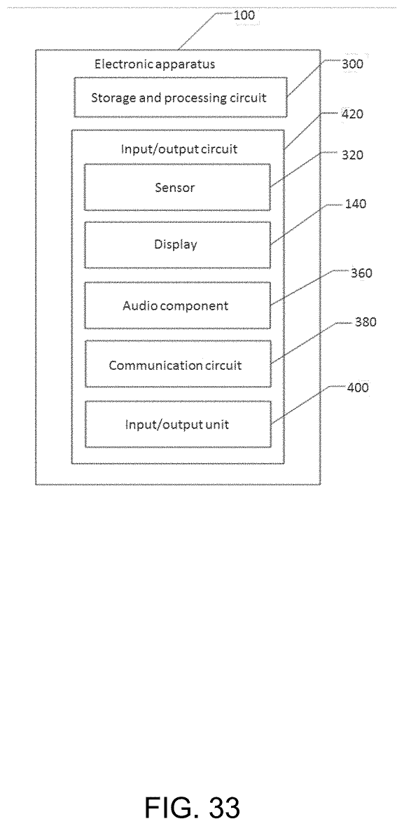

Reference is made to FIG. 33, which is a schematic diagram showing a hardware structure of an electronic apparatus according to an embodiment of the present invention.

The electronic apparatus may be any one of various types of computer system devices that are non-movable or movable or portable and perform wireless or wired communication. Particularly, the electronic apparatus may be a desktop computer, a server, a mobile phone or a smart phone (for example, an iPhone™-based phone, an Android™-based phone), a portable game device (for example, Nintendo DS™, PlayStation Portable™, Gameboy Advance™, iPhone™), a laptop computer, a PDA, a portable Internet device, a music player and a data storage device, and other handheld devices such as watches, earphones, pendants, and earphones. The electronic apparatus may further be other wearable devices (for example, head-mounted devices (HMD), such as electronic glasses, electronic clothes, electronic bracelets, electronic necklaces, electronic tattoos, electronic apparatuses or smart watches).

The electronic apparatus may be any one of multiple electronic apparatuses, including but not limited to a cellular phone, a smart phone, other wireless communication devices, a personal digital assistant, an audio player, other media players, a music recorder, a video recorder, a camera, other media recorders, a radio, a medical device, a vehicle transportation instrument, a calculator, a programmable remote control, a pager, a laptop computer, a desktop computer, a printer, a netbook computer, a personal digital assistant (PDA), a portable multimedia player (PMP), a motion picture experts group (MPEG-1 or MPEG-2) audio layer 3 (MP3) player, a portable medical device and a digital camera, and a combination thereof.

In some cases, the electronic apparatus may perform multiple functions (for example, playing music, displaying videos, storing pictures, and receiving and transmitting phone calls). If desired, the electronic apparatus may be a portable device such as a cell phone, a media player, other handheld devices, a wrist watch device, a pendant device, an earpiece device, or other compact portable devices.

As shown in FIG. 33, the electronic apparatus 100 may include a control circuit, wherein the control circuit may include a storage and processing circuit 300. The storage and processing circuit 300 may include a memory, such as a hard disk drive memory, a non-transitory or non-volatile memory (such as a flash memory or other electronically programmable restricted deletion memory configured to form a solid-state drive), and a volatile memory (for example, a static or dynamic random access memory and the like), and the like, which are not limited in the embodiment of the present invention. The processing circuit in the storage and processing circuit 300 may be configured to control the operation of the electronic apparatus 100. The processing circuit may be implemented based on one or more microprocessors, microcontrollers, digital signal processors, baseband processors, power management units, audio codec chips, application specific integrated circuits, display driver integrated circuits, and the like.

The storage and processing circuit 300 may be configured to run software in the electronic apparatus 100, such as an Internet browsing application, a Voice over Internet Protocol (VOIP) telephone calling application, an email application, a media playback application, an operating system function, and the like. The software may be configured to perform some control operations, for example, image capture based on a camera, ambient light measurement based on an ambient light sensor, proximity sensor measurement based on a proximity sensor, and information display function realized based on a status indicator such as a status indicator lamp of a LED, touch event detection based on a touch sensor, a function associated with displaying information on multiple (for example, layered) displays, an operation associated with performing a wireless communication function, an operation associated with acquiring and generating an audio signal, a control operation associated with acquiring and processing of button press event data, and other functions in the electronic apparatus 100, which are not limited in the embodiment of the present invention.

Further, the memory stores an executable program code, and a processor coupled with the memory calls the executable program code stored in the memory to perform the method for controlling the perfusion of the syringe pump described in the embodiments shown in FIG. 26 to FIG. 31 above.

Among them, the executable program code includes various modules in the apparatus for controlling the perfusion of the syringe pump described in the embodiment shown in FIG. 32, for example, a control module 201, an analysis module 202 and an adjustment module 203.

The electronic apparatus 100 may further include an input/output circuit 420. The input/output circuit 420 may be configured to enable the electronic apparatus 100 to input and output data, that is, to allow the electronic apparatus 100 to receive data from an external device and further allow the electronic apparatus 100 to output data from the electronic apparatus 100 to the external device. The input/output circuit 420 may further include a sensor 320. The sensor 320 may include an ambient light sensor, a light-based or capacitive proximity sensor, and a touch sensor (for example, a light-based touch sensor and/or a capacitive touch sensor, wherein the touch sensor may be a part of a touch display screen, or may be independently used as a touch sensor), an acceleration sensor, other sensors and the like.

The input/output circuit 420 may further include one or more displays, such as a display 140. The display 140 may include one or a combination of a liquid crystal display, an organic light emitting diode display, an electronic ink display, a plasma display, and a display using other display technologies. The display 140 may include a touch sensor array (i.e., the display 140 may be a touch display screen). The touch sensor can be a capacitive touch sensor formed by an array of transparent touch sensor electrodes (for example, indium tin oxide (ITO) electrodes), or a touch sensor formed using other touch technologies, such as sonic touch, pressure-sensitive touch, resistance touch, optical touch, and the like, which are not limited in the embodiment of the present invention.

The electronic apparatus 100 may further include an audio component 360. The audio component 360 may be configured to provide audio input and output functions for the electronic apparatus 100. The audio component 360 in the electronic apparatus 100 may include a speaker, a microphone, a buzzer, a tone generator, and other components for generating and detecting sounds.

The communication circuit 380 may be configured to provide the electronic apparatus 100 with the ability to communicate with an external device. The communication circuit 380 may include an analog and digital input/output interface circuit, and a wireless communication circuit based on a radio frequency signal and/or an optical signal. The wireless communication circuit in the communication circuit 380 may include a radio frequency transceiver circuit, a power amplifier circuit, a low noise amplifier, a switch, a filter and an antenna. For example, the wireless communication circuit in the communication circuit 380 may include a circuit for supporting near field communication (NFC) by transmitting and receiving a near-field coupled electromagnetic signal. For example, the communication circuit 380 may include a near-field communication antenna and a near-field communication transceiver. The communication circuit 380 may further include a cellular phone transceiver and an antenna, a wireless local area network transceiver circuit and an antenna, and the like.

The electronic apparatus 100 may further include a battery, a power management circuit and other input/output units 400. The input/output unit 400 may include a button, a joystick, a click wheel, a scroll wheel, a touch pad, a keypad, a keyboard, a camera, a light emitting diode, and other status indicators.

The user may input a command through the input/output circuit 420 to control the operation of the electronic apparatus 100, and may use the output data of the input/output circuit 420 to realize the reception of status information and other outputs from the electronic apparatus 100.

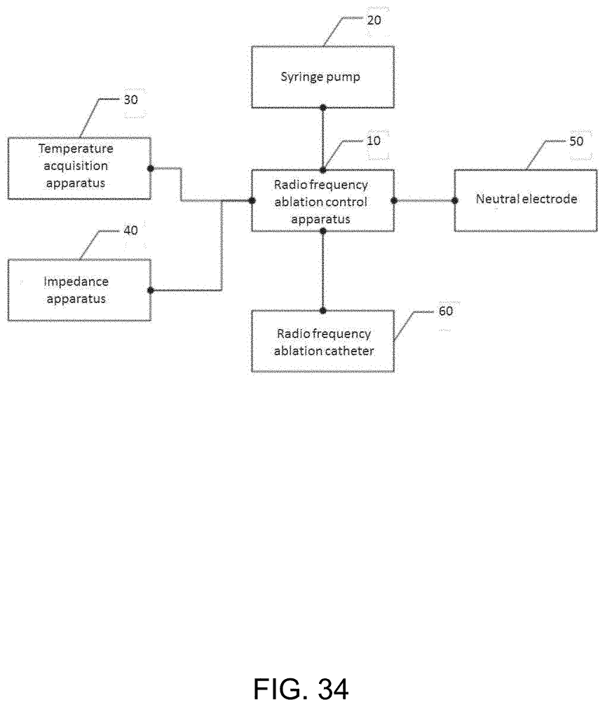

Further, as shown in FIG. 34, embodiments of the present invention further provide a radio frequency ablation system. The radio frequency ablation system includes a radio frequency ablation control apparatus 10, a syringe pump 20, a temperature acquisition apparatus 30, an impedance acquisition apparatus 40, a neutral electrode 50 and a radio frequency ablation catheter 60.

As shown in FIG. 23, the radio frequency ablation catheter 60, the temperature acquisition apparatus 30, the impedance acquisition apparatus 40 and the neutral electrode 50 are electrically connected with the radio frequency ablation control apparatus 10. The radio frequency ablation control apparatus 10 is further electrically coupled with the syringe pump 20.

The radio frequency ablation control apparatus 10 is configured to perform steps in the method for controlling the perfusion of a syringe pump according to the embodiments shown in FIG. 26 to FIG. 31.

The radio frequency ablation catheter 60 is configured to perform an ablation operation on the ablation object according to a control instruction of the radio frequency ablation control apparatus 10.

The temperature acquisition apparatus 30 is configured to acquire a temperature value of the ablation object and transmit it to the radio frequency ablation control apparatus 10.

The impedance acquisition apparatus 40 is configured to acquire an impedance value of the ablation object and transmit it to the radio frequency ablation control apparatus 10.

Further, embodiments of the present invention further provide a computer-readable storage medium. The computer-readable storage medium may be provided in the electronic apparatus in each of the foregoing embodiments, and the computer-readable storage medium may be a memory in the storage and processing circuit 300 in the embodiment as shown in FIG. 33. A computer program is stored on the computer-readable storage medium, and when being executed by the processor, realizes the method for controlling the perfusion of a syringe pump described in the foregoing embodiments as shown in FIG. 26 to FIG. 31. Further, the computer storable medium may further be a U disk, a mobile hard disk, a read-only memory (ROM), a RAM, a magnetic disk or an optical disk, and other various media that may store the program code.

In the several embodiments provided in the present invention, it should be understood that the disclosed apparatus and method may be implemented in other ways. For example, the apparatus embodiments described above are merely illustrative. For example, the division of the modules is only a logical function division, or other divisions in practical implementations, for example, multiple modules or components may be combined or may be integrated into another system, or some features may be ignored or not performed. In addition, the displayed or discussed mutual coupling or direct coupling or communication connection may be indirect coupling or communication connection through some interfaces, apparatuses or modules, and may be in electrical, mechanical or other forms.

The modules described as separate components may or may not be physically separated, and the components displayed as modules may or may not be physical modules, that is, they may be located in one place, or they may be distributed onto multiple network modules. Some or all of the modules may be selected according to actual needs to achieve the objectives of the solutions of the embodiments.

In addition, the functional modules in the various embodiments of the present invention may be integrated into one processing module, or each module may exist alone physically, or two or more modules may be integrated into one module. The above-mentioned integrated modules may be implemented in the form of hardware or a software functional module.

If the integrated module is implemented in the form of the software function module and sold or used as an independent product, it may be stored in a computer-readable storage medium. Based on such understanding, the technical solution of the present invention substantively, or a part thereof making a contribution to the prior art, or all or part of the technical solution may be embodied in the form of a software product stored in a readable storage medium, and the readable storage medium includes several instructions to enable a computer device (which may be a personal computer, a server, or a network device) to execute all or part of the steps of the methods described in the various embodiments of the present invention. The above-mentioned readable storage medium includes a U disk, a mobile hard disk, a ROM, a RAM, a magnetic disk or an optical disk, and other media that may store the program code.

It should be noted that for the foregoing method embodiments, for simplicity of description, they are all expressed as a series of action combinations, but those skilled in the art should appreciate that the present invention is not limited by the described sequence of actions. Because according to the present invention, some steps may be performed in other sequences or simultaneously, and secondly, those skilled in the art should further appreciate that the embodiments described in the specification are all preferred embodiments, and the involved actions and modules are not necessarily all required by the present invention.

Data Adjustment Method in Radio-Frequency Operation and Radio-Frequency Host

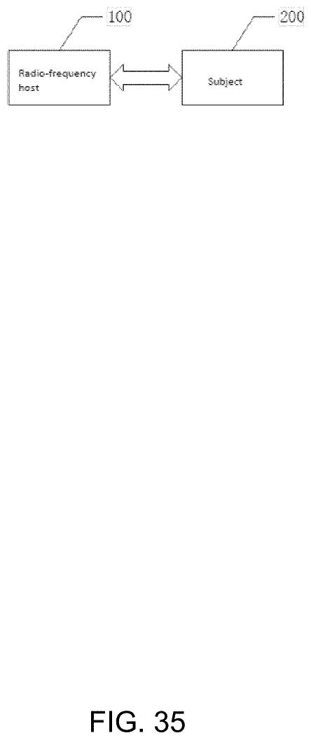

FIG. 35 is a schematic diagram showing an application scenario of a data adjustment method in a radio-frequency operation provided in an embodiment of the present invention. The data adjustment method in a radio-frequency operation includes, during the radio-frequency operation, outputting a radio-frequency signal at a set power, detecting physical characteristic data of a subject of the radio-frequency operation in real time, and determining whether to adjust the radio-frequency output power or the physical characteristic data according to the change of the physical characteristic data. As a result, the data of the radio-frequency operation tends to be more reasonable, to improve the success rate and safety of the radio-frequency operation.

Particularly, an implementation body of the data adjustment method is a radio-frequency host that may be specifically a radio-frequency ablation instrument or other devices. As shown in FIG. 35, a radio-frequency host 100 is connected to a subject 200, and then a radio-frequency operation is started, in which the radio-frequency host 100 transmits a radio-frequency signal to the subject 200 by a radio-frequency generator. In the radio-frequency operation, as the nature of the subject 200 changes, physical characteristic data also changes. The subject 200 can be any object that needs the radio-frequency operation. For example, when the radio-frequency host 100 is a radio-frequency ablation instrument, the subject 200 can be an organism that needs to ablate abnormal tissues in the body.

The radio-frequency host 100 has an input interface that can be externally connected to a movable storage such as U disk, or externally connected to an input device such as keyboard and mouse, to read data from the removable storage or acquire data inputted by a user from the input device. The radio-frequency host 100 may also be connected to a server over a network, to obtain, from the server, big data from all radio-frequency hosts connected to the server, wherein the big data includes various historical data related to the radio-frequency operation.

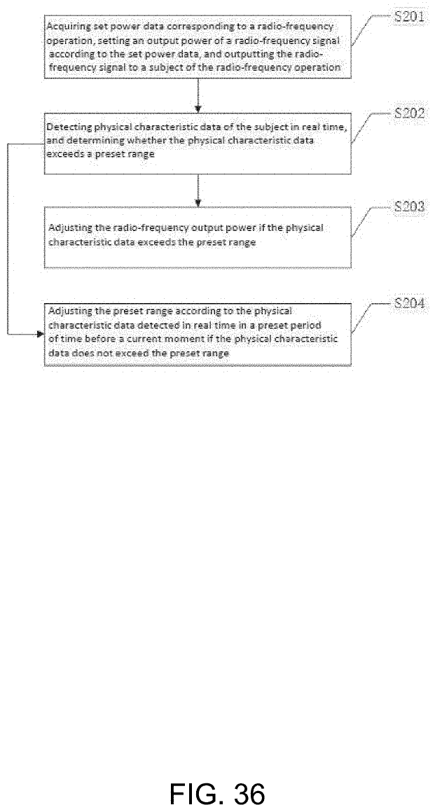

FIG. 36 is a schematic flow chart of a data adjustment method in a radio-frequency operation provided in an embodiment of the present invention. The method is applicable to the radio-frequency host as shown in FIG. 35. As shown in FIG. 36, the method includes specifically:

Step S201: acquiring set power data corresponding to a radio-frequency operation, setting an output power of a radio-frequency signal according to the set power data, and outputting the radio-frequency signal to a subject of the radio-frequency operation.

Particularly, the set power data can be obtained by obtaining, from a server, historical radio-frequency operation data of all radio-frequency hosts in a network, or obtained from set data entered by a user into the radio-frequency host.

Step S202: detecting physical characteristic data of the subject in real time, and determining whether the physical characteristic data exceeds a preset range, wherein the physical characteristic data includes the temperature and impedance of the subject.

In the radio-frequency operation, the radio-frequency signal outputted on the subject has radio-frequency energy, and a site receiving the radio-frequency operation has changed physical characteristic data under the action of the radio-frequency energy.

The preset range is a numerical interval defined by a lowest value and a highest value, and the method of obtaining the lowest value and the highest value is the same as the method of obtaining the set power data in Step S201. That is, the lowest value and the highest value can be obtained by obtaining, from a server, historical radio-frequency operation data of all radio-frequency hosts in a network, or obtained from set data entered by a user into the radio-frequency host.

Step S203: adjusting the radio-frequency output power if the physical characteristic data exceeds the preset range.

If the physical characteristic data is higher than the highest value of the preset range or lower than the lowest value of the preset range, it is determined to exceed the preset range. Then, the radio-frequency output power is adjusted, to reduce or increase the physical characteristic data.

In this embodiment, the calculation of the actual power detected in real time requires the measurement of the corresponding voltage and current, and then the real-time power is calculated according to a product of the voltage and current.

Step S204: adjusting the preset range according to the physical characteristic data detected in real time in a preset period of time before a current moment if the physical characteristic data does not exceed the preset range.

If the physical characteristic data does not exceed the preset range, the preset range is adjusted according to the physical characteristic data detected in real time in a preset period of time before a current moment. The adjusted physical characteristic data can be used as historical radio-frequency operation data, and set a data basis for a preset range of the physical characteristic data of a next radio-frequency operation, thus making the data be of great referential value, and improving the accuracy of the radio-frequency operation.

In the embodiments of the present invention, set power data corresponding to a radio-frequency operation is acquired, an output power of a radio-frequency signal is set according to the set power data, and the radio-frequency signal is outputted physical characteristic data of a subject of the radio-frequency operation is detected in real time during the radio-frequency operation, whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; and if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

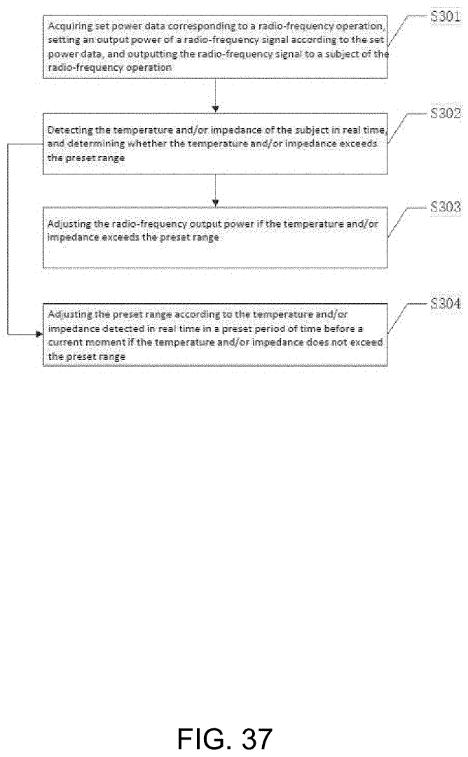

FIG. 37 is a schematic flow chart of a data adjustment method in a radio-frequency operation provided in another embodiment of the present invention. The method is applicable to the radio-frequency host as shown in FIG. 35. As shown in FIG. 37, the method includes specifically:

Step S301: acquiring set power data corresponding to a radio-frequency operation, setting an output power of a radio-frequency signal according to the set power data, and outputting the radio-frequency signal to a subject of the radio-frequency operation.

Particularly, the set power data can be obtained through the following two methods.

In a first method, historical radio-frequency operation data corresponding to the task and subject of the radio-frequency operation is obtained from a server. Then the historical radio-frequency operation data is classified according to the task of the radio-frequency operation and the nature of the subject. For example, the historical radio-frequency operation data where task No. 1 is performed and the subject is A is classified into one category, the historical radio-frequency operation data where task No. 2 is performed and the subject is A is classified into one category, the historical radio-frequency operation data where task No. 1 is performed and the subject is B is classified into one category, and so on. Because of the same task, and the same nature of the subject, the corresponding relationship between each category of historical radio-frequency operation data and the radio-frequency operation time is also the same.

Therefore, when the radio-frequency operation is performed, radio-frequency power data is queried from the corresponding historical radio-frequency operation data according to the task and subject of the current radio-frequency operation, the queried radio-frequency power data is used as the set power data, the output power of the radio-frequency signal in various periods of time of the radio-frequency operation is set according to the corresponding relationship between the set power data and the radio-frequency operation time, and the radio-frequency signal having the output power is outputted to the subject. Particularly, the output power of the radio-frequency signal in the historical radio-frequency operation data is determined as the set power data, wherein the set power data is specifically a change trend curve representing the corresponding relationship between the radio-frequency operation time and the output power. From the change trend curve, the output power in an operation time corresponding to the current stage of the current radio-frequency operation is acquired, and the acquired output power is set as the output power of the radio-frequency signal.

In a second method, the set power data can be obtained from the set data entered by a user into the radio-frequency host. Particularly, the set power data is acquired from the set data in the removable storage connected to the radio-frequency host, or the set power data is acquired from the set data inputted via an input device of the radio-frequency host. The set power data is a numerical interval including a maximum value of the set power and a minimum value of the set power, and

a median value of the numerical interval is set as the output power of the radio-frequency signal. The radio-frequency signal having the output power is outputted to the subject.

Step S302: detecting the temperature and/or impedance of the subject in real time, and determining whether the temperature and/or impedance exceeds the preset range.

Step S303: adjusting the radio-frequency output power if the temperature and/or impedance exceeds the preset range.

Particularly, the radio-frequency output power can be adjusted as follows. If the temperature or impedance of the subject detected in real time is greater than the maximum value of the preset range, the output power of the radio-frequency signal is reduced to a preset first target power; and

if both the temperature and the impedance of the subject detected in real time are less than the minimum value of the preset range, the output power of the radio-frequency signal is increased to a preset second target power.

Due to the high temperature generated by the radio-frequency energy, the impedance of the site of the subject receiving the radio-frequency operation is caused to increase. Accordingly, the temperature and/or impedance of the subject detected is detected in real time. If they exceed the preset range, and generally are greater than the maximum value of the preset range, the output power of the radio-frequency signal is reduced to the preset first target power, If the temperature and/or impedance of the subject detected still exceeds the preset range, the output power of the radio-frequency signal is further reduced to a next target power lower than the first target power. The target power for each reduction is preset in the radio-frequency host.

If a radio-frequency probe provided on the radio-frequency host is a multi-electrode radio-frequency probe, the radio-frequency output power may also be adjusted as follows. If the temperature or impedance of the subject detected in real time is greater than the maximum value of the preset range, the total power needed to be set is determined according to the minimum impedance of each electrode of the multi-electrode radio-frequency probe, and the real-time total power of the radio-frequency probe of the radio-frequency host is detected. The power adjustment is calculated by the default proportional integral differential (PID) algorithm according to the total power needed to be set and the real-time total power, and the target power is calculated according to the power adjustment and the current output power of the radio-frequency signal. Then, the radio-frequency output power is reduced to the target power.

Particularly, the impedances of multiple electrodes of the multi-electrode radio-frequency probe are detected, and an individual electrode with the smallest impedance is determined. According to the impedance of the individual electrode, the preset power of the individual electrode, and the impedances of other electrodes of the multi-electrode radio-frequency probe than the individual electrode, the powers of other electrodes are calculated, and the sum of the power of each electrode is taken as the total power needed to be set.

The power calculation formula is P=U²/R. Since each electrode of the multi-electrode radio-frequency probe is connected to the same voltage output, and each electrode has the same voltage at the site of the radio-frequency operation. The power of each electrode depends on the impedance R, and the power P increases with the decrease of R. The power of each individual electrode is delimited by the set total power, and can be equal to, but cannot exceed the set total power. The set total power is the sum of the power of each electrode.

Particularly, the current total power needed to be set is calculated according to the impedance of the electrode;

According to the formula P=U²/R, it can be deduced that the relationship between the power Pₗᵢₘ of the electrode with the smallest impedance and the power Pₙ of other individual electrodes is:

$$\frac{{P}_{li m}}{{P}_{n}} = \frac{{U}^{2} / {R}_{l im}}{{U}^{2} / {R}_{n}} = \frac{{R}_{n}}{{R}_{l im}} ,$$

that is,

$${P}_{n} = \frac{{P}_{lim} {R}_{lim}}{{R}_{n}} .$$

Pₗᵢₘ is the power of the known electrode with the smallest impedance, and according to Rₗᵢₘ and the impedances Rₙ of other individual electrodes, Pₙ corresponding to each individual electrode can be obtained. The total power P needed to be set is calculated by the formula

$$P = {\sum }_{i = 1}^{n} {P}_{i} .$$

According to the currently measured real-time total power and the total power P needed to be set, the total power increment ΔP can be obtained according to the PID algorithm. The PID algorithm is accomplished by

$$\begin{matrix}u ( k ) = {K}_{P} \{ err ( k ) + \frac{T}{{T}_{I}} {\sum }_{j = 0}^{k} err ( j ) + \frac{{T}_{D}}{T} [ err ( k ) - err ( k - 1 ) ] \} ; or & Formula 1 \\ u ( k ) = {K}_{P} err ( k ) + {K}_{I} {\sum }_{j = 0}^{k} err ( j ) + {K}_{D} [ err ( k ) - err ( k - 1 ) ] ; & Formula 2\end{matrix}$$

wherein K_{P},

$${K}_{I} = {K}_{P} \frac{T}{{T}_{I}} , and {K}_{D} = {K}_{P} \frac{{T}_{D}}{T}$$

are respectively the proportional coefficient, integral coefficient and differential coefficient of the PID algorithm, T is the sampling time, T_{I} is the integration time (also referred to as the integral coefficient), T_{D} is the differential time (also referred to as the differential coefficient), err(k) is the difference between the total power needed to be set and the real-time total power, and u(k) is the output.

By using the incremental PID algorithm ΔP=u(k)−u(k−1), it can be obtained from Formula 2 above:

$$Δ P = {K}_{P} [ e r r ( k ) - e r r ( k - 1 ) ] + {K}_{I} err ( k ) + {K}_{D} [ e r r ( k ) - 2 e r r ( k - 1 ) + e r r ( k - 2 ) ]$$

The output adjustment is calculated according to ΔP, and the adjustment has a one-to-one mapping relationship with ΔP, because the power adjustment is achieved by controlling a voltage signal from a power board, the output voltage corresponds to an input digital signal of a digital-to-analog converter, and the adjustment actually corresponds to this digital signal. The mapping relationship enables a corresponding relationship between the output and ΔP, for example, the output of 1 means that the corresponding power increment ΔP is 0.1 w. In this way, the control of ΔP is achieved according to the mapping relationship.

The current power is increased by a value of ΔP to obtain the target power. When ΔP is a negative value, the increment ΔP means to reduce the radio-frequency output power, to lower the temperature. Otherwise, when ΔP is a positive value, it means to increase the radio-frequency output power, to increase the temperature.

The radio-frequency output power is adjusted to the target power and then outputted.

If the temperature or impedance of the subject detected in real time is less than the minimum value of the preset range, the method to adjust the power is as described above.

Step S304: adjusting the preset range according to the temperature and/or impedance detected in real time in a preset period of time before a current moment if the temperature and/or impedance does not exceed the preset range.

Particularly, according to the various temperatures and/or impedances detected in real time in the preset period of time, and a default selection algorithm, a target value is selected from various temperatures and/or impedances in the preset time to update the end values of the preset range, wherein the end values include a minimum and a maximum value.

More specifically, the preset period of time is 10 sec. Taking temperature as an example, a minimum value among various temperatures in 10 seconds before the current moment is selected as the minimum value of the preset range, and a maximum value among various temperatures is selected as the maximum value of the preset range; or a median value of various temperatures in 10 seconds before the current moment is calculated, and the to-be-updated end values corresponding to the median value is calculated according to the median value with reference to the difference between the median value and the end values of the preset range before updating, wherein calculated end values are the end values of the updated preset range.

In the embodiments of the present invention, set power data corresponding to a radio-frequency operation is acquired, an output power of a radio-frequency signal is set according to the set power data, and the radio-frequency signal is outputted, the temperature and impedance of a subject of the radio-frequency operation is detected in real time during the radio-frequency operation, and whether the temperature and/or impedance exceeds a preset range is determined, wherein if the temperature or impedance is greater than the maximum value of the preset range, the output power of the radio-frequency signal is reduced, to reduce the risk of the radio-frequency operation damaging the subject and improve the safety of the radio-frequency operation; if the temperature and impedance are both lower than the minimum value of the preset range, the output power of the radio-frequency signal is increased, to improve the effect of the radio-frequency operation; and further, if the temperature and/or impedance does not exceed the preset range, the reasonableness of the preset range is automatically updated, to provide a more accurate data basis for subsequent radio-frequency operations, and improve the reasonableness and success rate of the radio-frequency operation.

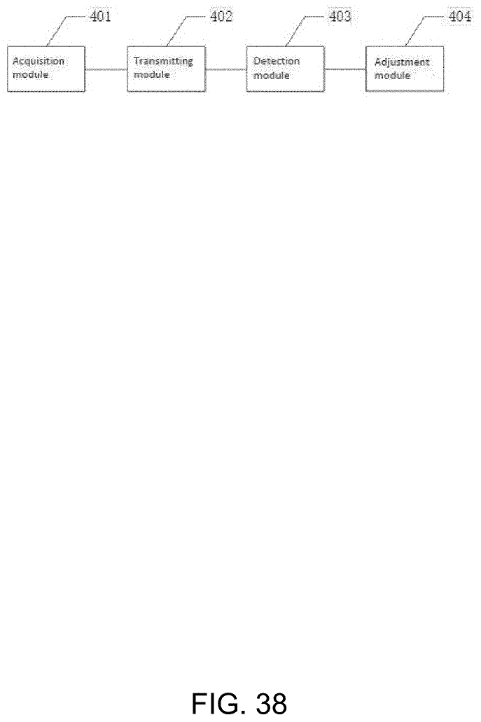

FIG. 38 is a schematic structural diagram of a radio-frequency host provided in an embodiment of the present invention. For ease of description, only the parts relevant to the embodiments of the present invention are shown. The radio-frequency host is a radio-frequency host for implementing the data adjustment method in a radio-frequency operation described in the above embodiments. The radio-frequency host includes: an acquisition module 401, configured to acquire set power data corresponding to a radio-frequency operation; a transmitting module 402, configured to set an output power of a radio-frequency signal according to the set power data, and output the radio-frequency signal to a subject of the radio-frequency operation; a detection module 403, configured to detect physical characteristic data of the subject in real time, and determine whether the physical characteristic data exceeds a preset range; and an adjustment module 404, configured to adjust the radio-frequency output power if the physical characteristic data exceeds the preset range, and adjust the preset range according to the physical characteristic data detected in real time in a preset period of time before a current moment if the physical characteristic data does not exceed the preset range.

The various modules in the radio-frequency host serve to implement the following functions. Set power data corresponding to a radio-frequency operation is acquired, an output power of a radio-frequency signal is set according to the set power data, and the radio-frequency signal is outputted, physical characteristic data of a subject of the radio-frequency operation is detected in real time during the radio-frequency operation, and whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

Further, the detection module 403 is further configured to detect the temperature and/or impedance of the subject in real time.

Further, the adjustment module 404 is further configured to reduce the radio-frequency output power to a preset first target power, if the temperature or impedance of the subject detected in real time is greater than the maximum value of the preset range; and increase the radio-frequency output power to a preset second target power if the temperature and impedance of the subject detected in real time are both less than the minimum value of the preset range.

If a radio-frequency probe provided on the radio-frequency host is a multi-electrode radio-frequency probe, the detection module 403 is further configured to determine the total power needed to be set according to the minimum impedance of each electrode of the multi-electrode radio-frequency probe, if the temperature or impedance of the subject detected in real time is greater than the maximum value of the preset range, and detect the real-time total power of the radio-frequency probe of the radio-frequency host; and the adjustment module 404 is further configured to calculate a power adjustment by default PID algorithm according to the total power needed to be set and the real-time total power, calculate a target power according to the power adjustment and the current output power of the radio-frequency signal, and reduce the radio-frequency output power to the target power.

The adjustment module 403 is also configured to select a target value from various temperatures and/or impedances to update end values of the preset range according to the various temperatures and/or impedances detected in real time in the preset period of time and a default selection algorithm.

The acquisition module 401 is further configured to acquire historical radio-frequency operation data corresponding to the task and subject of the radio-frequency operation; and determine the output power of the radio-frequency signal in the historical radio-frequency operation data as the set power data, wherein the set power data is a change trend curve representing the corresponding relationship between the radio-frequency operation time and the output power.

The transmitting module 402 is further configured to acquire the output power in an operation time corresponding to the current stage of the current radio-frequency operation, and set the acquired output power as the output power of the radio-frequency signal.

The acquisition module 401 is further configured to acquire the set power data from an externally connected removable storage, or acquire the set power data inputted from an input device, wherein the set power data is a numerical interval including a maximum value of the set power and a minimum value of the set power.

The transmitting module 402 is further configured to set a median value of the numerical interval as the output power of the radio-frequency signal.

In the embodiments of the present invention, set power data corresponding to a radio-frequency operation is acquired, an output power of a radio-frequency signal is set according to the set power data, and the radio-frequency signal is outputted, the temperature and impedance of a subject of the radio-frequency operation is detected in real time during the radio-frequency operation, and whether the temperature and/or impedance exceeds a preset range is determined, wherein if the temperature or impedance is greater than the maximum value of the preset range, the radio-frequency output power is reduced, to reduce the risk of the radio-frequency operation damaging the subject and improve the safety of the radio-frequency operation; if the temperature and impedance are both lower than the minimum value of the preset range, the output power of the radio-frequency signal is increased, to improve the effect of the radio-frequency operation; and further, if the temperature and/or impedance does not exceed the preset range, the reasonableness of the preset range is automatically updated, to provide a more accurate data basis for subsequent radio-frequency operations, and improve the reasonableness and success rate of the radio-frequency operation.

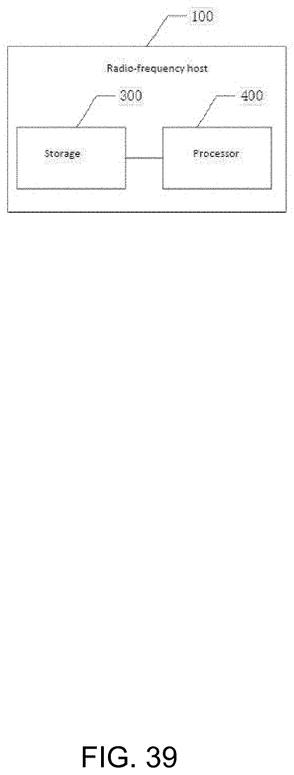

Further, as shown in FIG. 39, an embodiment of the present invention also provides a radio-frequency host, which includes a storage 300 and a processor 400, wherein the processor 400 may be a central processor in the radio-frequency host provided in the above embodiments. The storage 300 is, for example, hard drive storage, a non-volatile storage (such as flash memory or other storages that are used to form solid-state drives and are electronically programmable to confine the deletion, etc.), and a volatile storage (such as static or dynamic random access storage), which is not limited in the embodiments of the present invention.

The storage 300 stores an executable program code; and the processor 400 is 300 coupled to the storage 300, and configured to call the executable program code stored in the storage, and implement the data adjustment method in a radio-frequency operation as described above.

Further, an embodiment of the present invention further provides a computer-readable storage medium. which can be provided in the radio-frequency host in each of the above embodiments, and may be the storage 300 in the embodiment shown in FIG. 39. A computer program is stored in the computer-readable storage medium, and when the program is executed by a processor, the data adjustment method in a radio-frequency operation according to the embodiments shown in FIG. 36 and FIG. 37 is implemented. Further, the computer-readable storage medium may also be a U disk, a removable hard disk, a read-only storage (ROM, Read-Only Memory), RAM, a magnetic disk or an optical disk and other media that can store program codes.

In the above embodiments, emphasis has been placed on the description of various embodiments. Parts of an embodiment that are not described in detail may be found in the description of other embodiments.

The data adjustment method in a radio-frequency operation and the radio-frequency host provided in the present invention have been described above. Changes can be made to the specific implementation and the scope of the present invention by those skilled in the art according to the idea of the embodiments of the present invention. Therefore, the disclosure of this specification should not be construed as a limitation of the present invention.

Method for Protecting Radio Frequency Operation Abnormality, Radio Frequency Mainframe, and Radio Frequency Operation System (The '914 Patent)

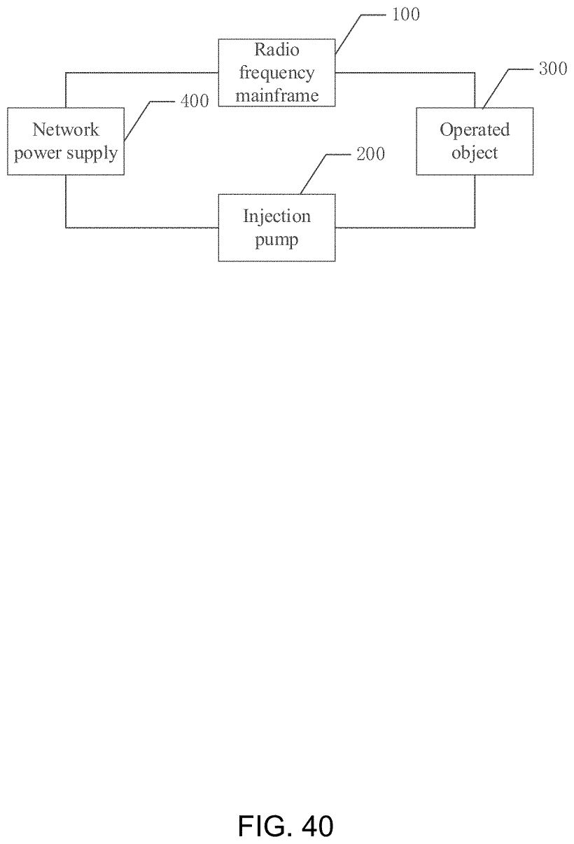

Referring to FIG. 40, which is a schematic diagram of an application scene of a method for protecting radio frequency operation abnormality provided by an embodiment of the present invention. The method for protecting radio frequency operation abnormality can be configured to: when a radio frequency mainframe continuously outputs energy, if it is detected that a radio frequency operation appears an abnormal state, protect the radio frequency mainframe and a radio frequency operated object from being damaged through multiple manners, thereby improve safety of radio frequency operation.

Specifically, as shown in FIG. 40, a radio frequency mainframe 100, an injection pump 200, and an operated object 300 are interconnected to form a radio frequency operating system, which is powered by a network power supply 400. The network power supply 400 is a common A4920V voltage, and a power supply system of the radio frequency mainframe 100 itself performs necessary processing and splitting for the network power supply 400 and then outputs power to other apparatuses or modules of the radio frequency mainframe 100. Among them, the radio frequency mainframe 100 may specifically be a radio frequency ablation apparatus or other equipment, which outputs radio frequency energy to the operating object 300. During radio frequency operations, according to requirements of the operations, the injection pump 200 injects liquid into the operating object 300 for cooling, impedance reduction, etc.

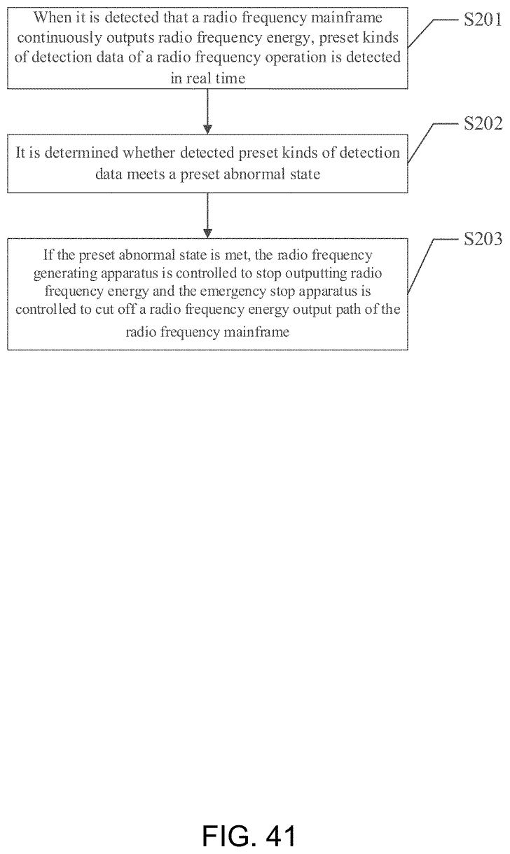

Referring to FIG. 41, which is a schematic flow chart of a method for protecting radio frequency operation abnormality provided by an embodiment of the present invention. The method can be applied in the radio frequency mainframe as shown in FIG. 40. As shown in FIG. 41, the method specifically comprises the follows.

Step S201, when it is detected that a radio frequency mainframe continuously outputs radio frequency energy, preset kinds of detection data of a radio frequency operation is detected in real time.

An executing main body of this embodiment is a radio frequency mainframe, the radio frequency mainframe includes at least a detecting apparatus, a controlling apparatus, a radio frequency generating apparatus, and an emergency stop apparatus, the controlling apparatus is connected to the detecting apparatus, the radio frequency generating apparatus, and the emergency stop apparatus respectively, and the detecting apparatus is further connected to the radio frequency generating apparatus.

The detecting apparatus is used to detect various data of a radio frequency operation process, including data of radio frequency energy output, an impedance and a temperature of an operated object, voltages and currents of circuits, etc. In addition, the detecting apparatus is further provided therein with a processor that is independent of the controlling apparatus of the radio frequency mainframe, including a single-chip microcomputer, an MCU (Microcontroller Unit), a CPU (Central Processing Unit), etc., which can realize functions of data collection, data distribution, data analysis, and so on.

The radio frequency apparatus is used to emit radio frequency energy acting on an operated object.

The emergency stop apparatus is used to stop a radio frequency operation quickly when an emergency state set in a system occurs.

The controlling apparatus is a processor of the radio frequency mainframe, which is used to acquire data, analyzed data, and control starting and stopping of functions of all apparatuses or modules in the radio frequency mainframe according to analysis result.

Specifically, the radio frequency energy data includes output power and output time of radio frequency energy, the detecting apparatus detects data of radio frequency energy emitted by the radio frequency generating apparatus; when the output power of radio frequency energy reaches preset working power and the output time reaches a preset output time length, it is determined that the radio frequency main frame continuously outputs radio frequency energy to the operated object, and a stable radio frequency operation stage is entered.

Step S202, it is determined whether detected preset kinds of detection data meets a preset abnormal state.

The preset abnormal state includes an abnormality determination standard for the preset kinds of detection data.

Furthermore, information of the abnormal state is also set in the controlling apparatus.

Step S203, if the preset abnormal state is met, the radio frequency generating apparatus is controlled to stop outputting radio frequency energy and the emergency stop apparatus is controlled to cut off a radio frequency energy output path of the radio frequency mainframe.

When the detection data of the detecting apparatus meets the preset abnormal state, dual protection including the following two manners is performed for a radio frequency operation appearing abnormality.

One manner is controlling the radio frequency generating apparatus to stop outputting radio frequency energy, and the other is to control the emergency stop apparatus to cut off a radio frequency energy output path of the radio frequency mainframe. It can be prevented that unexpected failure of any one manner causes radio frequency energy to output continuously and brings damage to the operated object and the radio frequency mainframe.

In this embodiment of the present invention, when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

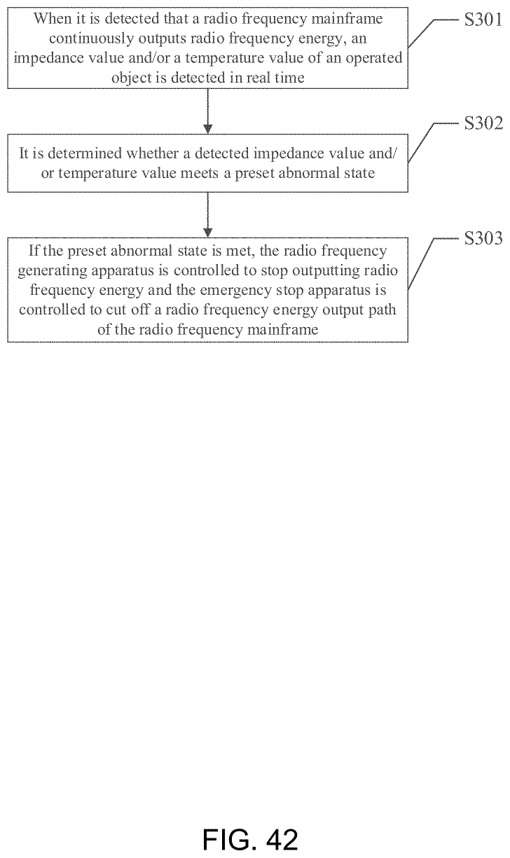

Referring to FIG. 42, which is an implementation flow chart of a method for protecting radio frequency operation abnormality provided by another embodiment of the present invention. The method can be applied in the radio frequency mainframe as shown in FIG. 40. As shown in FIG. 42, the method specifically comprises the follows.

Step S301, when it is detected that a radio frequency mainframe continuously outputs radio frequency energy, an impedance value and/or a temperature value of an operated object is detected in real time.

A too high impedance value or temperature value of the operating object may cause irreversible damage to the operated object, and is an important protection direction. During a radio frequency operation process, an impedance value or a temperature value of an operating object is detected in real time, or the impedance value and the temperature value are detected at the same time.

Step 302, it is determined whether a detected impedance value and/or temperature value meets a preset abnormal state.

Specifically, it is determined whether a detected impedance value of an operated object is higher than a first preset impedance threshold, and whether a duration time length of being higher than the first preset impedance threshold is larger than a preset time length, wherein the preset time length is preferably 3 seconds; and/or whether a temperature value of an operated object is higher than a first preset temperature threshold, and whether a duration time length of being higher than the first preset temperature threshold is larger than the preset time length.

Both the first preset impedance threshold and the first preset temperature threshold are upper limit values, and specifically relate to content of the present radio frequency operation and a type of the operated object, which are not specifically limited.

If the impedance value of the operated object is higher than the first preset impedance threshold, and the duration time length of being higher than the first preset impedance threshold is larger than the preset time length, or if the temperature value of the operated object is higher than the first preset temperature threshold, and the duration time length of being higher than the first temperature impedance threshold is larger than the preset time length, it is determined that the preset abnormal state is met. That is, when any one value of the impedance value and the temperature value of the operated object is higher than an upper limit value, it can be determined that the current radio frequency operation appears the preset abnormal state.

Step S303, if the preset abnormal state is met, the radio frequency generating apparatus is controlled to stop outputting radio frequency energy and the emergency stop apparatus is controlled to cut off a radio frequency energy output path of the radio frequency mainframe.

Specifically, on one hand, detected detection data of the preset kind is sent to the controlling apparatus, the controlling apparatus can analyze that the preset abnormal state occurs according to the detection data, send stop information for stopping generating and outputting radio frequency energy to the radio frequency generating apparatus, so as to control the radio frequency generating apparatus to stop outputting radio frequency energy.

Alternatively, after determining that the preset abnormal state occurs, the detecting apparatus directly sends abnormal state prompt information to the controlling apparatus; the controlling apparatus sends stop information for stopping generating and outputting radio frequency energy to the radio frequency generating apparatus according to the prompt information.

On the other hand, the detecting apparatus controls the emergency stop apparatus to cut off a radio frequency energy output path of the radio frequency mainframe; specifically, cutting information is send to the emergency stop apparatus connected to the detecting apparatus, and the emergency stop apparatus cuts off a radio frequency energy output path between the radio frequency mainframe and the operated object.

As described above, by the detecting apparatus and the controlling apparatus, which are two different apparatuses, the radio frequency generating apparatus and the emergency stop apparatus are respectively controlled to simultaneously stop delivering radio frequency energy to the operated object, so that failure risk of completing control by the same one apparatus is avoided, a succeeding rate of stopping delivering is improved, and safety of protecting the operated object and the radio frequency mainframe is further improved.

Other technical details of the above steps refer to description of the embodiment shown in aforementioned FIG. 41, and are not repeated here.

In this embodiment of the present invention, when a radio frequency mainframe continuously outputs radio frequency energy, an impedance value and/or a temperature value of a radio frequency operated object is detected in real time; if any one of the impedance value and the temperature value is higher than a preset upper limit value, a radio frequency generating apparatus is controlled to stop outputting radio frequency energy and an emergency stop apparatus is controlled to cut off a radio frequency energy output path of the radio frequency mainframe. The above two manners of protection are performed at the same time, a succeeding rate of protection is improved, and safety of radio frequency operations is improved.

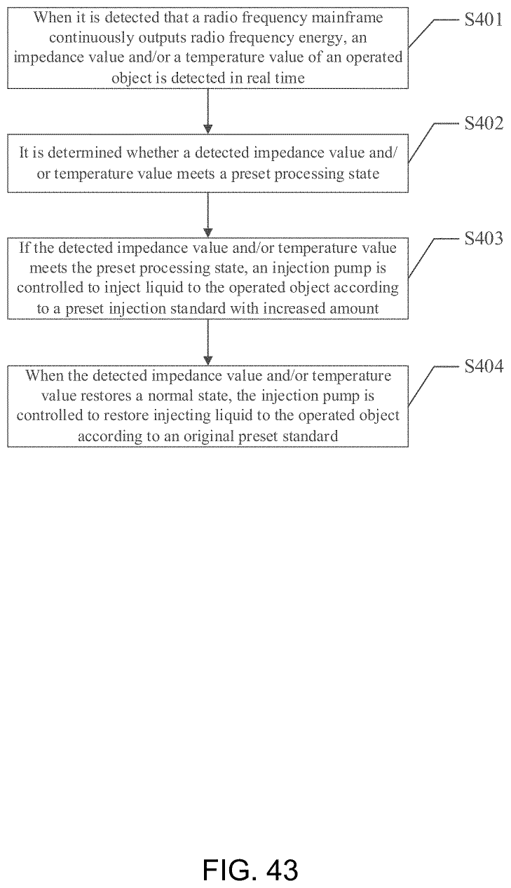

Referring to FIG. 43, which is an implementation flow chart of a method for protecting radio frequency operation abnormality provided by another embodiment of the present invention. The method can be applied in the radio frequency mainframe as shown in FIG. 40. As shown in FIG. 43, the method specifically comprises the follows.

Step 401, when it is detected that a radio frequency mainframe continuously outputs radio frequency energy, an impedance value and/or a temperature value of an operated object is detected in real time.

Step S402, it is determined whether a detected impedance value and/or temperature value meets a preset processing state.

It is detected in real time whether the impedance value of the operated object is higher than a second preset impedance threshold, or it is detected in real time whether an increasing ratio of the impedance value of the operated object is higher than a first preset ratio; if being higher than the second preset impedance threshold or higher than the first preset ratio, it is determined that the preset processing state is met. That is, although no preset abnormal state occurs, when the impedance value of the operated object is higher than a normal value or its increasing ratio is larger than a normal ratio, it is represented that the operated object appears abnormality, but an extent requiring stopping radio frequency energy output is not reached, thus it is determined that the preset processing state is met.

It is detected in real time whether the temperature value of the operated object is higher than a second preset temperature threshold, or it is detected in real time whether an increasing ratio of the temperature value of the operated object is higher than a second preset ratio; if being higher than the second preset temperature threshold or higher than the second preset ratio, it is determined that the preset processing state is met. That is, although no preset abnormal state occurs, when the temperature value of the operated object is higher than a normal value or its increasing ratio is larger than a normal ratio, it is represented that the operated object appears abnormality, but an extent requiring stopping radio frequency energy output is not reached, thus it is determined that the preset processing state is met.

Step S403, if the detected impedance value and/or temperature value meets the preset processing state, an injection pump is controlled to inject liquid to the operated object according to a preset injection standard with increased amount.

When the impedance value and/or the temperature value of the operated object appears abnormal increase, but does not reach the extent requiring stopping radio frequency energy output, an injection pump is controlled to inject liquid used to decrease the impedance value and/or the temperature value to the operated object according to a first preset injection standard with increased amount. Increasing injection amount can decrease the impedance value and the temperature value of the operated object.

Step S404, when the detected impedance value and/or temperature value restores a normal state, the injection pump is controlled to restore injecting liquid to the operated object according to an original preset standard.

When it is detected that the impedance value of the operated object is lower than a third preset impedance threshold, and/or the temperature value of the operated object is lower than a third preset temperature threshold, it is determined that the impedance value and/or the temperature value of the operated object has restored to a normal value. Thus, liquid is injected to the operated object according to the original preset injection standard with decreased amount, and injection amount according to the impedance value and/or the temperature value in a normal state is restored.

In this embodiment of the present invention, when it is detected that a radio frequency mainframe continuously outputs radio frequency energy, an impedance value and/or a temperature value of an operated object is detected in real time. If the impedance value and/or the temperature value increases to meet a preset processing state, an injection pump is controlled to increase injection mount to decrease the impedance value and/or the temperature value; if the impedance value and/or the temperature value restores to a normal value, the injection pump is controlled to restore to an original injection amount to keep the impedance value and/or the temperature value of the operated object being at the normal value. By the above-mentioned dynamic adjustment for abnormality of the impedance value and/or the temperature value that does not reaching the extent of stopping radio frequency energy output, it is possible to perform protection for the radio frequency object and the radio frequency mainframe in advance, and improve safety of radio frequency operations.

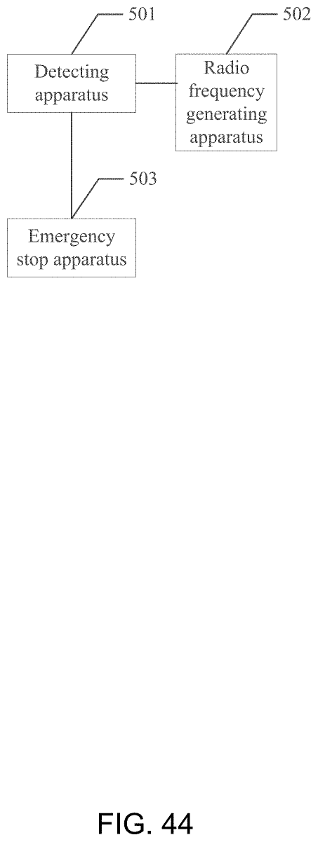

Referring to FIG. 44, which is a structural schematic diagram of a radio frequency mainframe provided by an embodiment of the present invention. In order to facilitate illustration, only parts relating to embodiments of the present invention are shown. The radio frequency mainframe is the radio frequency mainframe shown in above FIG. 40-FIG. 43, which comprises a detecting apparatus 501, a radio frequency generating apparatus 502, and an emergency stop apparatus 503.

Among them, the detecting apparatus 501 is configured to: when it is detected that the radio frequency mainframe continuously outputs radio frequency energy, detect preset kinds of detection data of a radio frequency operation in real time; the detecting apparatus 501 is further configured to: determine whether detected preset kinds of detection data meets a preset abnormal state; and the detecting apparatus 501 is further configured to: if the preset abnormal state is met, control the radio frequency generating apparatus 502 to stop outputting radio frequency energy and control the emergency stop apparatus 503 to cut off a radio frequency energy output path.

In this embodiment of the present invention, when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

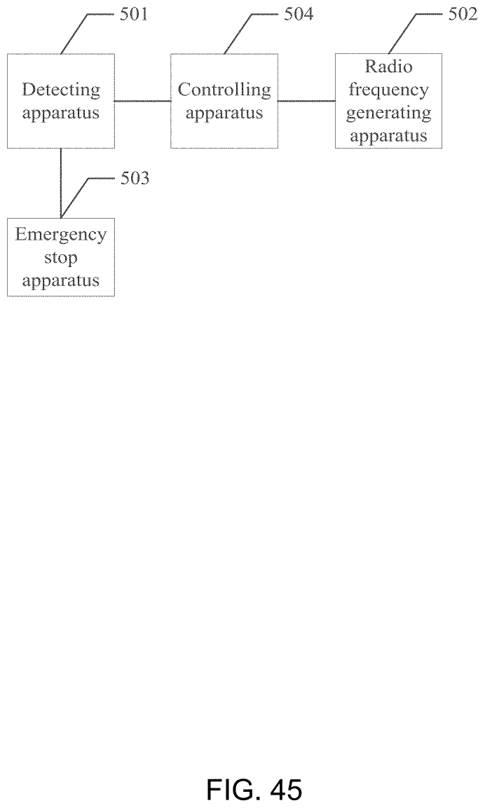

Referring to FIG. 45, which is a structural schematic diagram of a radio frequency mainframe provided by another embodiment of the present invention. The radio frequency mainframe is the radio frequency mainframe shown in above FIG. 40-FIG. 44, which differs from the embodiment shown in above FIG. 44 as follows.

Furthermore, the radio frequency mainframe further comprises a controlling apparatus 504.

The detecting apparatus 501 is further configured to transmit detection data meeting the preset abnormal state to the controlling apparatus 504 and thereby trigger the controlling apparatus 504 to control the radio frequency generating apparatus 502 to stop outputting radio frequency energy. The controlling apparatus 504 is configured to transmit information for stopping outputting radio frequency energy to the radio frequency generating apparatus 502 according to received detection data.

The controlling apparatus 504 can be further configured to analyze that the preset abnormal state occurs according to the detection data, and send stop information for stopping generating and outputting radio frequency energy to the radio frequency generating apparatus 502.

In another aspect, the detecting apparatus 501 is further configured to: after determining that the preset abnormal state occurs, directly send abnormal state prompt information to the controlling apparatus 504; the controlling apparatus 504 is further configured to send stop information for stopping generating and outputting radio frequency energy to the radio frequency generating apparatus according to the prompt information.

The detecting apparatus 501 is further configured to send cutting information to the emergency stop apparatus 503, and thereby cut off a radio frequency energy output path between the radio frequency mainframe and the operated object.

Furthermore, the detecting apparatus 501 is further configured to detect an impedance value and/or a temperature value of the operated object of the radio frequency operation in real time.

The detecting apparatus 501 is further configured to determine whether the impedance value of the operated object is higher than a first preset impedance threshold, and whether a duration time length of being higher than the first preset impedance threshold is larger than a preset time length, and/or whether the temperature value of the operated object is higher than a first preset temperature threshold, and whether a duration time length of being higher than the first preset temperature threshold is larger than the preset time length.

The detecting apparatus 501 or the controlling apparatus 504 is further configured to: if the impedance value of the operated object is higher than the first preset impedance threshold, and the duration time length of being higher than the first preset impedance threshold is larger than the preset time length, or if the temperature value of the operated object is higher than the first preset temperature threshold, and the duration time length of being higher than the first temperature impedance threshold is larger than the preset time length, determine that the preset abnormal state is met.

The detecting apparatus 501 is further configured to: detect in real time whether the impedance value of the operated object is higher than a second preset impedance threshold, or detected in real time whether an increasing ratio of the impedance value of the operated object is higher than a first preset ratio; if being higher than the second preset impedance threshold or higher than the first preset ratio, send the aforesaid detection information or prompt information to the controlling apparatus 504.

The controlling apparatus 504 is further configured to: according to the detection information or prompt information, send controlling instruction to an injection pump to control injection pump to inject liquid used to decrease the impedance value to the operated object according to a first preset injection standard with increased amount.

The detecting apparatus 501 is further configured to: detect in real time whether the temperature value of the operated object is higher than a second preset temperature threshold, or it is detected in real time whether an increasing ratio of the temperature value of the operated object is higher than a second preset ratio; if being higher than the second preset temperature threshold or higher than the second preset ratio, transmit the aforesaid detection information or prompt information to the controlling apparatus 504 for control.

The controlling apparatus 504 is further configured to: according to the detection information or prompt information, transmit controlling instruction to the injection pump and thereby make the injection pump inject liquid used to decrease the temperature to the operated object according to a first preset injection standard with increased amount.

In this embodiment of the present invention, when a radio frequency mainframe continuously outputs radio frequency energy, an impedance value and/or a temperature value of a radio frequency operated object is detected in real time; if any one of the impedance value and the temperature value is higher than a preset upper limit value, a radio frequency generating apparatus is controlled to stop outputting radio frequency energy and an emergency stop apparatus is controlled to cut off a radio frequency energy output path of the radio frequency mainframe. The above two manners of protection are performed at the same time, a succeeding rate of protection is improved, and safety of radio frequency operations is improved.

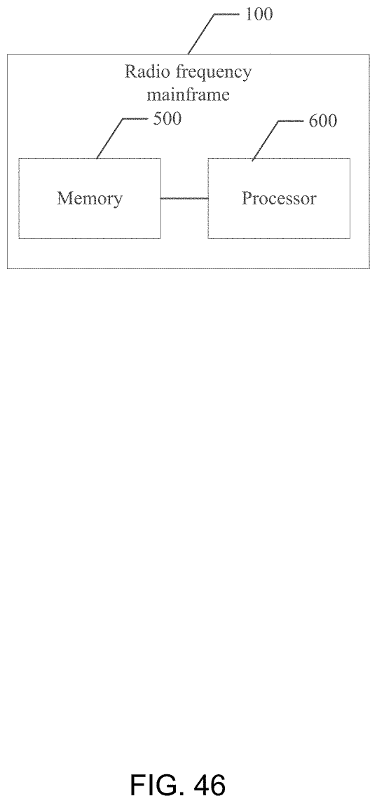

As shown in FIG. 46, an embodiment of the present invention further provides a radio frequency mainframe, which comprises a memory 500 and a processor 600, the processor 600 can be the detecting apparatus in the aforementioned embodiments, and can also be the controlling apparatus. The memory 500 is, for example, a hard disk drive memory, a non-volatile memory (such as a flash memory or other electronically programmable and deletion-restricted memory used to form a solid state drive, etc.), a volatile memory (such as a static or dynamic random access memory, etc.), and so on. The embodiments of the present invention are not limited.

The memory 500 stores executable program codes. A processor 600 coupled with the memory 500 calls the executable program codes stored in the memory to execute the above-described method for protecting radio frequency operation abnormality.

Further, an embodiment of the present invention further provides a computer-readable storage medium, the computer-readable storage medium can be set in the radio frequency mainframes in the above embodiments, and the computer-readable storage medium can be the memory 500 in the embodiment in the above embodiment shown in FIG. 46. The computer-readable storage medium stores a computer program, and the program, when being executed by a processor, implements the method for protecting radio frequency operation abnormality described in the embodiments shown in above FIG. 41, FIG. 42, and FIG. 43. Further, the computer-readable storage medium can also be a U-disk, a mobile hard disk, a read-only memory (ROM), a RAM, a magnetic disk or a CD-ROM, and other media that can store program codes.

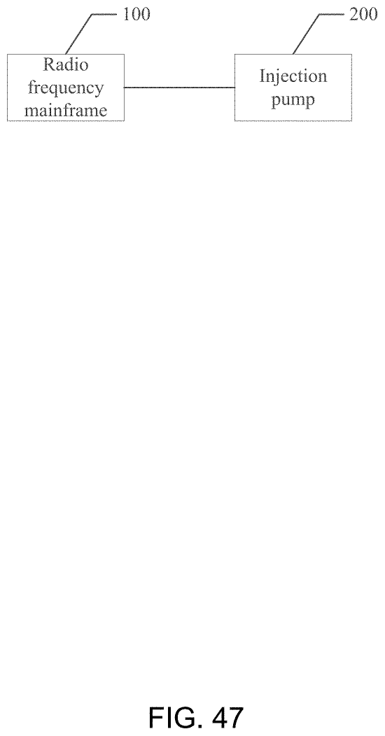

Further, referring to FIG. 47, an embodiment of the present invention further provides a radio frequency operation system, which comprises a radio frequency mainframe 100 and an injection pump 200.

The radio frequency mainframe 100 is configured to implement the method for protecting radio frequency operation abnormality described as FIG. 41, FIG. 42, and FIG. 43. The injection pump 200 is configured to inject liquid with a preset function to a radio frequency operated object under control of the radio frequency mainframe.

Other technical details refer to description of above-described embodiments.

Method and Apparatus for Dynamically Adjusting Radio Frequency Parameter and Radio Frequency Host

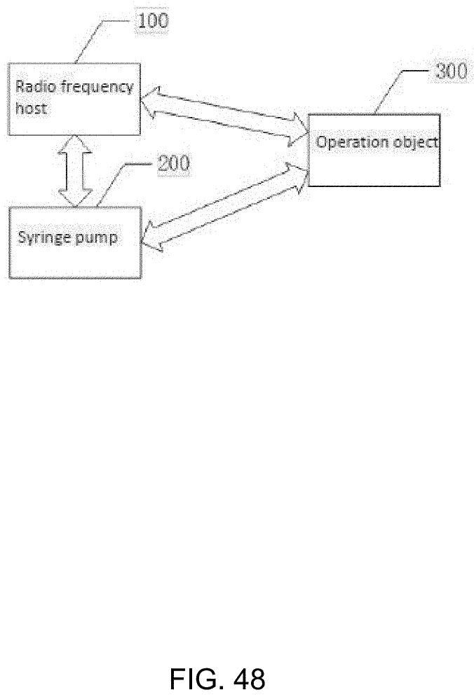

FIG. 48 is a schematic diagram showing an application scenario of a method for dynamically adjusting a radio frequency parameter according to an embodiment of the present invention. The method for dynamically adjusting the radio frequency parameter may be used to compare radio frequency data with a standard range and a limit range corresponding to a current operation stage by detecting the radio frequency data during a radio frequency operation, and confirm whether a problem occurs in the radio frequency operation, thereby interfering the radio frequency operation having the problem, such that the radio frequency operation may continue to be performed smoothly, the serious problem may be interrupted in time, and the safety of the radio frequency operation is improved.

Particularly, an execution main body of the method is a radio frequency host, and the radio frequency host may particularly be a device such as a radio frequency ablation instrument. As shown in FIG. 48, the radio frequency host 100 is connected with a syringe pump 200, and the radio frequency host 100 and the syringe pump 200 are connected with the operation object 300 as well. When the radio frequency operation starts, the radio frequency host 100 sends radio frequency energy to the operation object 300 by a radio frequency generation apparatus. The radio frequency host 100 controls the injection pump 200 to inject the operation object 300 with a cooling liquid. The radio frequency host 100 has radio frequency data standard ranges and radio frequency data limit ranges of various stages of an initial stage, a middle stage and a final stage of performing the radio frequency operation on the operation object 300. During the radio frequency operation, when characters of the operation object 300 change, the radio frequency data acting on the operation object will change therewith.

Further, the radio frequency data standard range and the radio frequency data limit range may be a numerical range, including the maximum value and the minimum value. If the real-time radio frequency data of the operation object 300 is greater than the maximum value or less than the minimum value of the standard range, the radio frequency data is enabled to be within the standard range by controlling an injection volume of the syringe pump. The injection volume may be controlled by controlling an injection flow rate. If the real-time radio frequency data of the operation object 300 is greater than the maximum value or less than the minimum value of the limit range, it is confirmed that a problem occurs in the radio frequency host 100 or the operation object 300, and the radio frequency operation has to be stopped. The radio frequency data standard range and the radio frequency data limit range of the radio frequency data may further be a radio frequency data change rate, that is, a radio frequency data slope. Within a preset detection duration, the real-time radio frequency data forms an excessive slope, which exceeds a preset slope, the purpose of adjusting the radio frequency data is achieved by adjusting the injection volume of the syringe pump, or the radio frequency operation is stopped to eliminate failures. Accordingly, the safety of the radio frequency operation is improved.

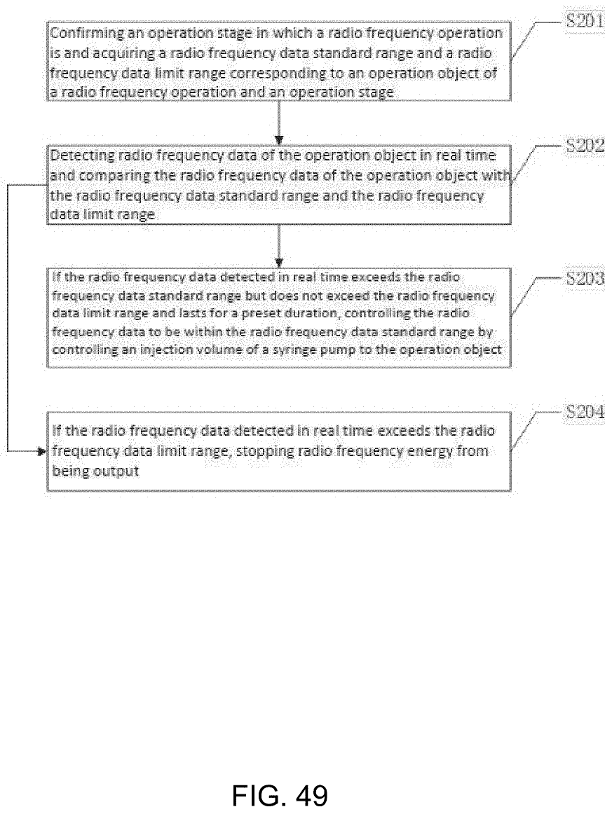

Reference is made to FIG. 49, which is a schematic flow chart of a method for dynamically adjusting a radio frequency parameter according to an embodiment of the present invention. The method may be applied to a radio frequency host as shown in FIG. 48. As shown in FIG. 49, the method particularly includes the following steps.

In step S201, a current operation stage in which a radio frequency operation is FIG'S confirmed, and a radio frequency data standard range and a radio frequency data limit range corresponding to an operation object of the radio frequency operation and the operation stage are acquired.

The radio frequency data standard range is within the radio frequency data limit range, that is, the minimum value of the radio frequency data standard range is greater than the minimum value of the radio frequency data limit range, and the maximum value of the radio frequency data standard range is greater than the maximum value of the radio frequency data limit range.

Particularly, for different types of operation objects or individual differences of the same type of operation objects, the radio frequency data standard range will be different. For different radio frequency operation stages of the same operation object, the radio frequency data standard range and the radio frequency data limit range are different. The operation object may be any object performing the radio frequency operation. For example, during radio frequency ablation, the operation object may be an abnormal tissue of a biological body, and the abnormal tissue is eliminated or reduced by means of ablation.

The radio frequency host has information on the radio frequency data standard ranges and the radio frequency data limit ranges of a specific operation object at different operation stages inside, for being read by a detection apparatus of the radio frequency host, and based on the radio frequency data of the operation object of the current radio frequency operation and the operation stage detected in real time, and being compared with the radio frequency data standard range and the radio frequency data limit range, respectively.

In step S202, the radio frequency data of the operation object is detected in real time, and compared with the radio frequency data standard range and the radio frequency data limit range, respectively.

The radio frequency operation will generate the radio frequency data when acting on the operation object, wherein the radio frequency data may particularly include an impedance value, a temperature value, a current value and a voltage value. The radio frequency host detects the above radio frequency data of the operation object in real time, and these radio frequency data feeds back whether the current radio frequency operation is normal.

In step S203, if the radio frequency data detected in real time exceeds the radio frequency data standard range but does not exceed the radio frequency data limit range and lasts for a preset duration, the radio frequency data is controlled to be within the radio frequency data standard range by controlling the injection volume of the injection pump to the operation object.

When it is detected that the radio frequency data of the test object in the current operation stage exceeds the radio frequency data standard range and lasts for the preset duration, the radio frequency data is adjusted to reach the normal standard range by controlling the injection volume of the syringe pump, the instability of the radio frequency data due to accidental factors is further eliminated and the intelligence of detection is improved.

In step S204, if the radio frequency data detected in real time exceeds the radio frequency data limit range, the radio frequency energy is stopped from being output.

If the radio frequency data detected in real time exceeds the radio frequency data standard range, indicating that a serious problem occurs in the radio frequency operation, in order to protect the safety of the radio frequency host and the operation object, the radio frequency energy is immediately stopped from being output. Particularly, a detection module may send the radio frequency data to a processor of the radio frequency host, and the processor sends a stop signal to a radio frequency signal generation apparatus of the radio frequency host, such that the radio frequency signal generation apparatus is stopped from outputting the radio frequency signal.

In the embodiment of the present invention, the radio frequency data standard range corresponding to the operation object of the radio frequency operation and the current operation stage is acquired, and the radio frequency data detected in real time is compared with the radio frequency data standard range and the radio frequency data limit range, respectively. If the radio frequency detected in real time exceeds the radio frequency data standard range but does not exceed the radio frequency data limit range and lasts for the preset duration, the radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the radio frequency data detected in real time exceeds the radio frequency data limit range, it is confirmed that a problem exists in the radio frequency host of the current radio frequency operation or the operation object, and the radio frequency energy is stopped from being output. Therefore, the radio frequency host and the operation object are prevented from being damaged, and the safety of the radio frequency operation is improved.

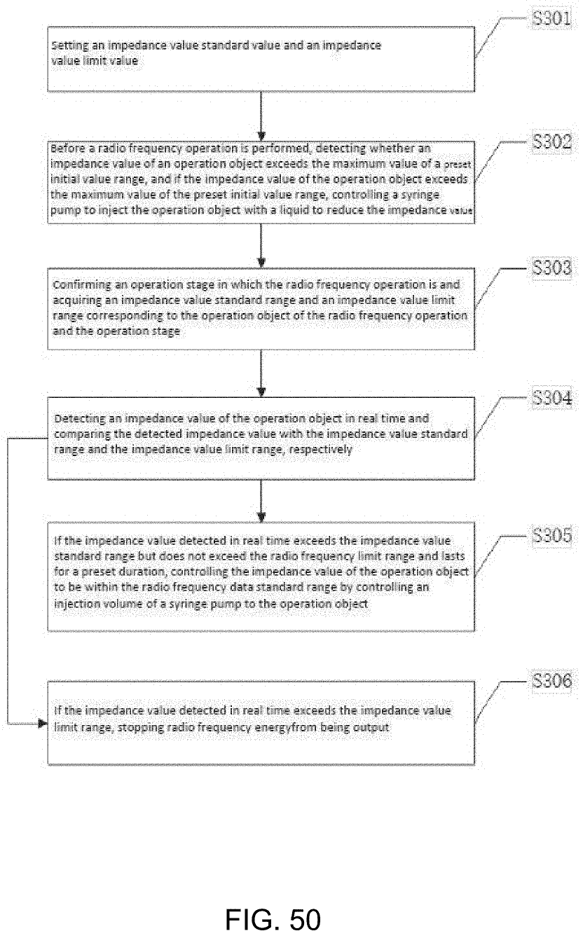

Reference is made to FIG. 50, which is a flow chart of an implementation of a method for dynamically adjusting a radio frequency parameter according to another embodiment of the present invention. The method may be applied to a radio frequency host as shown in FIG. 48. As shown in FIG. 50, the method particularly includes the following steps.

In step S301, an impedance value standard range and an impedance value limit range are set.

In response to a setting operation of a user, an input interface of the lowest value, the highest value and a change rate of the impedance value is displayed, wherein the setting operation may be a user input, or may be called from a memory of the radio frequency host or a database of a server connected with the radio frequency host according to an instruction from the user.

A first lowest value, a first highest value and a first change rate of the impedance value set by the user are acquired, the first lowest value and the first highest value input by the user are used as the lowest value and the highest value of a standard numerical interval, and the first change rate input by the user is used as the standard slope.

A second lowest value, a second highest value and a second change rate of the impedance value set by the user are acquired, the second lowest value and the second highest value input by the user are used as the lowest value and the highest value of a limit numerical interval, and the second change rate set by the user is used as the standard slope.

Among them, the first lowest value is higher than the second lowest value, the first highest value is lower than the second lowest value, the first change rate is lower than the second change rate, and the low change rate indicates that the value changes a little per unit time.

Particularly, the impedance value is set corresponding to a model of the radio frequency host, a task of the radio frequency operation and a nature of the operation object, and an operation position of the radio frequency operation on the operation object. Such a correspondence relationship is known and may be input by the user or stored in related devices such as the radio frequency host or the server in advance.

In step S302, before the radio frequency operation is performed, it is detected whether the impedance value of the operation object exceeds the highest value of a preset initial value range, and if the impedance value of the operation object exceeds the highest value of the preset initial value range, the syringe pump is controlled to inject the operation object with a liquid to reduce the impedance value.

If the detected impedance value of the operation object is higher than the highest value of the preset initial value range, the syringe pump is controlled to inject the operation object with the liquid to reduce the impedance value of the operation object, until the impedance value meets the preset initial value range. That is, before the radio frequency operation starts, the initial impedance value of the operation object is set to be within the normal initial value range, so as to reduce the influence on the detection and the determination based on the impedance value during the radio frequency operation due to the deviation of the initial impedance value from the normal range after the radio frequency operation starts.

The initial value range corresponds to the nature of the operation object and a specific position of the radio frequency operation on the operation object, and is a general range obtained based on actual measurement values of a plurality of operation objects. For example, when the radio frequency operation is radio frequency ablation, the operation object is a human body and a specific location is a lung tissue, and the initial value range is 250Ω to 350Ω (ohm).

In step S303, a current operation stage in which the radio frequency operation is FIG'S confirmed, and an impedance value standard range and an impedance value limit range corresponding to the operation object of the radio frequency operation and the operation stage are acquired.

The impedance value standard range may be a standard numerical interval of the impedance value, and the standard numerical interval is a numerical interval including the lowest value and the highest value. During the radio frequency operation, the impedance value standard range is preferably 150Ω to 500Ω, or a standard slope set according to the nature of the operation object, that is, a standard change rate of the impedance value without being adjusted is confirmed according to the nature of the operation object under a premise that the impedance value of the operation object is not less than 150Ω and is not greater than 500Ω during the radio frequency operation. No adjustment on a real-time change rate of the impedance value below the standard change rate is required for the operation object. The standard change rate is the standard slope.

The impedance value limit range may be a limit numerical interval of the impedance value, and the limit numerical interval is a numerical interval including the lowest value and the highest value. During the radio frequency operation, the impedance value limit range is preferably 50Ω to 600Ω, or is a limit slope set according to the nature of the operation object, that is, a limit change rate of a safe impedance value is confirmed according to the nature of the operation object under a premise that the impedance value of the operation object is not less than 50Ω and is not greater than 600Ω during the radio frequency operation. A real-time change rate of the impedance value below the standard change rate is safe for the operation object. The standard change rate is the standard slope.

Specific values of standard numerical interval, the standard slope, the limit numerical interval and the limit slope of the impedance value are related to the operation object and the operation stage in which the radio frequency operation is performed, and will be not particularly limited.

In step S304, the impedance value of the operation object is detected in real time, and the detected impedance value is compared with the impedance value standard range and the impedance value limit range, respectively.

The impedance value of the operation object may be directly detected by an impedance detection circuit, or a current value of the operation object may be detected by a current detection circuit, and a voltage value of the operation object may be detected by a voltage detection circuit. The impedance value of the operation object is calculated according to a calculation formula of the current value, the voltage value and the impedance value.

The impedance value detected in real time is compared with the standard numerical interval of the impedance value corresponding to the current operation stage of the operation object and/or the standard slope of the impedance value in real time, and compared with the limit numerical interval of the impedance value corresponding to the current operation stage of the operation object and/or the limit slope of the impedance value.

In step S305, if the impedance value detected in real time exceeds the impedance value standard range but does not exceed the radio frequency data limit range and lasts for the preset duration, the impedance value of the operation object is controlled to be within the radio frequency data standard range by controlling the injection volume of the injection pump to the operation object.

Particularly, if the impedance value of the operation object detected in real time is lower than the lowest value of the standard numerical interval and/or a decrease rate of the impedance value of the operation object is greater than the first standard slope and lasts for the preset duration, the injection pump is controlled to reduce the amount of the liquid injected into the operation object according to a preset first injection volume.

Any one or both of two cases where the impedance value of the operation object is lower than the lowest value of the standard numerical interval and lasts for the preset duration and the decrease rate of the impedance value of the operation object is greater than the first standard slope and lasts for the preset duration indicates or indicate that the impedance value of the operation object is too low or decreases too quickly and it is necessary to increase the impedance value. Therefore, the injection volume of the injection pump to the operation object is reduced, and the injection volume may be controlled by controlling an injection flow rate of the liquid. Within a fixed duration, the greater the flow rate is, the greater the injection volume is.

If the impedance value of the operation object detected in real time is higher than the highest value of the standard numerical interval and/or the increase rate of the impedance value of the operation object is greater than the second standard slope and lasts for the preset duration, the injection pump is controlled to increase the amount of the liquid injected into the operation object according to a preset second injection volume.

Any one or both of two cases where the impedance value of the operation object is higher than the highest value of the standard numerical interval and lasts for the preset duration and the increase rate of the impedance value of the operation object is greater than the second standard slope and lasts for the preset duration indicates or indicate that the impedance value of the operation object is too high or increases too rapidly and it is necessary to reduce the impedance value. Therefore, the injection volume of the injection pump to the operation object is increased.

Further, the impedance value may change due to accidental factors. In order to prevent the instability of a working process of the syringe pump due to frequent adjustment on the impedance value from influencing the effect of the radio frequency operation, the accidental factors may be eliminated after the preset duration is elapsed and the step of dynamically adjusting the impedance value of the operation object is started.

If the impedance value of the operation object may not be controlled to be within the radio frequency data standard range by controlling the injection volume of the injection pump to the operation object after a preset adjustment duration is elapsed, the radio frequency operation is stopped.

In step S306, if the impedance value detected in real time exceeds the impedance value limit range, the radio frequency energy is stopped from being output.

Particularly, the limit slope of the impedance value includes a first limit slope used to indicate a decrease rate of the impedance value and a second limit slope used to indicate the increase rate of the impedance value. The limit numerical interval, the first limit slope and the second limit slope are limit abnormal values in nature, that is, no matter what operation stage in which the impedance value of the operation object detected in real time is, the radio frequency energy has to be stopped immediately from being output to the operation object provided that at least one of the following first conditions is met, the first conditions may include: the impedance value is higher than the maximum value of the limit numerical interval, the impedance value is lower than the minimum value of the limit numerical interval, the decrease rate of the impedance value is greater than the first limit slope, and the increase rate of the impedance value is greater than the second limit slope. Among them, the decrease rate of the impedance value is a ratio of a decrement of the impedance value of the operation object per unit duration to the unit duration; and the increase rate of the impedance value is a ratio of an increment of the impedance value of the operation object per unit duration to the unit duration.

Further, while the radio frequency energy is stopped from being output to the operation object, a text indicator is displayed and an audible and visual alarm is given. Particularly, when the impedance value of the operation object detected in real time is lower than the lowest value of the limit numerical interval or the decrease rate of the impedance value of the operation object detected in real time is greater than the first limit slope, a first text indicator is displayed and the audible and visual alarm is given.

When the impedance value of the operation object detected in real time is higher than the highest value of the limit numerical interval or the increase rate of the impedance value of the operation object detected in real time is greater than the first limit slope, a second text indicator is displayed and the audible and visual alarm is given.

The first text indicator for example displays a text “LLL” on a display screen of the radio frequency host, and the second text indicator for example displays a text “HHH” on the display screen of the radio frequency host. The audible and visual alarm includes both an audible alarm and a visual alarm.

Further, when it is detected that the impedance value of the operation object exceeds the above non-limit abnormal value, the text indicator may be displayed and the audible and visual alarm may be given. Particularly, the text indicator and the audible and visual alarm may be different from those when the impedance value of the operation object exceeds the limit abnormal value in terms of contents and forms. The audible and visual alarm is distinguished from the above audible and visual alarm in the terms of forms.

Further, after it is detected that the impedance value of the operation object exceeds the above-mentioned non-limit abnormal value and lasts for the preset duration, the radio frequency energy is stopped from being output to the operation object, and the text indicator is displayed and the audible and visual alarm is given. At this time, the text indicator and the audible and visual alarm are the same as those when the impedance value of the operation object exceeds the limit abnormal value in the terms of contents and forms.

Further, if the detected impedance value of the operation object exceeds the radio frequency data standard range, a correspondence relationship between the impedance value of the operation object and the time of the radio frequency operation is displayed on a display interface in the form of a line with a first color; and if the detected impedance value of the operation object does not exceed the radio frequency data standard range, a correspondence relationship between the impedance value of the operation object and the time of the radio frequency operation is displayed on the display interface in the form of a line with a second color.

The reflectivity of the first color is higher than that of the second color, the greater the reflectivity is, the more the light dazzles, and the higher a reminding degree to human eyes is. For example, a red color reflects 67% of light, a yellow color reflects 65% of light, a green color reflects 47% of light, and a cyan color reflects 36% of light. The first color may be the red or yellow color, and the second color may be the green or cyan color.

For technical details of the above steps, reference is made to the description of the embodiment as shown in FIG. 49, which will be omitted here.

In the embodiment of the present invention, before the radio frequency operation is performed, the impedance value of the operation object is reduced to be within the normal initial value range in a fashion of injecting the liquid by the syringe pump, so as to improve the accuracy of detecting the impedance value during the subsequent radio frequency operation and improve the success rate of the radio frequency operation. The standard numerical interval of the impedance value and the standard change slope of the impedance value corresponding to the operation object of the radio frequency operation and the current operation stage are acquired. The impedance value of the operation object detected in real time is compared with the standard numerical interval and/or the standard slope and with the limit numerical interval and/or the limit slope in real time. The radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the impedance value of the operation object detected in real time exceeds the limit numerical interval and/or the change rate of the impedance value is greater than the limit slope, it is confirmed that a problem occurs in the radio frequency host of the current radio frequency operation or the operation object, the radio frequency energy is stopped from being output. As a result, the radio frequency host and the operation object are prevented from being damaged, and the safety of the radio frequency operation is improved. The text indicator is displayed and the audible and visual alarm is given, which further remind a radio frequency operator of paying attention to the safety of the radio frequency operation.

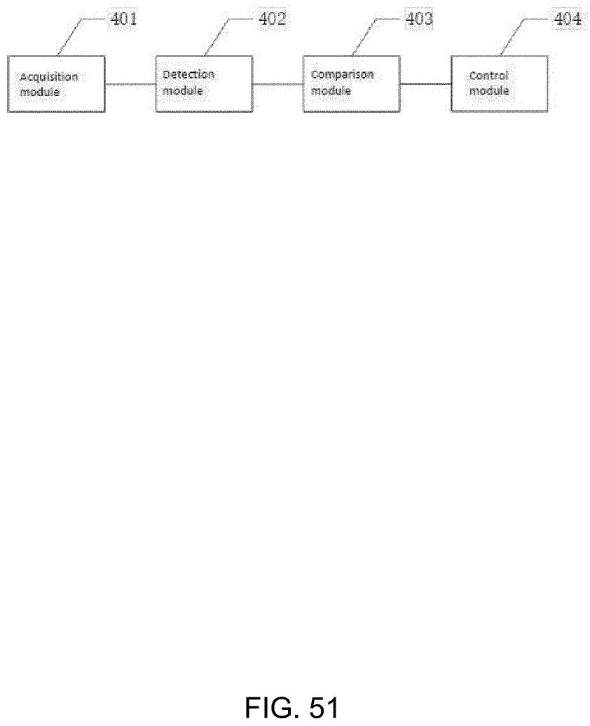

Reference is made to FIG. 51, which is a schematic diagram showing a structure of an apparatus for dynamically adjusting a radio frequency parameter according to an embodiment of the present invention. In order to facilitate the illustration, parts related to the embodiment of the present invention are only shown. The apparatus may be disposed in the above radio frequency host. The apparatus includes: an acquisition module 401, which is configured to confirm an operation stage in which a radio frequency operation is and acquire a radio frequency data standard range and a radio frequency data limit range corresponding to an operation object of the radio frequency operation and the operation stage, wherein the radio frequency data standard range is within the radio frequency data limit range; a detection module 402, which is configured to detect radio frequency data of the operation object in real time; a comparison module 403, which is configured to compare the detected radio frequency data with the radio frequency data standard range and the radio frequency data limit range; and a control module 404, which is configured to if the radio frequency data detected in real time exceeds the radio frequency data standard range but does not exceed the radio frequency data limit range and lasts for a preset duration, control the radio frequency data to be within the radio frequency data standard range by controlling an injection volume of the syringe pump to the operation object, and if the radio frequency data detected in real time exceeds the radio frequency data limit range, stop radio frequency energy from being output.

Further, the acquisition module 401 is further configured to acquire a standard numerical interval of an impedance value of the operation object, a standard change slope of the impedance value of the operation object, a limit numerical interval of the impedance value of the operation object, and a limit slope of the impedance value change of the operation object in the operation stage.

The acquisition module 401 is further configured to display an input interface of the lowest value, the highest value and the change rate of the impedance value in response to a setting operation of a user, acquire a first lowest value, a first highest value, a first change rate, a second lowest value, a second highest value and a second change rate use the first lowest value and the first highest value as the lowest value and the highest value of the standard numerical interval and use the first change rate input by the user as the standard slope.

The second lowest value and the second highest value are taken as the lowest value and the highest value of the limit numerical interval, and the second change rate input by the user is taken as the limit slope.

Further, the limit slope includes a first limit slope used to indicate a decrease rate of the impedance value and a second limit slope used to indicate an increase rate of the impedance value. The control module 404 is further configured to when the impedance value of the operation object detected in real time meets at least one of preset conditions, stop from outputting radio frequency energy to the operation object, wherein the preset conditions include: the impedance value of the operation object detected in real time exceeds the limit numerical interval, the decrease rate of the impedance value of the operation object detected in real time is greater than the first limit slope, and the increase rate of the impedance value of the operation object detected in real time is greater than the second limit slope.

Further, the apparatus further includes a pre-warning module (not shown in the drawing), wherein the pre-warning module is configured to when the impedance value of the operation object detected in real time is lower than the lowest value of the limit numerical interval or the decrease rate of the impedance value of the operation object detected in real time is greater than the first limit slope, display a first text indicator and give an audible and visual alarm; and when the impedance value of the operation object detected in real time is higher than the highest value of the limit numerical interval or the increase rate of the impedance value of the operation object detected in real time is greater than the second limit slope, display a second text indicator and give the audible and visual alarm.

Further, the standard slope includes a first standard slope used to indicate a decrease rate of the impedance value and a second standard slope used to indicate an increase rate of the impedance value. The control module 404 is further configured to if the impedance value of the operation object detected in real time is lower than the lowest value of the standard numerical interval and/or a decrease rate of the impedance value of the operation object is greater than the first standard slope and lasts for the preset duration, control the injection pump to reduce the amount of the liquid injected into the operation object according to a preset first injection volume; and if the impedance value of the operation object detected in real time is higher than the highest value of the standard numerical interval and/or an increase rate of the impedance value of the operation object is greater than the second standard slope and lasts for the preset duration, control the injection pump to increase the amount of the liquid injected into the operation object according to a preset second injection volume.

Further, the detection module 402 is further configured to before the radio frequency operation is performed, detect whether the impedance value of the operation object exceeds the highest value of a preset initial value range.

The control module 404 is further configured to if the impedance value of the operation object exceeds the highest value of the preset initial value range, control the syringe pump to inject the operation object with the liquid to reduce the impedance value, until the impedance value meets a preset initial value range.

Further, the apparatus further includes a display module (not shown in the drawing), wherein the display module is configured to if the detected impedance value of the operation object exceeds the radio frequency data standard range, display a correspondence relationship between the impedance value of the operation object and the time of the radio frequency operation on a display interface in the form of a line with a first color; if the detected impedance value of the operation object does not exceed the radio frequency data standard range, display a correspondence relationship between the impedance value of the operation object and the time of the radio frequency operation on the display interface in the form of a line with a second color; and wherein the reflectivity of the first color is higher than that of the second color.

In the embodiment of the present invention, before the radio frequency operation is performed, the impedance value of the operation object is reduced to be within the normal initial value range in a fashion of injecting the liquid by the syringe pump, so as to improve the accuracy of detecting the impedance value during the subsequent radio frequency operation and improve the success rate of the radio frequency operation. The standard numerical interval of the impedance value and the standard change slope of the impedance value corresponding to the operation object of the radio frequency operation and the current operation stage are acquired. The impedance value of the operation object detected in real time is compared with the standard numerical interval and/or the standard slope and with the limit numerical interval and/or the limit slope in real time. The radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the impedance value of the operation object detected in real time exceeds the limit numerical interval and/or the change rate of the impedance value is greater than the limit slope, it is confirmed that a problem occurs in the radio frequency host of the current radio frequency operation or the operation object, and the radio frequency energy is stopped from being output. As a result, the radio frequency host and the operation object are prevented from being damaged, and the safety of the radio frequency operation is improved. The text indicator is displayed and the audible and visual alarm is given, which further remind a radio frequency operator of paying attention to the safety of the radio frequency operation.

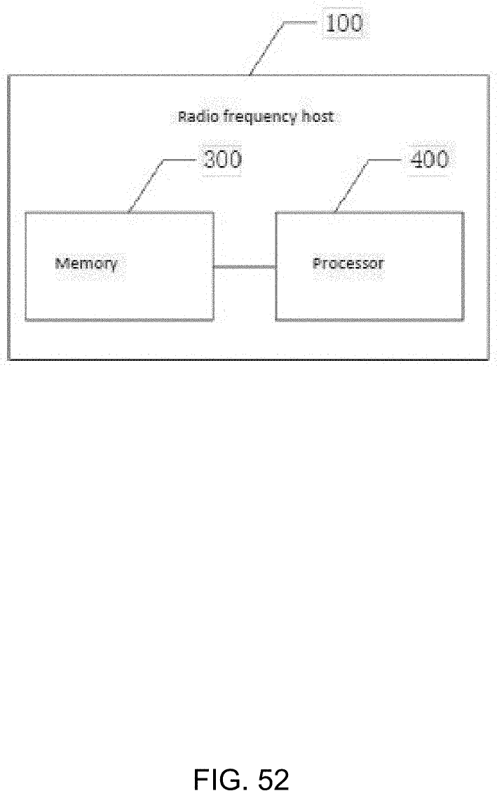

As shown in FIG. 52, embodiments of the present invention further provide a radio frequency host, including a memory 300 and a processor 400. The processor 400 may be a control module 404 in the apparatus for dynamically adjusting the radio frequency parameter in the foregoing embodiment. The memory 300 may be for example a hard disk drive memory, a non-volatile memory (such as a flash memory or another electronically programmable and restricted delete memory used to form a solid-state drive and the like), a volatile memory (such as a static or dynamic random access memory and the like) and the like, which will not be limited in the embodiment of the present invention.

The memory 300 stores an executable program code; and the processor 400 coupled with the memory 300 calls the executable program code stored in the memory to execute the method for dynamically adjusting the radio frequency parameter as described above.

Further, embodiments of the present invention further provide a computer-readable storage medium. The computer-readable storage medium may be provided in the radio frequency host in each of the foregoing embodiments, and the computer-readable storage medium may be a memory 300 in the embodiment as shown in FIG. 52. A computer program is stored on the computer-readable storage medium, and when being executed by the processor, implements the method for dynamically adjusting the radio frequency parameter according to the embodiments as shown in FIG. 49 and FIG. 50. Further, the computer-readable storable medium may further be a U disk, a mobile hard disk, a read-only memory (ROM), a RAM, a magnetic disk or an optical disk, and other various media that may store the program code.

It should be noted that for simplicity of description, the foregoing method embodiments are all expressed as a series of action combinations, but because according to the present invention, some steps may be performed in other sequences or simultaneously, those skilled in the art should appreciate that the present invention is not limited by the described sequence of actions. Secondly, those skilled in the art should further appreciate that the embodiments described in the specification are all preferred embodiments, and the involved actions and modules are not necessarily all required by the present invention.

In the above-mentioned embodiments, descriptions of the embodiments have particular emphasis respectively. For parts that are not described in detail in a certain embodiment, reference may be made to related descriptions of other embodiments.

The above is a description of the method and the apparatus for dynamically adjusting the radio frequency parameter and the radio frequency host according to the present invention. For those skilled in the art, changes may be made to specific implementations and application scopes according to the ideas of the embodiments of the present invention. In summary, the content of this specification should not be construed as a limitation on the present invention.

Method and Apparatus for Safety Control of Radio Frequency Operation, and Radio Frequency Mainframe

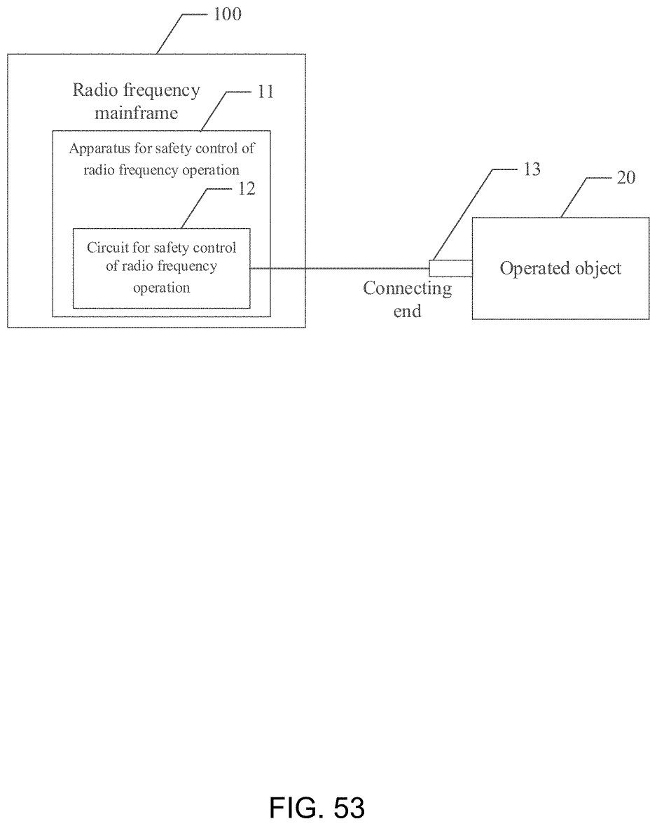

Referring to FIG. 53, which is a schematic diagram of an application scene of a method for safety control of radio frequency operation provided by an embodiment of the present invention; the method for safety control of radio frequency operation can be used to control safety problems relating to connection between a radio frequency mainframe and a radio frequency operated object through the radio frequency mainframe, and thereby improve safety and intelligence of radio frequency operations.

Specifically, the radio frequency mainframe may be a device such as a radio frequency ablation apparatus, and the radio frequency operated object may be any object that needs a radio frequency operation. For example, when the radio frequency mainframe is a radio frequency ablation apparatus, the radio frequency operated object may be an animal body that needs to ablate mutated tissues in the body. As shown in FIG. 53, a radio frequency mainframe 100 is connected with an operation object 200. The radio frequency mainframe 100 is provided therein with a radio frequency operation safety control device 11 and a radio frequency circuit 12. The radio frequency circuit 12 is used to detect whether a connection between the radio frequency mainframe 100 and the operated object 200 meets a standard, and the radio frequency circuit 12 has a connection terminal 13 (specifically, it may be a neutral electrode), the radio frequency mainframe 100 is connected with the operated object 200 through the connection terminal 13. The radio frequency mainframe 100 controls whether to output a radio frequency signal and the output power of the output radio frequency signal through the radio frequency operation safety control device 11.

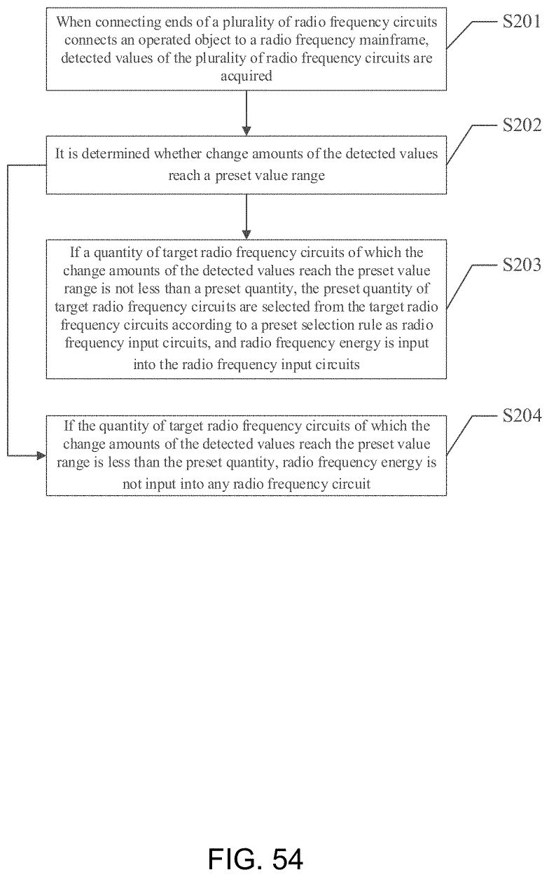

Referring to FIG. 54, which is a schematic flow chart of a method for safety control of radio frequency operation provided by an embodiment of the present invention. The method can be applied to the radio frequency mainframe shown in FIG. 53, as shown in FIG. 54, the method specifically comprises the follows.

Step S201, when connecting ends of a plurality of radio frequency circuits connects an operated object to a radio frequency mainframe, detected values of the plurality of radio frequency circuits are acquired.

Specifically, an execution subject of this embodiment is the radio frequency mainframe, and the radio frequency mainframe can detect whether a connecting end has been connected to the operation object. The connecting end is used to connect an operated object to the radio frequency mainframe, and whether tightness of the connection meets a connection standard is a prerequisite of whether the radio frequency mainframe can complete a radio frequency operation smoothly and safely.

The connecting end can be a neutral electrode; the operated object is an object or target for the radio frequency mainframe to perform a radio frequency operation.

The radio frequency circuit includes a detection circuit, which is used to detect whether a connection between a connecting end and an operated object meets a connection standard; when the connecting end is a neutral electrode, the detection circuit specifically detects whether an attachment degree between the neutral electrode and the operated object meets an attachment standard.

The radio frequency circuit further includes a radio frequency radio module; under control of the radio frequency mainframe, the radio frequency module inputs a radio frequency signal into the radio frequency circuit to execute a radio frequency operation for the operated object.

Step S202, it is determined whether change amounts of the detected values reach a preset value range.

Whether a change amount of a detected value reaches a preset value range is a determination standard for the radio frequency mainframe to determine whether a connection between a connecting end and an operated object meets a connection standard.

Specifically, the detected value can be an impedance value, and can also be a change value of a voltage value of a primary coil of a transformer.

If a change amount of the detected values reaches a preset value range, the radio frequency mainframe determines that the connection between the connecting end and the operated object meets the connection standard; if the change amount of the detected values does not reach the preset value range, the radio frequency mainframe determines that the connection between the connecting end and the operated object does not meet the connection standard.

Step S203, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

For example, if the preset quantity is 2, that is, target radio frequency circuits of which connections between connecting ends and the operated object meet the connection standard reach 2, 2 of the target radio frequency circuits are selected from these target radio frequency circuits according to a preset selection rule as radio frequency input circuits, and radio frequency energy is input thereto to be provided to the operated object.

It should be noted that each radio frequency circuit and its connecting end have their own preset numbers. According to these numbers, the radio frequency mainframe can know which operation area of the radio frequency operation the connecting terminal of the radio frequency circuit is connected to, and determine how many radio frequency circuits each operation area needs to connect, that is, how many connecting ends are connected, according to nature of the current radio frequency operation.

The selection rule is: according to an operation area and the number of connecting ends required by the current radio frequency operation, to select a preset quantity of target radio frequency circuits from the target radio frequency circuits meeting the connection standard as the radio frequency input circuits.

Distribution of the target radio frequency circuits should be capable of meeting selection requirement, and both the operation area and the quantity of the distribution are met.

Step S204, if the quantity of target radio frequency circuits of which the change amounts of the detected values reach the preset value range is less than the preset quantity, radio frequency energy is not input into any radio frequency circuit.

If the quantity of target radio frequency circuits of which the change amounts of the detected values reach the preset value range is less than the preset quantity, it is unable to meet requirement of the present radio frequency operation. Therefore, no radio frequency energy is output, that is, radio frequency energy is not input into any radio frequency circuit. It is also possible to simultaneously trigger an alarm module to issue an alarm, including flashing of an alarm light and tweeting of alarm sound.

In this embodiment of the present invention, when an operated object is connected to a radio frequency mainframe through connecting ends of radio frequency circuits, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation.

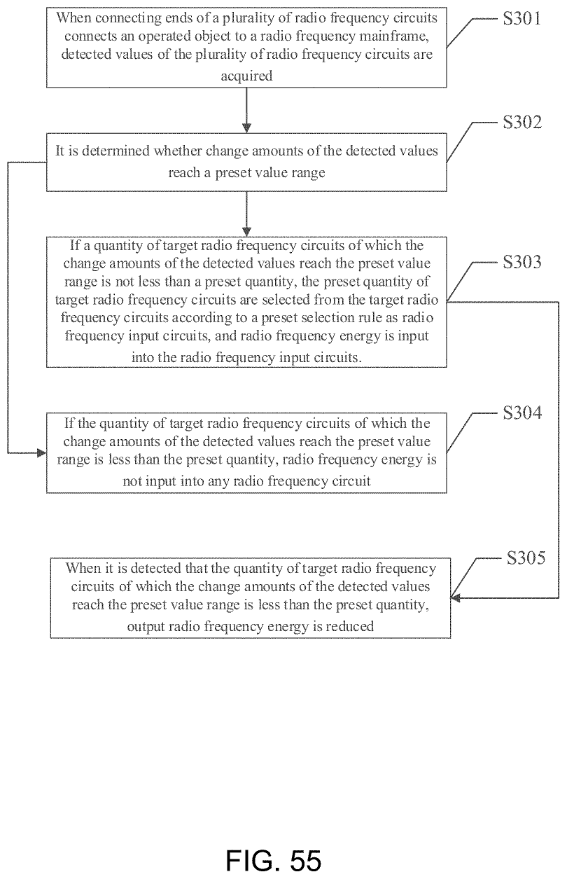

Referring to FIG. 55, which is an implementation flow chart of a method for safety control of radio frequency operation provided by another embodiment of the present invention. The method can be applied to the radio frequency mainframe shown in FIG. 53, as shown in FIG. 55, the method specifically comprises the follows.

Step S301, when connecting ends of a plurality of radio frequency circuits connects an operated object to a radio frequency mainframe, detected values of the plurality of radio frequency circuits are acquired.

Step S302, it is determined whether change amounts of the detected values reach a preset value range.

Specifically, a change amount of a detected value is a change amount of a voltage of a primary coil of a transformer in a radio frequency circuit.

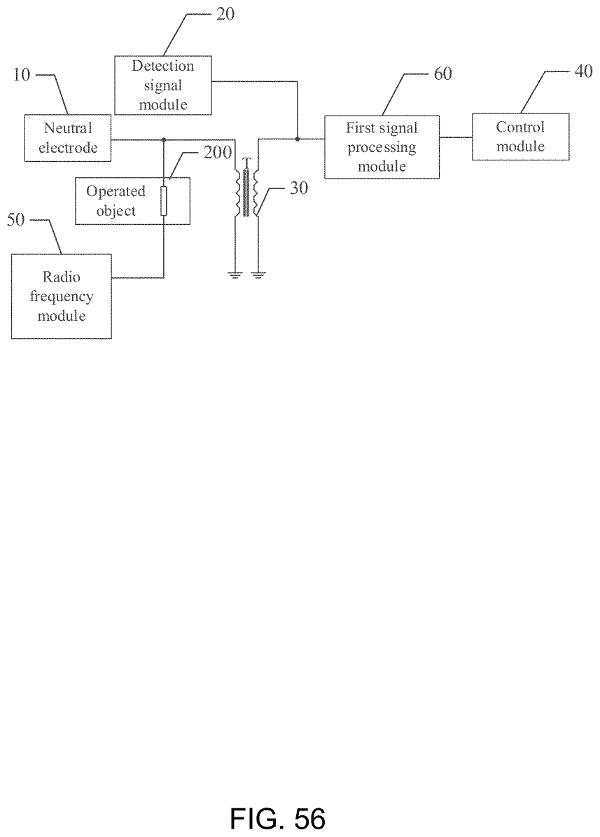

Referring to FIG. 56, FIG. 56 is a structural schematic diagram of a radio frequency circuit. Each radio frequency circuit has a connecting end, that is, a neutral electrode 10. In this embodiment, it is possible connect a plurality of radio frequency circuits to an operated object at the same time. Furthermore, each radio frequency further includes a detection signal module 20, a transformer 30, a control module 40, and a radio frequency module 50; wherein the detection signal module 20 and the neutral electrode module 10 are respectively connected to a primary coil and a secondary coil of the transformer 30, and the neutral electrode 10 forms a loop with the secondary coil of the transformer 30 when being attached to the operated object; the detection signal module 20 sends a detecting signal; the control module 40 can specifically be processor such as a CPU, which can be a CPU in the radio frequency mainframe and can also be an individual CPU of the radio frequency circuit, can control the detection signal module 20 to input the detecting signal into the circuit, and can also determine whether a connection between the neutral electrode 10 and the radio frequency mainframe meets a connection standard according to a lowering value of a voltage of the primary coil of the transformer 30, and control the radio frequency module 50 to output radio frequency energy to the operated object 200. The radio frequency circuit further includes a first signal processing module 60, the first signal processing module 60 includes a filter, a resonators, an amplifier, etc., so as to perform filtering, amplification, and so on for the detection signal, and the specific circuit structure is not particularly limited.

Specifically, when neutral electrodes of a plurality of radio frequency circuits are attached to the operated object to connect the operated object to the radio frequency mainframe, the control module controls the detection signal module to send a detecting signal to the transformer, and acquires voltage values of primary coils of transformers of a plurality of radio frequency circuits. When a voltage value lowers to the preset value range, it is determined that an attachment degree between a neutral electrode 10 and the operated object 200 meets a preset standard. The preset value range is preferably 1.6-2.0V (volt).

A specific implementation manner in a circuit can be that: it is possible to set high electric level signals and low electric level signals according to the preset value range, for example, a voltage signal of 2.3-2.8V is set to be a high electric level signal, and a voltage signal of 0.6-1.0V is set to be a low electric level signal. Thus, when the control module detects that a detecting signal changes from a high electric level signal to a low electric level signal, it can be determined that an attachment degree between a neutral electrode 10 and the operated object 200 meets the preset standard; if a change amount of a voltage value does not reach the preset value range, the signal electric level does not generate an obvious change, the control module 40 determines that an attachment degree between a neutral electrode 10 and the operated object 200 does not meet the preset standard.

Step S303, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

Among them, the selecting the preset quantity of target radio frequency circuits from the target radio frequency circuits according to a preset selection rule as radio frequency input circuits specifically comprises: acquiring preset numbers of the target radio frequency circuits; determining connecting areas corresponding to the target radio frequency circuits according to the numbers; and correspondingly selecting the radio frequency input circuits in the target radio frequency circuits according to operation areas of the current radio frequency operation and a quantity of circuits required by each operation area.

Step S304, if the quantity of target radio frequency circuits of which the change amounts of the detected values reach the preset value range is less than the preset quantity, radio frequency energy is not input into any radio frequency circuit.

Technical details of the above steps refer to the description of the embodiment shown in above FIG. 54, and are not repeated here.

Step S305, when it is detected that the quantity of target radio frequency circuits of which the change amounts of the detected values reach the preset value range is less than the preset quantity, output radio frequency energy is reduced.

After radio frequency energy is input, if it is detected that the quantity of target radio frequency circuits of which the change amounts of the detected values reach the preset value range is less than the preset quantity, it means that there is a problem in the connection between a connecting end currently inputting radio frequency energy and the operated object, and a connection standard is not met. If the quantity of radio frequency circuits that do not meet the connection standard does not reach a preset quantity, that is, requirement of the radio frequency operation is not met, output radio frequency energy is reduced to avoid causing damage to the radio frequency mainframe or the radio frequency operated object. At the same time, an alarm can be performed to prompt operating users to check the connection situation between the connection ends and the operated object. The alarm can be an audible and visual alarm or text display on a display screen of the radio frequency mainframe.

At this time, a method of detecting whether the change amounts of the detected values reaches the preset value range can be implemented by detecting a lowering amount of a voltage of a primary coil of a transformer, as in the above step S302, and can also be implement by detecting an output impedance value of a radio frequency input circuit, which are specifically as follows.

Specifically, reducing output radio frequency energy refers to lowering power of an output radio frequency signal. In this situation, radio frequency energy is reduced to be a value that is not equal to 0, and radio frequency energy with a safe strength is still output. Alternatively, the connection between the radio frequency input circuit and the operated object is cut off; in this situation, radio frequency energy is directly reduced to 0.

In this embodiment of the present invention, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect the change amounts of the detected values continuously and in real time; if it is detected that the quantity of target radio frequency circuits of which the change amounts of the detected values reach the preset value range is less than the preset quantity, output radio frequency energy is reduced, so as to reduce risk of damage to the radio frequency mainframe and the operated object, and further improve safety and intelligence of radio frequency operations.

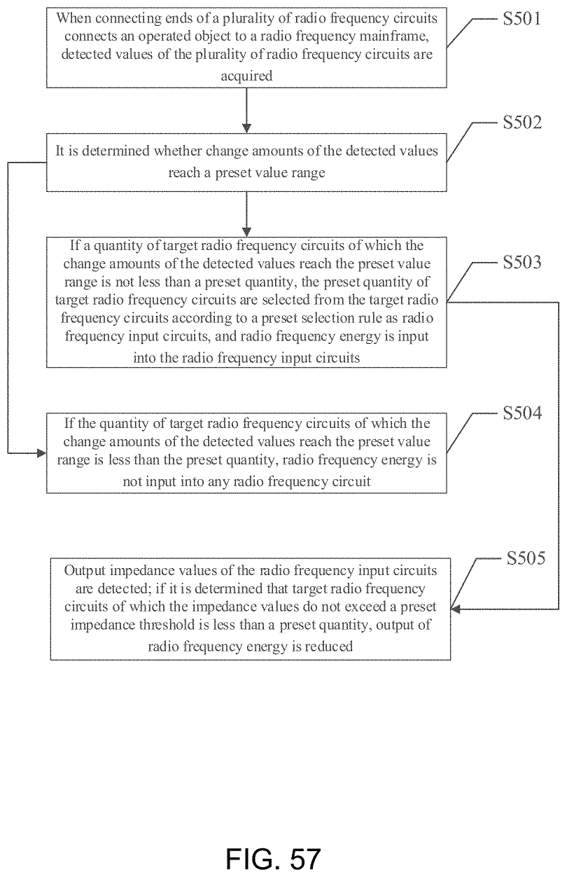

Referring to FIG. 57, which is an implementation flow chart of a method for safety control of radio frequency operation provided by another embodiment of the present invention. The method can be applied to the radio frequency mainframe shown in FIG. 53, as shown in FIG. 57, the method specifically comprises the follows.

Step S501, when connecting ends of a plurality of radio frequency circuits connects an operated object to a radio frequency mainframe, detected values of the plurality of radio frequency circuits are acquired.

Step S502, it is determined whether change amounts of the detected values reach a preset value range.

Step S503, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

Step S504, if the quantity of target radio frequency circuits of which the change amounts of the detected values reach the preset value range is less than the preset quantity, radio frequency energy is not input into any radio frequency circuit.

Step S505, output impedance values of the radio frequency input circuits are detected; if it is determined that target radio frequency circuits of which the impedance values do not exceed a preset impedance threshold is less than a preset quantity, output of radio frequency energy is reduced.

An output impedance value of each impedance detection circuit is detected, it is determined whether the impedance values exceed a preset impedance threshold, and a quantity of target radio frequency circuits of which the impedance values do not exceed the preset impedance threshold is counted. If the target radio frequency circuits of which the impedance values do not exceed the preset impedance threshold is less than the preset quantity, a frequency of an output radio frequency signal is lowered or a connection between a radio frequency input circuit and the operated object is cut off.

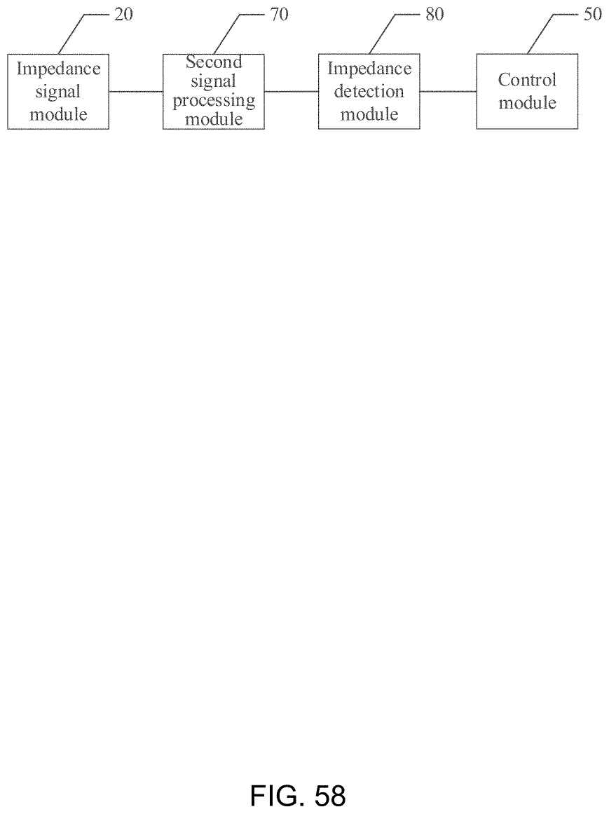

Referring to FIG. 58, FIG. 58 is a structural schematic diagram of an impedance detection circuit. Each impedance detection circuit is connected to an output end of a radio frequency input circuit to which a radio frequency signal is input, and each impedance detection circuit includes an impedance detection signal module 60, a second signal processing module 70, an impedance detection module 80, and the control module 40. The second signal processing module 70 includes a filter, a resonator, an amplifier, etc., so as to perform filtering, amplification, and so on for an impedance detection signal, and the specific circuit structure is not particularly limited. The impedance detection module 80 can include an impedance detection circuit configured to directly detect impedance values, or include a current detection circuit and a voltage detection circuit for indirectly calculating impedance values through detected currents and voltages, the specific circuit structure is not particularly limited.

In this embodiment of the present invention, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect impedance values of the radio frequency input circuit in real time; if it is detected that the quantity of target radio frequency circuits of which the impedance values exceed a preset impedance threshold and reaches a preset value range is less than a preset quantity, output radio frequency energy is reduced, so as to reduce risk of damage to the radio frequency mainframe and the operated object, and further improve safety and intelligence of radio frequency operations.

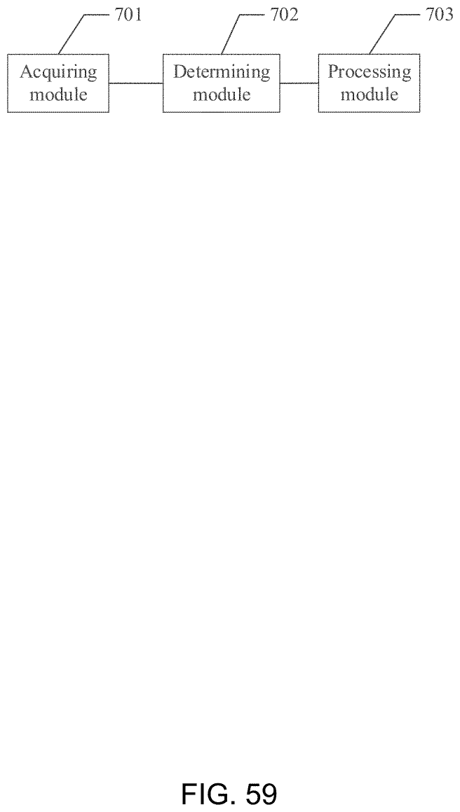

Referring to FIG. 59, which is a structural schematic diagram of an apparatus for safety control of radio frequency operation provided by an embodiment of the present invention. In order to facilitate description, only parts relating to embodiments of the present invention are shown. The apparatus can be arranged in the above-described radio frequency main frame, and the apparatus includes: an acquiring module 701 configured to: when connecting ends of a plurality of radio frequency circuits connects an operated object to a radio frequency mainframe, acquire detected values of the plurality of radio frequency circuits; a determining module 702 configured to determine whether change amounts of the detected values reach a preset value range; and a processing module 703 configured to: if a quantity of target radio frequency circuits of which the change amounts of the detected values reach the preset value range is not less than a preset quantity, select the preset quantity of target radio frequency circuits from the target radio frequency circuits according to a preset selection rule as radio frequency input circuits, and input radio frequency energy into the radio frequency input circuits; wherein the processing module 703 is further configured to: if the quantity of target radio frequency circuits of which the change amounts of the detected values reach the preset value range is less than the preset quantity, not input radio frequency energy into any radio frequency circuit.

Furthermore, the processing module 703 is further configured to: when it is detected that the quantity of target radio frequency circuits of which the change amounts of the detected values reach the preset value range is less than the preset quantity, reduce output radio frequency energy, specifically for lowering power of an output radio frequency signal; or cut off a connection between a radio frequency input circuit and an operated object.

The processing module 703 is further configured to: acquire preset numbers of the target radio frequency circuits; determine connecting areas corresponding to the target radio frequency circuits according to the numbers; and correspondingly select the radio frequency input circuits in the target radio frequency circuits according to operation areas of the current radio frequency operation and a quantity of circuits required by each operation area.

Furthermore, the connecting end is a neutral electrode, each radio frequency circuit has a neutral electrode, and each radio frequency circuit includes a detection signal module, a transformer, and a control module.

Thus, the processing module 703 is further configured to: when neutral electrodes of a plurality of radio frequency circuits are attached to the operated object to connect the operated object to the radio frequency mainframe, control the control module to control the detection signal module to send a detecting signal to the transformer, and acquire voltage values of primary coils of transformers of a plurality of radio frequency circuits.

Furthermore, the determining module 702 is further configured to determine whether the voltage values of primary coils of transformers of radio frequency circuits are lowered to reach a preset value range.

The processing module 703 is further configured to: detect an output impedance value of each radio frequency input circuit, determine whether the impedance values exceed a preset impedance threshold, and count a quantity of target radio frequency circuits of which the impedance values do not exceed the preset impedance threshold; and if the target radio frequency circuits of which the impedance values do not exceed the preset impedance threshold is less than the preset quantity, lower a frequency of an output radio frequency signal or cut off a connection between a radio frequency input circuit and the operated object.

Specifically, the processing module 703 detects the output impedance values by controlling impedance detection circuits. Among them, each impedance detection circuit is connected to an output end of a radio frequency input circuit, and each impedance detection circuit includes an impedance detection signal module, an impedance detection module, and the control module. The processing module 703, through the control module, control the impedance detection signal module to output an impedance detection signal, and the impedance detection module detects an output impedance value of each radio frequency input circuit.

In this embodiment of the present invention, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect the change amounts of the detected values or the detected values in real time; if it is detected that the change amounts of the detected values reach a preset value range, or the quantity of target radio frequency circuits of which the impedance values exceed a preset impedance threshold and reach a preset value range is less than a preset quantity, output radio frequency energy is reduced, so as to reduce risk of damage to the radio frequency mainframe and the operated object, and further improve safety and intelligence of radio frequency operations.

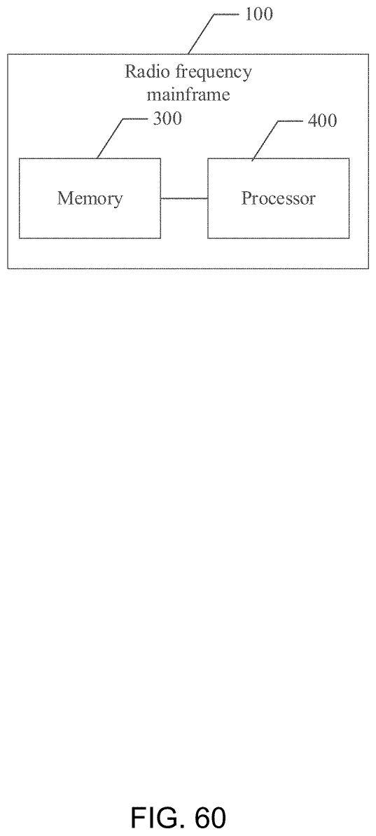

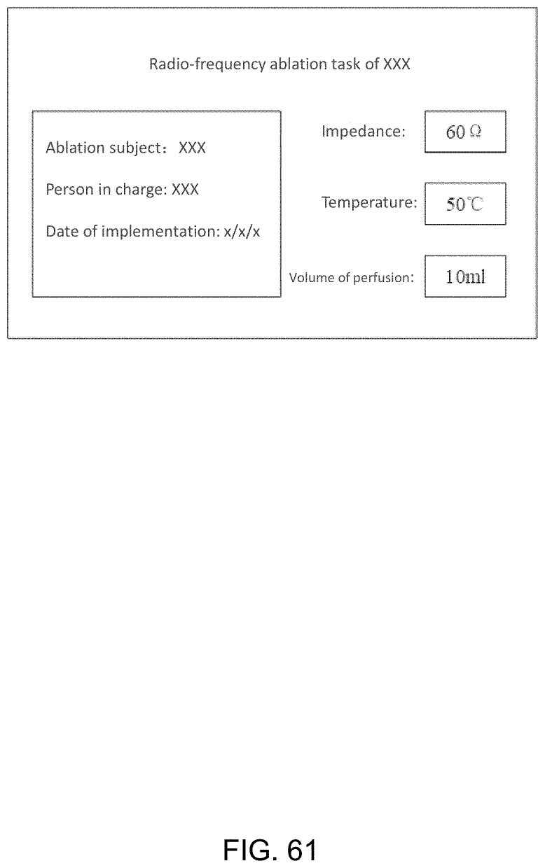

As shown in FIG. 60, an embodiment of the present invention further provides a radio frequency mainframe, which includes a memory 300 and a processor 400, the processor 400 can be an apparatus for safety control of radio frequency operation in the above embodiment, and can also be the processing module 703 in the apparatus for safety control of radio frequency operation. The memory 300 can be, for example, a hard disk drive memory, a non-volatile memory (such as a flash memory or other electronically programmable restricted deletion memory used to form solid-state drives, etc.), volatile memory (such as a static or dynamic random access memory, etc.), and so on, embodiments of the present invention do not limit here.

The memory 300 stores executable program codes; the processor 400 coupled with the memory 300 calls the executable program codes stored in the memory to execute the above-described method for safety control of radio frequency operation.

Further, an embodiment of the present invention further provides a computer-readable storage medium, the computer-readable storage medium can be set in the radio frequency mainframes in the above embodiments, and the computer-readable storage medium can be the memory 300 in the above embodiment shown in FIG. 60. The computer-readable storage medium stores a computer program, and the program, when being executed by a processor, implements the method for safety control of radio frequency operation described in the embodiments shown in above FIG. 54, FIG. 55, and FIG. 57. Further, the computer-readable storage medium can also be a U-disk, a mobile hard disk, a read-only memory (ROM), a RAM, a magnetic disk or a CD-ROM, and other media that can store program codes.

It should be noted that regarding the foregoing method embodiments, for simplicity of description, they are all expressed as a series of action combinations, but those skilled in the art should know that the present invention is not limited by the described sequence of actions. Because according to the present invention, certain steps can be performed in other orders or simultaneously. Secondly, those skilled in the art should also know that the embodiments described in the specification are all preferred embodiments, and the involved actions and modules are not necessarily all required by the present invention.

In the above-mentioned embodiments, the description of each embodiment has its own emphasis. For parts that are not described in detail in a certain embodiment, reference may be made to related descriptions of other embodiments.

Ablation Operation Prompting Method, Electronic Device and Computer-Readable Storage Medium

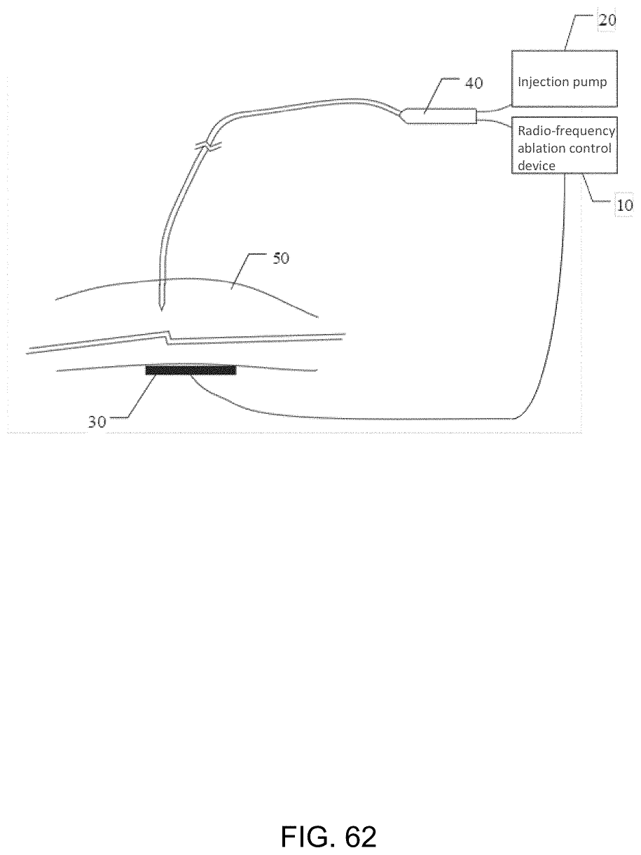

FIG. 62 is a schematic view showing an application scenario of an ablation operation prompting method provided in an embodiment of the present invention. The ablation operation prompting method can be implemented by a radio-frequency ablation control device 10 shown in FIG. 62, or implemented by other computer equipment that has established a data connection with the radio-frequency ablation control device 10.

As shown in FIG. 62, the radio-frequency ablation control device 10 is connected to a syringe pump 20, a neutral electrode 30 and a radio-frequency ablation catheter 40. The radio-frequency ablation control device 10 is provided with a built-in display screen (not shown).

Specifically, before an ablation task is implemented, an energy emitting end of the radio-frequency ablation catheter 40 for generating and outputting radio-frequency energy and an extension tube (not shown) of the syringe pump 20 are inserted into the body of an ablation subject 50 (such as an emphysema patient), reaching an ablation site. Then, the neutral electrode 30 is brought into contact with the skin surface of the ablation subject 50. A radio-frequency current flows through the radio-frequency ablation catheter 40, the tissue of the ablation subject and the neutral electrode 30, to form a circuit.

When an ablation task is triggered, the radio-frequency ablation catheter 40 is controlled by the radio-frequency ablation control device 10 to outputting radio-frequency energy to the ablation site by discharging to implement an ablation operation on the ablation site. Meanwhile the syringe pump 20 performs a perfusion operation on the ablation subject through the extension tube, wherein physiological saline is infused into the ablation site, to adjust the impedance and temperature of the ablation site.

At the same time, the radio-frequency ablation control device 10 acquires a locally pre-stored image of the ablation site and displays the image on the display screen.

Moreover, the radio-frequency ablation control device 10 also acquires position data of a currently-being-ablated target ablation point by for example, a camera (not shown) installed near the energy emitting end of the radio-frequency ablation catheter, and marks the target ablation point in the image according to the position data.

Further, the radio-frequency ablation control device 10 also acquires the elapsed ablation time and the temperature of the target ablation point in real time by a built-in timer and a temperature sensor (not shown) installed near the energy emitting end, determines the ablation status of the target ablation point according to the elapsed ablation time and the temperature, and then generate a schematic real-time dynamic change diagram of the target ablation point according to the ablation status, and display the schematic diagram on the screen, to indicate to a user the real-time ablation status change of the target ablation point.

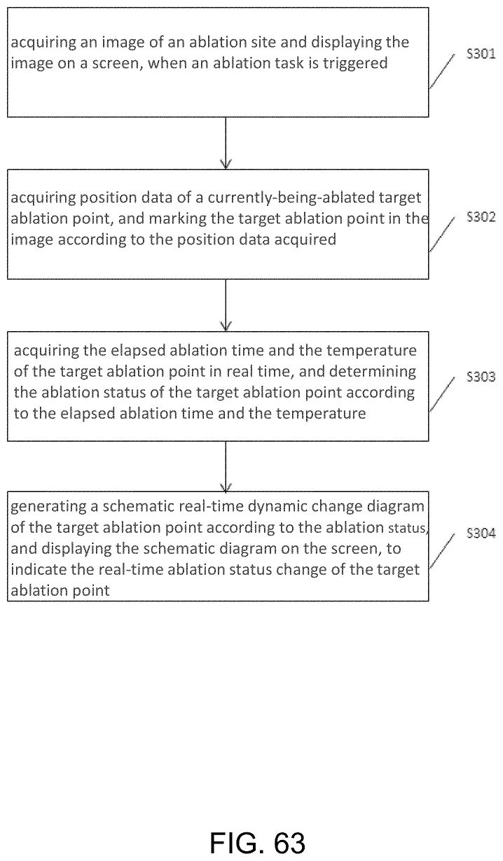

FIG. 63 shows a flow chart of an ablation operation prompting method provided in an embodiment of the present invention. The method can be implemented by a radio-frequency ablation control device 10 shown in FIG. 62, or implemented by other computer terminals connected thereto. For ease of description, in the following embodiments, the radio-frequency ablation control device 10 is used as an implementation body. As shown in FIG. 63, the method includes specifically:

Step S301: acquiring an image of an ablation site and displaying the image on a screen, when an ablation task is triggered.

Specifically, before Step S301 is implemented, besides the radio-frequency ablation control device, a main control device of an image acquisition system can also acquire a holographic image of an ablation site in each ablation subject intended to receive an ablation operation by an ultrasound medical imaging device or an endoscopic system in advance, and store the acquired holographic image in an image database. When an ablation task is triggered, identification information of a target ablation subject corresponding to the ablation task is acquired, and then the image of the ablation site in the target ablation subject is acquired according to the identification information by querying the image database, and displayed on a screen.

The screen may be a display screen built in the radio-frequency ablation control device, or an external display screen that has established a data connection with the radio-frequency ablation control device. The holographic image can be an original image taken, or a two-dimensional or three-dimensional static or dynamic image converted from the original image by calling PROE, AUTOCAD, Adobe Photoshop, Python Matplotlib, or other image processing programs.

It can be understood that in addition to PROE, AUTOCAD, Adobe Photoshop, and Python Matplotlib, there are still many other applications currently used for image processing. The program used can be determined according to actual needs, and is not particularly limited in the present invention.

Optionally, the preset prompting interaction interface can be used as a carrier of the image and various schematic diagrams involved in the various embodiments of the present invention, to facilitate the layout design and the management and positioning of each schematic diagram. For example, the image is displayed in a first preset area of the preset prompting interaction interface.

The preset prompting interaction interface is a graphical user interface (GUI). It can be understood that an application of the prompting interaction interface is preset in the radio-frequency ablation control device. Before the ablation operation is implemented, the application is automatically called, to display the prompting interaction interface on the screen. The preset prompting interaction interface includes multiple areas, respectively used to display different prompt information. The processing logic of the application can automatically divide areas, based on the number of information that needs to be displayed.

Specifically, the acquired image of the ablation site is stored in a first storage location corresponding to a first preset area of the prompting interaction interface. The application automatically refreshes the prompting interaction interface according to a preset period, and add the image to the first preset area for display, if the image is read from the first storage location when implement a refresh operation. Alternatively the acquired image of the ablation site is displayed in the first preset area of the preset prompting interaction interface in the form of overlapped images.

Step S302: acquiring position data of a currently-being-ablated target ablation point, and marking the target ablation point in the image according to the position data.

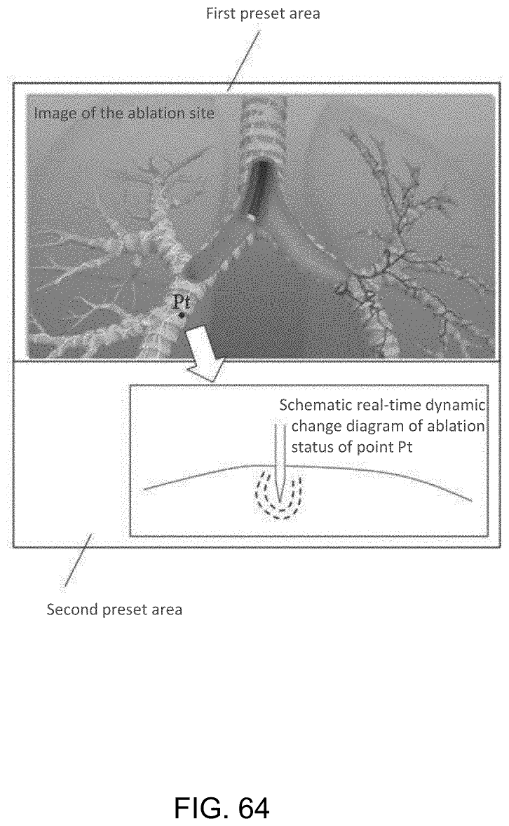

Specifically, position coordinate of the currently-being-ablated target ablation point in the image of the ablation site is acquired according to any one of the following three preset positioning methods. Then, the target ablation point is marked in the image of the ablation site according to the position data and a preset marking logic, wherein the marking logic is a general designation of the preset various operations required for marking as shown in FIG. 64, according to the position coordinate, a circular icon Pt is drawn at a corresponding position in the image and used as a mark.

The first preset positioning method is to obtain the position data of the target ablation point through an endoscope. Specifically, a picture of a current ablation operation captured by an endoscope is acquired and compared with the image, to obtain the position data of the target ablation point in the image.

It can be understood that a camera is provided at the tip of the endoscope; and when an ablation operation is implemented, the camera is inserted together with an ablation catheter into the body of an ablation subject, approaching the ablation site, to capture a picture of the ablation site in real time. The camera sends the captured pictures back to the radio-frequency ablation control device while the pictures are captured. The feature points in an area in the ablation site where the ablation operation is being performed in the picture returned by the endoscope are extracted and matched with the feature points in the image displayed in Step S301; and position data corresponding to a feature point with the highest matching degree is determined as the position data of the target ablation point. The position data is a position coordinate, and the coordinate system of the position coordinate is a two-dimensional or three-dimensional coordinate system established by using a centroid of the ablation site in the displayed image or an end point of the image as the origin.

The second preset positioning method is to obtain the position data of the target ablation point through an ultrasound medical imaging device. Specifically, an ultrasound image of the target ablation point is obtained by using an ultrasound medical imaging device; and according to the ultrasound image, the position data of the target ablation point in the image is obtained.

The working principle of the ultrasound medical imaging device is to irradiate the human body with ultrasonic waves, and obtain a visible image of the nature and structure of human tissues, by receiving and processing echoes carrying feature information of nature or structures of human tissues, such as a section shape of the ablation site. At present, many kinds of ultrasound medical imaging devices are available, and the ultrasound medical imaging device used is not particularly limited in the present invention. image recognition of the ultrasound image is performed, to determine the position data of the target ablation point in the ultrasound image. Then, the position data of the target ablation point in the displayed image is determined according to the corresponding relationship between each ablation site in the ultrasound image and each ablation site in the image displayed in Step S301.

The third preset positioning method is to obtain the position data of the target ablation point by electromagnetic navigation technology. Specifically, the actual position data of the target ablation point is obtained by for example electromagnetic navigation bronchoscopy (ENB), and then the position data of the target ablation point in the displayed image is determined according to the corresponding relationship between the real ablation site and each ablation site in the image displayed in Step S301.

Step S303: acquiring the elapsed ablation time and the temperature of the target ablation point in real time, and determining the ablation status of the target ablation point according to the elapsed ablation time and the temperature.

Specifically, in an ablation task, at least one ablation site needs to be ablated, each ablation site includes multiple ablation points, and for each ablation point, a corresponding ablation operation needs to be performed. A timer is preset in the radio-frequency ablation control device; and whenever an ablation operation of an ablation point is started, the timer records the elapsed ablation time of the ablation point. When the position of the ablation operation changes, the current timing is ended, and a new round of timing is restarted.

Moreover, during the timing, the temperature near the target ablation point is also obtained by a temperature sensor in real time. Then, according to the elapsed implementation time of the ablation operation (that is, the elapsed ablation time) and the temperature, the ablation status of the target ablation point, for example, the shape and size of the tissue ablated, is determined.

For the same ablation point, timing separately according to the temperature can be performed, that is, the durations at different temperatures are statistically counted. The shape and size of the tissue ablated can be determined according to the length, width and height of the ablated area starting from the target ablation point (hereinafter collectively referred to as the ablated length, width and height of the target ablation point). The ablated length, width and height of the target ablation point are calculated according to Formulas 1-3 below:

(time of a temperature continuously reaching a preset temperature/preset unit of ablation time)*preset ablation length=ablated length; Formula 1:

(time of a temperature continuously reaching a preset temperature/preset unit of ablation time)*preset ablation width=ablated width; Formula 2:

(time of a temperature continuously reaching the preset temperature/preset unit of ablation time)*preset ablation height=ablated height. Formula 3:

The preset temperature and the preset unit ablation time are respectively the critical temperature and the unit critical time allowing the ablation site to undergo qualitative changes (i.e., achieve the expected ablation effect). The preset ablation length, preset ablation height and preset ablation width are the length, width and height of the ablated area increased with the elapse of a preset unit of ablation time after the ablation site reaches the preset temperature. The preset temperature, preset unit of ablation time, and preset ablation length, width and height can be set according to the user's custom operation.

It is experimentally confirmed that after the temperature of the ablation site reaches the critical temperature, the increase in the length, width, and height of the ablated area is generally constant in every time interval. Therefore, according to the above Formulas 1 to 3, the ablated length, width and height of the target ablation point can be obtained. Then, according to the obtained ablated length, width and height and the coordinate of the target ablation point, the shape, size, and boundary coordinates of the ablated area around the target ablation point are obtained.

Step S304: generating a schematic real-time dynamic change diagram of the target ablation point according to the ablation status, and displaying the schematic diagram on the screen, to indicate the real-time ablation status change of the target ablation point.

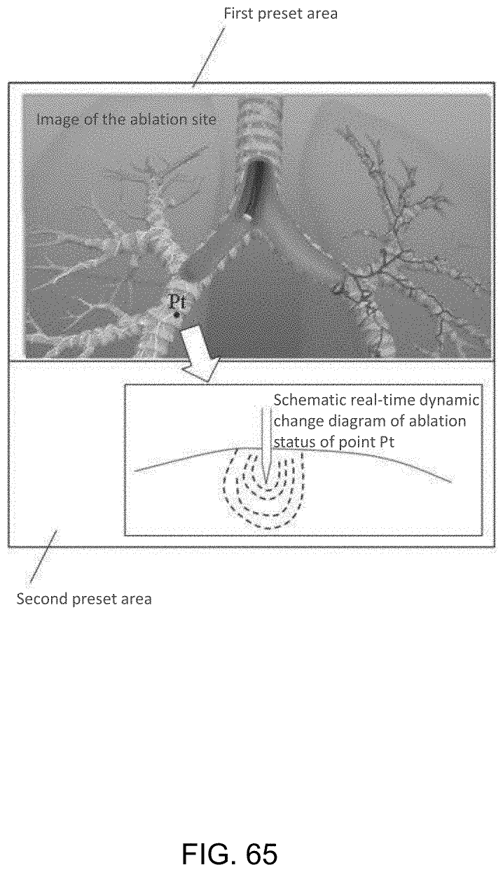

Specifically, by calling the above image processing program, and according to the coordinate of the target ablation point, the shape and size of the ablated area around the target ablation point, and the boundary coordinates of the ablated area, a schematic real-time dynamic change diagram of the ablation status of the target ablation point as shown in FIG. 64 and FIG. 65 is generated. The schematic real-time dynamic change diagram contains visualizations of the process of continuously expanding the boundary of the ablated area. Each circle of dotted lines in the schematic real-time dynamic change diagrams of the ablation status of the target ablation point as shown in FIG. 64 and FIG. 65 indicates the boundary of each expansion of the ablated area. Further, the boundary of the outermost circle can also be displayed in a flashing manner, to make the schematic diagram more indicative.

Optionally, the schematic real-time dynamic change diagram is displayed in a second preset area of the preset prompting interaction interface. The second preset area may be set within an area embraced by the first preset area. Alternatively, it may also be set near the first preset area.

Further, the positional relationship between the first preset area and the second preset area may also be determined according to the number of contents that needs to be displayed in the prompting interaction interface. For example, when there are more contents to be displayed, the generated schematic real-time dynamic change diagram is displayed by overlapping in the vicinity of the target ablation point in the image displayed in the first preset area, to save the space occupied. When there are less contents to be displayed, the schematic real-time dynamic change diagram is displayed in an area next to the first preset area, thereby improving the flexibility of information display.

In the embodiments of the present invention, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic real-time dynamic change diagram of the ablation status of the target ablation point is generated and displayed, so that the status changes of an ablation site can be displayed in real time and intuitively during the implementation of the ablation operation, thereby improving the effectiveness and relevance of information prompts.

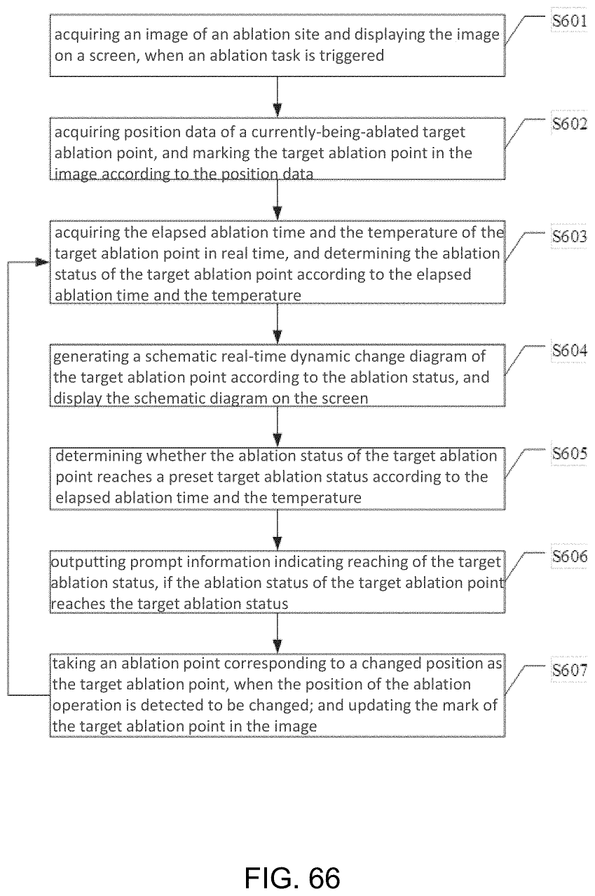

FIG. 66 shows a flow chart of an ablation operation prompting method provided in another embodiment of the present invention. The method can be implemented by a radio-frequency ablation control device 10 shown in FIG. 62, or implemented by other computer terminals connected thereto. For ease of description, in the following embodiments, the radio-frequency ablation control device 10 is used as an implementation body. As shown in FIG. 66, the method includes specifically:

Step S601: acquiring an image of an ablation site and displaying the image on a screen, when an ablation task is triggered.

Step S602: acquiring position data of a currently-being-ablated target ablation point, and marking the target ablation point in the image according to the position data.

Step S603: acquiring the elapsed ablation time and the temperature of the target ablation point in real time, and determining the ablation status of the target ablation point according to the elapsed ablation time and the temperature.

Step S604: generating a schematic real-time dynamic change diagram of the target ablation point according to the ablation status, and display the schematic diagram on the screen, to indicate the real-time ablation status change of the target ablation point.

Steps S601 to S604 are the same as Steps S301 to S304 in the embodiment shown in FIG. 63, and details may be made reference to the relevant description in the embodiment shown in FIG. 63, and will not be repeated here.

Step S605: determining whether the ablation status of the target ablation point reaches a preset target ablation status according to the elapsed ablation time and the temperature.

Step S606: outputting prompt information indicating reaching of the target ablation status, if the ablation status of the target ablation point reaches the target ablation status.

Specifically, according to the elapsed ablation time and the temperature of the target ablation point, the ablated length, width and height of the target ablation point are determined, and whether the ablated length, width and height of the target ablation point reach corresponding preset standard values respectively are determined. If they reach the corresponding preset standard values respectively, the ablation status of the target ablation point is determined to reach the preset target ablation status, and prompt information indicating reaching of the target ablation status is outputted. The prompt information indicating reaching of the target ablation status can be output in at least one form of a prompt text, a prompt graphic, a prompt sound, and a prompt light, etc.

Step S607: taking an ablation point corresponding to a changed position as the target ablation point, when the position of the ablation operation is detected to be changed; updating the mark of the target ablation point in the image and returning to Step S603, until the ablation operation is ended.

Specifically, the method described in Step S602 can be used, wherein the position data of the ablation operation is acquired in real time, and whether the position of the ablation operation has changed is detected according to the position data. If the position of the ablation operation is detected to be changed, it means that the ablation point has changed. Therefore, an ablation point corresponding to a changed position is used as a new target ablation point, and a changed position coordinate is used as the position data of the new target ablation point. Then according to the position data, the mark of the target ablation point is updated in the image displayed on the screen. For example, the mark of the previous target ablation point is hidden or deleted, and the mark of the new target ablation point is added to the image at the same time. The method of adding the mark of the new target ablation point is the same as that for the previous target ablation point and will not be repeated here.

Moreover, the process is returned to Step S603: acquiring the elapsed ablation time and the temperature of the target ablation point in real time, and determining the ablation status of the target ablation point according to the elapsed ablation time and the temperature, until the ablation operation is ended. The ablation operation can be automatically ended by the radio-frequency ablation control device when an abnormal event or other preset events are detected, or ended according to the user's operation.

Optionally, in another embodiment of the present invention, after Step S601 of acquiring an image of an ablation site and displaying the image on a screen when an ablation task is triggered. the method further includes: acquiring a first drawing parameter of a target operation track of the ablation operation, and drawing the target operation track at a corresponding position of the image according to the first drawing parameter; acquiring the position change data of the ablation point in real time, determining a second drawing parameter of the real-time operation track of the ablation operation according to the position change data, and drawing the real-time operation track at a corresponding position of the image according to the second drawing parameter; and analyzing in real time whether the amplitude of the real-time operation track deviating from the target operation track is greater than a preset amplitude, and outputting prompt information for track deviation warning when the real-time operation track deviates from the target operation track by an amplitude greater than the preset amplitude.

Specifically, the first drawing parameter of the target operation track of each ablation operation to be performed is stored in the radio-frequency ablation control device locally, or ENBW in a database server that has established a data connection with the radio-frequency ablation control device. The radio-frequency ablation control device can query the first drawing parameter of the target operation track of the corresponding ablation operation locally or from the database server according to the identification information of the triggered ablation task.

It can be understood that the ablation site is composed of multiple ablation points, the ablation operation needs to be performed on each ablation point one by one. The target operation track is a preset preferred route to be taken when the ablation operation is performed for each ablation point, and used to assist the user in the ablation operation, to achieve a better ablation effect.

Optionally, the first drawing parameter can be generated according to a route parameter input by the user, or calculated according to the preset ablation target and the shape and size of the ablation site.

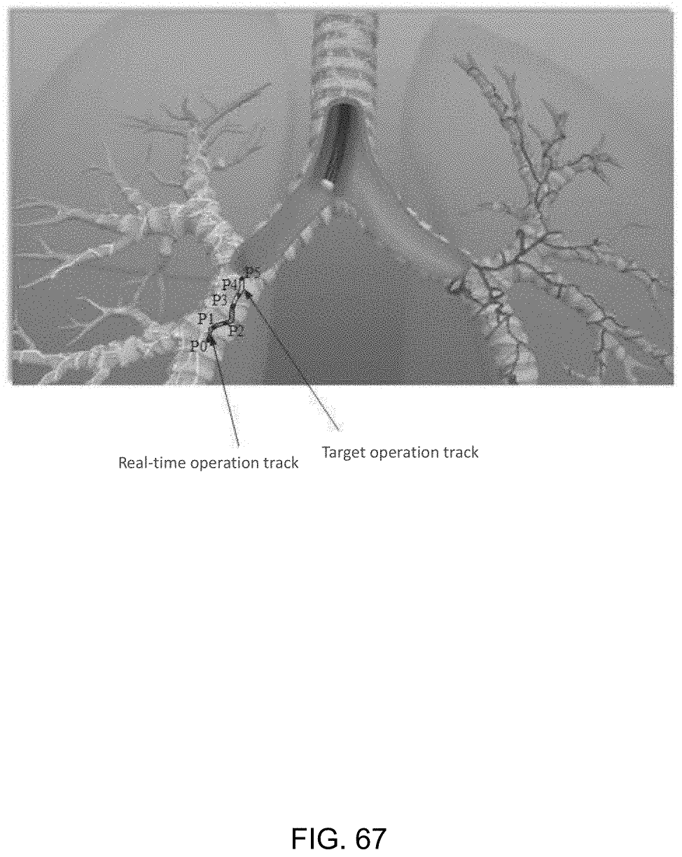

The first drawing parameter may specifically include, but is not limited to, position coordinates and drawing order of various ablation points (such as points P0 to P5 in FIG. 67) in the target operation track, wherein P0 is the starting point, and P5 is the end point), and the line characteristics of the target operation track. The line characteristics of the target operation track can include, but are not limited to, for example: line type (such as: dashed line, or solid line), shadow, thickness, and color, etc.

Further, the method described in Step S302 can be used, wherein the position data of the ablation operation is acquired in real time. Then, the position change data of the ablation operation is obtained according to the position data obtained in real time, wherein the position change data includes: the initial position coordinate of the ablation operation and the position coordinates after each position change. Whenever a position change of the ablation operation is detected, the changed position is marked as an ablation point and the position coordinate of the ablation point is recorded. Then, the second drawing parameter is determined according to the position coordinate of the marked ablation point and the preset drawing logic, and the real-time operation track is drawn according to the determined second drawing parameter (as shown in FIG. 67), to present a display effect of the real-time operation track dynamically extending over time.

The second drawing parameter can include, but is not limited to: position coordinates and drawing order of various ablation points (i.e. various marked ablation points) in the real-time operation track, and the line characteristics of the real-time operation track. The line characteristics of the real-time operation track can include, but are not limited to, for example: line type (such as: dashed line, or solid line), shadow, thickness, and color, etc.

The coordinate system of the above-mentioned position coordinates is a two-dimensional or three-dimensional coordinate system established by using a centroid of the ablation site in the displayed image or an end point of the image as the origin.

Optionally, different colors and/or different types of lines can be used respectively in drawing the target operation track and the real-time operation track, to highlight the difference between target operation track and the real-time operation track, thereby further improving the effectiveness of information prompts.

Further, when the real-time operation track is drawn, the position coordinate of each ablation point in the drawn real-time operation track is compared with the position coordinate of each corresponding ablation point in the target operation track (corresponding to the ablation point in the real-time operation track in the drawing order), to obtain the coordinate variation therebetween. Then, the number of ablation points with a coordinate variation that is greater than a preset variation is counted. If the number is greater than a preset number, the amplitude of the real-time operation track deviating from the target operation track is determined to be greater than a preset amplitude, and prompt information for track deviation warning is outputted. Optionally, the prompt information can be in the form of a text and/or a graphic, and displayed in the first preset area of the prompting interaction interface.

Therefore, the target operation track and the real-time operation track are drawn, and the amplitude of deviation therebetween is analyzed. When the amplitude of deviation between the two is greater than the preset amplitude, the prompt information is displayed, so as to avoid the adverse effect of the user's improper operation on the ablation effect, improve the universality and intelligence of information prompts, and further improve the effectiveness of information prompts.

Optionally, in another embodiment of the present invention, the method further includes: acquiring the temperature of the ablation site in real time when the ablation task is triggered; drawing a real-time temperature change curve according to the temperature acquired in real time and displaying it on the screen; and analyzing in real time whether the temperature exceeds a preset warning value, and outputting prompt information for temperature warning in a display area of the temperature change curve when the temperature exceeds the warning value.

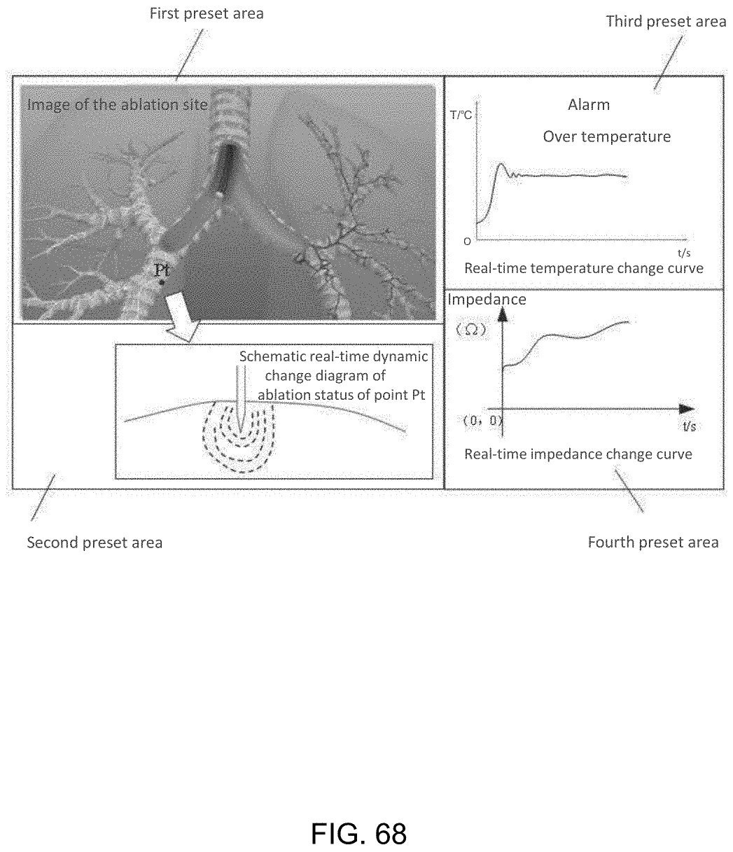

Specifically, the temperature of the ablation site can be obtained in real time by a temperature sensor set near the ablation site, and then a real-time temperature change curve as shown in FIG. 68 is drawn by calling a curve drawing function (such as: PLOT function) according to the temperature acquired in real time. The horizontal axis of the real-time temperature change curve is a time axis, and used to indicate the time at which each temperature value is obtained (t/s). The vertical axis of the real-time temperature change curve represents the temperature (T/° C.). Optionally, the real-time temperature change curve can be drawn in a third preset area of the prompting interaction interface.

It can be understood that in addition to the PLOT function, many more functions are available for curve drawing, and the function used can be selected according to actual needs in practical applications and is not particularly limited in the present invention.

Further, when the real-time temperature change curve is drawn, whether the obtained temperature exceeds a preset warning value is analyzed in real time, and outputting prompt information for temperature warning in a display area (for example, the third preset area) of the temperature change curve when the temperature exceeds the warning value. The prompt information for temperature warning can be displayed in the form of a text and/or a graphic, as shown FIG. 68.

Therefore, by drawing the real-time temperature change curve, and when the acquired temperature exceeds the warning value, warning information is outputted. This promotes the user to understand the temperature change of the ablation site in time and achieve the effect of temperature warning, thus further improving the universality and effectiveness of information prompts, and improving the safety of the ablation operation. In addition, by outputting the prompt information for temperature warning in the display area of the temperature change curve, the prompt information is made more directional.

Optionally, in another embodiment of the present invention, the method further includes: acquiring the impedance of the ablation site in real time when the ablation task is triggered; and drawing a real-time impedance change curve on a screen according to the impedance obtained in real time.

Specifically, by setting an impedance sensor installed at a tip of the radio-frequency ablation catheter, the impedance of the ablation site is acquired in real time; and a real-time impedance change curve as shown in FIG. 68 is drawn on the screen, by calling the above curve drawing function, according to the acquired impedance. The horizontal axis of the real-time impedance change curve represents time (t/s). The vertical axis of the real-time impedance change curve represents the impedance (a). Optionally, the real-time impedance change curve can be drawn in a fourth preset area of the prompting interaction interface.

Therefore, by drawing the real-time impedance change curve, the user is promoted to understand the impedance changes of the ablation site in time, to adjust the ablation operation in time, thus further improving the universality and effectiveness of information prompts, and improving the safety of the ablation operation.

Optionally, in another embodiment of the present invention, the method further includes: acquiring the impedance of the ablation site in real time, when the ablation task is triggered; determining a target interval corresponding to the impedance according to the impedance acquired in real time and the impedance ranges respectively corresponding to multiple preset ablation impedance prompting intervals; and drawing a schematic real-time impedance diagram on the screen according to the impedance, the impedance range and the target interval. The schematic real-time impedance diagram includes: description information of the multiple ablation impedance prompting intervals, description information of a preset reference impedance, and description information of the corresponding relationship between the impedance and the target interval. The multiple ablation impedance prompting intervals include: a non-ablatable impedance interval, an ablatable impedance interval, and an optimal ablatable impedance interval.

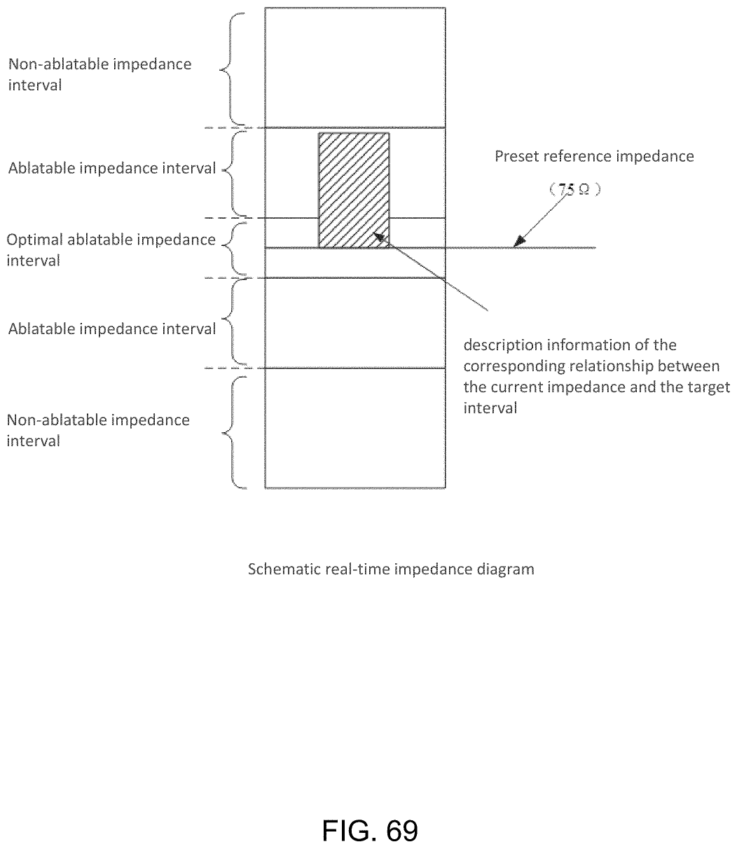

Further, as shown in FIG. 69, the description information of the multiple ablation impedance prompting intervals includes: multiple vertically laminated columns (6 columns in FIG. 69, and the number is not limited to 6 in actual applications) and the corresponding description texts and indicating graphics (such as the dashed line and curly brackets in FIG. 69) of the multiple ablation impedance prompting intervals; and the description information of the preset reference impedance includes: the description text and the indicating graphic (for example, arrow) of the preset reference impedance.

The multiple columns respectively correspond, from top to bottom, to an upper range of the non-ablatable impedance interval, an upper range of the ablatable impedance interval, an upper range of the optimal ablatable impedance interval, a lower range of the optimal ablatable impedance interval, a lower range of the ablatable impedance interval, and a lower range of the non-ablatable impedance interval.

The upper and lower limits of the non-ablatable impedance interval, ablatable impedance interval, and the optimal ablatable impedance interval can be preset in the radio-frequency ablation control device according to the user's custom operation. Optionally, the preset reference impedance may be a median value of the optimal ablation impedance interval or an average value of the upper limit and the lower limit. In this case, if the preset reference impedance is set according to the user's custom operation, the upper and lower limits of the non-ablatable impedance interval, the ablatable impedance interval, and optimal ablation impedance interval can be determined according to the preset reference impedance and the fluctuation range of each impedance interval preset by the user.

Further, the number of columns, the number and range of the ablation impedance prompting interval, and the correspondence between the column and each ablation impedance prompting interval may also be determined according to the number of electrodes on the radio-frequency ablation catheter, or set according to the user's custom operation.

Further, taking the column corresponding to the optimal ablatable impedance interval as a center, the height increases as the distance of the other columns in the multiple columns from the column increases. Therefore, by setting different sizes of columns to identify different ablation impedance intervals, the user is allowed to get to know the ablation impedance interval corresponding to the current impedance clearly, thereby further improving the eye-catching and intuitiveness of the prompt information, and improving the effectiveness of information prompts.

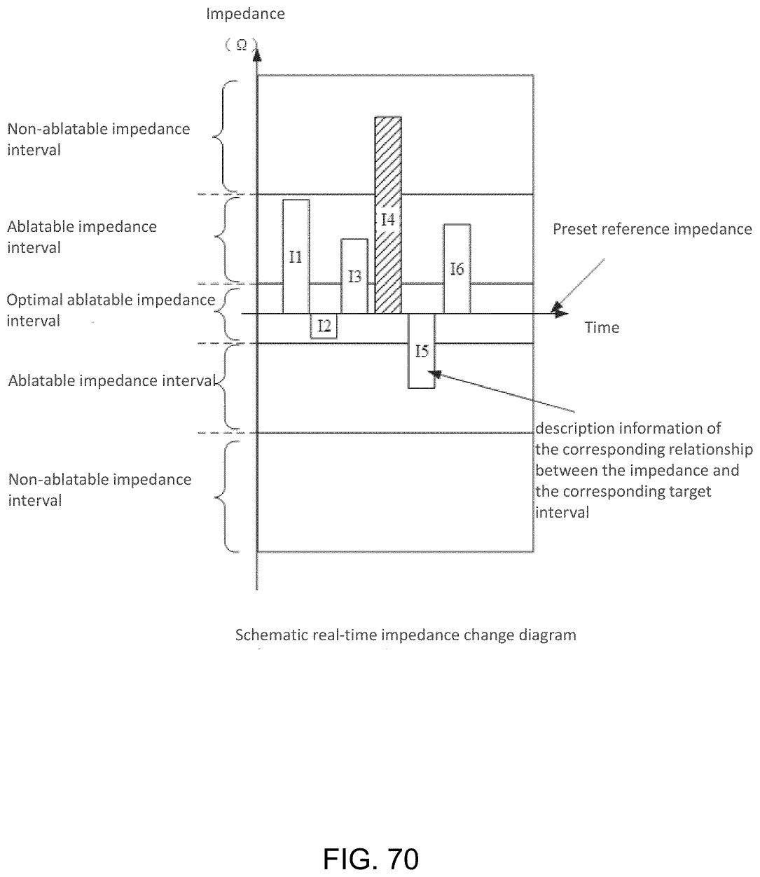

Optionally, in another embodiment of the present invention, the method further includes: acquiring the impedance of the ablation site in real time when the ablation task is triggered; determining a target interval corresponding to the impedance according to the impedance acquired in real time and the impedance ranges respectively corresponding to multiple preset ablation impedance prompting intervals; and drawing a schematic real-time impedance change diagram on the screen (as shown in FIG. 70) according to the impedance, the impedance range and the target interval. The schematic real-time impedance change diagram includes: two-dimensional coordinate axes, description information of the multiple ablation impedance prompting intervals, and description information of the corresponding relationship between each impedance and respective target interval. The multiple ablation impedance prompting intervals include: a non-ablatable impedance interval, an ablatable impedance interval, and an optimal ablatable impedance interval.

The horizontal axis of the two-dimensional coordinate axes is a time axis, indicating the acquisition time of each impedance. Moreover, the horizontal axis is also used to indicate the preset reference impedance. The vertical axis indicates, from bottom to top in a positive direction, an upper range of the optimal ablatable impedance interval, an upper range of the ablatable impedance interval, and an upper range of the non-ablatable impedance interval, and indicates, from top to bottom in a negative direction, a lower range of the optimal ablatable impedance interval, a lower range of the ablatable impedance interval, and a lower range of the non-ablatable impedance interval.

Optionally, the schematic real-time impedance change diagram is drawn on the preset prompting interaction interface.

Therefore, by drawing the schematic real-time impedance change diagram, the user is promoted to get to know the impedance changes of the ablation site in real time, and the user is reminded to adjust the ablation operation in time by an impedance warning, thus further improving the universality and effectiveness of information prompts.

Optionally, in another embodiment of the present invention, the description information of the corresponding relationship between the impedance and the target interval includes: graphics of different colors with preset shapes, wherein the different colors respectively correspond to different ablation impedance prompting intervals. For example, the red color corresponds to the non-ablatable impedance interval, the yellow color corresponds to the ablatable impedance interval, and the green color corresponds to the optimal ablatable impedance interval. Therefore, by using different colors, different ablation impedance intervals can be effectively distinguished, thereby further improving the effectiveness of information prompts.

Optionally, the height of the graphic is determined according to the difference between the impedance and the upper limit or lower limit of the corresponding target interval.

Preferably as shown in FIG. 70, the graphic has a bar shape, and as the impedance approaches the limit of the corresponding target interval, the length of the bar increases. The schematic real-time impedance change diagram can not only indicate the user with the real-time impedance change, but also indicate the user whether there is a safety risk in the current ablation operation. For example, the target interval corresponding to the impedance 12 in FIG. 70 is an optimal ablatable impedance interval, and indicating that the ablation operation at this time can achieve the best ablation effect; and the target interval corresponding to the impedance 14 is a non-ablatable impedance interval, indicating that the impedance of the ablation site is too high or too low, and there is a safety risk in the current ablation operation.

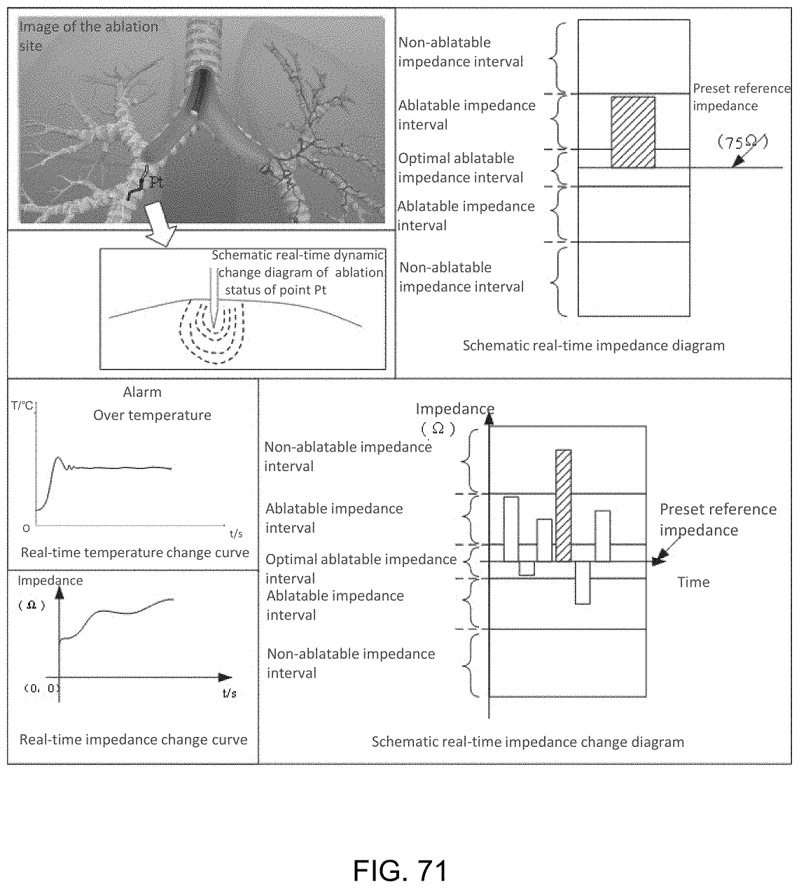

It should be noted that the schematic real-time impedance diagram, the schematic real-time impedance change diagram, the real-time impedance change curve, and other schematic diagrams do not conflict with each other. In practical use, one or more schematic diagrams illustrating a selective operation can be drawn and displayed according to the selective operation of the user. For example, as shown in FIG. 71, all the schematic diagrams are displayed on the preset prompting interaction interface. FIG. 64, FIG. 65, and FIGS. 67 to 71 are merely exemplary. In practical use, other more or less information, for example, specific temperature, impedance, system time, described information of the ablation subject, described information of the person in charge of the ablation operation, can be included in a different arrangement according to the practical needs or user's choice.

Further, the radio-frequency ablation catheter includes a single-electrode radio-frequency ablation catheter and a multi-electrode radio-frequency ablation catheter, wherein the single-electrode radio-frequency ablation catheter is correspondingly provided with a single impedance sensor, and the multi-electrode radio-frequency ablation catheter is correspondingly provided with multiple impedance sensors. The target interval can be determined according to the type of radio-frequency ablation catheter. Specifically, the determining a target interval corresponding to the impedance, according to the impedance acquired in real time and the impedance ranges respectively corresponding to multiple preset ablation impedance prompting intervals, includes: when the radio-frequency ablation catheter is a single-electrode radio-frequency ablation catheter, determining a target interval corresponding to the single impedance in real time according to the single impedance acquired in real time and the impedance ranges respectively corresponding to multiple ablation impedance prompting intervals; and when the radio-frequency ablation catheter is a multi-electrode radio-frequency ablation catheter, analyzing whether the multiple impedances obtained in real time correspond to the same ablation impedance prompting interval according to the impedance ranges respectively corresponding to multiple ablation impedance prompting intervals, wherein if the multiple impedances correspond to the same ablation impedance prompting interval, the corresponding ablation impedance prompting interval is used as the target interval, and if the multiple impedances correspond to multiple ablation impedance prompting intervals, an ablation impedance prompting interval that covers the most impedances is used as the target interval.

Specifically, before implementing an ablation task, the radio-frequency ablation control device acquires model information of the connected radio-frequency ablation catheter, determines the type of the radio-frequency ablation catheter according to the acquired model information. Then, the target interval is determined according to the type determined. Taking a six-electrode radio-frequency ablation catheter as an example, the six-electrode radio-frequency ablation catheter is assumed to be correspondingly provided with 6 impedance sensors, if the 6 impedances obtained by the 6 impedance sensors all fall in the optimal ablatable impedance interval, the corresponding target interval is determined to be the optimal ablatable impedance interval, and if 4 of the 6 impedances fall in the ablatable impedance interval and 2 impedances fall in the optimal ablatable impedance interval, the corresponding target interval is determined to be the ablatable impedance interval.

Optionally, in another embodiment of the present invention, after Step S601, the method further includes: when the prompt information outputted on the screen or the drawn schematic diagram has target information containing preset keywords, performing a screencapture operation, and saving the captured picture; and displaying all the pictures saved on the screen according to the priority of the target information, after the ablation task is ended.

Specifically, whether the prompt information outputted on the screen or the drawn schematic diagram has target information containing preset keywords is analyzed in real time, wherein the preset keywords have alert meaning, and may include, but is not limited to, for example: alarm, reminder, attention, and warning. If there is target information that contains the preset keywords, a screencapture operation is performed, and the captured picture is stored in the radio-frequency ablation control device locally or at a preset location of a cloud server. Then, after the ablation task is ended, all the pictures saved are displayed on the screen according to the timing sequence upon capture or the priority of the target information, to indicate to the user the abnormal conditions during the implementation of the entire ablation operation, thereby further improving the scope of application of information prompts, and improve the universality and intelligence of information prompts. A higher priority of the target information indicates a higher severity or importance of the corresponding prompt information.

Optionally, when the preset prompting interaction interface is used as a carrier, whether the prompt information outputted on the prompting interaction interface or the drawn schematic diagram has target information containing preset keywords is analyzed in real time.

It should be noted that for ease of description, the steps in each embodiment of the present invention are numbered in sequence; however, the numbering sequence does not constitute a restriction on the order of implementation. Some steps can be implemented at the same time, for example, Step S602 and Step S603 can be implemented at the same time, Step S604 and step S605 can also be implemented at the same time, and the generation and display of other schematic diagrams involved may also be implemented at the same time.

In the embodiments of the present invention, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic image showing the real-time dynamic change of the ablation status of the target ablation point is generated and displayed, so that the status changes of an ablation site can be displayed in real time and intuitively during the implementation of the ablation operation, thereby improving the effectiveness and relevance of information prompts.

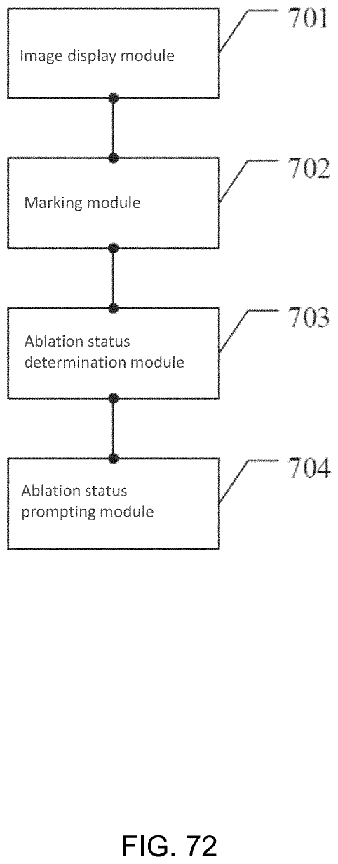

FIG. 72 is a schematic structural diagram of an ablation operation prompting device provided in an embodiment of the present invention. For ease of description, only the parts relevant to the embodiments of the application are shown. The device may be a computer terminal, or a software module configured on the computer terminal. As shown in FIG. 72, the device includes an image display module 701, a marking module 702, an ablation status determination module 703 and an ablation status prompting module 704.

The image display module 701 is configured to acquire an image of an ablation site and display the image on a screen, when an ablation task is triggered.

The marking module 702 is configured to acquire position data of a currently-being-ablated target ablation point, and mark the target ablation point in the image according to the position data.

The ablation status determination module 703 is configured to acquire the elapsed ablation time and the temperature of the target ablation point in real time, and determine the ablation status of the target ablation point according to the elapsed ablation time and the temperature.

The ablation status prompting module 704 is configured to generate a schematic diagram showing the real-time dynamic change of the target ablation point according to the ablation status, and display the schematic diagram on the screen, to indicate the real-time ablation status change of the target ablation point.

Optionally, the device further includes: a target operation track drawing module, configured to acquire a first drawing parameter of a target operation track of the ablation operation, and draw the target operation track at a corresponding position of the image according to the first drawing parameter; a real-time operation track drawing module, configured to acquire the position change data of the ablation point in real time, determine a second drawing parameter of a real-time operation track of the ablation operation according to the position change data, and draw the real-time operation track at a corresponding position of the image according to the second drawing parameter; and a track warning module, configured to analyze in real time whether the amplitude of the real-time operation track deviating from the target operation track is greater than a preset amplitude, and outputting prompt information for track deviation warning when the real-time operation track deviates from the target operation track by an amplitude greater than the preset amplitude.

Optionally, the device further includes: a temperature warning module, configured to acquire the temperature of the ablation site in real time, when the ablation task is triggered; draw a real-time temperature change curve on the screen according to the temperature acquired in real time; and analyze in real time whether the temperature exceeds a preset warning value, and outputting prompt information for temperature warning in a display area of the temperature change curve when the temperature exceeds the warning value.

Optionally, the device further includes: an impedance acquisition module, configured to acquire the impedance of the ablation site in real time, when the ablation task is triggered; and a real-time impedance change curve drawing module, configured to draw a real-time impedance change curve on the screen according to the impedance obtained in real time.

Optionally, the device further includes: a target interval determination module, configured to determine a target interval corresponding to the impedance, according to the impedance acquired in real time and the impedance ranges respectively corresponding to multiple preset ablation impedance prompting intervals; and a schematic real-time impedance diagram drawing module, configured to draw a schematic real-time impedance diagram on the screen according to the impedance, the impedance range and the target interval. The schematic real-time impedance diagram includes: description information of the multiple ablation impedance prompting intervals, description information of a preset reference impedance, and description information of the corresponding relationship between the impedance and the target interval. The multiple ablation impedance prompting intervals include: a non-ablatable impedance interval, an ablatable impedance interval, and an optimal ablatable impedance interval.

Optionally, the description information of the multiple ablation impedance prompting intervals includes: multiple vertically laminated columns and the corresponding description texts and indicating graphics of the multiple ablation impedance prompting intervals. The description information of the preset reference impedance includes: the description text and the indicating graphic of the preset reference impedance.

The multiple columns respectively correspond, from top to bottom, to an upper range of the non-ablatable impedance interval, an upper range of the ablatable impedance interval, an upper range of the optimal ablatable impedance interval, a lower range of the optimal ablatable impedance interval, a lower range of the ablatable impedance interval, and a lower range of the non-ablatable impedance interval.

Taking the column corresponding to the optimal ablatable impedance interval as a center, the height increases as the distance of the other columns in the multiple columns from the column increases.

Optionally, the device further includes: a schematic real-time impedance change diagram drawing module, configured to draw a schematic real-time impedance change diagram on the screen according to the impedance, the impedance range and the target interval. The schematic real-time impedance change diagram includes: two-dimensional coordinate axes, description information of the multiple ablation impedance prompting intervals, and description information of the corresponding relationship between each impedance and respective target interval. The multiple ablation impedance prompting intervals include: a non-ablatable impedance interval, an ablatable impedance interval, and an optimal ablatable impedance interval.

The horizontal axis of the two-dimensional coordinate axes is a time axis, indicating the acquisition time of each impedance and also the preset reference impedance. The vertical axis of the two-dimensional coordinate axes indicates, from bottom to top in a positive direction, an upper range of the optimal ablatable impedance interval, an upper range of the ablatable impedance interval, and an upper range of the non-ablatable impedance interval, and indicates, from top to bottom in a negative direction, a lower range of the optimal ablatable impedance interval, a lower range of the ablatable impedance interval, and a lower range of the non-ablatable impedance interval.

Optionally, the description information of the corresponding relationship includes: graphics of different colors with preset shapes, wherein the different colors respectively correspond to different ablation impedance prompting intervals.

The height of the graphic is determined according to the difference between the impedance and the upper limit or lower limit of the corresponding target interval.

Optionally, the target interval determination module is specifically configured to determine a target interval corresponding to the single impedance in real time according to the single impedance acquired in real time and the impedance ranges respectively corresponding to multiple ablation impedance prompting intervals when the radio-frequency ablation catheter is a single-electrode radio-frequency ablation catheter; and analyze whether the multiple impedances obtained in real time correspond to the same ablation impedance prompting interval according to the impedance ranges respectively corresponding to multiple ablation impedance prompting intervals, when the radio-frequency ablation catheter is a multi-electrode radio-frequency ablation catheter, determine the corresponding ablation impedance prompting interval as the target interval, if the multiple impedances correspond to the same ablation impedance prompting interval, and an ablation impedance prompting interval that covers the most impedances is used as the target interval.

Optionally, the marking module 702 includes: a first positioning module, configured to acquire a picture of a current ablation operation captured by an endoscope and compare the captured picture with the image, to obtain the position data of the target ablation point in the image.

Optionally, the marking module 702 further includes:

a second positioning module, configured to obtain an ultrasound image of the target ablation point; and acquire the position data of the target ablation point in the image according to the ultrasound image.

Optionally, the marking module 702 further includes: a third positioning module, configured to acquire the position data of the target ablation point, by electromagnetic navigation technology.

Optionally, the device further includes: a screencapture module, configured to perform a screencapture operation when the prompt information outputted on the screen or the drawn schematic diagram has target information containing preset keywords, and save the captured picture; and a display module, configured to display all the pictures saved on the screen according to the priority of the target information, after the ablation task is ended.

Optionally, the device further includes: an ablation status analyzing module, configured to determine whether the ablation status of the target ablation point reaches a preset target ablation status according to the elapsed ablation time and the temperature, and output prompt information indicating reaching of the target ablation status if the ablation status of the target ablation point reaches the target ablation status.

The marking module 702 is further configured to take an ablation point corresponding to a changed position as the target ablation point, when the position of the ablation operation is detected to be changed; update the mark in the image and return to implement the step of acquiring the elapsed ablation time and the temperature of the target ablation point in real time, and determining the ablation status of the target ablation point according to the elapsed ablation time and the temperature, until the ablation operation is ended.

The specific process for the above modules to implement their respective functions can be made reference to the relevant description in the embodiments shown in FIG. 63 to FIG. 71, and will not be repeated here.

In the embodiments of the present invention, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic image showing the real-time dynamic change of the ablation status of the target ablation point is generated and displayed, so that the status changes of an ablation site can be displayed in real time and intuitively during the implementation of the ablation operation, thereby improving the effectiveness and relevance of information prompts.

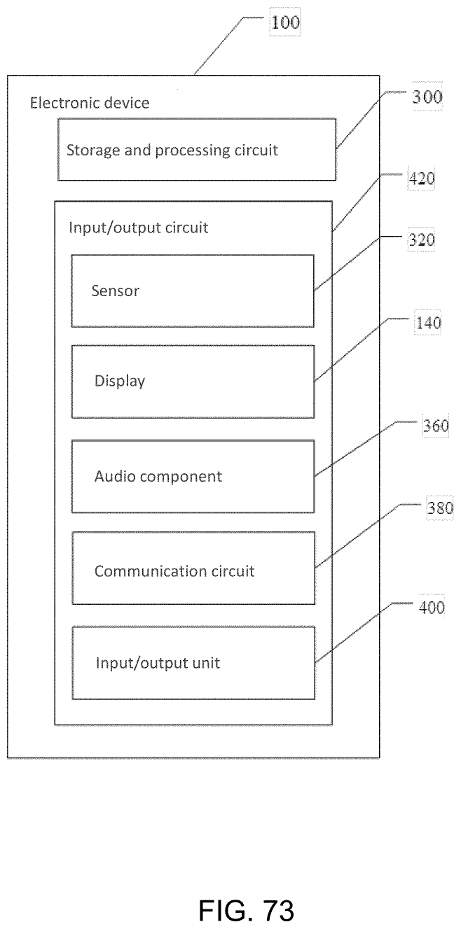

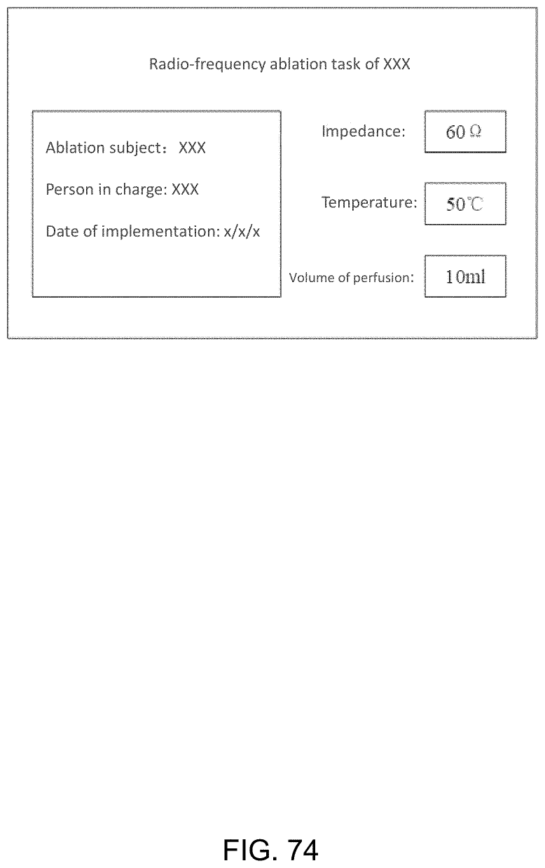

FIG. 73 is a schematic diagram showing a hardware structure of an electronic device provided in an embodiment of the present invention.

The electronic device may be any of various types of computer devices that are non-movable or movable or portable and capable of wireless or wired communication. Specifically, the electronic device can be a desktop computer, a server, a mobile phone or smart phone (e.g., a phone based on iPhone™, and Android™), a portable game device (e.g. Nintendo DS™, PlayStation Portable™, Gameboy Advance™, iPhone™), a laptop computer, PDA, a portable Internet device, a portable medical device, a smart camera, a music player, a data storage device, and other handheld devices such as watches, earphones, pendants, and headphones, etc. The electronic device may also be other wearable devices (for example, electronic glasses, electronic clothes, electronic bracelets, electronic necklaces and other head-mounted devices (HMD)).

In some cases, the electronic device can implement a variety of functions, for example: playing music, displaying videos, storing pictures and receiving and sending phone calls.

As shown in FIG. 73, the electronic device 100 includes a control circuit, and the control circuit includes a storage and processing circuit 300. The storage and processing circuit 300 includes a storage, for example, hard drive storage, non-volatile storage (such as flash memory or other storages that are used to form solid-state drives and are electronically programmable to confine the deletion, etc.), and volatile storage (such as static or dynamic random access storage) which is not limited in the embodiments of the present invention. The processing circuit in the storage and processing circuit 300 can be used to control the operation of the electronic device 100. The processing circuit can be implemented based on one or more microprocessors, microcontrollers, digital signal processors, baseband processors, power management units, audio codec chips, application specific integrated circuits, and display driver integrated circuits.

The storage and processing circuit 300 can be used to run the software in the electronic device 100, such as Internet browsing applications, Voice over Internet Protocol (VOIP) phone call applications, Email applications, media player applications, and functions of operating system. The software can be used to perform some control operations, for example, camera-based image acquisition, ambient light measurement based on an ambient light sensor, proximity sensor measurement based on a proximity sensor, information display function implemented by a status indicator based on a status indicator lamp such as light emitting diode, touch event detection based on a touch sensor, functions associated with displaying information on multiple (e.g. layered) displays, operations associated with performing wireless communication functions, operations associated with the collection and generation of audio signals, control operations associated with the collection and processing of button press event data, and other functions in electronic device 100, which are not limited in the embodiments of the present invention.

Further, the storage stores an executable program code. The processor coupled to the storage calls the executable program code stored in the storage, and implement the ablation operation prompting method described in the embodiments shown in FIGS. 63 to 71.

The executable program code includes various modules in the ablation operation prompting device described in the embodiment shown in FIG. 72, for example, the image display module 701, the marking module 702, the ablation status determination module 703, and the ablation status prompting module 704. The specific process for the above modules to implement their functions can be made reference to the relevant description in the embodiments shown in FIG. 72, and will not be repeated here.

The electronic device 100 may also include an input/output circuit 420. The input/output circuit 420 can be used to enable the electronic device 100 to implement the input and output of data, that is, the electronic device 100 is allowed to receive data from an external device and the electronic device 100 is also allowed to output data from the electronic device 100 to the external device. The input/output circuit 420 may further include a sensor 320. The sensor 320 may include one or a combination of an ambient light sensor, a proximity sensor based on light and capacitance, a touch sensor (e.g., light-based touch sensor and/or capacitive touch sensor, wherein, wherein the touch sensor may be part of the touch screen, or it can also be used independently as a touch sensor structure), an accelerometer, and other sensors, etc.

The input/output circuit 420 may also include one or more displays, for example, display 140. The display 140 may include a liquid crystal display, an organic light emitting diode display, an electronic ink display, a plasma display, and a display that uses other display technologies. The display 140 may include a touch sensor array (i.e., the display 140 may be a touch display screen). The touch sensor can be a capacitive touch sensor formed by an array of transparent touch sensor electrodes (such as indium tin oxide (ITO) electrodes), or a touch sensor formed using other touch technologies, for example, sonic touch, pressure sensitive touch, resistive touch, and optical touch, which is not limited in the embodiments of the present invention.

The electronic device 100 can also include an audio component 360. The audio component 360 can be used to provide audio input and output functions for the electronic device 100. The audio component 360 in the electronic device 100 includes a speaker, a microphone, a buzzer, and a tone generator and other components used to generate and detect sound.

A communication circuit 380 can be used to provide the electronic device 100 with an ability to communicate with an external device. The communication circuit 380 may include an analog and digital input/output interface circuit, and a wireless communication circuit based on radio-frequency signals and/or optical signals. The wireless communication circuit in the communication circuit 380 may include a radio-frequency transceiver circuit, a power amplifier circuit, a low-noise amplifier, a switch, a filter, and an antenna. For example, the wireless communication circuit in the communication circuit 380 may include a circuit for supporting near field communication (NFC) by transmitting and receiving near-field coupled electromagnetic signals. For example, the communication circuit 380 may include a near-field communication antenna and a near-field communication transceiver. The communication circuit 380 may also include a cellular phone transceiver and antenna, and a wireless LAN transceiver circuit and antenna, etc.

The electronic device 100 may also further include a battery, a power management circuit and other input/output units 400. The input/output unit 400 may include a button, a joystick, a click wheel, a scroll wheel, a touchpad, a keypad, a keyboard, a camera, a light-emitting diode and other status indicators, etc.

The user can control the operation of the electronic device 100 by inputting a command through the input/output circuit 420, and the output data from the input/output circuit 420 enables the receiving of status information and other outputs from the electronic device 100.

Further, an embodiment of the present invention further provides a non-transitory computer-readable storage medium. The non-transitory computer-readable storage medium can be configured in the server in each of the above embodiments, and a computer program is stored in the non-transitory computer-readable storage medium. When the program is executed by the processor, the ablation operation prompting method described in the embodiments shown in FIG. 63 to FIG. 71 is implemented.

In the embodiments described above, emphasis has been placed on the description of various embodiments. Parts of an embodiment that are not described or illustrated in detail may be found in the description of other embodiments.

Those of ordinary skill in the art will recognize that the exemplary modules/units and algorithm steps described in connection with the embodiments disclosed herein may be implemented by electronic hardware, or a combination of computer software and electronic hardware. Whether such functions are implemented by hardware or software depends on the particular application and design constraints of the technical solutions. Skilled artisans may implement the described functions in varying ways for each particular application. but such implementation is not intended to exceed the scope of the present invention.

In the embodiments provided in the present invention, it can be understood that the disclosed device/terminal and method may be implemented in other ways. For example, the device/terminal embodiments described above are merely illustrative. For example, a division of the modules or elements is merely division of logical functions, and there may be additional divisions in actual implementation. For example, multiple units or components may be combined or integrated into another system, or some features may be omitted or not performed. Alternatively, the couplings or direct couplings or communicative connections shown or discussed with respect to one another may be indirect couplings or communicative connections via some interfaces, devices or units may be electrical, mechanical or otherwise.

The units described as separate components may or may not be physically separate, and the components shown as units may or may not be physical units, i.e. may be located in one place, or may be distributed over a plurality of network elements. Some or all of the units may be selected to achieve the objectives of the solution of the present embodiment according to practical requirements.

In addition, the functional units in the various embodiments of the present invention may be integrated into one processing unit, may be physically separate from each other or may be integrated in one unit by two or more units. The integrated units described above can be implemented either in the form of hardware, or software functional units.

The integrated unit, if implemented in the form of a software functional unit and sold or used as a stand-alone product, may be stored in a computer readable storage medium. Based on such an understanding, all or part of the processes of the methods in the above-described embodiments implemented in the present invention, may also be implemented by a computer program instructing related hardware. The computer program may be stored in a computer-readable storage medium, and performs the steps of the various method embodiments described above when executed by the processor. The computer program includes a computer program code, which may be in the form of source code, object code, or executable file, or in some intermediate form. The computer readable medium may include: any entity or device capable of carrying the computer program code, recording media, U disks, removable hard disks, magnetic disks, optical disks, computer memory, read-only memory (ROM), random access memory (RAM), electric carrier wave signals, telecommunication signals and software distribution media. It should be noted that the computer readable medium may contain content that may be appropriately augmented or subtracted as required by legislation and patent practice within judicial jurisdictions, e.g., the computer-readable medium does not include electrical carrier wave signals and telecommunications signals in accordance with legislation and patent practices in some jurisdictions.

The above-described embodiments are merely illustrative of, and not intended to limit the technical solutions of the present invention. Although the present invention has been described in detail with reference to the foregoing embodiments, it should be understood by those of ordinary skill in the art that the technical solutions of the above-mentioned embodiments can still be modified, or some of the technical features thereof can be equivalently substituted; and such modifications and substitutions do not cause the nature of the corresponding technical solution to depart from the spirit and scope of the embodiments of the present disclosure and are intended to be included within the scope of the present invention.

Radio-Frequency Operation Prompting Method, Electronic Device, and Computer-Readable Storage Medium

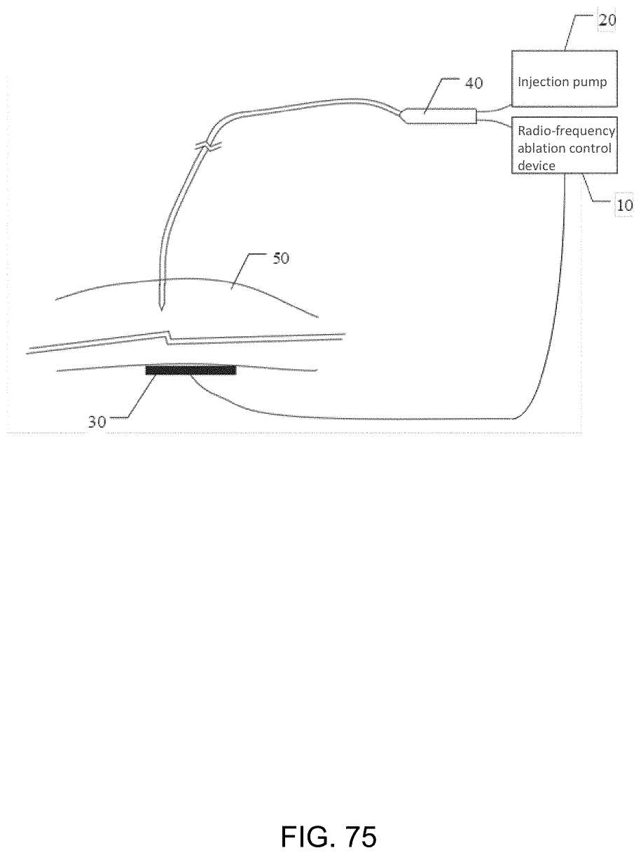

FIG. 75 is a schematic view showing an application scenario of a radio-frequency operation prompting method provided in an embodiment of the present invention. The radio-frequency operation prompting method can be implemented by a radio-frequency host 10 shown in FIG. 75, or implemented by other computer equipment that has established a data connection with the radio-frequency host 10.

As shown in FIG. 75, the radio-frequency host 10 is connected to a syringe pump 20, a neutral electrode 30 and a radio-frequency operation catheter 40. The radio-frequency host 10 is provided with a built-in display screen (not shown).

Specifically, before an operation task is implemented, an energy emitting end of the radio-frequency operation catheter 40 for generating and outputting radio-frequency energy and an extension tube (not shown) of the syringe pump 20 are inserted into the body of a subject 50 (such as an abnormal tissue mass). Then, the neutral electrode 30 is brought into contact with the skin surface of the subject 50. A radio-frequency current flows through the radio-frequency operation catheter 40, the subject 50 and the neutral electrode 30, to form a circuit.

When the operation task is triggered, the radio-frequency operation catheter 40 is controlled by the radio-frequency host 10 to output radio-frequency energy to an operation site by discharging, so as to implement a radio-frequency operation on the operation site. Moreover, the syringe pump 20 performs a perfusion operation on the subject through the extension tube, wherein physiological saline is infused into the operation site, to adjust the impedance and temperature of the operation site.

Moreover, the radio-frequency host 10 acquires physical characteristic data of an operating position in the subject of the radio-frequency operation in real time by multiple probes (not shown) provided at a tip of the radio-frequency operation catheter 40, obtains a physical characteristic field of the subject of the radio-frequency operation according to the physical characteristic data acquired in real time; and then obtains the change of range of a to-be-operated area in a target operating area according to an initial range of the target operating area in the subject of the radio-frequency operation and the change of value of the physical characteristic data in the physical characteristic field, and displays the change of range by a three-dimensional model.

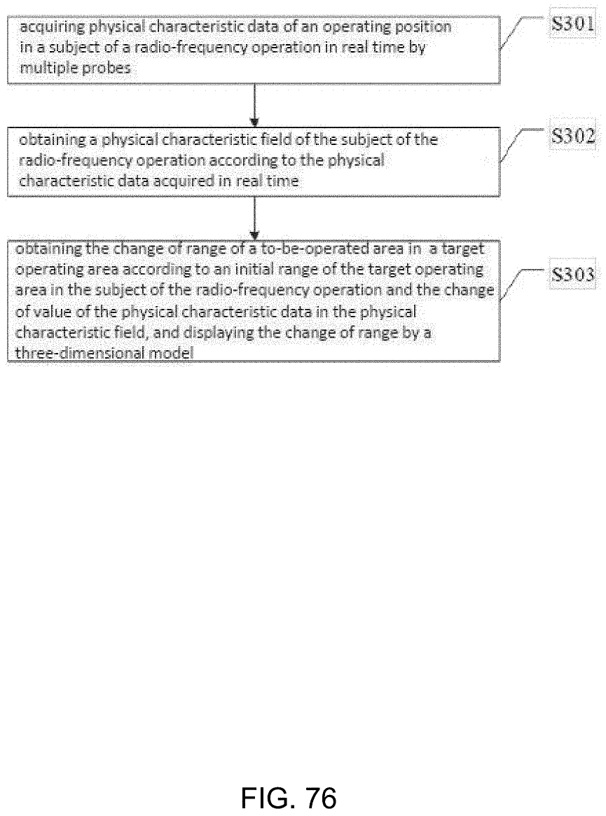

FIG. 76 shows a flow chart of a radio-frequency operation prompting method provided in an embodiment of the present invention. The method can be implemented by the radio-frequency host 10 shown in FIG. 75, or implemented by other computer terminals connected thereto. For ease of description, in the following embodiments, the radio-frequency host 10 is used as an implementation body. As shown in FIG. 76, the method includes specifically:

Step S301: acquiring physical characteristic data of an operating position in a subject of a radio-frequency operation in real time by multiple probes.

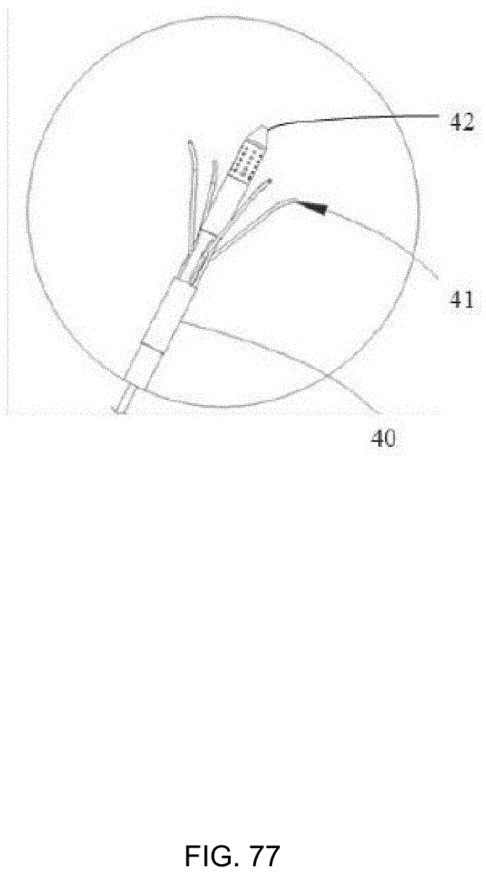

As shown in FIG. 77, multiple probes 41 are arranged around a central electrode 42 for outputting radio-frequency energy provided at a tip of the radio-frequency operation catheter 40, and located on different planes, to form a claw-shaped structure collectively. Each probe is provided with a physical characteristic data acquiring device, configured to acquire physical characteristic data of a pierced or touched position.

Particularly, when the radio-frequency operation catheter is controlled by the radio-frequency host to perform a radio-frequency operation, the probe comes into contact with an operating site of the subject of the radio-frequency operation along with the central electrode, to detect the physical characteristic data of different positions in the operating site in real time. The physical characteristic data can be specifically temperature or impedance, or both temperature and impedance.

The subject of the radio-frequency operation refers to any subject or object that can receive radio-frequency operation such as radio-frequency ablation. For example, when the radio-frequency operation is radio-frequency ablation, the subject of the radio-frequency operation may be a biological tissue, and the operating position may be an abnormal tissue in the biological tissue.

Step S302: obtaining a physical characteristic field of the subject of the radio-frequency operation according to the physical characteristic data acquired in real time.

Specifically, the physical characteristic data includes temperature data and impedance data, and correspondingly, the physical characteristic field can include a temperature field and a resistance field.

The temperature field is a set of temperatures at various points in the subject of the radio-frequency operation, reflecting the spatial and temporal distribution of temperature; and can generally be expressed as a function of spatial coordinates and time of an object. That is, t=f(x, y, z, τ), wherein x, y, and z are three rectangular coordinates in space, and r is the time coordinate. In the prior art, many specific algorithms for temperature field are available, which are not particularly limited in the present invention.

Similar to the temperature field, the impedance field is a set of impedances at various points in the subject of the radio-frequency operation, and is a function of time and spatial coordinates, reflecting the spatial and temporal distribution of impedance.

Step S303: obtaining the change of range of a to-be-operated area in a target operating area according to an initial range of the target operating area in the subject of the radio-frequency operation and the change of value of the physical characteristic data in the physical characteristic field, and displaying the change of range by a three-dimensional model.

The target operating area is an implementation area of the present radio-frequency operation in the subject of the radio-frequency operation, and the initial range of the target operating area can be obtained by X-ray scanning and other transmission scanning techniques.

The to-be-operated area is an area where the present radio-frequency operation has not been performed or the effect after the present radio-frequency operation has not reached the standard, and an area where the radio-frequency operation still needs to be performed.

The value of the physical characteristic data of each point in the physical characteristic field changes at any time as the radio-frequency operation progresses, and the value of the physical characteristic data represents the stage of the radio-frequency operation. Particularly, when the value of the physical characteristic data reaches a preset threshold, it indicates the end of the radio-frequency operation (i.e., achieving the desired effect). When the value of the physical characteristic data is less than the preset threshold, it indicates that the radio-frequency operation is not ended (i.e., not achieving the desired effect) or does not start. The area where the radio-frequency operation is not ended or does not start is the to-be-operated area. That is, according to the value of the physical characteristic data in the physical characteristic field, the range of the to-be-operated area can be determined. As the measured value of each point in the physical characteristic field changes, the range of the to-be-operated area changes accordingly, showing such a trend that as the past time of radio-frequency operation increases, the range of the to-be-operated area becomes smaller and smaller.

By default 3D model display software, the change of range of the to-be-operated area in the target operating area is displayed on a display interface of the radio-frequency host, to intuitively present it to a radio-frequency operation personnel to get to know the status of the radio-frequency operation.

In the embodiments of the present invention, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

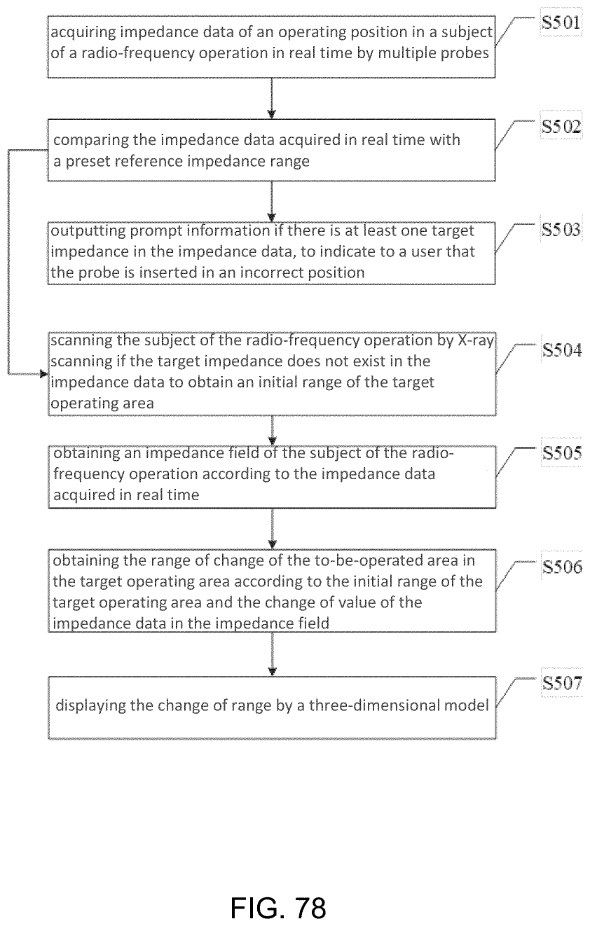

FIG. 78 shows a flow chart of a radio-frequency operation prompting method provided in another embodiment of the present invention. The method can be implemented by the radio-frequency host 10 shown in FIG. 75, or implemented by other computer terminals connected thereto. For ease of description, in the following embodiments, the radio-frequency host 10 is used as an implementation body. As shown in FIG. 78, the method includes specifically:

Step S501: acquiring impedance data of an operating position in a subject of a radio-frequency operation in real time by multiple probes.

Step S502: comparing the impedance data acquired in real time with a preset reference impedance range.

Step S503: outputting prompt information if there is at least one target impedance in the impedance data, to indicate to a user that the probe is inserted in an incorrect position.

As shown in FIG. 77, multiple probes 41 are arranged around a central electrode 42 provided at a tip of the radio-frequency operation catheter 40, and located on different planes, to form a claw-shaped structure collectively. Each probe is provided with an impedance acquiring device, configured to acquire the impedance data of a pierced or touched position.

It can be understood that the impedance of normal biological tissues and the impedance of abnormal biological tissues are different, and a reference impedance database is configured in the radio-frequency host 10. The reference impedance database is configured to store the reference impedance range corresponding to each of different types of abnormal biological tissues (e.g., tumors, inflammations, and cancers). By querying the reference impedance database, the reference impedance range corresponding to the type of abnormality in the subject of the current radio-frequency operation can be obtained. Optionally, the reference impedance database can also be configured in the cloud.

The value of the target impedance is not within the reference impedance range. The impedance data acquired by the multiple probes is compared with the reference impedance range queried, if there is at least one target impedance in the acquired impedance data, it means that the probe does not completely cover the site where the radio-frequency operation needs to be performed, and the expected result may not be achieved if the radio-frequency operation is performed at the current position. Consequently, preset prompt information is output on the display screen, to indicate to the user that the probe is inserted in an incorrect position.

Further, the prompt information also includes position information of a probe that obtains the target impedance, so that the user can determine a displacement direction of the probe according to the position information, making the information prompt more intelligent.

Further, after the prompt information is outputted, the process is returned to Step S501 every preset period of time until the target impedance does not exist in the impedance data; or in response to a control command triggered by the user by pressing a preset physical or virtual button, Step S501 is performed again.

Therefore, by using the reference impedance range, prompting is made when the probe is inserted in an incorrect position, to realize the navigation of the probe positioning operation. This can make the information prompt more intelligent, and increase the speed of probe positioning, thus shortening the overall time of radio-frequency operation, and improving the operation efficiency.

Step S504: scanning the subject of the radio-frequency operation by X-ray scanning if the target impedance does not exist in the impedance data to obtain an initial range of the target operating area.

Specifically, if the target impedance does not exist in the acquired impedance data, that is, all the acquired impedances fall into the reference impedance range, indicating that the probe completely covers the site where the radio-frequency operation needs to be performed, a three-dimensional image of a target site is obtained by scanning the target site of the subject of the radio-frequency operation by X-ray scanning. Then the three-dimensional image is recognized, to obtain the position coordinates of each probe in the three-dimensional image. Then, the range of coverage of the probe is determined according to the obtained position coordinates, and the range of coverage is determined as the initial range of the target operating area. The X-ray scanning includes, for example, Computed Tomography (CT).

Step S505: obtaining an impedance field of the subject of the radio-frequency operation according to the impedance data acquired in real time.

Specifically, the impedance field is a set of impedances at various points in the subject of the radio-frequency operation, and is a function of time and spatial coordinates, reflecting the spatial and temporal distribution of impedance.

Step S506: obtaining the change of range of the to-be-operated area in the target operating area according to the initial range of the target operating area and the change of value of the impedance data in the impedance field.

The to-be-operated area is an area where the present radio-frequency operation has not been performed or the effect after the present radio-frequency operation has not reached the standard, and an area where the radio-frequency operation still needs to be performed. The value of the impedance data of each point in the impedance field changes at any time as the radio-frequency operation progresses, and the value of the impedance data represents the stage of the radio-frequency operation. For example, when the value of the impedance data reaches a preset threshold, it indicates that the radio-frequency operation achieves the desired effect. When the value of the impedance data is less than the preset threshold, it indicates that the radio-frequency operation does not achieve the desired effect or does not start. The area where the radio-frequency operation does not achieve the desired effect or does not start is the to-be-operated area. That is, according to the value of the impedance data in the impedance field, the range of the to-be-operated area can be determined. As the measured value of each point in the impedance field changes, the range of the to-be-operated area changes accordingly, showing such a trend that as the past time of radio-frequency operation increases, the range of the to-be-operated area becomes smaller and smaller.

Specifically, the value of the impedance data of each point in the impedance field is compared with a preset threshold (i.e., preset impedance threshold), and the boundary of the to-be-operated area is determined according to the points where the value of the impedance data is greater than the preset threshold.

It can be understood that the points where the value of the impedance data is greater than the preset threshold are fitted together by using a default fitting algorithm, to obtain the range of the area in the target operating area that has achieved the expected effect. The initial range of the target operating area is compared with the range of the area that has achieved the expected effect, to obtain the range and boundary of the to-be-operated area. The preset fitting algorithm includes, but is not limited to, for example, the least square method or the Matlab curve fitting algorithm, which is not particularly limited in the present invention.

Further, when the value of the impedance data of the operating position is greater than the preset threshold, the range of a radiation area of the operating position is determined according to the value of the impedance data of the operating position, a detection angle of the probe corresponding to the operating position and a preset radiation distance; and the boundary of the to-be-operated area is determined according to the range of the radiation area.

It can be understood that since the radio-frequency energy outputted by the central electrode of the radio-frequency operation catheter is radiated into the biological tissue along a specific direction, the impedance change has a radiation range.

The detection angle of the probe, that is, the angle at which the probe used to detect the impedance of a certain operating position is pierced into or brought into contact with the operating position. According to the detection angle, the direction of radiation can be determined. According to the value of the impedance data of the operating position, the direction of the radiation, and the preset radiation distance, the range of the radiation area of the operating position, that is, the depth of the boundary of the range of the area that has achieved the desired effect, can be determined.

Step S507: displaying the change of range by a three-dimensional model.

Specifically, according to the three-dimensional image of the target operating area obtained by X-ray scanning and the change of range of the to-be-operated area in the target operating area, and by using algorithms such as Marching Cubes based on surface rendering, or Ray-casting, Shear-warp, Frequency Domain, and Splatting based on volume rendering, a three-dimensional model of change of range is established for the to-be-operated area, and displayed on a preset display interface.

In the Marching Cubes algorithm, a series of two-dimensional slice data is deemed as a three-dimensional data field, and then a three-dimensional surface mesh modeling a three-dimensional model is established by extracting the isosurface of the three-dimensional data. In this way, the three-dimensional model is established. The algorithm based on volume rendering is to directly convert the discrete data in a three-dimensional space into a final three-dimensional image, without generating intermediate geometric primitives. The central idea is to define an opacity for each voxel, take the transmission, emission and reflection of each voxel to light into account.

Optionally, in another embodiment of the present invention, the multiple probes are configured to acquire temperature data of an operating position in a subject of a radio-frequency operation in real time, and the method includes the following steps:

Step S701: acquiring temperature data of an operating position in a subject of a radio-frequency operation in real time by multiple probes.

Step S702: obtaining an initial range of a target operating area by scanning the subject of the radio-frequency operation by X-ray scanning.

Step S703: obtaining a temperature field of the subject of the radio-frequency operation according to the temperature data acquired in real time.

Step S704: obtaining the range of change of a to-be-operated area in the target operating area according to the initial range of the target operating area and the change of value of the temperature data in the temperature field.

Step S705: displaying the change of range by a three-dimensional model.

The Steps S701 to S705 are similar to Step S501 and Steps S504 to S507, and related description can be made reference to Step S501 and Steps S504 to S507, and will not be repeated here.

Optionally, in another embodiment of the present invention, the multiple probes are configured to acquire temperature data and impedance data of an operating position in a subject of a radio-frequency operation in real time, and the method includes the following steps:

Step S801: acquiring temperature data and impedance data of an operating position in a subject of a radio-frequency operation in real time by multiple probes.

Step S802: comparing the impedance data acquired in real time with a preset reference impedance range.

Step S803: outputting prompt information if there is at least one target impedance in the impedance data, to indicate to a user that the probe is inserted in an incorrect position.

Step S804: obtaining the initial range of the target operating area by scanning the subject of the radio-frequency operation by X-ray scanning, if the target impedance does not exist in the impedance data.

Step S805: obtaining an impedance field and a temperature field of the subject of the radio-frequency operation according to the impedance data and the temperature data acquired in real time.

Step S806: obtaining the range of change of a to-be-operated area in the target operating area according to the initial range of the target operating area, the change of value of the impedance data in the impedance field and the change of value of the temperature data in the temperature field.

Step S807: displaying the change of range by a three-dimensional model.

The Steps S801 to S805 and Step S807 are similar to Steps S501 to S505 and Step S507, and related description can be made reference to Step S501 and Steps S505 to S507, and will not be repeated here.

Unlikely, Step S805 specifically includes: comparing the value of the temperature data of each point in the temperature field with a preset temperature threshold, and determining a first boundary of the to-be-operated area according to points where the value of the temperature data is greater than the preset temperature threshold; and after a preset period of time, comparing the value of the impedance data at each point in the impedance field with a preset impedance threshold, and calibrating the first boundary of the to-be-operated area according to points where the value of the impedance data is greater than the preset impedance threshold, to obtain a second boundary, wherein the second boundary is determined as the boundary of the to-be-operated area.

The step of calibrating the first boundary of the to-be-operated area according to points where the value of the impedance data is greater than the preset impedance threshold to obtain a second boundary is that at the same point, if the value of the impedance data is greater than the preset impedance threshold, but the value of the temperature data is not greater than the preset temperature threshold, the impedance data prevails, and the location corresponding to this point is determined as the location that achieves the desired effect.

Similarly, the first boundary is determined by using the temperature field, and then the first boundary is calibrated by using the impedance field, to achieve a complementary effect, and make the final boundary determined more accurate.

Optionally, the method also includes: determining the unit change of the to-be-operated area periodically according to the change of range of the to-be-operated area; and determining the remaining time before the end of the current radio-frequency operation according to the unit change and the current volume of the to-be-operated area, and outputting the remaining time as prompt information on a display interface.

Specifically, an interval is determined according to a preset time, and the change of range of the to-be-operated area is divided by the past implementation time of the radio-frequency operation every preset period of time, to obtain the unit change of the to-be-operated area, for example: the volume of the to-be-operated area reduced per second. Then, the current volume of the to-be-operated area is divided by the unit change, to obtain the remaining time before the end of the current radio-frequency operation.

The initial volume of the target operating area minus the unit changes of the to-be-operated area is the current volume of the to-be-operated area. The initial volume of the target operating area can be determined based on the three-dimensional image of the target operating area obtained by X-ray scanning in Step S504.

Therefore, by prompting the remaining time before the end of the current radio-frequency operation, the user is allowed to get to know the process of radio-frequency operation, thus further improving the intelligence of information prompts.

In the embodiments of the present invention, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

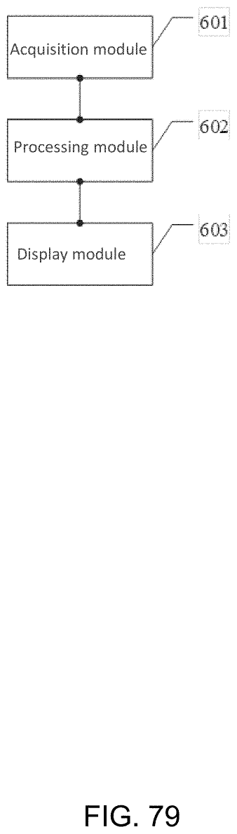

FIG. 79 is a schematic structural diagram of a radio-frequency operation prompting device provided in an embodiment of the present invention. For ease of description, only the parts relevant to the embodiments of the application are shown. The device may be a computer terminal, or a software module configured on the computer terminal. As shown in FIG. 79, the device includes an acquisition module 601, a processing module 602, and a display module 603.

The acquisition module 601 is configured to acquire physical characteristic data of an operating position in a subject of a radio-frequency operation in real time by multiple probes.

The processing module 602 is configured to obtain a physical characteristic field of the subject of the radio-frequency operation according to the physical characteristic data acquired in real time, and

obtain the change of range of a to-be-operated area in a target operating area according to an initial range of the target operating area in the subject of the radio-frequency operation and the change of value of the physical characteristic data in the physical characteristic field.

The display module 603 is configured to display the change of range by a three-dimensional model.

Further, the processing module 602 is further configured to compare the value of the physical characteristic data of each point in the physical characteristic field with a preset threshold; and determine a boundary of the range of the to-be-operated area according to points where the value of the physical characteristic data is greater than the preset threshold.

The processing module 602 is further configured to determine the range of a radiation area of the operating position according to the value of the physical characteristic data of the operating position, a detection angle of the probe corresponding to the operating position and a preset radiation distance, when the value of the physical characteristic data of the operating position is greater than the preset threshold. The value of the physical characteristic data in the radiation area is greater than the preset threshold; and the boundary of the to-be-operated area is determined according to the range of the radiation area.

Further, the physical characteristic data includes temperature data and/or impedance data, and the physical characteristic field includes a temperature field and/or a resistance field.

Further, when the physical characteristic data includes temperature data and impedance data, and the physical characteristic field includes a temperature field and a resistance field, the processing module 602 is further configured to compare the value of the temperature data of each point in the temperature field with a preset temperature threshold, and determine a first boundary of the to-be-operated area according to points where the value of the temperature data is greater than the preset temperature threshold; and

compare the value of the impedance data at each point in the impedance field with a preset impedance threshold after a preset period of time, and calibrate the first boundary of the to-be-operated area according to points where the value of the impedance data is greater than the preset impedance threshold to obtain a second boundary wherein the second boundary is determined as the boundary of the to-be-operated area.

Further, the processing module 602 is further configured to determine the unit change of the to-be-operated area periodically according to the change of range of the to-be-operated area; and determine the remaining time before the end of the current radio-frequency operation according to the unit change and the current volume of the to-be-operated area.

The display module 603 is configured to output the remaining time as prompt information on a display interface.

Further, when the physical characteristic data includes impedance data, the processing module 602 is further configured to compare the impedance data acquired in real time with a preset reference impedance range, and trigger the display module 603 to output prompt information if there is at least one target impedance in the impedance data, to indicate to a user that the probe is inserted in an incorrect position, and the value of the target impedance is not within the reference impedance range;

and trigger the step of obtaining a physical characteristic field of the subject of the radio-frequency operation, according to the physical characteristic data acquired in real time, if the target impedance does not exist in the impedance data.

Further, the processing module 602 is further configured to scan the subject of the radio-frequency operation by X-ray scanning, to obtain the initial range of the target operating area.

The specific process for the above modules to implement their respective functions can be made reference to the relevant description in the embodiments shown in FIG. 76 to FIG. 78, and will not be repeated here.

In the embodiments of the present invention, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

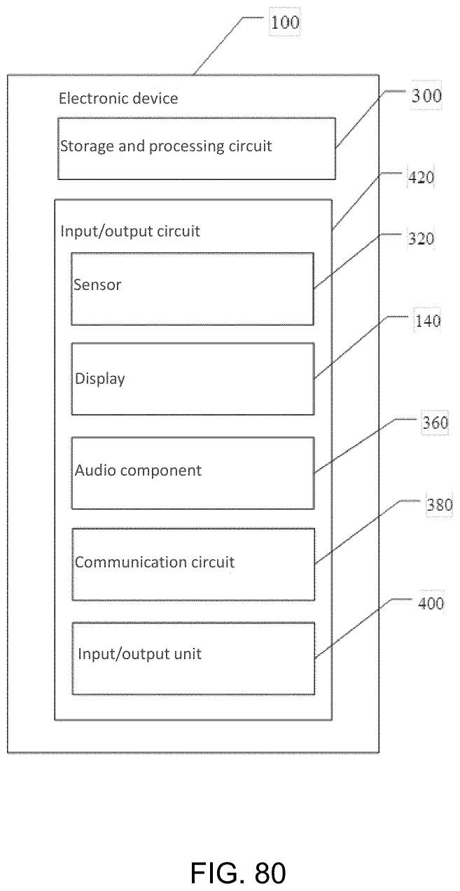

FIG. 80 is a schematic diagram showing a hardware structure of an electronic device provided in an embodiment of the present invention.

The electronic device may be any of various types of computer devices that are non-movable or movable or portable and capable of wireless or wired communication. Specifically, the electronic device can be a desktop computer, a server, a mobile phone or smart phone (e.g., a phone based on iPhone™, and Android™), a portable game device (e.g. Nintendo DS™, PlayStation Portable™, Gameboy Advance™, iPhone™), a laptop computer, PDA, a portable Internet device, a portable medical device, a smart camera, a music player, a data storage device, and other handheld devices such as watches, earphones, pendants, and headphones, etc. The electronic device may also be other wearable devices (for example, electronic glasses, electronic clothes, electronic bracelets, electronic necklaces and other head-mounted devices (HMD)).

As shown in FIG. 80, the electronic device 100 includes a control circuit, and the control circuit includes a storage and processing circuit 300. The storage and processing circuit 300 includes a storage, for example, hard drive storage, non-volatile storage (such as flash memory or other storages that are used to form solid-state drives and are electronically programmable to confine the deletion, etc.), and volatile storage (such as static or dynamic random access storage) which is not limited in the embodiments of the present invention. The processing circuit in the storage and processing circuit 300 can be used to control the operation of the electronic device 100. The processing circuit can be implemented based on one or more microprocessors, microcontrollers, digital signal processors, baseband processors, power management units, audio codec chips, application specific integrated circuits, and display driver integrated circuits.

The storage and processing circuit 300 can be used to run the software in the electronic device 100, such as Internet browsing applications, Voice over Internet Protocol (VOIP) phone call applications, Email applications, media player applications, and functions of operating system. The software can be used to perform some control operations. For example, camera-based image acquisition, ambient light measurement based on an ambient light sensor, proximity sensor measurement based on a proximity sensor, information display function implemented by a status indicator based on a status indicator lamp such as light emitting diode, touch event detection based on a touch sensor, functions associated with displaying information on multiple (e.g. layered) displays, operations associated with performing wireless communication functions, operations associated with the collection and generation of audio signals, control operations associated with the collection and processing of button press event data, and other functions in electronic device 100, which is not limited in the embodiments of the present invention.

Further, the storage stores an executable program code. The processor coupled to the storage calls the executable program code stored in the storage, and implements the radio-frequency operation prompting method described in various embodiments shown above.

The executable program code includes various modules in the radio-frequency operation prompting device described in the embodiment shown in FIG. 79, for example, the acquisition module 601, the processing module 602, and the display module 603. The specific process for the above modules to implement their functions can be made reference to the relevant description in the embodiments shown in FIG. 79, and will not be repeated here.

The electronic device 100 may also include an input/output circuit 420. The input/output circuit 420 can be used to enable the electronic device 100 to implement the input and output of data, that is, the electronic device 100 is allowed to receive data from an external device and the electronic device 100 is also allowed to output data from the electronic device 100 to the external device. The input/output circuit 420 may further include a sensor 320. The sensor 320 may include one or a combination of an ambient light sensor, a proximity sensor based on light and capacitance, a touch sensor (e.g., light-based touch sensor and/or capacitive touch sensor, wherein, the touch sensor may be part of the touch screen, or it can also be used independently as a touch sensor structure), an accelerometer, and other sensors, etc.

The input/output circuit 420 may also include one or more displays, for example, display 140. The display 140 may include a liquid crystal display, an organic light emitting diode display, an electronic ink display, a plasma display, and a display that uses other display technologies. The display 140 may include a touch sensor array (i.e., the display 140 may be a touch display screen). The touch sensor can be a capacitive touch sensor formed by an array of transparent touch sensor electrodes (such as indium tin oxide (ITO) electrodes), or a touch sensor formed using other touch technologies, for example, sonic touch, pressure sensitive touch, resistive touch, and optical touch, which is not limited in the embodiments of the present invention.

The electronic device 100 can also include an audio component 360. The audio component 360 can be used to provide audio input and output functions for the electronic device 100. The audio component 360 in the electronic device 100 includes a speaker, a microphone, a buzzer, and a tone generator and other components used to generate and detect sound.

A communication circuit 380 can be used to provide the electronic device 100 with an ability to communicate with an external device. The communication circuit 380 may include an analog and digital input/output interface circuit, and a wireless communication circuit based on radio-frequency signals and/or optical signals. The wireless communication circuit in the communication circuit 380 may include a radio-frequency transceiver circuit, a power amplifier circuit, a low-noise amplifier, a switch, a filter, and an antenna. For example, the wireless communication circuit in the communication circuit 380 may include a circuit for supporting near field communication (NFC) by transmitting and receiving near-field coupled electromagnetic signals. For example, the communication circuit 380 may include a near-field communication antenna and a near-field communication transceiver. The communication circuit 380 may also include a cellular phone transceiver and antenna, and a wireless LAN transceiver circuit and antenna, etc.

The electronic device 100 may also further include a battery, a power management circuit and other input/output units 400. The input/output unit 400 may include a button, a joystick, a click wheel, a scroll wheel, a touchpad, a keypad, a keyboard, a camera, a light-emitting diode and other status indicators, etc.

The user can control the operation of the electronic device 100 by inputting a command through the input/output circuit 420, and the output data from the input/output circuit 420 enables the receiving of status information and other outputs from the electronic device 100.

Further, an embodiment of the present invention further provides a non-transitory computer-readable storage medium. The non-transitory computer-readable storage medium can be configured in the server in each of the above embodiments, and a computer program is stored in the non-transitory computer-readable storage medium. When the program is executed by the processor, the radio-frequency operation prompting method described in the embodiments is implemented.

In the embodiments described above, emphasis has been placed on the description of various embodiments. Parts of an embodiment that are not described or illustrated in detail may be found in the description of other embodiments.

It can be recognized by those skilled in the art that the exemplary modules/units and algorithm steps described in connection with embodiments disclosed herein can be implemented by electronic hardware, or a combination of computer software and electronic hardware. Whether these functions are implemented by hardware or software depends on the specific constraints for application and design of the technical solution. For each specific application, different methods can be used by professional and technical personnel to implement the described functions. However, this implementation should not be considered as going beyond the scope of the present invention.

In the embodiments provided in the present invention, it can be understood that the disclosed device/terminal and method can be implemented in other ways. For example, the device/terminal embodiments described above are merely illustrative. For example, a division of the modules or elements is merely division of logical functions, and there may be additional divisions in actual implementation. For example, multiple units or components may be combined or integrated into another system, or some features may be omitted or not performed. Alternatively, the couplings or direct couplings or communicative connections shown or discussed with respect to one another may be indirect couplings or communicative connections via some interfaces, devices or units may be electrical, mechanical or otherwise.

The units described as separate components may or may not be physically separate, and the components shown as units may or may not be physical units, i.e. may be located in one place, or may be distributed over a plurality of network elements. Some or all of the units may be selected to achieve the objectives of the solution of the present embodiment according to practical requirements.

In addition, the functional units in the various embodiments of the present invention may be integrated into one processing unit, may be physically separate from each other or may be integrated in one unit by two or more units. The integrated units described above can be implemented either in the form of hardware, or software functional units.

The integrated unit, if implemented in the form of a software functional unit and sold or used as a stand-alone product, may be stored in a computer readable storage medium. Based on such an understanding, all or part of the processes of the methods in the above-described embodiments implemented in the present invention, may also be implemented by a computer program instructing related hardware. The computer program may be stored in a computer-readable storage medium, and performs the steps of the various method embodiments described above when executed by the processor. The computer program includes a computer program code, which may be in the form of source code, object code, or executable file, or in some intermediate form. The computer readable medium may include: any entity or device capable of carrying the computer program code, recording media, U disks, removable hard disks, magnetic disks, optical disks, computer memory, read-only memory (ROM), random access memory (RAM), electric carrier wave signals, telecommunication signals and software distribution media. It should be noted that the computer readable medium may contain content that may be appropriately augmented or subtracted as required by legislation and patent practice within judicial jurisdictions, e.g., the computer-readable medium does not include electrical carrier wave signals and telecommunications signals in accordance with legislation and patent practices in some jurisdictions.

Bronchoscopic Electrosurgical Ablation Method

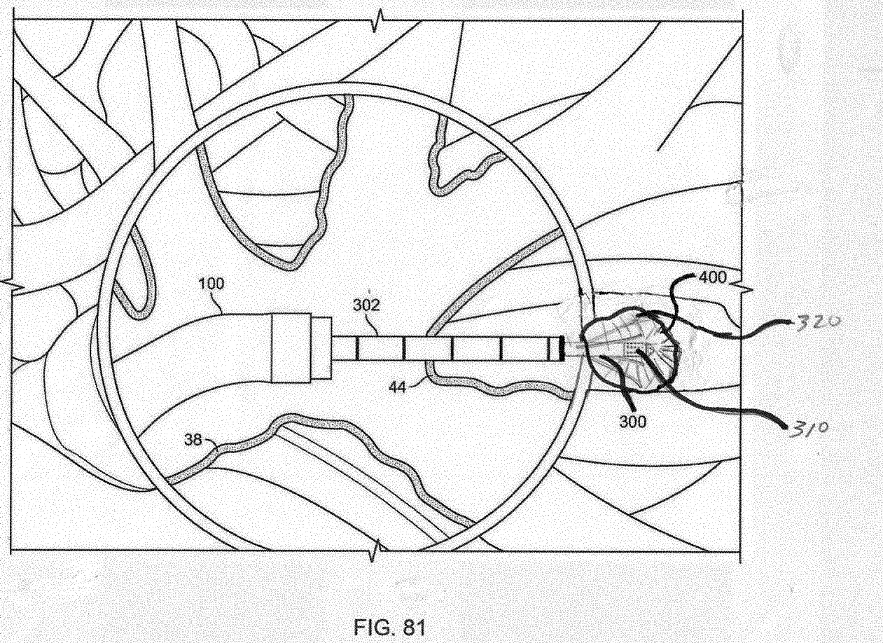

Embodiments of the invention include treatment of a lung tumor with radio frequency ablation. With reference to FIG. 81, a radio frequency probe 300 is shown advanced beyond the end of a sheath 302, and into the lung tumor 400. Although the distal tip 310 of the electrosurgical instrument 300 is shown penetrating the tumor 400, the invention is not so limited. Embodiments of the invention include, without limitation, making mere contact with the tumor, or partially inserting the tip, or contacting a pole or edge of the tumor.

Once the physician confirms the catheter tip 310 or RF electrode is properly positioned, the sensors 320 are extended from the shaft into the periphery of the tumor, away from the tip 310. Optionally, the sensors may be extended slightly beyond the tumor into the tumor margin or buffer. Position confirmation or tracking can be performed by a wide range of guidance and location techniques including, without limitation, fluoroscopy or use of electromagnetic sensors. Examples of tracking and guidance techniques are described in patent Nos. 6,380,732 and 9,265,468 and US Patent Publication no. 2016/0180529.

In embodiments, and with reference again to FIG. 81, the tip 310 and sensor members 320 are adapted to penetrate the lung tumor 400. The electrode acts as a heat source and initially heats the tissue where the electrodes make contact. The heat thermally conducts through the tumor from the heat source. When the tip is positioned within the tumor, the tumor is ablated from the heat source (or point) to the exterior (namely, “inside out”).

The sensors are spaced several millimeters (or centimeters depending on the tumor size) from the point source to advantageously detect properties of the boundary of the tumor and ablation zone. Without intending to being bound to theory, the sensor arrangement in the present invention more accurately monitors ablation than other techniques such as, for example, (a) detecting at the point source, and (b) detecting via external monitoring techniques because each of these other techniques are indirect and require extrapolation of the measured data to obtain the information at the actual tumor boundary.

As described above, in embodiments, the tip is also adapted to irrigate or perfuse the tumor. The sensors serve to measure temperature and or impedance during the ablation. In preferred embodiments, the power and perfusion flowrate is adjusted based on the monitoring. Optionally, the controller can be programmed to automatically adjust the power level for the electrode based on impedance, and once the sensors detect a threshold value, the power is halted, ending the procedure. The perfusion may also be adjusted to optimize impedance and ablation, as well as safely terminate the procedure by minimizing heat.

Although the radio frequency energy delivery catheter is shown as a single tip electrode with four claw sensor members, it may take a wide range of configurations and the electrode shape itself may also vary widely. Examples of electrode shapes include, without limitation, needle, hook, basket, loop, helix, coil, forceps or clamp, tubular, and snare or lasso. The electrode distal section may also be configured to flex, turn, and steer using mechanical or thermal action. Examples of RF ablation catheters, energy generators, and controllers are described in U.S. Pat. Nos. 6,692,494; 7,022,088 and US Patent Publication No. 2013/0046296. In each instance, except where doing so would thwart operation of the ablation electrode, a set or array of monitoring sensors may be extendable from the tip into the periphery of (or beyond) the tumor. An additional sensor or electrode may be placed on the ablation catheter shaft for additional sensing and/or directional ablation.

Tumor Outside the Airway—Create Access Channel

In the event the target is determined to be outside of the airway (e.g., in the parenchymal lung tissue), the method includes planning a bronchoscopic route to an exit or egress opening along the airway and in the vicinity of the target tissue, and to plan a route extension from the egress opening to the target tissue.

In embodiments, planning an exit or egress opening from the airway and to the target tissue outside of the airway is performed based on a number of constraints. Examples of constraints include, without limitation, proximity to the target tissue, avoiding obstacles such as blood vessels, and physical limitations of the instruments to be used during the procedure such as size, flexibility, and bend constraints. Exemplary techniques to plan the route are descried in U.S. Pat. Nos. 8,709,034 and 9,037,215.

Next, and with reference again to FIG. 81, the physician advances the bronchoscope 100 to a position along the airway 38 in the vicinity of the candidate exit opening.

Next, the physician creates an access pathway to the target tissue. Particularly, an opening is created through the airway wall 44, and tunnel to the tumor to create an access passage along the pre-planned route extension.

In an embodiment, the egress hole is created with a catheter fed through the bronchoscope having a sharp tip. An example of a needle catheter suitable to create the hole is described in U.S. Pat. No. 8,517,955.

In an embodiment, the hole is dilated. Openings may be enlarged using, for example, enlargeable members (e.g., balloon) or fixed tapered dilators.

An elongate tube 302 is advanced through the hole and to the tumor. The tube may be advanced over a wire or needle, or the tube may be navigated to the tumor. In embodiments, the tube includes a removable obturator to prohibit the tube from becoming filled with tissue as the tube is advanced through the tissue.

Once the tube 302 is in position, and the position is confirmed, the intermediate instruments such as an obturator, dilator, guidewire, or needles are retracted leaving an open channel through the tube. Position confirmation, or tracking, of the instruments for accessing the tumor can be performed by a wide range of guidance and location techniques including, without limitation, fluoroscopy or use of electromagnetic sensors. Examples of tracking and guidance techniques are described in U.S. Pat. Nos. 6,380,732 and 9,265,468 and US Patent Publication no. 2016/0180529.

Although a route extension has been described above, various techniques to create and install an access passageway to the tumor are described in the following patent publications including, without limitation, U.S. Pat. Nos. 8,784,400 and 8,709,034. See also, Anciano et al., Going Off Road The First Case Reports of the Use of the Transbronchial Access Tool With Electromagnetic Navigational Bronchoscopy, J Bronchol Intervent Pulmonol, Vol. 24, No. 3, July 2017.

Once the access passageway is installed (such as, e.g., the sheath 302 shown in FIG. 81), the procedure to treat the tumor may be performed as described above, except the ablation instruments are advanced to the tumor through the access passageway instead of along the airway.

The above-described embodiments are merely illustrative of, and not intended to limit the technical solutions of the present invention. Although the present invention has been described in detail with reference to the foregoing embodiments, it should be understood by those of skill in the art that modifications can be made to technical solutions described in the foregoing embodiments, or some of the technical features thereof can be equivalently substituted; and such modifications and substitutions do not cause the nature of the corresponding technical solution to depart from the spirit and scope of the embodiments of the present disclosure and are intended to be included within the scope of the present invention.

Although a number of embodiments have been disclosed above, it is to be understood that other modifications and variations can be made to the disclosed embodiments without departing from the subject invention.

1. An electrosurgical method for ablating a target lesion in the lung comprising:
identifying a lesion to ablate;
identifying an interior and a periphery of the lesion;
advancing an ablation electrode into the interior of the lesion;
commencing ablation from the ablation electrode; and
sensing information at the periphery of the lesion during ablation.

2. The method of claim 1, further comprising sensing information in the interior of the lesion.

3. The method of claim 1, further comprising guiding sensors into or beyond the periphery of the lesion.

4. The method of claim 1, wherein sensors are operable to sense temperature.

5. The method of claim 1, wherein sensors are operable to sense impedance.

6. The method of claim 1, wherein the ablation electrode applies RF energy.

7. The method of claim 1, wherein the ablation electrode is arranged with a neutral electrode in a monopolar configuration.

8. The method of claim 1, further comprising stopping ablation based on the sensed information.

9. The method of claim 1, further comprising adjusting ablation based on sensor information.

10. The method of claim 1, further comprising irrigating the ablation electrode.

11. The method of claim 10, further comprising adjusting the flowrate of irrigating based on the sensed information.

12. The method of claim 1, further comprising evaluating progress of the ablation of the lesion based on imaging.

13. The method of claim 12, wherein the imaging is based on the sensed information or an external imaging technique.

14. The method of claim 1, wherein the ablation electrode is inserted into the center of mass of the lesion.

15. The method of claim 1, wherein the lesion is a tumor.

16. The method of claim 10, wherein the irrigating comprises ejecting a liquid from a plurality of discrete ports arranged along the ablation electrode from the proximal end to the distal end.

17. The method of claim 15, wherein the discrete ports are configured to eject the liquid at an equal flowrate along the ablation electrode.

18. The method of claim 1, wherein the ablation electrode is arranged with a return electrode in a bipolar configuration.

19. A method for treating lung cancer comprising:
advancing an ablation catheter into a lung and to a tumor to be ablated;
positioning an active electrode of the ablation catheter inside or adjacent the tumor;
deploying a plurality of sensors in a peripheral region surrounding the tumor and spaced a distance from the active electrode;
irrigating the active electrode and tumor;
activating the active electrode while irrigating; and
adjusting the power delivered to the active electrode based on information detected from the sensors.

20. The method of claim 19, wherein the deploying comprises adjustably deploying the plurality of sensors according to the size of the tumor.

21. The method of claim 19, wherein the deploying comprises penetrating the lesion with a plurality of elongate supports, and wherein at least one sensor is arranged on each elongate support.

22. The method of claim 21, further comprising adjusting the irrigating based on information from the sensors.
